(19) United States
(12) Patent Application Publication
(10) Pub. No.: US 2026/0015627 A1
(43) Pub. Date: Jan. 15, 2026

(54) COMPOSITIONS AND METHODS FOR IMPROVED PROTEIN TRANSLATION FROM RECOMBINANT CIRCULAR RNAS

(71) Applicant: The Board of Trustees of the Leland Stanford Junior University, Stanford, CA (US)

(72) Inventors: Robert Chen, Stanford, CA (US); Sean Wang, Stanford, CA (US); Laura Amaya, Stanford, CA (US); Howard Y. Chang, Stanford, CA (US)

(21) Appl. No.: 18/847,095

(22) PCT Filed: Mar. 17, 2023
(86) PCT No.: PCT/US23/64606

§ 371 Date: Sep. 13, 2024

Related U.S. Application Data
(60) Provisional application No. 63/320,952, filed on Mar. 17, 2022.
(60) Provisional application No. 63/353,110, filed on Jun. 17, 2022.
(60) Provisional application No. 63/423,641, filed on Nov. 8, 2022.
(60) Provisional application No. 63/449,409, filed on Mar. 2, 2023.
(60) Provisional application No. 63/450,272, filed on Mar. 6, 2023.

(51) Int. Cl. C12N 15/85; A61K 39/00; A61P 37/04; C12N 15/115

(52) U.S. Cl. CPC C12N 15/85; A61K 39/0011; A61P 37/04; C12N 15/115; A61K 2039/53; C12N 2310/16; C12N 2840/203

(57) ABSTRACT

The present disclosure relates to compositions and methods for improving protein translation from recombination circular RNAs. In particular, provided herein are formulations for delivery of recombinant circular RNA (circRNA) molecules and circRNAs comprising viral and/or synthetic internal ribosome entry sites (IRESs), as well as methods for use thereof.

### STATEMENT OF RELATED APPLICATIONS

This application claims priority to U.S. Provisional Patent Application No. 63/320,952, filed Mar. 17, 2022, U.S. Provisional Patent Application No. 63/353,110, filed Jun. 17, 2022, U.S. Provisional Patent Application No. 63/450,272, filed Mar. 6, 2023, U.S. Provisional Patent Application No. 63/423,641, filed Nov. 8, 2022, and U.S. Provisional Patent Application No. 63/449,409, filed Mar. 2, 2023, the entire contents of which are incorporated herein by reference for all purposes.

### STATEMENT REGARDING FEDERALLY SPONSORED RESEARCH

This invention was made with Government support under contract CA209919 and contract number 5T32GM008412 awarded by the National Institutes of Health. The Government has certain rights in the invention.

### FIELD

The present disclosure relates to compositions and methods for improving protein translation from recombination circular RNAs. In particular, provided herein are formulations for delivery of recombinant circular RNA (circRNA) molecules and circRNAs comprising viral and/or synthetic internal ribosome entry sites (IRESs), as well as methods for use thereof.

### SEQUENCE LISTING

The text of the computer readable sequence listing filed herewith, titled “STDU2-40616-601_SQL”, created Mar. 16, 2023, having a file size of 33,377,290 bytes, is hereby incorporated by reference in its entirety.

### BACKGROUND

Circular RNAs (circRNAs) are a type of single-stranded RNA which, unlike linear RNA, comprises a covalently closed continuous loop. circRNAs occur naturally in mammalian cells, and play important roles in various biological processes. circRNAs innately possess greater stability and resistance to intra- and extracellular RNAses than mRNAs, making them attractive candidates for delivery of key payloads where long-lasting expression is necessary.

Recently, there has been an interest in using recombinant circRNAs to express a protein of interest, in vitro or in vivo. Introduction of an internal ribosome entry sequence (IRES) into a circular RNA allows translation of a protein encoded by a circRNA. However, IRES elements that exist in nature may or may not support translation from engineered circular RNAs, as IRES elements are often evolved in the context of linear RNA genomes.

Accordingly, there is in the need in the art to identify IRES elements that can drive protein translation from recombinant circRNAs. Further, there is a need for engineered IRES elements that improve the amount and/or duration of protein expression from a circRNA, as well as methods of delivering circRNAs to cells in vitro and in vivo.

### BRIEF SUMMARY

The present disclosure relates to compositions and methods for improving protein translation from recombination circular RNAs. In particular, provided herein are formulations for delivery of recombinant circular RNA (circRNA) molecules and circRNAs comprising viral and/or synthetic internal ribosome entry sites (IRESs), as well as methods for use thereof. Provided herein is a composition, comprising: a complex of a charge-altering releasable transporter (CART) and a circular RNA molecule comprising a protein-coding sequence. In some embodiments, the protein-coding sequence is operably linked to an internal ribosome entry site (IRES) sequence. In some embodiments, the IRES sequence is a viral sequence; and wherein the protein-coding sequence encodes a non-viral protein.

In some embodiments, provided herein is a method of inducing immunity in a subject, comprising administering a composition, comprising: a complex of a charge-altering releasable transporter (CART) and a circular RNA molecule to the subject such that the composition induces immunity in the subject. In some embodiments, the circular RNA molecule comprises a protein-coding sequence (e.g., an antigen or a therapeutic protein). In some embodiments, the composition further comprises an antigen. In some embodiments, the immunity is innate immunity or an antigen-specific T cell response.

Also provided is a method of immunizing a subject, comprising administering a composition, comprising: a) a complex of a charge-altering releasable transporter (CART) and a circular RNA molecule; and b) an antigen to the subject such that the subject is immunized against the antigen.

Further provided is a method of treating cancer in a subject, comprising administering a composition, comprising: a) a complex of a charge-altering releasable transporter (CART) and a circular RNA molecule; and b) an antigen. In some embodiments, the antigen is a tumor antigen. In some embodiments, the administering induces antigen-specific T-cell-based cellular immunity in the subject.

In some embodiments, the administering treats or prevents a disease or disorder (e.g., cancer) in the subject. In some embodiments, the delivering is intraperitoneally, intranasally, or intravenously. In some embodiments, the subject is a human or a non-human animal.

In some embodiments, the circular RNA is synthesized from a vector comprising self-splicing introns, 5′ PABP spacer, HBA1 3′ UTR, and an internal ribosome entry site (IRES) sequence.

In some embodiments, the CART is 1:1 mixture of oleyl (0) and nonenyl-substituted (N) carbonate monomers followed by a block of α-amino ester monomers (A). For example, a block length of 6 nonenyl and 6 oleyl carbonate units and 9 cationic amino-ester units. In some embodiments, the circular RNA and the CART are complexed at a 1:10 charge ratio. In some embodiments, the CART directs said circular RNA to immune cells. In some embodiments, the composition is a pharmaceutical composition.

Also provided is a composition, comprising: a circular RNA molecule comprising an internal ribosome entry site (IRES) sequence operably linked to a protein-coding sequence, a 5′ UTR, and a 3′ UTR; wherein the IRES sequence is a viral sequence; and wherein the protein-coding sequence encodes a non-viral protein.

In some embodiments, the 5′ UTR is ACUCACUAUUUGUUUUCGCGCCCAGUUGCAAAAAGUGUCG (SEQ ID NO: 33143) or AGCGUAACCUCCAUCCGAGUUGCAAGAGAGGGAAACGCAGUCUC and the 3′ UTR is a β-globin 3′ UTR or a truncated β-globin 3′ UTR.

Further provided is a composition, comprising: a circular RNA molecule comprising an internal ribosome entry site (IRES) sequence operably linked to a protein-coding sequence, wherein said IRES sequence comprises a locked nucleic acid (LNA) against a non-base-paired linker region between domains of said IRES; wherein the TRES sequence is a viral sequence; and wherein the protein-coding sequence encodes a non-viral protein. In some embodiments, the IRES is iCVB3 and said LNA is against a non-base-paired linker region between iCVB3 domains I and II.

Additionally provided is a composition, comprising: a circular RNA molecule comprising an iHRV-B3 internal ribosome entry site (IRES) sequence a 5′ UTR, and a 3′ UTR; operably linked to a protein-coding sequence, wherein said circular RNA comprises 5% m⁶A. In some embodiments, the non-viral protein is a mammalian protein. In some embodiments, the non-viral protein is a human protein.

In some embodiments, the IRES is a Type 1 IRES. In some embodiments, the IRES is an enterovirus IRES. In some embodiments, the IRES is a human rhinovirus (HRV) IRES. In some embodiments, the IRES is any one of the IRES listed in Table 7. In some embodiments, the IES is any one of the following IRES: iEMCV, iHCV, iCVB5, iSwineVesicular, iHRV-A2, iHRV-C3, iHRV-C11, iCVB1, iPV2, iHRV-B17, iEchoV-E15, iEV71, iHRV-A9, iSiminanV4, iEV-D94, iSimianA5, iPV3, iHRV-C54, iHRV-A100, iHiRV-B37, iHRV-B4, iHRV-B92, iHRV-B3, iHRV-A1, iEV107, or a fragment or derivative thereof. In some embodiments, the IRES is any one of the following IRES: iEV-B83, iHRV-A57, iHRV-B35, iHRV-B4, iEV-D68, iHRVB_R93, iHRV-B5, iHRVB-B52, iHRVB-B93, iHRV-B84, iHRV-B83 SC2220, iHRV-B72, iHRV-B69, iHRVB_SC0739, iHRV-B91, iHRV-B42, iHRV-B6, iHRV-B83, iHRV-B48, iHRV-B99, iHRV-B79, iHRV-B97, iHRV-B27, iHRVB_3039, iHRVB-B14, iCosV-B1, or a fragment or derivative thereof. In some embodiments, the IRES is iCVB3, or a fragment or derivative thereof. In some embodiments, the IRES is iHRV-B3, or a fragment or derivative thereof.

In some embodiments, the IRIS is a synthetic IRES sequence comprising an aptamer and a second aptamer.

In some embodiments, the aptamer is a wildtype aptamer. In some embodiments, the aptamer is an aptamer was designed and/or evolved to bind one or more DNA sequences. In some embodiments, the aptamer is a mutant aptamer. In some embodiments, the aptamer is modified to have an extended stem region.

In some embodiments, the aptamer is positioned within the secondary structure of the IRES so that is spatially proximal to portion of the IRES responsible for translation initiation. In some embodiments, the aptamer does not interrupt the native eIF4G binding site of the IRES and does not interrupt a native GRNA tetraloop within the IRES.

In some embodiments, the aptamer is an eIF4G-binding aptamer. In some embodiments, the eIF4G-binding aptamer comprises or is encoded by the sequence of SEQ ID NO: 33143. In some embodiments, the IRES is a Type 1 IRES. In some embodiments, the IRES is a modified enterovirus IRES. In some embodiments, the IRES is a modified human rhinovirus (HRV) IRES. In some embodiments, the IRES comprises or is encoded by the sequence of any one of SEQ ID NO: 33169-33173.

In some embodiments, synthetic IRES sequence is a modified iCVB3 IRES. In some embodiments, modified iCVB3 IRES comprises an aptamer inserted in domain I, II, III, IV, V, VI or VII thereof. In some embodiments, the modified iCVB3 IRES comprises an aptamer inserted in domain IV thereof. In some embodiments, the modified iCVB3 aptamer is modified to have an extended stem region. In some embodiments, the modified iCVB3 aptamer is positioned within the secondary structure of the IRES so that is spatially proximal to portion of the IRES responsible for translation initiation. In some embodiments, the modified iCVB3 aptamer does not interrupt the native eIF4G binding site of the IRES and does not interrupt a native GRNA tetraloop within the IRES.

In some embodiments, the synthetic IRES sequence is a modified iHRV-B3 IRES. In some embodiments, the modified iHRV-B3 IRES comprises an aptamer inserted in domain I, II, III, IV, V, or VI thereof. In some embodiments, the modified iHRV-B3 IRES comprises an aptamer inserted in domain IV thereof. In some embodiments, the modified iHRV-B3 IRES aptamer is modified to have an extended stem region. In some embodiments, the modified iHRV-B3 IRES aptamer is positioned within the secondary structure of the IRES so that is spatially proximal to portion of the IRES responsible for translation initiation. In some embodiments, the modified iHRV-B3 aptamer does not interrupt the native eIF4G binding site of the IRES and does not interrupt a native GRNA tetraloop within the IRES.

In some embodiments, the circular RNA comprises a least one 2-thiouridine (2ThioU) or at least one 2′-O-methylcitidine (2OMeC).

Also provided is a nucleic acid that encodes one or more of the circular RNA molecules described herein.

Also provided is a composition comprising one or more of the circular RNA molecules and/or the nucleic acids described herein.

Also provided are host cells comprising one or more of the circular RNA molecules and/or the nucleic acids described herein.

Also provided are methods for producing a protein in a cell, the method comprising contacting a cell with a circular RNA molecule or a nucleic acid described herein under conditions whereby the protein-coding nucleic acid sequence of the circular RNA is translated and the protein is produced in the cell.

Also provided are methods for producing a protein in vitro, the method comprising contacting a cell-free extract with a circular RNA molecule or a nucleic acid under conditions whereby the protein-coding nucleic acid sequence of the circular RNA is translated and the protein is produced.

Further provided is a method of delivering a protein to a subject, comprising: administering a composition described herein to a subject. In some embodiments, the protein is a therapeutic protein. In some embodiments, the administering treats or prevents a disease or disorder in the subject. In some embodiments, the delivering is intraperitoneally or intravenously. In some embodiments, the subject is a human or a non-human animal.

These and other embodiments will be described in further detail below, and in the appended drawings.

### BRIEF DESCRIPTION OF THE DRAWINGS

FIG. 1. A modular cloning platform for circRNA enables rapid design-build-test cycles. Schematic describing the modular cloning (MoClo) platform used to create template plasmids for circRNA synthesis. Parts 1-6 corresponding to the upstream intron and 5′ untranslated region (UTR), IRES, N-terminal (N′) tag, coding sequence (CDS), C-terminal (C′) tag, and 3′ UTR and downstream intron were individually cloned into parts plasmids via Golden Gate reactions (see FIG. 7). Parts plasmids and the circRNA backbone were then combined in a second Golden Gate reaction to create a circRNA plasmid. The circRNA backbone contains a CAG promoter enabling circRNA transcription after transient transfection in cellulo, a T7 promoter enabling in vitro transcription (IVT), homology sequences that assist with RNA circularization, low-structure regions that facilitate RNaseR processivity, and a bacterially expressed GFP dropout sequence to negatively select for incorrect assemblies. If a CDS without N′ or C′ tags was used, parts 3-5 were replaced with a single part. PCR products from circRNA plasmids were subsequently used as templates for in vitro transcription to synthesize RNA. Lastly, RNaseR cleanup was performed to digest linear RNAs and isolate circRNA.

FIG. 2. Optimization of RNA modifications and noncoding elements enable stronger circRNA translation. (A)NanoLuc activity after transfection of HeLa cells with circRNAs containing different RNA modifications. Data shown are mean SEM for n=3 biological replicates. ***P<0.001 by unpaired t-test compared to unmodified circRNA. (B) NanoLuc activity after transfection of HeLa cells with circRNAs containing either a 3′ or 5′ IRES and spacer sequences of varying lengths. When the IRES is 3′ to the NanoLuc reporter, translation through the td splicing scar is unavoidable. The predicted secondary structure of this scar is shown. Data shown are mean±SEM for n=3 biological replicates. (C) NanoLuc activity after transfection of HeLa cells with circRNAs containing different 5′ spacer sequences. Data shown are mean±SEM for n=3 biological replicates. *P=0.0213, **P=0.0051, ***P<0.001 by unpaired t-test compared to a random 50 bp spacer sequence. (D) NanoLuc activity after transfection of HeLa cells with circRNAs containing different 3′ UTR sequences. NanoLuc activity was normalized to constitutive firefly luciferase activity from the same sample, then divided by values from mock transfection. Data shown are mean±SEM for n=3 biological replicates. ***P=0.0012, ****P<0.001 by unpaired two-sided t-test compared to a random 50 nt spacer sequence. PR, protected region; MR, minimal region; BR, binding region.

FIG. 3. IRES truncations and the secondary structure of the IRES-coding sequence junction affect circRNA translation. (A) NanoLuc activity at 24 hours after transfection of HeLa cells with circRNAs containing deletions of different IRES domains starting from the 5′ end. Secondary structure and truncation points are indicated on the diagram. Data shown are mean±SEM for n=3 biological replicates. *P<0.05 by unpaired t-test compared to full-length (FL) iCVB3. (B) NanoLuc activity at 24 hours after transfection of HeLa cells with circRNAs containing deletions of individual IRES domains. NanoLuc activity was normalized to constitutive firefly luciferase activity from the same sample, then divided by values from mock transfection. Data shown are mean±SEM for n=3 biological replicates (C) NanoLuc activity at 24 hours after transfection of HeLa cells with circRNAs containing successive 10 bp deletions starting from the 3′ end of the IRES, immediately prior to the AUG start codon. (D) Correlations between the indicated properties and NanoLuc activity at 24 hours after transfection of HeLa cells with circRNAs containing different N-terminal leader sequences between the AUG start codon and NanoLuc reporter. Data shown are mean t SEM for n=3 biological replicates.

FIG. 4. A synthetic IRES containing an eIF4G-recruiting aptamer drives stronger circRNA translation. (A) NanoLuc activity at 24 hours after co-transfection of HeLa cells with circRNA and escalating doses (4.2-33.3 nM) of locked nucleic acids (LNAs) #1-3 or a non-targeting (NT) LNA. LNAs #1-3 were designed to be complementary to regions of iCVB3 as indicated in the schematic. NanoLuc activity was normalized to constitutive firefly luciferase activity from the same sample, then divided by values from mock transfection. Data shown are mean±SEM for n=3 biological replicates. *P=0.0233, **P<0.01, ***P=0.0001 by unpaired two-sided t-test compared to an equal dose of NT LNA. (B) NanoLuc activity at 24 hours after transfection of HeLa cells with circRNAs containing an eIF4G-recruiting aptamer (Apt-eIF4G), shown in inset. Apt-eIF4G was inserted into iCVB3 at 11 different positions as indicated in the schematic. Data shown are mean±SEM for n=3 biological replicates. **P=0.0017, ***P=0.0002 by unpaired t-test compared to wild-type iCVB3. (C) mNeonGreen fluorescence at 24 hours after electroporation of HeLa cells with mRNA or circRNAs containing successive optimizations. mRNA was synthesized with CleanCap reagent, 100% NIP incorporation, and a 100 bp poly(A) tail. Data shown are histograms for n>50,000 live singlet cells per condition and meant SEM for n=3 biological replicates. **P=0.0044, ***P=0.0006 by unpaired two-sided t-test. (D) The gating strategy is shown to analyze live singlet HEK293T cells after electroporation.

FIG. 5. Large-scale screens and IRES engineering expand the repertoire of strong IRESs (A)NanoLuc activity at 24 hours after transfection of HeLa, HepG2, and HEK293T cells with circRNAs containing the indicated IRESs. Data shown are mean±SEM for n=3 biological replicates. (B) NanoLuc activity after in vitro transcription-translation (IVTT) of circRNA plasmids containing shuffled IRESs. DNA shuffling was performed on human rhinovirus IRESs by fragmenting IRESs and cloning the resulting pool into circRNA plasmids. Purified plasmids were then subjected to IVTT using HeLa lysate. NanoLuc activity was divided by values from mock IVTT. Data shown are mean SEM for n=4 biological replicates. P<0.05, **P=0.0095, ****P<0.0001 by unpaired two-sided t-test compared to wild-type iHRV-B3. (C) NanoLuc activity at 24 hours after transfection of HeLa cells with circRNAs containing different insertions of Apt-eIF4G into an IRES of indeterminate structure (iHRV-B3). The putative secondary structure for iHRV-B3, predicted eIF4G and eIF4A binding sites, and locations of Apt-eIF4G insertions are shown. Versions (v1-v6) of each insertion were designed with different stem lengths. Double aptamer refers to insertion of Apt-eIF4G at both the distal and proximal loops. NanoLuc activity was normalized to constitutive firefly luciferase activity from the same sample, then divided by values from mock transfection. Data shown are mean±SEM for n=3 biological replicates. *P=0.0422, **P=0.0018, ***P=0.0003, ****P<0.0001 by unpaired two-sided t-test compared to wild-type iHRV-B3. (D) NanoLuc activity at 24 hours after transfection of HeLa cells with mRNA or circRNAs containing successive optimizations. mRNA was synthesized with CleanCap reagent, 100% N1′P incorporation, and a 120 nt poly(A) tail. NanoLuc activity was normalized to constitutive firefly luciferase activity from the same sample, then divided by values from mock transfection. Data shown are mean±SEM for n=4 biological replicates. **P=0.0051, ***P=0.0001, ****P<0.0001 by unpaired two-sided t-test. (E) AkaLuc activity at 24 hours after electroporation of HeLa cells with circRNAs encoding AkaLuc-P2A-CyOFP. CircRNA iCVB3-AkaLuc-P2A-CyOFP was synthesized with 5% m6A, upstream IRES topology, and random UTR spacers. AkaLuc activity was divided by values from mock electroporation. Sizes indicate lengths of the coding sequence for NanoLuc and AkaLuc-P2A-CyOFP. ****P<0.0001 by unpaired two-sided t-test.

FIG. 6. In vivo gene replacement validates engineered circRNAs with extended duration and intensity of translation. (A) CircRNA encoding NanoLuc was synthesized with the following optimizations: 5% m6A incorporation, upstream IRES topology, 5′ PABP spacer, HBA1 3′ UTR, and HRV-B3 IRES with proximal loop Apt-eIF4G insertion. CircRNAs were formulated for intraperitoneal delivery in mice using charge-altering releasable transporters (CARTs). Expression was assayed using an Ami HT optical imaging system following intraperitoneal injections of the fluorofurimazine substrate at the indicated time points. At 336 hours (14 days) after the first administration of circRNA NanoLuc, mice were redosed. (B) In vivo luminescence image of an untreated mouse (left) versus mice receiving circRNA NanoLuc (right) at 24 hours after dosing. (C) Quantification of luminescence per mouse at different time points after circNanoLuc administration. Redosing was performed at 336 hours (14 days). Data shown are mean±SEM for n=3 animals per condition. (D) An optimized circRNA encoding human erythropoietin (hEPO) was synthesized with the following optimizations: 5% m6A incorporation, upstream IRES topology, 5′ PABP spacer, HBA1 3′ UTR, and HRV-B3 IRES with proximal loop Apt-eIF4G insertion. a 120 nt poly(A) tail. Equimolar doses of circRNA and mRNA were formulated for intravenous delivery in mice using CARTs. Plasma hEPO was measured by ELISA in one cohort at the indicated time points. Reticulocytes were counted in a separate cohort at 168 hours (7 days). (E) Quantification of plasma hEPO expression at different time points after circRNA EPO or mRNA EPO administration. Data shown are mean±SEM for n=4 animals per condition. (F) Plasma hEPO expression normalized to the 24-hour level of each mouse at different time points after circRNA EPO or mRNA EPO administration. Data shown are mean±SEM for n=4 animals per condition. *P=0.0487, ***P=0.0001 by unpaired t-test with Bonferroni correction compared to mRNA. (G) Reticulocyte percentage among red blood cells at 168 hours after circRNA hEPO or mRNA hEPO administration. Data shown are mean±SEM for n=4 animals per condition. **P=0.0080 by unpaired two-sided t-test. ns, not significant. (H) Shows the gating strategy to analyze reticulocytes from peripheral mouse blood.

FIG. 7. Additional details for circRNA MoClo platform and NanoLuc assay, related to FIG. 1. (A) Part plasmids containing parts 1-6 flanked by the indicated overhangs were synthesized by combining a PCR product or premade DNA fragment with the parts entry vector in a BsmBI Golden Gate reaction. These parts corresponded to the upstream intron and 5′ untranslated region (UTR), IRES, N-terminal (N′) tag, coding sequence (CDS), C-terminal (C′) tag, and 3′ UTR and downstream intron. Parts plasmids and the circRNA backbone were subsequently combined in a BsaI Golden Gate reaction to create a circRNA plasmid. Fully assembled circRNA plasmids included the indicated restriction enzyme cut sites at key junctions to facilitate subcloning and in-frame glycine-serine linkers between parts 3, 4, and 5. Linkers were omitted if parts 3-5 were replaced with a single part. (B) Standard curve quantitation for the NanoLuc assay used to assess circRNA translation, demonstrating linearity across a 2²¹ (2 million)-fold dilution range. Data shown are mean for n=3 technical replicates.

FIG. 8. RNA modifications extend circRNA stability in vitro, related to FIG. 2. (A) NanoLuc activity at 24 hours after transfection of HeLa cells with circRNAs containing 10% incorporation of different RNA modifications. (B) Quantification of circRNA levels in HeLa cells at 24 hours after transfection with circRNAs containing the indicated RNA modifications. Data shown are mean±SEM for n=3 biological replicates. (C) Resistance of mRNA and circRNAs with indicated RNA modifications to degradation in escalating doses of fetal bovine serum (FBS). RNAs were incubated in the indicated percentages of FBS at 37° C. for 30 minutes, then briefly denatured in RNA loading buffer before gel electrophoresis. The same amount of ladder per gel and RNA per well were used to allow for comparisons between gels. (D) NanoLuc activity in supernatant after electroporation of HeLa cells with circRNA or mRNA encoding secreted NanoLuc. CircRNA was synthesized with 5% m6A incorporation and the HRV-B3 IRES. mRNA was synthesized with CleanCap reagent, 100% N1Ψ incorporation, and a 120 nt poly(A) tail. At the indicated hours (h) and days (d) post-electroporation, media was harvested to assay secreted NanoLuc and replaced. Data shown are mean±SEM for n=3 biological replicates. (E) NanoLuc activity at 24 hours after transfection of HeLa cells with circRNAs containing the indicated number of stop codons. Data shown are mean±SEM for n=3 biological replicates. (F) mNeonGreen fluorescence at 24 hours after electroporation of HeLa cells with unmodified circRNA or circRNA containing 5% m6A. Mean mNeonGreen expression was measured by flow cytometry and normalized by values from mock electroporation. Data shown are histograms for n>50,000 live singlet cells per condition and mean±SEM for n=3 biological replicates.

FIG. 9. eIF4G-binding site deletions are translation-lethal and irrecoverable, related to FIG. 4. NanoLuc activity at 24 hours after transfection of HeLa cells with circRNAs containing wild-type iCVB3, iCVB3 with Apt-eIF4G insertion, iCVB3 with eIF4G footprint deletions, or iCVB3 with eIF4G footprint deletions and attempted rescue with Apt-eIF4G. Sub-domain deletions (v1-v4) differed in the position where the stem loop was truncated, but at a minimum all ablated the eIF4G footprint.

FIG. 10. Additional large-scale screening and validation of IRESs, related to FIG. 5. (A) NanoLuc activity after in vitro transcription-translation (IVTT) of circRNA plasmids containing enterovirus (EV) or human rhinovirus B (HRV-B) IRESs. All known EV and HRV-B IRES sequences were cloned into circRNA plasmids. Purified plasmids were then subjected to IVTT using HeLa lysate (B) NanoLuc activity at 24 hours after transfection of HeLa cells with circRNAs or linear RNAs containing strong IRESs from the IVTT-based screen. Linear RNA sequences were identical to those of circRNAs with the exclusion of self-splicing introns. Data shown are mean±SEM for n=3 biological replicates. (C) NanoLuc activity at 24 hours after transfection of HeLa, HepG2, HEK293T, and KG-I cells with circRNAs containing the indicated IRESs. Values for HeLa, HepG2, and HEK293T cells are the same as in FIG. 5a.

FIG. 11. (A) TapeStation gel electrophoresis depicting the size of circRNAs encoding NanoLuc and possessing the indicated number of stop codons. (B) Western blot depicting NanoLuc protein in HeLa lysate at 24 hours after electroporation with circRNAs encoding NanoLuc and possessing the indicated number of stop codons. Each lane was loaded with 10 μg of total protein.

FIG. 12. In silico RNA structure prediction can inform IRES engineering. RNA structure predictions for synthetic IRESs synIRES01-11 at the site of aptamer insertion. For inter-domain insertions (synIRES01, 03, 05, 09, and 11), structure prediction was performed on Apt-eIF4G and the adjacent iCVB3 domains. For loop insertions (synIRES02, 04, 06, 07, 08, and 10), structure prediction was performed on Apt-eIF4G and the iCVB3 domain containing the insertion. In each structure, nucleotides corresponding to Apt-eIF4G are shown in white.

FIG. 13. iHRV-B3 enhances the activity of circRNA encoding Cre. Percentage of eGFP-positive cells at 24 hours after transfection of CreR-293T cells with mRNA or circRNAs encoding Cre recombinase. In this cell line, DsRed is expressed before Cre recombination and eGFP is expressed after. eGFP and DsRed expression were measured by flow cytometry. Data shown are mean±SEM for n=2 for biological replicates for mock and n=3 biological replicates for all other conditions. *P=0.0147 by unpaired two-sided t-test.

FIG. 14. CircRNA optimizations do not adversely affect circRNA synthesis. (A) Effect of successive circRNA optimizations on total RNA production when starting from 1 μg of in vitro transcription template. (B) Effect of successive circRNA optimizations on circRNA percentage, defined as the percentage of RNA remaining after digestion with RNaseR.

FIG. 15. Circular RNA uptake is specific to myeloid cells. a, Schematic representation of experimental model for circRNA uptake measurements. b, CircRNA and linRNA uptake measured by flow cytometry in distinct hematopoietic cell subsets from human peripheral blood after 2 hours incubation. c, Fluorescent intensity of human myeloid immune subsets after distinct incubation time points with fluorescently labeled circRNA (n=3, bars represent SEM). d, Confocal microscopy of labeled circRNA (increasing concentrations) and miRNA (last image) in human macrophages after 2 hours incubation. e, Percentage of fluorescently labeled cells after circRNA uptake in mouse (top) and human (bottom) cell lines (n=5, bars represent SEM).

FIG. 16. Circular RNA uptake is a fast and active process. a, Time course analysis of circRNA uptake measured by flow cytometry in RAW264 cells (n=5, bars represent SEM). b, Saturation curve of circRNA uptake in RAW264 cells after 24 hours incubation. Logarithmic and linear scale displayed for accurate visualization (n=3, bars represent SEM). c, Competitor assay showing fold change of circRNA uptake after normalization to cy5-circRNA control in RAW264 cells (n=3, bars represent SEM). d, Fluorescent intensity of RAW264 cells after incubation or lipofectamine transfection with fluorescently labeled circRNA at increasing concentrations comparing temperature effect (representative sample on the left and summary quantification on the right) (n=4, bars represent SEM, One sample t and Wilcoxon test **P<0.001, ****P<0.0001). e, Increasing concentration of sodium azide effect on circRNA uptake in RAW264 cells (representative sample on the left and summary quantification on the right) (n=3, bars represent SEM). One-way ANOVA followed by Tukey's test was applied in c and e *P<0.05, **P<0.01, ***P<0.001, ****P<0.0001. Differences between groups were considered significant for P values<0.05. NS, not significant FIG. 17. Circular RNA is translated and presented to the immune system. a, Nanoluciferase intensity measured in RAW264 cells after transfection (left) and uptake (right) of circNanoLuc at different time points. (n=3, bars represent SEM). b, Fluorescent intensity of SIINFEKL bound to H-2 Kb of MHC class I antibody compared to control after 24 incubation with cirOVA, Ova protein, or SIINFEKL control in MutuDC cells (n=4, bars represent Min and Max). c, Proliferation assay comparing antigen specific T cell proliferation level of OT-I cells co-cultured with MutuDC cells incubated with Ova protein, SIINFEKL, and circOVA or non translatable circRNA control (representative sample on the right and summary quantification on the left) (n=4, bars represent SEM). d, circOVA titration with and without CART to determine the minimum amount required to induce antigen-specific T cell proliferation. e, qRT-PCR quantification of immune receptors and cytokines. f, flow cytometry quantification of secreted inflammatory cytokines and g, flow cytometry quantification of activation markers after circOVA uptake or transfection compared to CpG in MutuDC cells (n=4, bars represent Min and Max). One-way ANOVA followed by Tukey's test was applied in b and g *P<0.05, **P<0.01, ***P<0.001, ****P<0.0001. Differences between groups were considered significant for P values<0.05. NS, not significant.

FIG. 18. Circular RNA in vivo delivery tropism and innate recognition. a, Schematic representation of in vivo circRNA delivery and monitoring. 50 ug of AF488-circRNA was delivered subcutaneously, serum samples were collected 6 and 24 hours after delivery. b, Absolute fraction of fluorescently positive innate cell subsets that take up circRNA (n=5, bars represent Min and Max). c, Quantification of innate cell subsets proportions and d, fluorescent intensity of activation marker CD86 in distinct innate immune cell subsets in lymph nodes, 24 hours after s.c. delivery of fluorescently labeled circRNA (n=5, bars represent Min and Max). e, Time course analysis of cytokines in serum after circRNA delivery measured by Luminex (n=5). One-way ANOVA followed by Tukey's test was applied in b-e *P<0.05, **P<0.01, ***P<0.001, ****P<0.0001.

FIG. 19. Adjuvant effect of circRNA by different routes of delivery. a, Schematic representation of circRNA immunization strategy via different delivery routes and monitoring of immune responses. Percentage of Ova-specific T cell responses in lung, spleen, and lymph nodes after b, 7 days or c, 779 30 days post-boost (n=5, bars represent Min and Max). d, Frequency of CD69+ and CD69+CD103+CD8 TRM in lungs at Day 30 post-boost (as percentage of antigen-specific CD8 T cells) (n=5, bars represent Min and Max). One-way ANOVA followed by Tukey's test was applied in b-e, n=5, *P<0.05, **P<0.01, ***P<0.001, ****P<0.0001. Differences between groups were considered significant for P values<0.05. NS, not significant.

FIG. 20. Circular RNA delivery in vivo activates T cell specific responses. a, Schematic representation of immunization strategy and monitoring of adaptive immune responses. b, Percentage of Ova-specific T cell responses in lung and spleen at day 42 (representative sample). c, Quantification of Ova-specific T cells in lung and spleen at day 7 and day 42 (n=5, bars represent Min and Max). d, Time course analysis of anti-Ova IgG antibodies in serum measured by ELISA (n=5, bars represent Min and Max). One-way ANOVA followed by Tukey's test was applied in c and d *P<0.05, **P<0.01, ***P<0.001, ****P<0.0001.

FIG. 21. circRNA uptake validation in different cell lines. a, Gating strategy used to define myeloid human cell population after uptake of circRNA. b, Histogram representation of fluorescent intensity shift after circRNA uptake in human immune cell subsets corresponding to FIG. 1c (representative sample). c, Flow cytometry quantification of increasing concentration of circRNA and linRNA in human PBMC-derived macrophages. d, CircRNA is taken up by human classical monocytes in a dose and time dependent manner (n=4, bars represent SEM). e, circRNA is taken up by J774 and RAW cells in a dose dependent manner (n=4, bars represent SEM). f, Histogram representation of fluorescence intensity shift after circRNA uptake in human liver and lung cell lines (representative sample).

FIG. 22. circRNA and linRNA uptake. a, circRNA uptake is inhibited at cold temperatures in human liver and lung cell lines (n=3, bars represent SEM). b, ATP inhibitors negatively inhibit circRNA uptake in RAW264 cells (n=4, bars represent SEM). c, Maturation signals promote circRNA uptake in RAW264 cells (n=3, bars represent SEM). d, qRT-PCR quantification of circRNA compared to mRNA after uptake or transfection in RAW264 cells (n=3, bars represent SEM). e, Representative sample of circRNA uptake compared to mRNA in distinct cell types measured by flow cytometry. f, Representative sample of circRNA uptake measurement comparison between cy5-circRNA or pHrodo-circRNA at distinct concentrations in RAW264 cells.

FIG. 23. circRNA and linRNA translation. a, Nanoluciferase intensity measured in HepG2 cells shows similar translation efficiency after uptake of mRNA (left) or circRNA (right) encoding Nanoluciferase protein. b, Detection of Ovalbumin protein after transfection of 293T with circOVA by western blot and ELISA. c, Percentage of fluorescently labeled cells after circRNA uptake in RAW264 cells (n=4, bars represent SEM).

FIG. 24. Transcriptome analysis after in vitro 825 delivery of circRNA. a, PCA analysis of normalized and transformed t 826 ranscriptome counts grouped by condition: Untreated, circRNA and CART-cirRNA (n=2). b, Volcano plot with the log 2 fold changes in gene expression between circRNA uptake and circRNA transfection with CART. c, Heatmap of normalized expression data showing differentially regulated genes following circRNA uptake or circRNA transfection with CART compared to untreated cells. Functional analysis of the top differentially expressed genes and their linkages with biological concepts (GO terms) after d, circRNA uptake and e, circRNA transfection with CART.

FIG. 25. Transcriptome analysis after in vitro 825 delivery of circRNA. a, PCA analysis of normalized and transformed t 826 ranscriptome counts grouped by condition: Untreated, circRNA and CART-cirRNA (n=2). b, Volcano plot with the log 2 fold changes in gene expression between circRNA uptake and circRNA transfection with CART. c, Heatmap of normalized expression data showing differentially regulated genes following circRNA uptake or circRNA transfection with CART compared to untreated cells. Functional analysis of the top differentially expressed genes and their linkages with biological concepts (GO terms) after d, circRNA uptake and e, circRNA transfection with CART.

FIG. 26. Innate response to circRNA immunization 842 with and without CART. a, Frequencies of innate cell subsets in the draining inguinal LNs 24 h after s.c. delivery of circRNA, circRNA delivered in CART (CART-circOVA), and CART alone. b, Frequencies of circRNA+ cells as % of given cell subsets (indicating c 845 ircRNA uptake). c, CD86 expression on innate cell subsets in the four immunization groups.

FIG. 27 Adjuvant effect of circRNA 848 by different routes of delivery. a, Gating strategy used to distinguish adaptive immune subsets after delivery of circRNA. b, Anti-Ova IgG and c, IgA antibodies in serum measured by ELISA at day 30 post-boost after circRNA immunization by different delivery routes (n=5, bars represent Min and Max). d, Percentage of Ova-specific TRM cells (gated as CD69+) in lung (as % of CD45+ live cells). e, Frequency of Class I tetramer+CD8 T cells. f, anti-853 Ova IgG (reciprocal EC50 titers shown), and g, IgA antibodies (endpoint titer shown) in serum measured by ELISA at day 30 post-boost of i.n. delivery of circRNA compared to Poly(IC) (n=5, bars represent Min and Max). One-way ANOVA followed by Tukey's test was applied in b-e, n=5. No values are displayed as no significant differences were found.

FIG. 28. Adaptive immune responses after immunization with circRNA. a, Gating strategy used through immunization experiments to measure antigen specific T cell responses. b, Percentage of Ova-specific T cell in lung after i.n. immunization with naked circOVA (n=5, bars represent SEM). c, Percentage of Ova-specific T cells in peripheral blood at day 7 post-prime (n=4, bars represent SEM). d, Proportion of SLEC and MPEC subsets at day 7 post-prime and day 21 post-boost (n=5, bars represent Min and Max).

FIG. 29. a, Schematic representation of immunization strategy and monitoring of tumor volume after inoculation with B16-F10-OVA cells. b, Tumor volume monitoring over 22 days (n=5, bars represent SEM).

FIG. 30. Adaptive immune responses after immunization with circOVA. a, Representative bioluminescence images and b, quantification of tumors from control mice and mice vaccinated with circOVA. (n=4, bars represent SD). Unpaired t-test was applied *p<0.05.

### DETAILED DESCRIPTION

Protein translation in eukaryotic cells typically relies on the m⁷G cap present at the 5′ end of mRNAs. However, several cap-independent translation mechanisms have been identified. For example, some viral mRNAs employ alternative mechanisms of translation initiation based on internal ribosome entry via an internal ribosome entry sequence (IRES). Cap-independent translation of proteins typically suffers from lower translation strength, as compared to cap-dependent (mRNA translation).

Provided herein are viral and synthetic IRES that can drive expression of a protein (e.g., a non-viral protein) from a circular RNA, along with optimization of circRNAs comprising such IRESs. The viral and synthetic IRES described herein satisfy an unmet need in the field of cap-independent translation. The IRESs identified may also be used for polycistronic mRNA gene delivery. Because the IRESs described herein drive expression at a wide range of strengths and some in a cell type-dependent manner, the choice of IRES can be used to independently control expression levels of the two or more proteins in a single transcript. This expression level tunability offers an additional layer of control over just dosing leveling.

Further provided herein are methods of delivery of circRNAs to cells (e.g., in vitro or in vivo) and research, screening, and clinical uses thereof.

### Definitions

To facilitate an understanding of the present technology, a number of terms and phrases are defined below. Additional definitions are set forth throughout the detailed description.

The use of the terms “a” and “an” and “the” and “at least one” and similar referents in the context of describing the invention (especially in the context of the following claims) are to be construed to cover both the singular and the plural, unless otherwise indicated herein or clearly contradicted by context.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Nomenclature for nucleotides, nucleic acids, nucleosides, and amino acids used herein is consistent with International Union of Pure and Applied Chemistry (IUPAC) standards (see, e.g., bioinformatics.org/sms/iupac.html).

When referring to a nucleic acid sequence or protein sequence, the term “identity” is used to denote similarity between two sequences. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12, 387-395 (1984), or by inspection. Another algorithm is the BLAST algorithm, described in Altschul et al., J Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity. Further, an additional useful algorithm is gapped BLAST as reported by Altschul et al, (1997) Nucleic Acids Res. 25, 3389-3402. Unless otherwise indicated, percent identity is determined herein using the algorithm available at the internet address: blast.ncbi.nlm.nih.gov/Blast.cgi.

The terms “internal ribosome entry site,” “internal ribosome entry sequence,” “IRES” and “IRES sequence region” are used interchangeably herein and refer to cis elements of viral or human cellular RNAs (e.g., messenger RNA (mRNA) and/or circRNAs) that bypass the steps of canonical eukaryotic cap-dependent translation initiation. The canonical cap-dependent mechanism used by the vast majority of eukaryotic mRNAs requires an m⁷G cap at the 5′ end of the mRNA, initiator Met-tRNAmet, more than a dozen initiation factor proteins, directional scanning, and GTP hydrolysis to place a translationally competent ribosome at the start codon. IRESs typically are comprised of a long and highly structured 5′-UTR which mediates the translation initiation complex binding and catalyzes the formation of a functional ribosome. “Aptamers” are short, single-stranded DNA or RNA molecules that can selectively bind to a specific target. The target may be, for example, a protein, peptide, carbohydrate, small molecule, toxin, or a live cell. Some aptamers can bind DNA, RNA, self-aptamers or other non-self aptamers. Aptamers assume a variety of shapes due to their tendency to form helices and single-stranded loops. Illustrative DNA and RNA aptamers are listed in the Aptamer database (scicrunch.org/resources/Any/record/nlx_144509-1/SCR_001781/resolver?q=*&l=). The terms “coding sequence,” “coding sequence region,” “coding region,” and “CDS” when referring to nucleic acid sequences may be used to refer to the portion of a DNA or RNA sequence, for example, that is or may be translated to protein. The terms “reading frame,” “open reading frame,” and “ORF,” may be used herein to refer to a nucleotide sequence that begins with an initiation codon (e.g., ATG) and, in some embodiments, ends with a termination codon (e.g., TAA, TAG, or TGA). Open reading frames may contain introns and exons, and as such, all CDSs are ORFs, but not all ORF are CDSs.

The terms “complementary” and “complementarity” refers to the relationship between two nucleic acid sequences or nucleic acid monomers having the capacity to form hydrogen bond(s) with one another by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., about 50%, about 60%, about 70%, about 80%, about 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., at least 65%, at least 70%, at least 75%, at least 80%, at least 85%, at least 90%, at least 95%, at least 97%, at least 98%, at least 99%, or 100%) over a region of at least 8 nucleotides (e.g., at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, or more nucleotides), or if the two nucleic acid sequences hybridize under at least moderate, or, in some embodiments high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook, J., Molecular Cloning: A Laboratory Manual, Cold Spring Harbor Laboratory Press; 4th edition (Jun. 15, 2012). High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 MNaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (optionally in combination with EDTA). Additional details and an explanation of stringency of hybridization reactions are provided in, e.g., Sambrook, supra; and Ausubel et al., eds., Short Protocols in Molecular Biology, 5th ed., John Wiley & Sons, Inc., Hoboken, N.J. (2002). The term “hybridization” or “hybridized” when referring to nucleic acid sequences is the association formed between and/or among sequences having complementarity.

The term “secondary structure,” or “secondary structure element” or “secondary structure sequence region” as used herein in reference to nucleic acid sequences (e.g., RNA, DNA, etc), refers to any non-linear conformation of nucleotide or ribonucleotide units. Such non-linear conformations may include base-pairing interactions within a single nucleic acid polymer or between two polymers. Single-stranded RNA typically forms complex and intricate base-pairing interactions due to its increased ability to form hydrogen bonds stemming from the extra hydroxyl group in the ribose sugar. Examples of secondary structures or secondary structure elements include but are not limited to, for example, stem-loops, hairpin structures, bulges, internal loops, multiloops, coils, random coils, helices, partial helices and pseudoknots. In some embodiments, the term “secondary structure” may refer to a SuRE element. The term “SuRE” stands for stem-loop structured RNA element (SuRE).

The term “free energy,” as used herein, refers to the energy released by folding an unfolded polynucleotide (e.g., RNA or DNA, etc.) molecule, or, conversely, the amount of energy that must be added in order to unfold a folded polynucleotide (e.g., RNA or DNA, etc.) The “minimum free energy (MFE)” of a polynucleotide (e.g., DNA, RNA, etc.) describes the lowest value of free energy observed for the polynucleotide when assessed for various secondary structures thereof. The MFE of an RNA molecule may be used to predict RNA or DNA secondary structure and is affected by the number, composition, and arrangement of the RNA or RNA nucleotides. The more negative free energy a structure has, the more likely is its formation since more stored energy is released by formation of the structure.

The term “melting temperature (Tm)” refers to the temperature at which about 50% of double-stranded nucleic acid structures (e.g., DNA/DNA, DNA/RNA, or RNA/RNA duplexes) denature and dissociate to single-stranded structures.

The term “recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may act to modulate production of a desired product by various mechanisms. Alternatively, DNA sequences encoding RNA that is not translated may also be considered recombinant. Thus, the term “recombinant” nucleic acid also refers to a nucleic acid which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, the artificial combination may be performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may comprise a naturally occurring amino acid sequence.

The terms “operably linked” and “operatively linked,” as used herein, refer to an arrangement of elements that are configured so as to perform, function or be structured in such a manner as to be suitable for an intended purpose. For example, a given promoter operably linked to a coding sequence is capable of effecting the expression of the coding sequence when the proper enzymes are present. Expression is meant to include the transcription of any one or more of a recombinant nucleic acid encoding a circular RNA, or mRNA from a DNA or RNA template and can further include translation of a protein from a recombinant circular RNA comprising an IRES sequence (e.g., a non-native IRES). Thus, for example, intervening untranslated yet transcribed sequences can be present between a promoter sequence and a coding sequence and the promoter sequence can still be considered to be “operably linked” to the coding sequence.

### Circular RNAs

The instant disclosure provides recombinant circular RNA molecules comprising an internal ribosome entry site (IRES) sequence operably linked to a protein-coding sequence, and DNA sequences encoding the same. In some embodiments, the protein coding sequence encodes a non-viral protein. For example, in some embodiments, the protein coding sequence encodes an animal protein, a plant protein, a bacterial protein, a fungal protein, or an artificial protein. In some embodiments, the protein coding sequence encodes a mammalian protein, such as a human protein.

Recombinant circRNA molecules may be generated or engineered according to several methods. For example, recombinant circRNA molecules may be generated by back-splicing of linear RNAs. For example, in some embodiments, a recombinant circular RNA is produced by back-splicing of a downstream 5′ splice site (splice donor) to an upstream 3′ splice site (splice acceptor). The splice donor and/or splice acceptor may be found, for example, in a human intron or portion thereof that is typically used for circRNA production at endogenous loci. In some embodiments, a recombinant circular RNA is produced by contacting a cell with a DNA plasmid, wherein the DNA plasmid encodes a linear RNA, and the linear RNA is back-spliced to produce a recombinant circular RNA. In some embodiments, the DNA plasmid comprises introns from the mammalian ZKSCAN1 gene.

In some embodiments, circular RNAs can be generated by a non-mammalian splicing method. For example, linear RNAs containing various types of introns, including self-splicing group I introns, self-splicing group II introns, spliceosomal introns, and tRNA introns can be circularized. In particular, group I and group II introns have the advantage that they can be readily used for production of circular RNAs in vitro as well as in vivo because of their ability to undergo self-splicing due to their autocatalytic ribozyme activity.

Alternatively, circular RNAs can be produced in vitro from a linear RNA by chemical or enzymatic ligation of the 5′ and 3′ ends of the RNA. Chemical ligation can be performed, for example, using cyanogen bromide (BrCN) or ethyl-3-(3′-dimethylaminopropyl) carbodiimide (EDC) for activation of a nucleotide phosphomonoester group to allow phosphodiester bond formation (Sokolova, FEBS Lett, 232:153-155 (1988); Dolinnaya et al., Nucleic Acids Res., 19: 3067-3072 (1991); Fedorova, Nucleosides Nucleotides Nucleic Acids, 15: 1137-1147 (1996)). Alternatively, enzymatic ligation can be used to circularize RNA. Exemplary ligases that can be used include T4 DNA ligase (T4 Dnl), T4 RNA ligase 1 (T4 Rnl 1), and T4 RNA ligase 2 (T4 Rnl 2).

In some embodiments, splint ligation may be used to generate circular RNA. Splint ligation involves the use of an oligonucleotide splint that hybridizes with the two ends of a linear RNA to bring the ends of the linear RNA together for ligation. Hybridization of the splint, which can be either a deoxyribo-oligonucleotide or a ribooligonucleotide, orients the 5′-phosphate and 3′-OH of the RNA ends for ligation. Subsequent ligation can be performed using either chemical or enzymatic techniques, as described above. Enzymatic ligation can be performed, for example, with T4 DNA ligase (DNA splint required), T4 RNA ligase 1 (RNA splint required) or T4 RNA ligase 2 (DNA or RNA splint). Chemical ligation, such as with BrCN or EDC, is more efficient in some cases than enzymatic ligation if the structure of the hybridized splint-RNA complex interferes with enzymatic activity (see, e.g., Dolinnaya et al. Nucleic Acids Res, 21(23): 5403-5407 (1993); Petkovic et al., Nucleic Acids Res, 43(4): 2454-2465 (2015)).

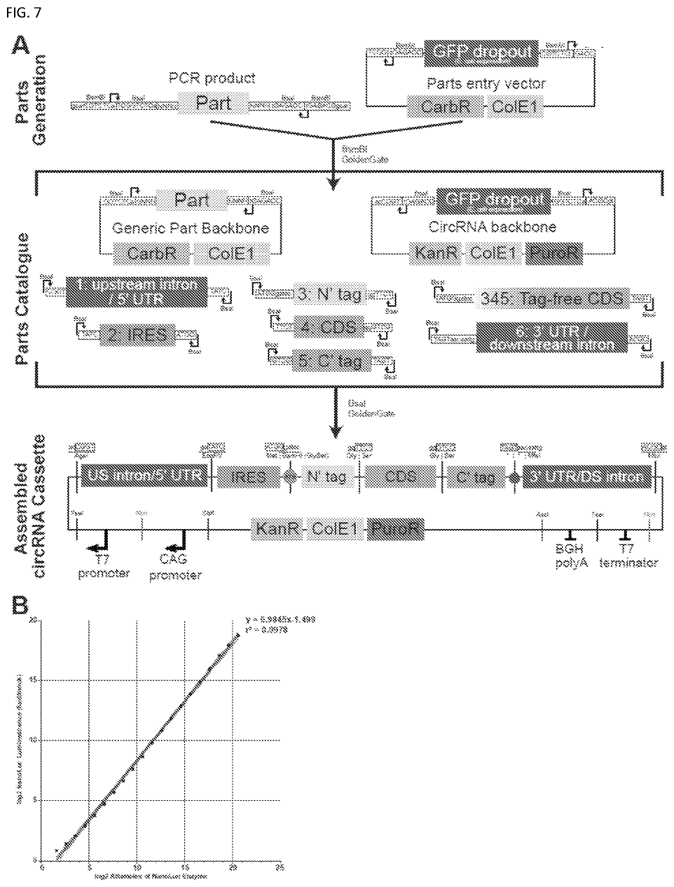

In some embodiments, the modular cloning platform shown in FIG. 7 is used to generate plasmids for use in generating cirRNAs. This platform allows for the rapid synthesis of large libraries. The platform can be used for random library generation, as demonstrated with IRES shuffling. Independent libraries can also be modularly assembled to produce rich RNA element data sets, such as combining shuffled 5′ UTR and shuffled 3′ UTR regions to flank a reporter gene. strengths. This approach can vastly expand the repertoire of usable IRESs and may enable delivery of circRNAs with finely tuned translational activities that parallel physiological expression. Translation from the a given IRES can differ by 100-fold depending on whether the RNA was circular or linear. This is consistent with a recent screen for sequences driving cap-independent translation in circular and linear RNAs and indicates that there are mechanisms of translational control unique to circRNAs (Chen, C. K. et al. Structured elements drive extensive circular RNA translation. Mol. Cell 81, 4300-4318.e13 (2021), the contents of which are herein incorporated by reference in their entirety).

In some embodiments, circRNAs comprise optimized 3′ and/or 5′ UTRs. In some embodiments, the 5′ UTR comprises or is ACUCACUAUUUGUUUUCGCGCCCAGUUGCAAAAAGUGUCG and the 3′ UTR comprises or is a β-globin 3′ UTR or a truncated β-globin 3′ UTR or a sequence at least 90% (e.g., at least 95%, 96%, 97%, 98%, or 99%) identical thereof). In some embodiments, the β-globin 3′ UTR is truncated after the AAUAAA polyadenylation signal. In some embodiments, 10, 20, 30, 40, 5-, 60, 70, 80, 90, or 100 nucleotides are truncated 3′ of the polyadenylation site.

While circular RNAs generally are more stable than their linear counterparts, primarily due to the absence of free ends necessary for exonuclease-mediated degradation, additional modifications may be made to the recombinant circRNA described herein to further improve stability. Still other kinds of modifications may improve circularization efficiency, purification of circRNA, and/or protein expression from circRNA. For example, the recombinant circRNA may be engineered to include “homology arms” (i.e., 9-19 nucleotides in length placed at the 5′ and 3′ ends of a precursor RNA with the aim of bringing the 5′ and 3′ splice sites into proximity of one another), spacer sequences, and/or a phosphorothioate (PS) cap (Wesselhoeft et al., Nat. Commun., 9: 2629 (2018)). The recombinant circRNA also may be engineered to include 2′-O-methyl-, -fluoro- or —O-methoxyethyl conjugates, phosphorothioate backbones, or 2′,4′-cyclic 2′-O-ethyl modifications to increase the stability thereof (Holdt et al., Front Physiol., 9: 1262 (2018); Krützfeldt et al., Nature, 438(7068): 685-9 (2005); and Crooke et al., Cell Metab., 27(4): 714-739 (2018)). The recombinant circRNA molecule also may comprise one or more modifications that reduce the innate immunogenicity of the circRNA molecule in a host, such as at least one N6-methyladenosine (m⁶A).

In some embodiments, the recombinant circRNA molecule comprises at least one 2-thiouridine (2ThioU) or at least one 2′-O-methylcytidine (2OMeC). 2-thiouridine is a modified nucleobase found in tRNAs that has been shown to stabilize U:A base pairs and destabilize U:G wobble pairs (Rodriguez-Hernandez et al., J. Mol. Biol. 2013; 425:3888-3906). Methylation of 2′-hydroxyl groups is one of the most common posttranscriptional modifications of naturally occurring stable RNA molecules (Satoh et al., RNA 2000. 6: 680-686). For example, methylation of tRNA at the 2′-OH position of the ribose sugar is generally thought to increase the stability of tRNA via mechanisms that protect against spontaneous hydrolysis or nuclease digestion (e.g., in non-helical regions) and reinforce intra-loop interactions that stabilize the tertiary structure of the molecule (Endres et al., PLoS ONE 15 (2): e0229103). Any number of nucleotides (e.g., uridine and/or cytidine) in a particular circRNA molecule generated as described herein may be modified (e.g., replaced) with a corresponding number of 2-thiouridine (2ThioU) or 2′-O-methylcytidine (2OMeC). Ideally, at least one nucleotide in the circRNA molecule is replaced with a 2ThioU or a 2OMeC. In some embodiments, at least 1% (e.g., 1%, 2%, 3%, 4%, 5%, 6%, 7%, 8%, 9% or more) of the nucleotides in the recombinant circular RNA molecule are replaced with 2ThioU or a 2OMeC. In other embodiments, at least 10% (e.g., 10%, 11%, 12%, 13%, 14%, 15%, 20%, 30%, 40%, 50%, 60%, 70%, 80%, 90%, or more) of the nucleotides in the recombinant circular RNA molecule are replaced with 2ThioU or 2OMeC. For example, the recombinant circRNA molecule comprises about 2% to about 5% (e.g., 2.5%, 3%, 3.5%, 4%, or 4.5%) 2-thiouridine or 2-O-methylcytidine. In some embodiments, the recombinant circRNA molecule comprises about 2.5% 2ThioU or 2OMeC. In other embodiments, all (i.e., 100%) of the uridine nucleotides in the recombinant circular RNA molecule may be replaced with 2ThioU, or all (i.e., 100%) of the cytidine nucleotides in the recombinant circRNA molecule may be replaced with 2OMeC. It will be appreciated that the number of 2ThioU or 2OMeC modifications introduced into a recombinant circular RNA molecule will depend upon the particular use of the circRNA.

In some embodiments, a DNA sequence encoding a circular RNA molecule comprises sequences that encode at least two introns and at least one exon. The term “exon,” as used herein, refers to a nucleic acid sequence present in a gene which is represented in the mature form of an RNA molecule after excision of introns during transcription. Exons may be translated into protein (e.g., in the case of messenger RNA (mRNA)). The term “intron,” as used herein, refers to a nucleic acid sequence present in a given gene which is removed by RNA splicing during maturation of the final RNA product. Introns are generally found between exons. During transcription, introns are removed from precursor messenger RNA (pre-mRNA), and exons are joined via RNA splicing. In some embodiments, the recombinant circular RNA molecule comprises a nucleic acid sequence which includes one or more exons and one or more introns. Accordingly, circular RNAs can be generated using either an endogenous or exogenous intron, as described in WO 2017/222911. As used herein, the term “endogenous intron” means an intron sequence that is native to the host cell in which the circRNA is produced. For example, a human intron is an endogenous intron when the circRNA is expressed in a human cell. An “exogenous intron” means an intron that is heterologous to the host cell in which the circRNA is generated. For example, a bacterial intron would be an exogenous intron when the circRNA is expressed in a human cell. Numerous intron sequences from a wide variety of organisms and viruses are known and include sequences derived from genes encoding proteins, ribosomal RNA (rRNA), or transfer RNA (tRNA). Representative intron sequences are available in various databases, including the Group I Intron Sequence and Structure Database (rna.whu.edu.cn/gissd/), the Database for Bacterial Group II Introns (webapps2.ucalgary.ca/-groupii/index.html), the Database for Mobile Group II Introns (fp.ucalgary.ca/group2introns), the Yeast Intron DataBase (emblS16 heidelberg.de/Externallnfo/seraphin/yidb.html), the Ares Lab Yeast Intron Database (compbio.soe.ucsc.edu/yeast_introns.html), the Ul2 Intron Database (genome.crg.es/cgibin/u12db/u12db.cgi), and the Exon-Intron Database (bpg.utoledo.edu/-afedorov/lab/eid.html).

In some embodiments, a nucleic acid (e.g., a DNA) encoding a circular RNA molecule comprises a self-splicing group I intron. Group I introns are a distinct class of RNA self-splicing introns which catalyze their own excision from mRNA, tRNA, and rRNA precursors in a wide range of organisms. All known group I introns present in eukaryote nuclei interrupt functional ribosomal RNA genes located in ribosomal DNA loci. Nuclear group I introns appear widespread among eukaryotic microorganisms, and the plasmodial slime molds (myxomycetes) contain an abundance of self-splicing introns. The self-splicing group I intron included in the DNA encoding the circular RNA molecule may be obtained or derived from any organism, such as, for example, bacteria, bacteriophages, and eukaryotic viruses. Self-splicing group I introns also may be found in certain cellular organelles, such as mitochondria and chloroplasts, and such self-splicing introns may be incorporated into the nucleic acid encoding a circular RNA molecule.

In some embodiments, a nucleic acid encoding a recombinant circular RNA molecule comprises a self-splicing group I intron of the phage T4 thymidylate synthase (td) gene. The group I intron of phage T4 thymidylate synthase (td) gene is well characterized to circularize while the exons linearly splice together (Chandry and Belfort, Genes Dev., 1: 1028-1037 (1987); Ford and Ares, Proc. Natl. Acad Sci. USA, 91: 3117-3121 (1994); and Perriman and Ares, RNA, 4: 1047-1054 (1998)). When the td intron order is permuted (i.e., 5′ half placed at the 3′ position and vice versa) flanking any exon sequence, the exon is circularized via two autocatalytic transesterification reactions (Ford and Ares, supra; Puttaraju and Been, Nucleic Acids Symp. Ser., 33: 49-51 (1995)).

In some embodiments, a nucleic acid (e.g., a DNA) encoding the recombinant circular RNA molecule comprises a ZKSCAN1 intron. The ZKSCAN1 intron is described in, for example, Yao, Z., et al., Mol. Oncol. (2017) 11(4):422-437. In some embodiments, a nucleic acid encoding the recombinant circular RNA molecule comprises a miniZKSCAN1 intron. The recombinant circular RNA molecule may be of any length or size. For example, the recombinant circular RNA molecule may comprise between about 200 nucleotides and about 10,000 nucleotides (e.g., about 300, about 400, about 500, about 600, about 700, about 800, about 900, about 1,000, about 2,000, about 3,000, about 4,000, about 5,000, about 6,000, about 7,000, about 8,000, or about 9,000 nucleotides, or a range defined by any two of the foregoing values). In some embodiments, the recombinant circular RNA molecule comprises between about 500 and about 6,000 nucleotides (about 550, about 650, about 750, about 850, about 950, about 1,100, about 1,200, about 1,300, about 1,400, about 1,500, about 1,600, about 1,700, about 1,800, about 1,900, about 2,100, about 2,200, about 2,300, about 2,400, about 2,500, about 2,600, about 2,700, about 2,800, about 2,900, about 3,100, about 3,300, about 3,500, about 3,700, about 3,800, about 3,900, about 4,100, about 4,300, about 4,500, about 4,700, about 4,900, about 5,100, about 5,300, about 5,500, about 5,700, or about 5,900 nucleotides, or a range defined by any two of the foregoing values). In one embodiment, the recombinant circular RNA molecule comprises about 1,500 nucleotides.

In some embodiments, a recombinant circular RNA molecule comprises an internal ribosome entry site (TRES) sequence operably linked to a protein-coding sequence; wherein the IRES sequence is a viral sequence; and wherein the protein-coding sequence encodes a non-viral protein.

In some embodiments, a recombinant circular RNA molecule comprises a protein-coding nucleic acid sequence region and an internal ribosome entry site (IRES) sequence region operably linked to the protein-coding nucleic acid sequence region, wherein the IRES comprises: at least one sequence region having secondary structure element; and a sequence region that is complementary to an 18S ribosomal RNA (rRNA); wherein the IRES has a minimum free energy (MFE) of less than −18.9 kJ/mol and a melting temperature of at least 35.0° C. In some embodiments, the IRES sequence is linked to the protein-coding nucleic acid sequence region in a non-native configuration.

The disclosure also provides a recombinant circular RNA molecule comprising a protein-coding nucleic acid sequence region and an internal ribosome entry site (TRES) sequence region operably linked to the protein-coding nucleic acid sequence; wherein the IRES is encoded by any one of the nucleic acid sequences listed in Table 1A or Table 1B, or a nucleic acid sequence that has at least 90% or at least 95% identity or homology thereto. In some embodiments, the IRES sequence is linked to the protein-coding nucleic acid sequence region in a non-native configuration.

### Internal Ribosome Entry Sequences

The recombinant circular RNAs described herein comprise an internal ribosome entry site (IRES). These IRES sequences may be operably linked to a protein-coding sequence of the circRNA. Inclusion of an IRES permits the translation of one or more open reading frames from a circular RNA. The IRES attracts a eukaryotic ribosomal translation initiation complex and promotes translation initiation.

Provided herein are various IRES sequences which, when present in a circRNA, drive translation of a protein encoded by the circRNA. In some embodiments, the IRES of a circRNA may be operably linked to a protein-coding nucleic acid sequence. In some embodiments, the IRES of a circRNA is operably linked to a protein-coding nucleic acid sequence in a non-native configuration. In some embodiments, the IRES is a human IRES. In some embodiments, the IRES is a viral IRES. In some embodiments, the IRES is a type 1 IRES.

As used herein, the term “non-native configuration” refers to a linkage between an IRES and a protein-coding nucleic acid that does not occur in a naturally occurring circRNA molecule. For example, a viral IRES may be operably linked to a protein-coding nucleic acid sequence in a circular RNA, or an IRES that is not found in naturally occurring circRNA molecules may be operably linked to a protein-coding nucleic acid sequence in a circRNA. In some embodiments, an IRES that is found in naturally occurring circRNA molecules operably linked to a certain protein-coding nucleic acid is operably linked to a different protein-coding nucleic acid (i.e., a nucleic acid to which the IRES is not operably linked in any naturally-occurring circRNA). In some embodiments, an IRES that is found in naturally occurring linear mRNAs is operably linked to a protein coding sequence in a circular RNA.

A number of linear IRES sequences are known and may be included in a recombinant circular RNA molecule as described herein. For example, linear IRES sequences may be derived from a wide variety of viruses, such as from leader sequences of picornaviruses (e.g., encephalomyocarditis virus (EMCV) UTR) (Jang et al., J. Virol., 63: 1651-1660 (1989)), the polio leader sequence, the hepatitis A virus leader, the hepatitis C virus IRES, human rhinovirus type 2 IRES (Dobrikova et al., Proc. Natl. Acad. Sci., 100(25): 15125-15130 (2003)), an IRES element from the foot and mouth disease virus (Ramesh et al., Nucl. Acid Res., 24: 2697-2700 (1996)), and a giardiavirus IRES (Garlapati et al., J. Biol. Chem., 279(5): 3389-3397 (2004)). A variety of nonviral IRES sequences also can be included in a circular RNA molecule, including but not limited to, IRES sequences from yeast, the human angiotensin II type 1 receptor IRES (Martin et al., Mol. Cell Endocrinol., 212: 51-61 (2003)), fibroblast growth factor IRESs (e.g., FGF-1 IRES and FGF-2 IRES, Martineau et al., Mol. Cell. Biol., 24(17): 7622-7635 (2004)), vascular endothelial growth factor IRES (Baranick et al., Proc. Natl. Acad Sci. U.S.A., 105(12): 4733-4738 (2008); Stein et al., Mol. Cell. Biol., 18(6): 3112-3119 (1998); Bert et al., RATA, 12(6): 1074-1083(2006)), and insulin-like growth factor 2 IRES (Pedersen et al., Biochem. J., 363(Pt 1): 37-44 (2002)).

IRES sequences and vectors encoding IRES elements are commercially available from a variety of sources, such as, for example, Clontech (Mountain View, CA), Invivogen (San Diego, CA), Addgene (Cambridge, MA) and GeneCopoeia (Rockville, MD), and IRESite: The database of experimentally verified IRES structures (iresite.org). Notably, these databases focus on activity of IES sequences in mRNA (i.e., linear RNAs), and do not focus on circRNA IRES activity profiles.

### Viral IRES Sequences

In some embodiments, the circRNAs described herein comprise viral IRES sequence. The viral IRES sequence may be operably linked to a protein-coding sequence in a non-native configuration. For example, the viral IRES sequence may be operably linked to a sequence that encodes a non-viral protein. In some embodiments, the protein coding sequence encodes an animal protein, a plant protein, a bacterial protein, a fungal protein, or an artificial protein. In some embodiments, the protein coding sequence encodes a mammalian protein, such as a human protein. In some embodiments, the viral IRES sequence, when placed into a circular RNA, drives potent translation of a protein encoded by the circular RNA.

Table 7 below provides a non-limiting list of viral IRES that may be used in a circRNA to drive expression of a protein encoded by the circular RNA. Also provided in Table 7 are GenBank Accession Nos. for the genomic sequences from which the viral IRES were identified, Sequences encoding the viral IRES are provided in the SEQUENCE APPENDIX.

TABLE 7
Illustrative viral IRES sequences

| Viral IRES | GenBank Accession No. |
|---|---|
| iPV1 | NC_002058 |
| iPV2 | D00625 |
| iPV3 | AJ293918 |
| iHRV1 | NC_038311 |
| iHRV2 | X02316 |
| iHRV-B3 | NC_038312 |
| iCVA1 | AF499635 |
| iCVA16 | KC117318 |
| iCVB2 | AF081485 |
| iCVB3 | NC_038307 |
| iEV-A71 | KT354875 |
| iEV-A120 | LK021688 |
| iEV71 | HM003207 |
| iEV-B107 | AB426609 |
| iHRV-A9 | FJ445177 |
| iHRV-A21 | FJ445121 |
| iHRV-A57 | JN614995 |
| iHRV-A89 | NC_001617 |
| iHRV-A100 | FJ445175 |
| iHRV-B4 | DQ473490 |
| iHRV-B17 | EF173420 |
| iHRV-B26 | FJ445124 |
| iHRV-B37 | EF173423 |
| iHRV-B92 | FJ445169 |
| iHRV-C3 | EF186077 |
| iHRV-C11 | EU840952 |
| iHRV-C54 | KP282614 |
| iCVB1 | M16560 |
| iCVB5 | AF114383 |
| iEchoV1 | AF029859 |
| iEchoV-E11 | X80059 |
| iEchoV-E15 | AY302541 |
| iCVA20 | AF499642 |
| iEV-D94 | DQ916376 |
| iSimianEV-A | NC_003988 |
| iSimianA5 | AF326751 |
| iSimianV4 | NC_038309 |
| iSwineVesciular | MK038999 |
| iHRV-B3_SC2220 | KY684757 |
| iHRV-B5 | FJ445112 |
| iHRV-B6 | DQ473486 |
| iHRV-B14 | MN306034 |
| iHRV-B27 | FJ445186 |
| iHRV-B35 | DQ473487 |
| iHRV-B42 | FJ445130 |
| iHRV-B48 | DQ473488 |
| iHRV-B52 | FJ445188 |
| iHRV-B69 | FJ445151 |
| iHRV-B70 | DQ473489 |
| iHRV-B72 | FJ445153 |
| iHRV-B79 | FJ445155 |
| iHRV-B83 | MN306025 |
| iHRV-B84 | FJ445162 |
| iHRV-B86 | FJ445164 |
| iHRV-B91 | FJ445168 |
| iHRV-B93 | EF173425 |
| iHRV-B97 | FJ445172 |
| iHRV-B99 | FJ445174 |
| iHRVB_SC0739 | MN306037 |
| iHRVB_G001-19 | MN212904 |
| iHR_VB_R93 | KF958309 |
| iHR_VB_BCH214 | GU568096 |
| iHR_VB_3039 | KF958308 |
| iEV-B69 | AY302560 |
| iEV-B73 | AF241359 |
| iEV-B74 | AY556057 |
| iEV-B75 | AY556070 |
| iEV-B77 | AJ493062 |
| iEV-B79 | AB426610 |
| iEV-B80 | AY843298 |
| iEV-B81 | AY843299 |
| iEV-B82 | AY843300 |
| iEV-B83 | AY843301 |
| iEV-B84 | DQ902712 |
| iEV-B85 | AY843303 |
| iEV-B86 | AY843304 |
| iEV-B87 | KC292019 |
| iEV-B88 | AY843306 |
| iEV-B93 | MN580134 |
| iEV-B97 | AY843307 |
| iEV-B98 | AB426608 |
| iEV-B100 | DQ902713 |
| iEV-B101 | AY843308 |
| iEV-B106 | KF990476 |
| iEV-B107_TN94-0349 | AB426609 |
| iEV-B111 | KF312882 |
| iEV-B113 | KC344833 |
| iEV-D68 | NC_038308 |
| iEV-D70 | NC_001430 |
| iEV-D94 | DQ916376 |
| iEV-D111 | MK032895 |
| iHCosV-B1 | NC_012801 |
| iEcho-E7 | LS451295 |
| iEcho-E9 | AF524866 |
| iPV3_PAK1019536 | KY941934.1 |
| iPV3_NIE1219535 | KY941933.1 |
| iPV3_SWI10947 | FJ914252.2 |
| iPV3_FIN84 | FJ842158.1 |

In some embodiments, a circRNA comprises any one of the IRES in Table 7, or a fragment or derivative thereof. In some embodiments, a circRNA comprises an IRES encoded by any one of SEQ ID NO: 33145-33169, or a fragment or derivative thereof. In some embodiments, the IRES is a Type 1 IRES. Type I IRES elements occur in the RNA genome of enterovirus species, including poliovirus (PV), coxsackievirus B3 (CVB3), enterovirus 71 (EV71), and human rhinovirus (HRV). In some embodiments, the IRES is an enterovirus IRES. In some embodiments, the IRES is an HRV IRES.

In some embodiments, a circRNA comprises any one of the following IRES: iCVA20; iEchoV-E11, iSimianEV-A, iCovidl9, iHRV-A57, iEchoVl1, iCrPV, iHRV-A89, iHRV-B26, iBEV, iEchoV1, iHRV-A21, iPV1, iCVB3, iEMCV, iHCV, iCVB5, iSwineVesicular, iHRV-A2, iHRV-C3, iHRV-C11, iCVB1, iPV2, iHRV-B17, iEchoV-E15, iEV71, iHRV-A9, iSiminanV4, iEV-D94, iSimianA5, iPV3, iHRV-C54, iHRV-A100, iHRV-B37, iHRV-B4, iHRV-B92, iHRV-B3, iHRV-A1, iEV107, or a fragment or derivative thereof.

In some embodiments, a circRNA comprises any one of the following IRES: iEMCV, iHCV, iCVB5, iSwineVesicular, iHRV-A2, iHRV-C3, iHRV-C11, iCVB1, iPV2, iHRV-B17, iEchoV-E15, iEV71, iHRV-A9, iSiminanV4, iEV-D94, iSimianA5, iPV3, iHRV-C54, iHRV-A100, iHRV-B37, iHRV-B4, iHRV-B92, iHRV-B3, iHRV-A1, iEV107, or a fragment or derivative thereof.

In some embodiments, a circRNA comprises any of the following IRES: iEV-B79, iEV-B77, iPV3_SWI10947, iHRV-B26, iHRV-B37, iHRV-A89, iEV-B86, iEV-B113, iEV-B87, iHRVA021, iEV-B88, iHRV-C11, iEV-B93, iEVD70, iEV-B111, iHRV-B92, iEV-B69, iEV-B73, iEV-B107, iEV107, iHRV-C54, iEV-B100, iHRVB_BCH214, iEV-B98, iPV3_NIE21219535, iEV-D111, iEcho-E9, iEV-B82, iEV-D94, iEV-B75, iEV97, iEV-B84, iHRV-C3, iHRV-A1, iEcho-E7, iEV-B81, iPV3_PAK1019536, iHRV-A9, iEV-B106, iHRV-A100, iPV3_FIN84, iEV-B85, iHRV-B86, iEV-B101, iHRV-B3, iHRV-B17, iHRVB_G001-10, iHRV-B70, iEV-B74, iEV-B80, iCVB3, iEV-B83, iHRV-A57, iHRV-B35, iHRV-B4, iEV-D68, iHRVB_R93, iHRV-B5, iHRVB-B52, iHRVB-B93, iHRV-B84, iHRV-B83_SC2220, iHRV-B72, iHRV-B69, iHRVB_SC0739, iHRV-B91, iHRV-B42, iHRV-B6, iHRV-B83, iHRV-B48, iIHRV-B99, iHRV-B79, iHRV-B97, iHRV-B27, iHRVB_3039, iHRVB-B14, iCosV-B1. In some embodiments, a circRNA comprises any of the following IRES: iEV-B83, iHRV-A57, iHRV-B35, iHRV-B4, iEV-D68, iHRVB_R93, iHRV-B5, iHRVB-B52, iHRVB-B93, iHRV-B84, iHRV-B83_SC2220, iHRV-B72, iHRV-B69, iHRVB_SC0739, iHRV-B91, iHRV-B42, iHRV-B6, iHRV-B83, iHRV-B48, iHRV-B99, iHRV-B79, iHRV-B97, iHRV-B27, iHRVB_3039, iHRVB-B14, iCosV-B1, or a fragment or derivative thereof.

In some embodiments, a circRNA comprises the iCVB3 IRES. In some embodiments, a circRNA comprises a fragment or derivative of the iCVB3 IRES. In some embodiments, a circRNA comprises the iHRV-B3 IRES. In some embodiments, a circRNA comprises a fragment or derivative of the iHRV-B3 IRES.

### Synthetic IRES

In some embodiments, a circRNA comprises a synthetic IRES. A “synthetic IRES” is an IRES that is modified relative to a wildtype IRES in order to modulate its structure and/or activity. For example, in some embodiments, an IRES that is modified to incorporate an aptamer sequence is a synthetic IRES.

In some embodiments, a synthetic IRES comprises an aptamer. In some embodiments, a synthetic IRES comprises a first aptamer and a second aptamer. In some embodiments, a synthetic comprises two, three, four, five, six, seven, eight, nine, ten, or more aptamers. In some embodiments, the aptamer is a wildtype aptamer. In some embodiments, the aptamer is a fragment of a wildtype aptamer. In some embodiments, the aptamer is an aptamer that was designed to bind DNA or RNA. Synthetic aptamers can be created that bind a specific DNA or RNA sequence by evolution through one or more rounds of evolution using, for example, SELEX technology.

In some embodiments, the aptamer is a modified version of a known aptamer (e.g., a mutant aptamer). In some embodiments, the aptamer is modified to have an extended stem region. For example, the length of the stem region may be extended by about 10% to about 25%, about 25% to about 50%, about 50% to about 75%, about 75% to about 100%, about 125%, about 150%, about 175%, about 200% or more. In some embodiments, the length of the stem region is extended by about 1, about 2, about 3, about 4, about 5, about 6, about 7, about 8, about 9, about 10 base pairs. As will be understood by those of skill in the art, extension of a stem region by 1 base pair comprises adding 2 nucleotides to the aptamer sequence. Accordingly, an aptamer which comprises a stem region extended by 3 base pairs have a nucleotide sequence that is 6 nucleotides longer than the same aptamer in which the stem region is not extended. The aptamer may be inserted into the IRES sequence in any location which is permissive to such changes. In some embodiments, the aptamer is positioned within the secondary structure of the IRES so that is spatially proximal to portion of the IRES responsible for translation initiation. In some embodiments, the aptamer is located in a position where it can bind to one or more translation initiation factors, such as eIF4G. In some embodiments, the aptamer does not interrupt the native eIF4G binding site of the IRES. In some embodiments, the IRES does not interrupt a native GRNA tetraloop within the IRES.

In some embodiments, the aptamer is an eIF4G-binding aptamer, such as any one of the aptamers listed in Table 6. In some embodiments, the aptamer is a fragment or derivative of any of the aptamers listed in Table 6. In some embodiments, the eIF4G-binding aptamer comprises or is encoded by the sequence of SEQ ID NO: 33143. In some embodiments, the eIF4G-binding aptamer comprises the sequence of SEQ ID NO: 33178.

In some embodiments, RNA aptamers function to enable small molecule control over circRNA translation or directing circRNAs toward specific intracellular targets. Additionally, incorporation of RNA aptamers may provide an avenue for cell type-specific expression of circRNAs.

TABLE 6
eIF4G-Binding Aptamers

| Aptamer | | | SEQ ID |
|---|---|---|---|
| ID | DNA Sequence | RNA Sequence | NOs: |
| A | TAA TAC GAC TCA CTA | UAAUACGACUCACUAUAGG | 33170, |
| | TAG GG ACTC ACT ATT TGT | GACUCACUAUUUGUUUUCG | 33174 |
| | TTT CGC GCC CAG TTG | CGCCCAGUUGCAAAAAGUG | |
| | CAA AAA GTG TCG CCG | UCGCCGCAUCUAGAGGGCC | |
| | CAT CTA GAG GGC C | | |
| B | TAA TAC GAC TCA CTA | UAAUACGACUCACUAUAGG | 33171, |
| | TAG GGT CCA G?C CCC | GUCCAGACCCCAACAGACU | 33175 |
| | AAC AGA CTC CAT AAC | CCAUAACUAAUAUGUCGCA | |
| | TAA TAT GTC GCA AAA | AAACCGCAUCUAGAGGGCC | |
| | CCG CAT CTA GAG GGC C | | |
| C | TAA TAC GAC TCA CTA | UAAUACGACUCACUAUAGG | 33172, |
| | TAG GGT CCG TAG AAA | GUCCGUAGAAACGCGUUAA | 33176 |
| | CGC GTT AAG GTG AAA | GGUGAAAGUUUGAGGGCUC | |
| | GTT TGA GGG CTC CTC | CUCACCGCAUCUAGAGGGC | |
| | ACC GCA TCT AGA GGG CC | C | |
| D | TAA TAC GAC TCA CTA | UAAUACGACUCACUAUAGG | 33173, |
| | TAG GGT CCG CGG CGC | GUCCGCGGCGCCAUCUCAU | 33177 |
| | CAT CTC ATG TTT AGT TGT | GUUUAGUUGUCCUAUGUCG | |
| | CCT ATG TCG AGC CCG | AGCCCGCAUCUAGAGGGCC | |
| | CAT CTA GAG GGC C | | |
| E | ACTCACTATTTGTTTTCGCG | ACUCACUAUUUGUUUUCGC | 33143, |
| | CCCAGTTGCAAAAAGTGTC | GCCCAGUUGCAAAAAGUGU | 33178 |
| | G | CG | |

In some embodiments, the IRES is a type I IRES. In some embodiments, the IRES is an enterovirus IRES. In some embodiments, the IRES is an HRV IREs.

SEQ ID NO: 33145-33169 shown in the SEQUENCE APPENDIX provide illustrative IRES sequences, wherein the IRES sequences comprise an aptamer. The aptamer insertion is shown in capital letters.

In some embodiments, a synthetic IRES sequence comprises a modified iCVB3 IRES. In some embodiments, the modified iCVB3 IRES comprises an aptamer inserted in domain I, II, III, IV, V, VI, or VII thereof. In some embodiments the modified iCVB3 IRES comprises an aptamer inserted in domain I, II, III, IV, V, VI, or VII thereof, in a location that minimally disrupts the native RNA structure. In some embodiments, the modified iCVB3 IRES comprises an aptamer inserted in domain IV thereof. In some embodiments, the aptamer is modified to have an extended stem region. The stem region may be extended, for example, by 1, 2, 3, 4, 5, 6, or more base pairs. In some embodiments, the aptamer is positioned within the secondary structure of the IRES so that is spatially proximal to portion of the IRES responsible for translation initiation. In some embodiments, the aptamer does not interrupt the native eIF4G binding site of the IRES and/or does not interrupt a native GRNA tetraloop within the IRES. In some embodiments, a synthetic IRES sequence comprises a modified iHRV-133 IRES. In some embodiments, the modified iHRV-B3 IRES comprises an aptamer inserted in domain I, II, III, IV, V, VI, or VII thereof. In some embodiments, the modified iHRV-B3 IRES comprises an aptamer inserted in domain IV thereof. In some embodiments, the aptamer is modified to have an extended stem region. The stem region may be extended, for example, by 1, 2, 3, 4, 5, 6, or more base pairs. In some embodiments, the aptamer is positioned within the secondary structure of the IRES so that is spatially proximal to portion of the IRES responsible for translation initiation. In some embodiments, the aptamer does not interrupt the native eIF4G binding site of the IRES and/or does not interrupt a native GRNA tetraloop within the IRES.

### IRES Elements and Features

In some embodiments, a circRNA comprises an IRES, such as a synthetic or viral IRES, that comprises one or more of the IRES elements or features described below. In some embodiments, a circRNA comprises an IRES that comprises at least one RNA secondary structure element. Intramolecular RNA base pairing is often the basis of RNA secondary structure and in some circumstances be a critical determinant of overall macromolecular folding. In conjunction with cofactors and RNA binding proteins (RBPs), secondary structure elements can form higher order tertiary structures and thereby confer catalytic, regulatory, and scaffolding functions to RNA. Thus, the IRES may comprise any RNA secondary structure element that imparts such structural or functional determinants. In some embodiments, the RNA secondary structure may be formed from the nucleotides at about position 40 to about position 60 of the IRES, relative to the 5′ end thereof. The most common RNA secondary structures are helices, loops, bulges, and junctions, with stem-loops or hairpin loops being the most common element of RNA secondary structure. A stem-loop is formed when the RNA chains fold back on themselves to form a double helical tract called the stem, with the unpaired nucleotides forming a single-stranded region called the loop. Bulges and internal loops are formed by separation of the double helical tract on either one strand (bulge) or on both strands (internal loops) by unpaired nucleotides. A tetraloop is a four-base pairs hairpin RNA structure. There are three common families of tetraloop in ribosomal RNA: UNCG (SEQ ID NO: 33179), GNRA (SEQ ID NO: 33180), and CUUG (SEQ ID NO: 33181) (N is one of the four nucleotides and R is a purine). Pseudoknots are formed when nucleotides from the hairpin loop pair with a single stranded region outside of the hairpin to form a helical segment. RNA secondary structure is further described in, e.g., Vandivier et al., Annu Rev Plant Biol., 67: 463-488 (2016); and Tinoco and Bustamante, supra). In some embodiments, the IRES of the recombinant circRNA molecule comprises at least one stem-loop structure. The at least one RNA secondary structure element may be located at any position of the IRES, so long as translation is efficiently initiated from the TRES. In some embodiments, the stem portion of the stem-loop may comprise from 3-7 base pairs, 4, 5, 6, 7, 8, 9, 10, 11 or 12 base pairs or more. The loop portion of the stem-loop may comprise from 3-12 nucleotides, including 4, 5, 6, 7, 8, 9, 10, 11, 12 or more nucleotides. The stem-loop structure may also have on either side of the stem one or more bulges (mismatches). In some embodiments, the RNA secondary structure element is formed from the nucleotides at about position 40 to about position 60 of the IRES, wherein the first nucleic acid at the 5′ end of the IRES is considered to be position 1. In some embodiments, the sequence that is complementary to an 18S rRNA is located 5′ to the at least one RNA secondary structure element (i.e., in the range of about position 1 to about position 40 of the IRES). In some embodiments, the sequence that is complementary to an 18S rRNA is located 3′ to the a least one RNA secondary structure element (i.e., in the range of about position 61 to the end of the IRES). Sequences encoding exemplary secondary structure-forming RNA sequences that may be included in the IRES described herein are set forth in Table 2.

In some embodiments, the at least one RNA secondary structure element of the IRES is a stem-loop. In some embodiments, the at least one RNA secondary structure element is encoded by any one of the nucleic acid sequences listed in Table 2. In some embodiments, the at least one RNA secondary structure element is encoded by a nucleic acid sequence having at least 80%, at least 85%, at least 90%, at least 91%, at least 92%, at least 93%, at least 94%, at least 95%, at least 96%, at least 97%, at least 98%, or at least 99% sequence identity relative to any one of the nucleic acid sequences listed in Table 2. In some embodiments, the at least one RNA secondary structure element is encoded by a nucleic acid sequence having at least 1, at least 2, at least 3, at least 4, at least 5, at least 6, at least 7, at least 8, at least 9 at least 10, or more nucleotide substitutions relative to any one of the nucleic acid sequences listed in Table 2.

RNA secondary structure typically can be predicted from experimental thermodynamic data coupled with chemical mapping, nuclear magnetic resonance (NMR) spectroscopy, and/or sequence comparison. In some embodiments, the RNA secondary structure is predicted by a machine-learning/deep-learning algorithm (e.g., CNN) (See, Zhao, Q., et al., “Review of Machine-Learning Methods for RNA Secondary Structure Prediction,” Sep. 1, 2020 (available on the world wide web at: arxiv.org/abs/2009.08868). A variety of algorithms and software packages for RNA secondary structure prediction and analysis are known in the art and can be used in the context of the present disclosure (see, e.g., Hofacker I. L. (2014) Energy-Directed RNA Structure Prediction, In: Gorodkin J., Ruzzo W. (eds) RNA Sequence, Structure, and Function: Computational and Bioinformatic Methods. Methods in Molecular Biology (Methods and Protocols), vol 1097. Humana Press, Totowa, NJ; Mathews et al., supra; Mathews, et al. “RNA secondary structure prediction,” Current Protocols in Nucleic Acid Chemistry, Chapter 11 (2007): Unit 11.2. doi:10.1002/0471142700.ncI102s28; Lorenz et al., Methods, 103: 86-98 (2016); Mathews et al., ColdSpring Harb PerspectBiol., 2(12): a003665 (2010)). In some embodiments, the IRES of the recombinant circRNA may comprise a nucleic acid sequence that is complementary to 18S ribosomal RNA (rRNA). Eukaryotic ribosomes, also known as “80S” ribosomes, have two unequal subunits, designated small subunit (40S) (also referred to as “SSU”) and large subunit (60S) (also referred to as “LSU”) according to their sedimentation coefficients. Both subunits contain dozens of ribosomal proteins arranged on a scaffold composed of ribosomal RNA (rRNA). In eukaryotes, eukaryotic 80S ribosomes contain greater than 5500 nucleotides of rRNA: 18S rRNA in the small subunit, and 5S, 5.8S, and 25S rRNA in the large subunit. The small subunit monitors the complementarity between tRNA anticodon and mRNA, while the large subunit catalyzes peptide bond formation. Ribosomes typically contain about 60% rRNA and about 40% protein. Although the primary structure of rRNA sequences can vary across organisms, base-pairing within these sequences commonly forms stem-loop configurations.

In some embodiments, the IRES sequence comprises a locked nucleic acid (LNA) against a non-base-paired linker region between domains of the IRES.

In some embodiments, the IRES of the recombinant circRNA may comprise any nucleic acid sequence that is complementary to any eukaryotic 18S rRNA sequence. In some embodiments, the nucleic acid sequence that is complementary to 18S rRNA is encoded by any one of the nucleic acid sequences set forth in Table 3. In some embodiments, the nucleic acid sequence that is complementary to 18S rRNA is encoded by a nucleic acid sequence that has at least 80%, at least 85%, at least 90%, at least 91%, at least 92%, at least 93%, at least 94%, at least 95%, at least 96%, at least 97%, at least 98%, or at least 99% identity or homology to a sequence set forth in Table 3. In some embodiments, the nucleic acid sequence that is complementary to 18S rRNA is encoded by a nucleic acid sequence that has at least 1, at least 2, at least 3, at least 4, at least 5, at least 6, at least 7, at least 8, at least 9, at least 10, or more nucleotide substitutions relative to a nucleic acid sequence set forth in Table 3.

TABLE 3
Illustrative DNA sequences that encode RNA
sequences that are complementary to 18S RNA

| SEQ ID | |
|---|---|
| NO: | Nucleic Acid Sequence |
| 34425 | CACCTACGGAAACCTTGTTACGACTTTTACTTCCTCTAGATAGTCAAGTTC |
| | GACCGTCTTCTCAGCGCTCCGCCAGGGCCGTGGGCCGACCCCGGCGGGGC |
| | CGATCCGAGGGCCTCACTAAACCATCCAATCGGTAGTAGCGACGGGCGGT |
| | GTGTACAAAGGGCAGGGACTTAATCAACGCAAGCTTATGACCCGCACTTA |
| | CTGGGAATTCCTCGTTCATGGGGAATAATTGCAATCCCC |
| 34426 | GGGACGCCGACCGCTCGGGGGTCGCGTAACTAGTTAGCATGCCAGAGTCT |
| | CGTTCGTTATCGGAATTAACCAGACAAATCGCTCCACCAACTAAGAACGG |
| | CCATGCACCACCACCC |
| 34427 | GTTTATGGTCGGAACTACGACGGTATCTGATCGTCTTCGAACCTCCGACTT |
| | TCGTTCTTGATTAATGAAAACATTCTTGGCAAATGCTTTCGCTCTGGTCCG |
| | TCTTGCGCCGGTCCAAGAATTTCACCTCTAGCGGCGCAATACGAATGCCCC |
| | CGGCCGTCCCTCTTAATCATGGCCTCAGTTCCGAAAACCAACAAAATAGAA |
| | CCGCGGTCCTATTCCATTATTCCTAGCTGCGGTATCCAGGCGGCTCGGGCC |
| | TGCTTTGAACACTCTAATTTTTTCAAAGTAAACGCTTCGGG |
| 34428 | AGAGCATCGAGGGGGCGCCGAGAGGCAAGGGGGGGGACGGGCGGTGGC |
| | TCGCCTCGCGGCGGACCGCCCGCCCGCTCCC |
| 34429 | TACGAGCTTT |
| 34430 | ATACGCTATTGGAGCTGGAATTACCGCGGCTGCTGGCACCAGACTTGCCCT |
| | CCAATGGATCCTCGTTAAAGGATTTAAAGTGGACTCATTCCAATTACAGGG |
| | CCTCGAAAGAGTCCTGTA |
| 34431 | GGGGGCGTGCGATCGGCCCGAGGTTATCTAGAGTCACCAAAGCCGCCGGC |
| | GCCCGCCCCCCGGCCGGGGCCGGAGAGGGG |

The most commonly used criterion for RNA secondary structure prediction is the minimum free energy (MFE), since, according to thermodynamics, the MFE structure is not only the most stable, but also the most probable one in thermodynamic equilibrium. The MFE of an RNA or DNA molecule is affected by three properties of nucleotides in the RNA/DNA sequence: number, composition, and arrangement. For example, longer sequences are on average more stable because they can form more stacking and hydrogen bond interactions, guanine-cytosine (GC)-rich RNAs are typically more stable than adenine-uracil (AU)-rich sequences, and nucleotide order influences the folding structure stability because it determines the number and the extension of loops and double-helix conformations. It has been found that mRNAs and microRNA precursors, unlike other non-coding RNAs, have greater negative MFE than expected given their nucleotide numbers and compositions. Thus, free energy also can be employed as a criterion for the identification of functional RNAs.

The IRES of the recombinant circRNA molecule may comprise a minimum free energy (MFE) of less than about −15 kJ/mol (e.g., less than about −16 kJ/mol, less than about −17 kJ/mol, less than about −18.5 kJ/mol, less than about −19 kJ/mol, less than about −18.9 kJ/mol, less than about −20 kJ/mol, less than about −30 kJ/mol). In some embodiments, the MFE is greater than about −90 kJ/mol (e.g., greater than about −85 kJ/mol, greater than about −80 kJ/mol, greater than about −70 kJ/mol, greater than about −60 kJ/mol, greater than about −50 kJ/mol, greater than about −40 kJ/mol). In some embodiments, the IRES has a has a minimum free energy (MFE) of about −18.9 kJ/mol or less. In some embodiments, the IRES has a MFE in the range of about −15.9 kJ/mol to about −79.9 kJ/mol. In some embodiments, the IRES may comprise a MFE in the range of about −12.55 kJ/mol to about −100.15 kJ/mol. In some embodiments, the IRES is a viral IRES and has an MFE in the range of about −15.9 kJ/mol to about −79.9 kJ/mol. In some embodiments, the IRES is a human IRES and has a MFE in the range of about −12.55 kJ/mol to about −100.15 kJ/mol.

In some embodiments, the at least one secondary structure element of an IRES of may comprise a minimum free energy (MFE) of less than about −0.4 kJ/mol, less than about −0.5 kJ/mol, less than about −0.6 kJ/mol, less than about −0.7 kJ/mol, less than about −0.8 kJ/mol, less than about −0.9 kJ/mol, or less than about −1.0 kJ/mol. In some embodiments, the at least one secondary structure element of the IRES may comprise a MFE of less than about −0.7 kJ/mol. In some embodiments, the RNA sequence comprising the nucleotides at about position 40 to about position 60 of an IRES of a circRNA described herein may comprise a minimum free energy (MFE) of less than about −0.4 kJ/mol, less than about −0.5 kJ/mol, less than about −0.6 kJ/mol, less than about −0.7 kJ/mol, less than about −0.8 kJ/mol, less than about −0.9 kJ/mol, or less than about −1.0 kJ/mol. In some embodiments, the RNA sequence comprising the nucleotides at about position 40 to about position 60 of the IRES may comprise an MFE of less than about −0.7 kJ/mol.

As discussed, above, the minimum free energy of a particular RNA (e.g., an RNA produced from a DNA sequence) may be determined using a variety of computational methods and algorithms. The most commonly used software programs, employed to predict the secondary RNA or DNA structures by MFE algorithms, make use of the so-called nearest-neighbor energy model. This model uses free energy rules based on empirical thermodynamic parameters (Mathews et al., J Mol Biol, 288: 911-940 (1999); and Mathews et al., Proc Natl Acad Sci USA, 101: 7287-7292 (2004)) and computes the overall stability of an RNA or DNA structure by adding independent contributions of local free energy interactions due to adjacent base pairs and loop regions. In sequences with homogeneous nucleotide arrangements and compositions, the additive and independent nature of the local free energy contributions suggests a linear relationship between computed MFE and sequence length (Trotta, E., PLoS One, 9(11): e113380 (2014)). Algorithms for determining MFE are further described in, e.g., Hajiaghayi et al., BMC Bioinformatics, 13: 22 (2012); Mathews, D. H., Bioinformatics, Volume 21, Issue 10: 2246-2253 (2005); and Doshi et al., BMC Bioinformatics, 5: 105 (2004) doi 10.1186/1471-2105-5-105). One of ordinary skill in the art will appreciate that the melting temperature (Tm) of a particular circRNA molecule may also be indicative of stability. Indeed, RNA sequences with high Tm generally contain thermo-stable functionally important RNA structures (see, e.g., Nucleic Acids Res., 45(10): 6109-6118 (2017)). Thus, in some embodiments, the IRES of the recombinant circRNA molecule has a melting temperature of at least 35.0° C. In some embodiments, the IRES of the recombinant circRNA molecule has a melting temperature of at least 35.0° C., but not more than about 85° C. In some embodiments, in some embodiments, the RNA secondary structure has a melting temperature of at least 35° C., at least 36° C., at least 37° C., at least 38° C., at least 39° C., at least 40° C., at least 41° C., at least 42° C., at least 43° C., at least 44° C., at least 45° C., at least 46° C., at least 47° C., at least 48° C., at least 49° C. or greater. In some embodiments, the melting temperature is not more than about 85° C., not more than about 75° C., not more than about 70° C., not more than about 65° C., not more than about 60° C., not more than about 55° C., not more than about 50° C. or less.

The melting temperature of a particular nucleic acid molecule can be determined using thermodynamic analyses and algorithms described herein and known in the art (see, e.g., Kibbe W. A., Nucleic Acids Res., 35(Web Server issue): W43-W46 (2007). doi:10.1093/nar/gkm234; and Dumousseau et al., BMC Bioinformatics, 13: 101 (2012). doi.org/10.1186/1471-2105-13-101).

In some embodiments, the IRES comprises at least one RNA secondary structure element; and a nucleic acid sequence that is complementary to an 18S ribosomal RNA (rRNA); wherein the IRES has a minimum free energy (MFE) of −18.9 kJ/mol or less and a melting temperature of at least 35.0° C. In some embodiments, the RNA secondary structure element of the IRES has a has a minimum free energy (MFE) of less than −18.9 kJ/mol, and is formed from the nucleotides at about position 40 to about position 60 of the IRES, wherein the first nucleic acid at the 5′ end of the IRES is considered to be position 1. In some embodiments, the RNA secondary structure element has a melting temperature of at least 35.0° C., and is formed from the nucleotides at about position 40 to about position 60 of the IRES, wherein the first nucleic acid at the 5′ end of the IRES is considered to be position 1.

Because circRNA molecules are often generated from linear RNAs by back-splicing of a downstream 5′ splice site (splice donor) to an upstream 3′ splice site (splice acceptor), the recombinant circular RNA molecule may further comprise a back-splice junction. In some embodiments, the IRES may be located within about 100 to about 200 nucleotides of the back-splice junction. In addition, it has been observed that regions of RNA with higher G-C content have more stable secondary structures than RNA strands with lower G-C content. Thus, in some embodiments, the IRES of the recombinant circRNA molecule may further comprise a minimum level of G-C base pairs. For example, the non-native IRES of the recombinant circRNA molecule may comprise a G-C content of at least 25% (e.g., at least 30%, at least 35%, at least 40%, at least 45% or more), but not more than about 75% (e.g., about 70%, about 65%, about 60%, about 55%, about 50% or less). In some embodiments, the IRES has a G-C content of at least 25%.

G-C content of a given nucleic acid sequence may be measured using any method known in the art, such as, for example chemical mapping methods (see, e.g., Cheng et al., PNAS, 114 (37): 9876-9881 (2017); and Tian, S. and Das, R., Quarterly Reviews of Biophysics, 49: e7 doi:10.1017/S0033583516000020 (2016)).

Exemplary sequences encoding IRESs for use in the circRNA molecules of the present disclosure are set forth in Table TA and Table 1B. Thus, the disclosure further provides a recombinant circular RNA molecule comprising a protein-coding nucleic acid sequence and an IRES operably linked to the protein-coding nucleic acid sequence in a non-native configuration; wherein the IRES is encoded by any one of the nucleic acid sequences listed in Table 1A or Table 1B.

In some embodiments, the IRES is encoded by any one of the nucleic acid sequences set forth in Table 1A. In some embodiments, the IRES is encoded by a nucleic acid sequence that has at least 80%, at least 85%, at least 90%, at least 91%, at least 92%, at least 93%, at least 94%, at least 95%, at least 96%, at least 97%, at least 98, or at least 99% identity to one or the nucleic acid sequences of Table 1A. In some embodiments, the IRES is encoded by a nucleic acid sequence that has at least 1, at least 2, at least 3, at least 4, at least 5, at least 6, at least 7, at least 8, at least 9, at least 10 or more nucleotide substitutions relative to any one of the sequences in Table 1A.

In some embodiments, the IRES is encoded by any one of the nucleic acid sequences set forth in Table 1B. In some embodiments, the IRES is encoded by a nucleic acid sequence that has at least 80%, at least 85%, at least 90%, at least 91%, at least 92%, at least 93%, at least 94%, at least 95%, at least 96%, at least 97%, at least 98, or at least 99% identity or homology to one or the nucleic acid sequences of Table 1B. In some embodiments, the IRES is encoded by a nucleic acid sequence that has at least 1, at least 2, at least 3, at least 4, at least 5, at least 6, at least 7, at least 8, at least 9, at least 10 or more nucleotide substitutions relative to any one of the sequences in Table 1B.

In some embodiments, the IRES is encoded by the nucleic acid sequences denoted Index 876 (SEQ ID NO: 553), 6063 (SEQ ID NO: 2292), 7005 (SEQ ID NO: 2624), 8228 (SEQ ID NO: 3064), or 8778 (SEQ ID NO: 3266) in Table 1B. In some embodiments, the IRES is encoded by the nucleic acid sequence of SEQ ID NO: 32956, In some embodiments, the IRES is encoded by any one of the nucleic acid sequences set forth in Table 5. In some embodiments, the IRES is encoded by a nucleic acid sequence that has at least 80%, at least 85%, at least 90%, at least 91%, at least 92%, at least 93%, at least 94%, at least 95%, at least 96%, at least 97%, at least 98, or at least 99% identity or homology to one or the nucleic acid sequences of Table 5. In some embodiments, the IRES is encoded by a nucleic acid sequence that has at least 1, at least 2, at least 3, at least 4, at least 5, at least 6, at least 7, at least 8, at least 9, at least 10 or more nucleotide substitutions relative to any one of the sequences in Table 5.

TABLE 5
Illustrative Sequences Encoding IRES sequences

| Oligo- | | SEQ |
|---|---|---|
| nucleotide | | ID |
| Identifier | Sequence | NO: |
| 4173 | CATCCTCCTGCTGAAGTGACAAGCCACGCTGCTTCTGGA | 38403 |
| | GCCAAAGCTGACCAAGAAGAACAAATCCACCCCAGATC | |
| | TAGACTCAGGTCACCTCCTGAAGCCCTCGTTCAGGGTCG | |
| | ATATCCCCACATCAAGGACGGTGAGGATCTTAAAGACCA | |
| | CTCAACAGAAAGTAAAAAA | |
| 2125 | GCAGAAGGGAAACGTGAAGAAGGTGAAGATGGCGGTGG | 38404 |
| | CCAGGGCCGGGGTCTTGGGAGTCCAGTGGCTGCAAAGG | |
| | GCATCCCGGAACGTGATGCCGCTGGGCGCACGGACAGC | |
| | CTCCCACATGACCAAGGACATGTTCCCGGGGCCCTATCC | |
| | TAGGACCCCAGAAGAACGGGC | |
| 6742 | AGACTCGGGAACTGCCTGAATGTGGTTTGGGACACGAG | 38405 |
| | ACCTCATCATATTGATGAGCGAACAAACAAGAACATTTC | |
| | CTCCCTCCCCTCCTTTGAATTGAAATGGCACATTAAGACT | |
| | TGTCACGGCTTCTCACTGGGACTGGAGACCTCGTTCCTTC | |
| | ACCCCGCGTGTCGCCAG | |
| 8839 | AGGTGGGCGGTGGCGGTAGGTGGGTGGCCAGCGACTGG | 38406 |
| | CAACGCGGTGGCTTAGACGCGCGAGATCCGGCCAGGGT | |
| | CAGAGTACTGTAAGATTGATGTTAAAGGCATGGTGTTCA | |
| | CCCCACTTCATCAGCGTACATAAGTTATCTCTTCTTTTGG | |
| | ACCCTTATTTTATGCCATA | |
| 8279 | GGAGGCAGGTTGATGTGTTTGTGCTTCCTTCTACAGCCA | 38407 |
| | ATATGAAAAGGCCTAGTAAGTGGGGTCGGGAGGCGGGC | |
| | GTGGAGGGACCCACGTCTGGAAGTTGCTGCAGCCACCAC | |
| | GACGCTCTTCTACGGCTACGGCTTTGTCTCTGCTGAGTTA | |
| | AAGAAAGCAAGTAAACGC | |
| 1637 | ATTTATAAGCAATAGTAGCTTATTTGGCTCACAGTTCTG | 38408 |
| | GAGGCTGAGAAGATCGTGAGGCTGCATCTGGCAAGGGC | |
| | CTTCTTGCTGCTTCATAACATGGCAGAAGACATCATGCG | |
| | GGTGTGTGTCTGGGGAAGAGACTTACAGAAGTGGAGTT | |
| | GCTGAGTCAAAGATCTAACC | |
| 27147 | TCTTTTGGTGCAGAAGGTGACGGGAAACAGGCCGCAGA | 38409 |
| | CCTGAACTTCCAACCGTATGTAGGCGAGAAGCCGGTGCC | |
| | GATACTCCCACTATCCCACAATGTCCCACTGGGCCCCAG | |
| | AGTGGAAGAGGGCGGAGGCTAATCCAAGAGACCTTGGG | |
| | GCCAGCTGGGATGTCAGGGG | |
| 7408 | ATCCACCATGAAGCAGAATCTAAAGAAAAAGGTCCAAG | 38410 |
| | CATGAAGGAACCAAATCCAATTTCTCCACCAATTTTCCA | |
| | TGGTCTGGACACCCTAACCGTGATGGGCATTGCGTTTGC | |
| | AGCCTTTGTGATCGGAGCACTCCTGACGGGGGCCTTGTG | |
| | GTACATCTATTCTCACACA | |
| 29135 | CAGATTAGAGGACGGGCGCTTTGGAGCCGGCCCCAGGC | 38411 |
| | AGCGTGTGTCGGTCGCCTAGTCTGGAGAACTAGTCCTCG | |
| | ACTCACGTGCAAGGATGATGCTGAAAGGAATAACAAGG | |
| | CTTATCTCTAGGATCCATAAGGCCAAGCATGGGGATCAG | |
| | CACGAGGGTCAGCACTACAA | |
| 7806 | CTCTCGGGGGCCAGGGGTCATCGGTTTGACCCCTGACCT | 38412 |
| | ATAAGCCAAGATACCCCATAAACACACTCAGAAAGCAG | |
| | AGAAAAAGGACAAGAGTCTGTGTTTGAGAGGGGGTCTG | |
| | CCATTCCTGCTTGGGGACTGGTGGGGAAGAGGGCCAGG | |
| | ACATCTTCTGAGCCAGGACGT | |
| 661 | AGTCTGGCTGCGGCGGCAGAGCTGAAGTGAGCGGAGCC | 38413 |
| | ACCAGGAGGCCATGTCGGGTGAGGACGCTGAGGTCCGG | |
| | GCAGTCTCTGAAGATGTCTCCAATGGAAGCAGTGGCTCG | |
| | CCCAGCCCTGGGGACACACTGCCCTGGAACCTTGGGAAA | |
| | ACGCAGCGGAGCCGGCGCAG | |
| 1258 | ATCTGCCTCAGCCTCTTTGTCACTGCCTGGACCATTGTCC | 38414 |
| | CTGCTGTTTCTCAGACAGCATAGAGGCTGAGGCTGGGGC | |
| | CAGGACCCAGACAGAGACACACGGTCACTGCAGCTGAA | |
| | GCCGCTGCCCCTGCTACAGGCACCACCAGGACCAGCTGA | |
| | TCATTCCAGCCCACAGCA | |
| 6757 | CTCAGGCTGTGGCCCCACCTGCCCCCAAGCCCACCCAGG | 38415 |
| | CTGGGGAAGGAACGCTGTCAGAGGCCCTGCTGCAGCTG | |
| | CAGTTTGATGATGAAGACCTGGGGGCCTTGCTTGGCAAC | |
| | AGCACAGACCCAGCTGTGTTCACAGACCTGGCATCCGTC | |
| | GACAACTCCGAGTTTCAGC | |
| 5825 | CTCAAGGTTTTGAAGAGACAGGGGACACGCTCTATGCCC | 38416 |
| | CCTATTCCACACATTTTCAGCTGCAGAACCAGCCTCCCC | |
| | AAAAGGAATTCTCCATCCCACGGGCCCACGCTTGGCCGA | |
| | GCCCTTACAAAGACTATGAAGTAAAGAAAGAGGGCAGC | |
| | TAACTCTGTCCTGAAGAGT | |
| 27115 | GCTTCCGTAGAGGCCAGCTGGTTGCTACGCAGCAACAGG | 38417 |
| | AAACATTCCGCGCTACCGAGTACTTTCTACTCCCGACCA | |
| | GGCATTGCTCTCTCTGGAGACCCTCGGCGGTGGTTGCTG | |
| | TATTTTGACTTGGAGTAAGGATGACTTTTCGGGCCACAG | |
| | ATAGTGAATTTGACCTGA | |
| 3130 | CTGGAGGGCCGCACTCCCGTTCCAGCCAGGCTGAGCCTT | 38418 |
| | CTGTCCCCTGCCTCTGGGGCCTGGGAACCCCCCTTCTTCT | |
| | TTCTCCTGAATGGCACCCCCGCCCTAGAATCCAGACACC | |
| | GAGTTTCCCACTGTGGCTGGTTCAAGGAGCTCCCTGGTG | |
| | ACAGTCTGTGGCTGAGC | |
| 5065 | TAGAGTGGGGGGTGGAAGATGATTCTCGCCTGTTGCTGG | 38419 |
| | GGATTTATGAACATGGCTATGGAAACTGGGAGTTAATTA | |
| | AAACAGACCCAGAGCTTAAATTAACTGACAAAATTCTGC | |
| | CGGTGGAGACAGATAAAAAGCCTCAGGGGAAGCAGCTA | |
| | CAGACCCGAGCGGATTACT | |
| 8066 | AGCGGCTGCAGCATCCAGCCAGCTTGGATGTCTGGCCTG | 38420 |
| | TGAGCCTGGGGAAACTATTATTAATAATATTTACTGTTG | |
| | ATAATATTGGGGAAAACAGCCCTTAACTCTGAGGTTTCT | |
| | GCTGTGCTCCTTTCCAAAACAGACTTCCAGGACTCTGAA | |
| | GAAACAGTTACAAGCAGG | |
| 1952 | AAGGGAGGAGGCGCCGAGCTGACCGGGCGACGCCGCGG | 38421 |
| | GAGGTTCTGGAAACGCCGGGAGCTGCGAGTGTCCAGAC | |
| | ACTTCCCTCTGTGACCATGAAACTCTGGGTGTCTGCATTG | |
| | CTGATGGCCTGGTTTGGTGTCCTGAGCTGTGTGCAGGCC | |
| | GAATTCTTCACCTCTATTG | |
| 5020 | GCGAACTGAAGGTGGCCCAAGGGAAGGCTCTGCAAGAG | 38422 |
| | AATTTGGCCCTCCTGACCCAGACCCTAGCTGAAAGAGAA | |
| | GAGGAGGTGGAGACTCTGCGGGGACAAATCCAGGAACT | |
| | GGAGAAGCAACGGGAAATGCAGAAGGCTGCTTTGGAAT | |
| | TGCTGTCTCTGGACCTGAAGA | |
| 8766 | AGGAAGCCGAGGAAGGCTGTGTTTCTGACTCACACGGG | 38423 |
| | GGAGTCGGGGGAGTCGTAAACAACCCTGAAGAGAACAG | |
| | CCAGGCCTGGCGCTCTGGGCTTGGTCCTGGGACACCCGT | |
| | TTGACACTGTAAAGGTGAGGCTGCAGACCCAGACCACCT | |
| | ACCGGGGCATCGTTGATTGC | |
| 27433 | CTGCCTCCCCCCCGCTCCCGCTCCCCTGAGCCCAGCCAG | 38424 |
| | ACCCCGCGCCGCCCGCGCCCCGCTCGACTCCGGAGGCTC | |
| | CCGCAGCCCCGGCGTCCGCCCCGCTGCCCCCTCCCCCGG | |
| | GGGCCATGGGGGCGCCCCCGGGCTACCGGCCCTCAGCTT | |
| | GGGTGCATCTCCTCCACC | |
| 27818 | CTCAGTGAAGCAACGAGGATGCCGGGGAGAGGGAAGGG | 38425 |
| | GCTGGGCTCTGGGCGGTGCCAAGTCTGTGAGGGGGCGC | |
| | GGTCACCGCCCAGGGTTCCCACGAACGCCAAGGCGGCC | |
| | ACGTCCTGCTCCCCCTGGTGAAGAAGCTGCCCTGGGCTT | |
| | GTCGTCCTAGGGTCTCCAGAC | |
| 8861 | CTGATGTGAGCTAGTTTGTCTGGTTGAGTTGGATGTTTAA | 38426 |
| | ATAGAAGGCAGAACAACAACAGGTACTCCACATCAGCA | |
| | TTCTCAAGACTGGAGAAGTTAGGCCTCAGACATCCCAAG | |
| | CCTTCTCCTTTCATTGGAAACTTGACATTTTTCCGCCAGG | |
| | GTTTTTGGGAAAGCCAA | |
| 8040 | GCCACATTCCACACTAACGTGTTGGTGAATTCTTCTGGG | 38427 |
| | CATTGCCAGTACCTGCCTCCAGGCATATTCAAGAGTTCC | |
| | TGCTACATCGATGTACGCTGGTTTCCCTTTGATGTGCAGC | |
| | ACTGCAAACTGAAGTTTGGGTCCTGGTCTTACGGAGGCT | |
| | GGTCCTTGGATCTGCAG | |
| 27907 | TCCTGCCTCGGCAACCCCGGGCCCTGAGGGCAGGCCCCA | 38428 |
| | ACCGCGGAGGAGCAGGAGAGGGCGGAGGCCGGCGGGC | |
| | CATGCCCTGGTCGTCCCGCGGCGCCCTCCTTCGGGACCT | |
| | GGTCCTGGGCGTGCTGGGCACCGCCGCCTTCCTGCTCGA | |
| | CCTGGGCACCGACCTGTGGG | |
| 2392 | AAGTTTGAAACTGGTAACTTCGGGAGTTGAGCCACGAGC | 38429 |
| | TGTTGTGCATCCAGAGGTGGAATTGGGGCCCGGCATTCC | |
| | CTCCTCGTCCCGGGCTGGCCCTTGCCCCCACCCTGCAACT | |
| | CCTGGTTGAGATGGGCTCAGCCAAGAGCGTCCCAGTCAC | |
| | ACCAGCGCGGCCTCCGC | |
| 7802 | AGCCTTCGCTGGATCTCCTTCCCGAACTCACAAATCCTG | 38430 |
| | ACGAGCTCCTGTCTTATCTGGACCCCCCCGACCTGCCGA | |
| | GCAATAGTAACGATGACCTCCTGTCTCTATTTGAGAACA | |
| | ACTGAGGGCCACCCGGTCGGGGCCATCCCTCCACACTCT | |
| | GCATCCTACCCCACCTAC | |
| 7655 | TTTTTAGAGGTTGGTTGTTGTGTTTTAGGATTCTGTCCAT | 38431 |
| | TTTCTTTTAAAGTTATAAACACGTACTTGTGCGAATTATT | |
| | TTTTTAAAGTGATTTGCCATTTTTGAAAGCGTATTTAATG | |
| | ATAGAATACTATCGAGCCAACATGTACTGACATGGAAA | |
| | GATGTCAAAGATATGT | |
| 981 | AGCCAGCGGACGTCCAGGAACCGGGATGCCTCCAGCAG | 38432 |
| | TGAGGCGGTCAGCCTGCAGCATGGGATGGCTGTGGATCT | |
| | TTGGGGCAGCCCTGGGGCAGTGTCTGGGCTACAGTTCAC | |
| | AGCAGCAAAGGGTGCCATTTCTTCAGCCTCCCGGTCAAA | |
| | GTCAACTGCAAGCGAGTTA | |
| 5027 | GGGACCAGGAACTGGAGGCTCTGCAGCAAGAACAGCAG | 38433 |
| | CAGGCCCAGGGACAGGAGGAGAGGGTGAAGGAAAAGG | |
| | CAGACGCCCTCCAGGGAGCTCTGGAGCAAGCCCATATG | |
| | ACACTGAAGGAGCGTCATGGAGAGCTTCAGGACCACAA | |
| | GGAACAGGCACGAAGGCTGGAGG | |
| 7683 | CTACTCCGGAGCCTGAGGTGGGAGGATCGCTTGAGTCTG | 38434 |
| | GGAGGCAGAGGCTGCATTGAGCTATGATCATGGCACTGC | |
| | ATTCCAGCCTGGGTGACAGTGCAAGACCTTGTCTCAGAA | |
| | TAAATAAAGTATGTGATGAAGATGTGCATACATTATATG | |
| | CAAATACTGTTTTTTTTT | |
| 8650 | CACCAATTCGGCTGGCGTCTCCGAGACCGCGGACTCCCG | 38435 |
| | TAGGGTCCCCGTGGCCCCGAGTTGTAGTCGGGACACCCC | |
| | GGCCGCGGGTGATCGTCGGGTCTCCACGCGCCCGGGTCG | |
| | CTGACGCGGATCCGGCCTCGGCGCCTTCTCAGGGCGCCC | |
| | TGCAAGGCCGCAGGCAGG | |
| 6898 | CCTCCTCTCTGGCTAATCAACAGCTGCCCCCAGCCTGTG | 38436 |
| | GTGCCAGGCAACTCAGCAAGCTGAAAAGGTTCCTTACTA | |
| | CCCTGCAGCAGTTTGGCAATGACATTTCACCCGAGATAG | |
| | GAGAAAGAGTTCGCACCCTCGTTCTGGGACTAGTGAACT | |
| | CCACTTTGACAATTGAAG | |
| 2042 | TTTAGTGTCACAGGAGACACGAGGGCAAAGTGCTGAGG | 38437 |
| | AAACTCTAGAGCAACATCGGAATGCATGGTGAAGCATC | |
| | AGATGGTGAAGGAGAAGGAGACACAGAAGTGATGCAGC | |
| | AGGAGACAGTTCCAGTTCCTGTACCTTCAGAGAAAACCA | |
| | AACAGCCTAAAGAATGTTTTT | |
| 1686 | GCCACTTCTCTTCCCTTCATTCTTCGCCAGGCTCTCTGCT | 38438 |
| | GACTCAAGTTCTTCAGTTCACGATCTTCTAGTTGCAGCG | |
| | ATGAGTGCACGAGTGAGATCAAGATCCAGAGGAAGAGG | |
| | AGATGGTCAGGAGGCTCCCGATGTGGTTGCATTCGTGGC | |
| | TCCCGGTGAATCTCAGCA | |
| 5902 | CTTCAACTTGGCAACAGTTGCCTGGGGTAGCTCTACACA | 38439 |
| | ACTCTGTCCAGCCCACAGCAATGATTCCAGAGGCCATGG | |
| | GGAGTGGACAGCAGCTAGCTGACTGGAGGAATGCCCAC | |
| | TCTCATGGCAACCAGTACAGCACTATCATGCAGCAGCCA | |
| | TCCTTGCTGACTAACCATG | |
| 3399 | CACCCTGACCCAAGCCGAGACAGGTTCCAAACCTCAACC | 38440 |
| | TGCAGCCGGAAGGGGGAAGTGAAACTCGGCTGGGGGTG | |
| | GGGGCTCAGAAGCCGCCCCAGAAAGCACTGAAAGCCAC | |
| | AGCACGTACACCCACTCCAGGGATCTGCCAGCACCCTGT | |
| | GGGGCCCAGACTACAGGCTG | |
| 6560 | GGGACGCCATTGTGATTGGGGGGGGGATCCAGGGCTGCT | 38441 |
| | TCACTGCATACCACCTGGCCAAACACAGGAAGAGGATC | |
| | CTCCTGCTGGAGCAGTTCTTTCTACCACACTCCCGAGGA | |
| | AGCTCCCATGGACAAAGCCGGATAATCCGAAAGGCGTA | |
| | CCTGGAAGACTTTTACACCC | |
| 2890 | CACCGTAGTGCTTAGAGGCCGAAAAAGTACAGCCCCTTC | 38442 |
| | CGGGCTCCGCGGTACGGGAAGACAGCTTTGGGATGTCG | |
| | GAAGATCCTAGAGGTCCCAAGGTACCACGAAAGGCAGT | |
| | GGTAGAAAAGCCAGCTCGGGCAGCAGAGCGAGAGGCCC | |
| | GGGCCCTGCTGGAGAAGAACC | |
| 4343 | ATTGGCTGAGCCCGGCTGTCAGTCCTTTCGCGCCTCGGC | 38443 |
| | GGCGCGGCATAGCCCGGCTCGGCCTGTAAAGCAGTCTCA | |
| | AGCCTGCCGCAGGGAGAAGATGGCGGTCGCCGTGAGAA | |
| | CTTTGCAGGAACAGCTGGAAAAGGCCAAAGAGAGTCTT | |
| | AAGAACGTGGATGAGAACAT | |
| 6138 | ACAAGGAGACTGGGAGGTGTCTCAAGTGCCTGTACCAC | 38444 |
| | ACGGAAGGGGAACACTGTCAGTTCTGCCGGTTTGGATAC | |
| | TATGGTGATGCCCTCCAGCAGGACTGTCGAAAGTGTGTC | |
| | TGTAATTACCTGGGCACCGTGCAAGAGCACTGTAACGGC | |
| | TCTGACTGCCAGTGCGACA | |
| 2241 | GAGGAAGGAGAGAGTGGAGGAGGAGGGCTTTGGGTTAG | 38445 |
| | GGAGAGTGCTTTCGTTTGTTTTAAATGGGAGAAACTGGA | |
| | GCATGTTGCCAAGGGCAGAGAGCCAGCAGAGAGGGGTG | |
| | AATGGAAGAAGGAGCGAGAAGGGGGTTACTGACGAAGC | |
| | CTTATCCTGGAGGAGAGAAGG | |
| 5715 | CAAGTGGTCCTGTCGAAGTATTTATCACTGAGACTCCGA | 38446 |
| | GTCAGCCCAACTCCCACCCCATCCAGTGGAATGCACCAC | |
| | AGCCATCTCACATTTCCAAGTACATTCTCAGGTGGAGAC | |
| | CTAAAAATTCTGTAGGCCGTTGGAAGGAAGCTACCATAC | |
| | CAGGCCACTTAAACTCCT | |
| 3456 | CTAGACCAAAGGGTGCGGCTGCTGCAGAGGTGGCTGAT | 38447 |
| | GCAGGTTTCCATCCTTGGGGAATGACCACGCAACTGGGC | |
| | CCAGCCCTGGTGCTGGGGGTGGCCCTGTGCCTGGGTTGT | |
| | GGCCAGCCCCTACCACAGGTCCCTGAACGCCCCTTCTCT | |
| | GTGCTGTGGAATGTACCCT | |
| 5260 | ATCTCCAGCCATTGCTCCTCCAACAGAGACCATGGCTCC | 38448 |
| | TCCAGTCAGGGATCCTGTTCCTGGGAAACCCACGGTCAC | |
| | CATCCGGACTCGAGGCGCCATTATTCAAACCCCAACCCT | |
| | AGGCCCCATCCAGCCTACTCGGGTGTCAGAAGCTGGCAC | |
| | CACAGTTCCTGGCCAGAT | |
| 1288 | GGAGGCAGGCGGTGCCGCGGCGCCGGGACCCGACTCAT | 38449 |
| | CCGGTGCTTGCGTGTGGTGGTGAGCGCAGCGCCGAGGAT | |
| | GAGGAGGTGCAACAGCGGCTCCGGGCCGCCGCCGTCGC | |
| | TGCTGCTGCTGCTGCTGTGGCTGCTCGCGGTTCCCGGCG | |
| | CTAACGCGGCCCCGCGGTCG | |
| 8756 | CACCCCCACCGACCTCTCGGAGACAGGAATCCGCTCTGC | 38450 |
| | CCCTGCATCCTCCTCTGCTCACCCTTCTCTCAGTAGTGTT | |
| | TGTCAGACACCAGTCCGGACCTCAGCTCTCGATCTAGTT | |
| | CCCGAGTAAGATTTAGCTCCCGGGAAAGCGTGCCTGAAA | |
| | CAAGCCGGAGTGAGCCT | |
| 8952 | CCTGTACCATAACCAGGAGGCTGATGGTTGCTCAGGCCT | 38451 |
| | CCCTCAAACTAGCCAATGAGAAGTGCACGGGTCCTGGTC | |
| | GCCTAGGAAACCGCGTGACAACAAGATGGCGGCGCTGC | |
| | GGGACGGCTAGCGGCCCTGCGTGGGAGTTCCTGACAGCT | |
| | GGATTCTAGAAGTAGAACT | |
| 2759 | TTCCCCGGAAGTGTTGGTTAAAGCCCCTCCAATCAGCGG | 38452 |
| | CTCGGTGCGGCAAGTTTGAATTTCGTGGAGGCTCGGGTT | |
| | GTGAGGGTTCCTGCTTCGGAGTCGGCGGTGGTCGTCCAG | |
| | ACCGAGTGTTCTTTACTTTTTGTTTGGTTGAGGTTTCACG | |
| | CTAGAAGGTGGCTCAGG | |
| 1241 | CTGTCCATACACTCTCTCATCATCCTGTTCCTTGGATTGG | 38453 |
| | ACTTCACTAAGCAATTTATCACTCACCTTCAGACTTACAT | |
| | GTGGGAGTTTTCACAACAGTAGTTTTGGAATCATTAGAA | |
| | CTTGGATTGATTTCATCATTTAACAGAAACAAACAGCCC | |
| | AAATTACTTTATCACC | |
| 28013 | GAGCTCCAGCCTCCAGGCACCCGGGATCCAGCGCCGCCG | 38454 |
| | CTCATAACACCCGCGACCCCGCAGCTAAGCGCAGCTCCC | |
| | GACGCAATGGACCCGGCGCTGGCAGCCCAGATGAGCGA | |
| | GGCTGTGGCCGAGAAGATGCTCCAGTACCGGCGGGACA | |
| | CAGCAGGCTGGAAGATTTGC | |
| 4681 | TGTTGACTCATGCAAATGAGGTATCTGAACTGCAGCTTC | 38455 |
| | AGTATTAGCAGAGCCACAGGCCGCCTCTGTGGCATCACC | |
| | AGGGTTTCTCTGAAGAAGAGGGTCTGCATTTTCCTAAAC | |
| | CCAGTGCTGCTCTCCCATCTCCCATCTTCCTCTCGCAGCT | |
| | TGATGAGCCCCGGTGTG | |
| 5602 | TGCCCCAGCCCTCAGGGCCGCCAGCAAGGAAGAAATTT | 38456 |
| | GTGATACCCCTCGACGAGGATGAGGTCCCTCCTGGAGTG | |
| | GCCAAGCCCTTATTCCGATCTACACAGAGCCTTCCCACT | |
| | GTGGACACCTCGGCCCAGGCGGCCCCTCAGACCTACGCC | |
| | GAATATGCCATCTCACAGC | |
| 1945 | GAGAGGCCGGCGTCTCTCCCCCAGTTTGCCGTTCACCCG | 38457 |
| | GAGCGCTCGGGACTTGCCGATAGTGGTGACGGCGGCAA | |
| | CATGTCTGTGGCTTTCGCGGCCCCGAGGCAGCGAGGCAA | |
| | GGGGGAGATCACTCCCGCTGCGATTCAGAAGATGTTGGA | |
| | TGACAATAACCATCTTATT | |
| 5229 | AACCCCAACCCTAGGCCCCATCCAGCCTACTCGGGTGTC | 38458 |
| | AGAAGCTGGCACCACAGTTCCTGGCCAGATTCGCCCAAC | |
| | GATGACCATTCCTGGCTATGTGGAGCCTACTGCAGTTGC | |
| | TACCCCTCCCACAACCACCACCAAGAAGCCACGAGTATC | |
| | CACACCAAAACCAGCAAC | |
| 7542 | CTGCAGCATCTTCTACGTCGGGACCACAGCCTCCGCCTC | 38459 |
| | CACAAAGCCTGAACCTCCTTTCGCAGGCTCAGCTGCAGG | |
| | CACAGCCTCTTGCGCCAGGCGGAACTCAAATGAAAAAG | |
| | AAAAGTGGCTTCCAGATAACTAGCGTTACTCCTGCTCAG | |
| | ATCTCCGCTAGTATCAGCT | |
| 6866 | CCAATACCTGGGATCCATTGCCTCTCCTTCTGTGCACCCA | 38460 |
| | GCAACGCCCATTTCACCTGGACGTGCCAGCGGCATGACA | |
| | ACCCTCTCTGCAGAACTTTCCAGTCGACTCTCAACGGCA | |
| | CCCGACCTGACAGCGTTCAGCGACCCGCGCCAGTTCCCC | |
| | GCGCTGCCCTCCATCTC | |
| 1849 | TCTCTTGCTTCAACAGTGTTTGGACGGAACAGATCCGGG | 38461 |
| | GACTCTCTTCCAGCCTCCGACCGCCCTCCGATTTCCTCTC | |
| | CGCTTGCAACCTCCGGGACCATCTTCTCGGCCATCTCCTG | |
| | CTTCTGGGACCTGCCAGCACCGTTTTTGTGGTTAGCTCCT | |
| | TCTTGCCAACCAACC | |
| 2558 | AGTTACCTCTCCCCTTTCACGTAGTTTTCATTTGTGGTGA | 38462 |
| | GATTCTCTCCCAGGCCACAAGACATTTCCTGCTCGGAAC | |
| | CTTGTTTACTAATTTCCACTGCTTTTAAGGCCCTGCACTG | |
| | AAAATGCAAGCTCAGGCGCCGGTGGTCGTTGTGACCCAA | |
| | CCTGGAGTCGGTCCCG | |
| 2109 | ATCAGCCCAAAGGTAGGCTCAGGCTCCGACGGTGGCCG | 38463 |
| | GCGGGGGTCACGAGGCTTCGTAGTGGAGGAACGGGTTT | |
| | GGCGTGTGGGACGCAGCTGCCTCTGTACTGGGGAGTCAC | |
| | GGAGTGGCCGGGCTCCAGGGACATGGCGGCGGCCTCTG | |
| | CGGTGTCGGTGCTGCTGGTGG | |
| 297 | ATCCGGAGGCAGCCTGCAGGAAGCCGTAGCGCCGGTAC | 38464 |
| | GTGCCCCTCTCCTGTCTGGAGGCGGGTGTAGAAGTCCGA | |
| | CCGCGGAAGCCAGACTGCTGTCCAGTCGGCGAGCGCGT | |
| | ACCATTCAGCATCGGCTCCGCCCGAGTCCCACCTTCCTC | |
| | AGGCTCTGATTGGCTGACAC | |
| 2621 | CAACAGAAGCCAAGAAGGAAGCCGTCTATCTTGTGGCG | 38465 |
| | ATCATGTATAAGCTGGCCTCCTGCTGTTTGCTTTTCATAG | |
| | GATTCTTAAATCCTCTCTTATCTCTTCCTCTCCTTGACTCC | |
| | AGGGAAATATCCTTTCAACTCTCAGCACCTCATGAAGAC | |
| | GCGCGCTTAACTCCGG | |
| 6574 | TGGCTGACACATTCCTGGAGCACATGTGCCGCCTGGACA | 38466 |
| | TTGATTCACCACCCATCACAGCCCGGAACACTGGCATCA | |
| | TCTGTACCATTGGCCCAGCTTCCCGATCAGTGGAGCTGA | |
| | AGAAGGGAGCCACTCTCAAAATCACGCTGGATAACGCC | |
| | TACATGGAAAAGTGTGACG | |
| 1033 | GACATTTACATCCTGCCCAGCGACAACTCTGGACAAGTC | 38467 |
| | AGTCCCCCAGAGTCTCCAACTGTGACCACTTCCTGGCAG | |
| | TCTGAGAGCTTACCTGTGTCACTGTCAGCTAGCCAGAGT | |
| | TGGCACACAGAAAGCCTGCCAGTGTCACTAGGCCCTGAG | |
| | TCCTGGCAGCAGATTGCA | |
| 8789 | CCTCTGACTTGACCCAATGAAAGAAGCATATGGCACTTG | 38468 |
| | TGAAGATAAATGTTACTCCTCCCTTTTTAATTGGAACTTC | |
| | TGCTTAGGACCTGTGTATGACGTTTCACCTGTGATCTGTT | |
| | CTTTCGGTAGCCACTGACTTTGAGTTACAGGAAGGTCTC | |
| | CGAAGATTTGTGTCAA | |
| 6392 | CTTCCCTGAGGAGGACGACCCCACCAACTGGCTGCGTTG | 38469 |
| | CTACTACTACGAAGACACCATCAGCACCATCAAGTCTGT | |
| | GGCCTGGGAGGGAGGGGCCTGTCCAGCCTTCCTGCCATC | |
| | CCTACGACCACTGCCCCTCACATCACCTTCTCATGGGTCC | |
| | CTCTCCCACTCCAAAGC | |
| 6885 | ATTTATGATTTCAACATGGATACATATTTCAGTTCTTTCT | 38470 |
| | TTTTCTCACTATCTGAAAATACATTTCCCTCCCTCTCTTC | |
| | CCCCCAATATCTCCCTTTTTTTCTCTCTTCCTCTATCTTCC | |
| | AAACCCCACTTTCTCCCTCCTCCTTTTCCTGTGTTCTCTTA | |
| | AGCAGATAGCAC | |
| 561 | CCCTTCCGGCTGGCCCCGCTCAGTCACCCGCAGCAGGCG | 38471 |
| | TGCAGTTTCCCGGCTCTCCGCGCGGCCGGGGAAGGTCAG | |
| | CGCCGTAATGGCGTTCTTGGCGTCGGGACCCTACCTGAC | |
| | CCATCAGCAAAAGGTGTTGCGGCTTTATAAGCGGGCGCT | |
| | ACGCCACCTCGAGTCGTG | |
| 7556 | ATACAAATTTGCCTTTGGCACAACAGATACCACTAAGTT | 38472 |
| | CTACCCAGTTCTCCGCACAATCATTAGCTCAGGCAATTG | |
| | GAAGCCAAATTGAAGATGCCAGGCGTGCAGCGGAGCCC | |
| | TCCTTAGTTGGCTTACCTCAGACTATCAGTGGTGACAGT | |
| | GGGGGAATGTCAGCAGTTT | |
| 8800 | TCCGTCCTCCAGGATCTGGGGAGAAAGAGCCCCATCCCT | 38473 |
| | TCTCTCTCTGCCACCATTTCGGACACCCCGCAGGGACTC | |
| | GTTTTGGGATTCGCACTGACTTCAAGGAAGGACGCGAAC | |
| | CCTTCTCTGACCCCAGCTCGGGCGGCCACCTGTCTTTGCC | |
| | GCGGTGACCCTTCTCTC | |
| 5781 | GTTTACTTCCCCTGCCAGCATTGGGGTGCTCTCTAAGCA | 38474 |
| | ACAGTAGGCGGAGAGTGGTCTGGCGTATTAAAAACAAA | |
| | GGATCGTCAAGTGGGCCTTCCCAGGCATTGCTTTGACTT | |
| | AGTACATGTAGAGGATGTGGCAGTTCTCTCCGTCCCTGC | |
| | CACTGCTGGTTTCTTTGTT | |
| 2930 | TATTTTAAGCCCAGTCTTCCCTGGGCCACCTTTAGCAGAT | 38475 |
| | CCTCGTGCGCCCCCGCCCCCTGGCCGTGAAACTCAGCCT | |
| | CTATCCAGCAGCGACGACAAGTAAAGTGGCCCGCCGGTT | |
| | TCTGAGCCTTCTGCCCTGCGGGGACACGGTCTGCACCCT | |
| | GCCCGCGGCCACGGACC | |
| 3426 | CCAGCGGAAGTAATTCTTTCGACTGCCCCGGAACCCACC | 38476 |
| | GGAGCAGGCAGCTGGGGGTGGGGGGGCGGCCCTGGGAT | |
| | AGGGGCTGTGGCAGTACGCGGGGACCCGGCTGCGGTGG | |
| | CTGCGGGACTGACGAATGGAGAGGAGAAAACCTATGGT | |
| | GGCTGTGAAGGACCTGATGCC | |
| 5800 | ATGGGGAAGGTGAAGGTCGGAGTCAACGGATTTGGTCG | 38477 |
| | TATTGGGCGCCTGGTCACCAGGGCTGCTTTTAACTCTGG | |
| | TAAAGTGGATATTGTTGCCATCAATGACCCCTTCATTGA | |
| | CCTCAACTACATGGTTTACATGTTCCAATATGATTCCACC | |
| | CATGGCAAATTCCATGGC | |
| 27433 | CTGCCTCCCCCCCGCTCCCGCTCCCCTGAGCCCAGCCAG | 38478 |
| | ACCCCGCGCCGCCCGCGCCCCGCTCGACTCCGGAGGCTC | |
| | CCGCAGCCCCGGCGTCCGCCCCGCTGCCCCCTCCCCCGG | |
| | GGGCCATGGGGGCGCCCCCGGGCTACCGGCCCTCAGCTT | |
| | GGGTGCATCTCCTCCACC | |
| 424 | ATTTCTGGGAAGCAGTCGATATTATCTGTACGCCTAGAA | 38479 |
| | CAGTGCCCTCTGCAGCTGAATAACCCTTTTAACGAGTAT | |
| | TCCAAATTTGATGGCAAGGGTCATGTAGGTACAACAGCA | |
| | ACCAAGAAGATCGATGTCTACCTCCCTCTGCACTCGAGC | |
| | CAGGACAGACTGCTGCCA | |
| 28259 | CTCTGGTGATACAGAAGAAAAGACAGTCTCCATTTTCAA | 38480 |
| | ACAGTCCCTCCTGGGAGAACACAGACAGGCAGAGGATT | |
| | ACAACACAAGGCAGCAAGCACTGGGAGACGAAAGTTTT | |
| | GGCATCTGTTCCCTGGCTGTGCCAAGATGGGCGATTGGA | |
| | GCTTCCTGGGAAATTTCCTG | |
| 6972 | GTCTACTATGTCTGAACTGTGGTTTCTTGTTTATCCTTTTT | 38481 |
| | TCCTTAGTTGGACTGTAATGTATGGTCTGTCAACCTGTGA | |
| | ATCTTTAAAGTATGATTCAGGTATTGTTGTATTCTTTACT | |
| | GTGTAATAAAAAAGTTGAAAAAAATCTGGATCCTCTGTC | |
| | TCCCTCGTCCCCGG | |
| 417 | TTTCGTGACCTTGTCCAGTAGAAGGCTATTTAATTTTCAC | 38482 |
| | AACTGCTTGAATTTTGACATACAAGATGAAGCAAGATGC | |
| | CTCAAGAAATGCTGCCTACACTGTGGATTGTGAAGATTA | |
| | TGTGCATGTGGTAGAATTTAATCCCTTTGAGAATGGGGA | |
| | TTCAGGAAACCTAATTG | |
| 8182 | TGTCTGCACCAGCCGGCCTCCAGCCTGGCTGGACCCTGC | 38483 |
| | TGCCTGTGTGGCCCGGAGCCAGAGGCCCCCACACTCCCA | |
| | GCTGCTCTTCTACAGATGCCATCAACGAGCAGGACTCTG | |
| | GGTGGCTCCACTGTCTAAGGGCTCTGCGAGTGACCCGGC | |
| | GGGCGAGCTCCGTGCTGC | |
| 7572 | CTTACGTAGTAATTACATTTCCTTGAAAAAACTATAGTG | 38484 |
| | AATAGAAATCCCTAGCCATTTCATTTTTTATGTTTTTAAT | |
| | GAAGATCTTTAAAATACCATAGGTGGTAATCGTGGAAAA | |
| | TTTGAAAAATCTCATGTCAGTGTATTAAGATGGTGGAGA | |
| | AGTTTTTTTCTCCATTA | |
| 1415 | ATTCATGAAAATCCACTACTCCAGACAGACGGCTTTGGA | 38485 |
| | ATCCACCAGCTACATCCAGCTCCCTGAGGCAGAGTTGAG | |
| | AATGGAGAGAATGTTACCTCTCCTGGCTCTGGGGCTCTT | |
| | GGCGGCTGGGTTCTGCCCTGCTGTCCTCTGCCACCCTAA | |
| | CAGCCCACTTGACGAGGA | |
| 7327 | GAGCTTCCAGGGATATTTGAGGCACCATCCCTGCCATTG | 38486 |
| | CCGGGCACTCGCGGCGCTGCTAACGGCCTGGTCACATGC | |
| | TCTCCGGAGAGCTACGGGAGGGCGCTGGGTAACCTCTAT | |
| | CCGAGCCGCGGCCGCGAGGAGGAGGGAAAAGGCGAGC | |
| | AAAAAGGAAGAGTGGGAGGA | |
| 705 | GCCCCCTCCCCTGACTATCAAAGCAGCGGCCGGCTGTTG | 38487 |
| | GGGTCCACCACGCCTTCCACCTGCCCCACTGCTTCTTCGC | |
| | TTCTCTCTTGGAAAGTCCAGTCTCTCCTCGGCTTGCAATG | |
| | GACCCCAACTGCTCCTGCGCCGCTGGTGTCTCCTGCACC | |
| | TGCGCTGGTTCCTGCA | |
| 8561 | AGTCGCATAGTGGTTTTTCCGCTCGCGTCGCTGTGTGAA | 38488 |
| | AGTTGGCTCGCCGCTCTTTGCACGCCCTCCCTGGAGGCC | |
| | GACCCGAGACGCCAAGCTGGAGAGACCGTGCCTCCCCG | |
| | AGGCCGGCCGCCCCGCGAGCACAGCCTCCGCCCCCGTTG | |
| | CACTGCCGGGCTGGGCAAT | |
| 1679 | GGTGGCGGGAAGAGGAGGCGCGAGAATGGAGGTGGAG | 38489 |
| | GCCGTCTGTGGTGGCGCGGGCGAGGTGGAGGCCCAGGA | |
| | CTCTGACCCTGCCCCTGCCTTCAGCAAGGCCCCCGGCAG | |
| | CGCCGGCCACTACGAACTGCCGTGGGTTGAAAAATATAG | |
| | GCCAGTAAAGCTGAATGAAAT | |
| 5635 | TATTTCCCTCTACTGAGTAAACCCTATCTGTGATTCCCCC | 38490 |
| | AAACATCTGGCATGGCTCCCTTTTGTCCTTCCTGTGCCCT | |
| | GCAAATATTAGCAAAGAAGCTTCATGCCAGGTTAGGAA | |
| | GGCAGCATTCCATGACCAGAAACAGGGACAAAGAAATC | |
| | CCCCCTTCAGAACAGAGG | |
| 5583 | TGTTACTTAAGAATGCTTTCCAGGTGGAAAGTTCCTTAA | 38491 |
| | GTTTGAGGCTTCAAATTCCATACAGCACATTAAAATCCC | |
| | ATTCATGAGTTTGAAATACTGCTCTGTTGTCTTGGAAATA | |
| | CCAATCAGATTGTTGGCTGAAGTGATGTGGATAAAGAAG | |
| | GGATCTTAGAAAAACTA | |
| 9027 | ACGAAGTTCCGGAAATGGAATTACCGAGTCAGATGAGA | 38492 |
| | AACACAAGTCATCAATGAATTAACAAGCAACACATTGTC | |
| | ACATCCAGCTGTTGAAGAGTGGTTAACTCAGCTTGATCT | |
| | CCGAAGACTCTTCTGCACCCCACCCCGCCCCCAACTGCT | |
| | TTCCAAAGAGAAACAGAAA | |

The IRES may be of any length or size. For example, the IRES may be about 100 nucleotides to about 600 nucleotides in length (e.g., about 200, about 225, about 250, about 275, about 300, about 325, about 350, about 375, about 400, about 425, about 450, about 475, about 500, about 525, about 550, or about 575 nucleotides in length, or a range defined by any two of the foregoing values). In some embodiments, the IRES may be about 200 nucleotides to about 800 nucleotides in length (about 200, about 210, about 220, about 240, about 260, about 280, about 320, about 340, about 360, about 380, about 420, about 440, about 460, about 480, about 500, about 520, about 540, about 560, about 580, about 600, about 620, about 640, about 660, about 680, about 700, about 720, about 740, about 760, about 780, or about 800 nucleotides in length, or a range defined by any two of the foregoing values). In some embodiments, the IRES may be about 200 to about 400, about 400 to about 600, about 600 to about 700, or about 600 to about 800 nucleotides in length. In some embodiments, the IRES is about 210 nucleotides in length. In some embodiments, the IRES may be about 100 to about 3000 nucleotides in length.

In some embodiments, a circular RNA molecule comprises of an IRES sequence that consists of a sequence encoded by a DNA sequence from Table 1A or Table 1B. In some embodiments, a circular RNA molecule comprises an IRES sequence encoded by a DNA sequence from Table 1A or Table 1B, wherein the IRES sequence additionally comprises up to 1000 additional nucleotides. In some embodiments, the IRES sequence is encoded by a sequence from Table 1A or 1B and additionally comprises up to 1000 additional nucleotides located at the 5′ end of that sequence. In some embodiments, the IRES sequence is encoded by a sequence from Table 1A or 1B and additionally comprises up to 1000 additional nucleotides located at the 3′ end of that sequence. In some embodiments, the IRES sequence is encoded by a sequence from Table 1A or 1B and additionally comprises up to 1000 additional nucleotides located at the 5′ end of that sequence and up to 1000 additional nucleotides located at the 5′ end of that sequence.

In some embodiments, a circular RNA molecule comprises an internal ribosome entry site (IRES) sequence region, wherein the IRES sequence region comprises a sequence encoded by a DNA sequence from Table 1A or Table 1B, and wherein the sequence encoded by a DNA sequence from Table 1A or Table 1B has a minimum free energy (MFE) of less than −18.9 kJ/mol and a melting temperature of at least 35.0° C.

In some embodiments, a circular RNA molecule comprises an internal ribosome entry site (IRES) sequence region, wherein the IRES sequence region comprises a sequence encoded by a DNA sequence from Table 1A or Table 1B, and wherein the IRES sequence region has a minimum free energy (MFE) of less than −18.9 kJ/mol and a melting temperature of at least 35.0° C., over its entire length.

In some embodiments, a circular RNA molecule comprises an internal ribosome entry site (IRES) sequence region, wherein the IRES sequence region comprises a sequence encoded by a DNA sequence from Table TA or Table 1B, and additionally comprises up to 1000 additional nucleotides located at the 5′ end of and up to 1000 additional nucleotides located at the 5′ end, and wherein the IRES sequence region has a minimum free energy (MFE) of less than −18.9 kJ/mol and a melting temperature of at least 35.0° C., over its entire length. In some embodiments, the recombinant circular RNA molecule comprises a protein-coding nucleic acid sequence operably linked to the IRES, optionally in a non-native configuration. Any protein or polypeptide of interest (e.g., a peptide, polypeptide, protein fragment, protein complex, fusion protein, recombinant protein, phosphoprotein, glycoprotein, or lipoprotein) may be encoded by the protein-coding nucleic acid sequence. In some embodiments, the protein coding-nucleic acid sequence encodes a therapeutic protein. Examples of suitable therapeutic proteins include cytokines, toxins, tumor suppressor proteins, growth factors, hormones, receptors, mitogens, immunoglobulins, neuropeptides, neurotransmitters, and enzymes. Alternatively, the protein-coding nucleic acid sequence can encode an antigen of a pathogen (e.g., a bacterium, virus, fungus, protist, or parasite), and the circRNA can be used as, or as one component of, a vaccine. Therapeutic proteins, and examples thereof, are further described in, e.g., Dimitrov, D. S., Methods Mol Biol., 899: 1-26 (2012); and Lagasse et al., F1000Research, 6: 113 (2017).

Ideally, the IRES is “in-frame” with respect to the protein-coding nucleic acid sequence, that is, the IRES is positioned in the circRNA molecule in the correct reading frame for the encoded protein. Examples of IRES elements that were found to be in-frame with one or more coding sequences are set forth in Table 4. In some embodiments, however, the TRES may be “out of frame” with respect to the protein-coding nucleic acid sequence, such that the position of the IRES disrupts the ORF of the protein-coding nucleic acid sequence. In other embodiments, the IRES may overlap with one or more ORFs of the protein-coding nucleic acid sequence. In addition, while in some embodiments the protein-coding nucleic acid sequence comprises at least one stop codon, in other embodiments the protein-coding nucleic acid sequence may lack a stop codon. The instant inventors have found that a circRNA molecule comprising a protein-coding nucleic acid sequence having an in frame non-native IRES and lacking a stop codon can initiate a recursive (i.e., infinite loop) translation mechanism. Such recursive translation may produce a concatenated protein multimer (e.g., >200 kDa). This particular circRNA design allows for the production of repeating ORF units up to 10 times the size of the single ORF. Without being bound to any particular theory, use of the circRNAs described herein for recursive gene encoding may represent a novel “data compression” algorithm for genes, addressing the gene size limitation associated with many current gene therapy applications.

In some embodiments, the IRES comprises (i) at least one RNA secondary structure element and (ii) a sequence that is complementary to an 18S rRNA. In some embodiments, the IRES comprises (i) at least one RNA secondary structure element and (ii) a sequence that is complementary to an 18S rRNA, wherein the RNA secondary structure of the IRES is formed from the nucleotides at about position 40 to about position 60 of the IRES, wherein the first nucleic acid at the 5′ end of the IRES is considered to be position 1. The relative location of the at least one RNA secondary structure and the sequence that is complementary to an 18S RNA may vary. For example, in some embodiments, the IRES comprises (i) at least one RNA secondary structure element and (ii) a sequence that is complementary to an 18S rRNA, and wherein the at least one RNA secondary structure is located 5′ to the sequence that is complementary to an 18S rRNA. In some embodiments, the IRES comprises (i) at least one RNA secondary structure element and (ii) a sequence that is complementary to an 18S rRNA, and wherein the at least one RNA secondary structure element is located 3′ to the sequence that is complementary to an 18S rRNA).

In some embodiments, the circular RNA may comprise one or more IRES RNA control elements. These elements may, in come embodiments, act as a conditional “off” switch. For example, the IRES RNA control element may be a miRNA binding site. miRNA binding to the circRNA may lead to degradation of the circRNA, destroying its activity.

### DNA Molecules and Host Cells

In some embodiments, the disclosure provides a DNA molecule comprising a nucleic acid sequence encoding any one of the recombinant circRNA molecules disclosed herein. Accordingly, described herein are DNA sequences that may be used to encode circular RNAs. In some embodiments, a DNA sequence encodes a circular RNA comprising an IRES. In some embodiments, a DNA sequence encodes a circular RNA comprising a protein-coding nucleic acid. In some embodiments, the DNA sequence encodes a circular RNA molecule; wherein the circular RNA molecule comprises a protein-coding nucleic acid sequence and an internal ribosome entry site (IRES) operably linked to the protein-coding nucleic acid sequence in a non-native configuration. In some embodiments, the DNA sequence encodes a protein coding-nucleic acid sequence, wherein the protein is a therapeutic protein.

The DNA sequences disclosed herein may, in some embodiments, comprise at least one non-coding functional sequence. For example, the non-coding functional sequence may be a microRNA (miRNA) sponge. A microRNA sponge may comprise a complementary binding site to a miRNA of interest. In some embodiments, a sponge's binding sites are specific to the miRNA seed region, which allows them to block a whole family of related miRNAs. In some embodiments, the miRNA sponge is selected from any one of the miRNA sponges shown in the table below.

| miRNA | Binding sites | Cell context |
|---|---|---|
| 92, empty | four bulged | rat cerebellar neuronal cultures |
| | sites | |
| miR-155, empty | eight bulged | B cell lymphoma cell line |
| | sites | |
| 15a, empty | two perfect | human prostate cell lines, xenograft |
| | sites | |
| 113, empty | two perfect | mouse cardiac myocytes, heart |
| | sites | |
| 326, empty | seven bulged | tail vein injection, TH cells |
| | sites | |
| 16, 20, 21, | four to nine | mammalian cell lines |
| 30-5p, CXCR4 | bulged sites | |
| 16, 20, CXCR4 | four to nine | mammalian cell lines |
| | bulged sites | |
| let-7, 22, 124, 125, | five to seven | rat hippocampal neuronal cultures |
| 132, 143, empty | bulged sties | |
| 183, CXCR4 | seven bulged | HEK293T cell line |
| | sites | |
| A.t. 156, 319, | one bulged site | transgenic Arabidopsis plants |
| empty | | |
| 16, CXCR4 | seven to nine | human multiple myeloma cell |
| | bulged sites | lines, xenograft |
| 16, 23a, 142-3p. | four perfect or | U937 cell line, mouse hematopoietic |
| 221, 223, | four | stem/progenitor cells, transplant |
| nonspecific | bulged sites | |
| KSHV miR-K1, | nine bulged | BC-3 B cell lymphoma cell line |
| CXCR4, empty | sites | |
| 21, 140-3p, | one to two | mammalian cell lines |
| 140-5p, empty | perfect sites | |
| 133, empty | three perfect | mouse cardiac myocytes |
| | sites | |
| 204, 211, empty | two perfect | ST2 cell line, osteogenic and |
| | sites | adipogenic differentiation |
| 96/182/183, empty | four bulged | mouse retina |
| | sites each | |
| let-7, 16, empty | six to nine | lung cancer cell line |
| | bulged sites | |
| D.m. 7, 8, 9a, 276a | 10 bulged sites | Transgenic fruitflies |
| 9, 10b, CXCR4 | eight bulged | 4T1 breast cancer cell line, xenograft |
| | sites | |
| herpesvirus miR-K12-7, K12-8, | six bulged sites | human B cell lines |
| BARTI-5p, BART4, empty | | |
| 144, 451, empty | four perfect | mouse hematopoietic stem/progenitor |
| | sites | cells, transplant |
| let-7, 29, 125, | 16 bulged sites | neural stem cells |
| let-7/125, | | |
| 128, empty | | |
| 21, empty | two perfect | mouse cardiocytes |
| | sites | |
| 18a, 19b, 20a, | one perfect site | K562 cell line |
| nonspecific | | |
| 145/146, empty | eight to nine | mouse hematopoietic stem/progenitor |
| | bulged | cells, transplant |
| | sites each | |
| 31, CXCR4 | seven bulged | MCF7-Ras breast cancer cell line, |
| | sites | xenograft |

In some embodiments, the non-coding sequence may be an RNA binding protein site. RNA binding proteins and binding sites therefore are listed in numerous databases known to those of skill in the art, including RBPDB (rbpdb.ccbr.utoronto.ca). In some embodiments, the RNA binding protein comprises one or more RNA-binding domains, selected from RNA-binding domain (RBD, also known as RNP domain and RNA recognition motif, RRM), K-homology (KH) domain (type I and type II), RGG (Arg-Gly-Gly) box, Sm domain; DEAD/DEAH box, zinc finger (ZnF, mostly C-x8-X-x5-X-x3-H), double stranded RNA-binding domain (dsRBD), cold-shock domain; Pumilio/FBF (PUF or Pum-HD) domain, and the Piwi/Argonaute/Zwille (PAZ) domain.

In some embodiments, the DNA sequence comprises an aptamer. Aptamers are short, single-stranded DNA molecules that can selectively bind to a specific target. The target may be, for example, a protein, peptide, carbohydrate, small molecule, toxin, or a live cell. Some aptamers can bind DNA, RNA, self-aptamers or other non-self aptamers. Aptamers assume a variety of shapes due to their tendency to form helices and single-stranded loops. Illustrative DNA and RNA aptamers are listed in the Aptamer database (scicrunch.org/resources/Any/record/nlx_144509-1/SCR_001781/resolver?q=*&l=). In some embodiments, the DNA sequence encodes a circular RNA molecule that comprises between about 200 nucleotides and about 10,000 nucleotides.

In some embodiments, the DNA sequence encodes a circular RNA molecule that comprises a spacer between the IRES and a start codon of the protein-coding nucleic acid sequence or at another location (e.g., upstream of the IRES). The spacer may be of any length (e.g., 10 to 100 nucleotide, 10 to 90 nucleotides, 10 to 80 nucleotides, 10 to 70 nucleotides, 10 to 60 nucleotides, 10 to 50 nucleotides, 10 to 40 nucleotides, 10 to 30 nucleotides, 10 to 20 nucleotides, 20 to 100 nucleotides, 20 to 90 nucleotides, 20 to 80 nucleotides, 20 to 70 nucleotides, 20 to 60 nucleotides, 20 to 50 nucleotides, 20 to 40 nucleotides, 20 to 30 nucleotides, 30 to 100 nucleotides, 30 to 90 nucleotides, 30 to 80 nucleotides, 30 to 70 nucleotides, 30 to 60 nucleotides, 30 to 50 nucleotides, 30 to 40 nucleotides, 40 to 100 nucleotides, 40 to 90 nucleotides, 40 to 80 nucleotides, 40 to 70 nucleotides, 40 to 60 nucleotides, 40 to 50 nucleotides, 50 to 100 nucleotides, 50 to 90 nucleotides, 50 to 80 nucleotides, 50 to 70 nucleotides, 50 to 60 nucleotides, 60 to 100 nucleotides, 60 to 90 nucleotides, 60 to 80 nucleotides, 60 to 70 nucleotides, or 50 nucleotides). For example, in some embodiments, the length of the spacer is selected to optimize translation of the protein-coding nucleic acid sequence.

In some embodiments, the DNA sequence encodes a circular RNA molecule comprising an IRES that is configured to promote rolling circle translation. In some embodiments, the DNA sequence encodes a circular RNA comprising a protein-coding nucleic acid sequence that lacks a stop codon. In some embodiments, the DNA sequence encodes a circular RNA molecule comprising (i) an IRES that is configured to promote rolling circle translation, and (ii) a protein-coding nucleic acid sequence that lacks a stop codon.

The DNA sequences described herein may be comprised in one or more vectors. For example, in some embodiments, a viral vector comprises a DNA sequence encoding a circular RNA. The viral vector may be, for example, an adeno-associated virus (AAV) vector, an adenovirus vector, a retrovirus vector, a lentivirus vector, a vaccinia or a herpesvirus vector. In some embodiments, the viral vector is an AAV. As used herein, the term “adeno-associated virus” (AAV), includes but is not limited to, AAV1, AAV2, AAV3 (including types 3 A and 3B), AAV4, AAV5, AAV6, AAV7, AAV8, AAV9, AAV10, AAV11, AAV12, avian AAV, bovine AAV, canine AAV, equine AAV, ovine AAV, and any other AAV now known or later discovered. In some embodiments, the AAV vector may be a modified form (i.e., a form comprising one or more amino acid modifications relative thereto) of one or more of AAV1, AAV2, AAV3 (including types 3 A and 3B), AAV4, AAV5, AAV6, AAV7, AAV8, AAV9. AAV10, AAV11, AAV12, avian AAV, bovine AAV, canine AAV, equine AAV, or ovine AAV. Various AAV serotypes and variants thereof are described, e.g., BERNARD N. FIELDS et al, VIROLOGY, volume 2. chapter 69 (4th ed., Lippincott-Raven Publishers). A number of relatively new AAV serotypes and clades have been identified (see, e.g., Gao et al, (2004) J Virology 78:6381-6388; Moris et al. (2004) Virology 33-:375-383). The genomic sequences of various serotypes of AAV, as well as the sequences of the native terminal repeats (TRs), Rep proteins, and capsid subunits are known in the art. Such sequences may be found in the literature or in public databases such as the GenBank© Database. See, e.g. , GenBank Accession Numbers NC 044927, NC 002077, NC_001401, NC_001729, NC 001863, NC 001829, NC_001862, NC_000883, NC 001701, NC 001510, NC_006152, NC 006261, AF063497, U89790, AF043303, AF028705, AF028704, J02275, JO 1901, J02275, X01457, AF288061, AH009962, AY028226, AY028223, NC 001358, NC 001540, AF513851, AF513852, AY530579; the disclosures of which are incorporated by reference herein for teaching parvovirus and AAV nucleic acid and amino acid sequences. See also, e.g., Srivistava et al. (1983) J Virology 45:555; Chiorini et al. (1998) J. Virology 71:6823; Chiorini et al (1999) J Virology 73:1309; Bantel-Schaal et al. (1999) J. Virology 73:939; Xiao et al. (1999) J. Virology 73:3994; Muramatsu et al. (1996) Virology 221:208; Shade et al. (1986) J Virol. 58:921; Gao et al. (2002) Proc. Nat. Acad. Sci. USA 99: 1 1854; Moris et al. (2004) Virology 33-:375-383; international patent publications WO 00/28061, WO 99/61601, WO 98/11244; and U.S. Pat. No. 6,156,303; the disclosures of which are incorporated by reference herein.

In some embodiments, a DNA sequence described herein is comprised in an AAV2 vector, or a variant thereof. In some embodiments, a DNA sequence described herein is comprised an AAV4 vector, or a variant thereof. In some embodiments, a DNA sequence described herein is comprised in an AAV8 vector, or a variant thereof. In some embodiments, a DNA sequence described herein is comprised in an AAV9 vector, or a variant thereof. In some embodiments, a DNA sequence described herein is comprised in a viral-like particle (VLP). Viral like particles are molecules that closely resemble viruses, but are non-infectious because they contain little or no viral genetic material. They can be naturally occurring or synthesized through the individual expression of viral structural proteins, which can then self-assemble into a virus-lie structure. Combinations of structural capsid proteins from different viruses can be used to create VLPs. For example VLPs may be derived from the, AAVs, retrovirus, Flaviviridae, paramyoxoviridae, or bacteriophages. VLPs can be produced in multiple cell culture systems, including bacteria, mammalian cell lines, insect cell lines, yeast and plant cells.

In some embodiments, a DNA sequence described herein is comprised in a non-viral vector. The non-viral vector may be, for example, a plasmid comprises the DNA sequence. In some embodiments, the non-viral vector is a closed-ended DNA. A closed-ended DNA is a non-viral, capsid-free DNA vector with covalently closed ends (see, e.g., WO2019/169233). In some embodiments, a mini-intronic plasmid vector comprises a DNA sequence described herein. Mini-intronic plasmids are expression systems that contain a bacterial replication origin and selectable marker maintaining the juxtaposition of the 5′ and the 3′ ends of transgene expression cassette as in a minicircle (see, e.g., Lu, J., et al., Mol Ther (2013) 21(5) 954-963).

In some embodiments, a DNA sequence described herein is comprised in a lipid nanoparticle. Lipid nanoparticles (or LNPs) are submicron-sized lipid emulsions, and may offer one or more of the following advantages: (i) control and/or targeted drug release, (ii) high stability, (iii) biodegradability of the lipids used, (iv) avoid organic solvents, (v) easy to scale-up and sterilize, (vi) less expensive than polymeric/surfactant based carriers, (vii) easier to validate and gain regulatory approval. In some embodiments, the lipid nanoparticles range in diameter between about 10 and about 1000 nm.

In some embodiments, a DNA sequence encodes a circular RNA molecule, wherein the circular RNA molecule comprises a protein-coding nucleic acid sequence and an internal ribosome entry site (IRES) operably linked to the protein-coding nucleic acid sequence in a non-native configuration wherein the IRES comprises: at least one RNA secondary structure; and a sequence that is complementary to an 18S ribosomal RNA (rRNA).

In some embodiments, a DNA sequence encodes a circular RNA molecule, wherein the circular RNA molecule comprises a protein-coding nucleic acid sequence and an internal ribosome entry site (IRES) operably linked to the protein-coding nucleic acid sequence in a non-native configuration wherein the IRES comprises: at least one RNA secondary structure element; and a sequence that is complementary to an 18S ribosomal RNA (rRNA); wherein the IRES has a minimum free energy (MFE) of less than −18.9 kJ/mol and a melting temperature of at least 35.0° C.; and wherein the RNA secondary structure element is formed from the nucleotides at about position 40 to about position 60 of the IRES, wherein the first nucleic acid at the 5′ end of the IRES is considered to be position 1.

In some embodiments, a DNA sequence comprises a nucleic acid sequence encoding a circular RNA molecule; wherein the circular RNA molecule comprises a protein-coding nucleic acid sequence and an internal ribosome entry site (IRES) operably linked to the protein-coding nucleic acid sequence in a non-native configuration; wherein the IRES is encoded by any one of the nucleic acid sequences listed in Table 1A or Table 1B, or a nucleic acid sequence that is at least 90% or at least 95% identical thereto.

Also provided herein are cells comprising a recombinant circRNA molecule, a DNA molecule, or a vector described herein. Any prokaryotic or eukaryotic cell that can be contacted with and stably maintain the recombinant circRNA molecule, DNA molecule encoding the recombinant circRNA molecule, or vector comprising the recombinant circRNA molecule may be used in the context of the present disclosure. Examples of prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. In some embodiments, the host cell is a eukaryotic cell. Suitable eukaryotic cells are known in the art and include, for example, yeast cells, insect cells, and mammalian cells. Examples of yeast cells include those from the genera Hansenula, Kluyveromyces, Pichia, Rhinosporidium, Saccharomyces, and Schizosaccharomyces. Suitable insect cells include Sf-9 and HIS cells (Invitrogen, Carlsbad, Calif.) and are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993).

In some embodiments, the cell is a mammalian cell. A number of mammalian cells are known in the art, many of which are available from the American Type Culture Collection (ATCC, Manassas, Va.). Examples of mammalian cells include, but are not limited to, HeLa cells, HepG2 cells, Chinese hamster ovary cells (CHO) (e.g., ATCC No. CCL61), CHO DHFR-cells (Urlaub et al., Proc. Natl. Acad. Sci. USA, 97: 4216-4220 (1980)), human embryonic kidney (HEK) 293 or 293T cells (e.g., ATCC No. CRL1573), and 3T3 cells (e.g., ATCC No. CCL92). Other mammalian cell lines are the monkey COS-1 (e.g., ATCC No. CRL1650) and COS-7 cell lines (e.g., ATCC No. CRL1651), as well as the CV-1 cell line (e.g., ATCC No. CCL70). Further exemplary mammalian host cells include primate cell lines and rodent cell lines, including transformed cell lines. Normal diploid cells, cell strains derived from in vitro culture of primary tissue, as well as primary explants also are suitable. Other mammalian cell lines include, but are not limited to, mouse neuroblastoma N2A cells, HeLa, mouse L-929 cells, and BHK or HaK hamster cell lines, all of which are available from the American Type Culture Collection (ATCC; Manassas, VA). Methods for selecting mammalian cells and methods for transformation, culture, amplification, screening, and purification of such cells are well known in the art (see, e.g., Ausubel et al., supra). In some embodiments, the mammalian cell is a human cell.

### Method of Producing a Protein

The disclosure further provides a method of producing a protein in a cell, which comprises contacting a cell with the above-described recombinant circular RNA molecule, the above-described DNA molecule comprising a nucleic acid sequence encoding the recombinant circRNA molecule, or a vector comprising the recombinant circRNA molecule under conditions whereby the protein-coding nucleic acid sequence is translated and the protein is produced in the cell.

In some embodiments, a method of producing a protein in a cell comprises contacting a cell with a DNA sequence described herein, or a vector comprising the DNA sequence, under conditions whereby the protein-coding nucleic acid sequence is translated and the protein is produced in the cell. Also provided is a protein produced by the disclosed methods.

In some embodiments, production of the protein is tissue-specific. For example, the protein may be selectively produced in one or more of the following tissues: muscle, liver, kidney, brain, lung, skin, pancreas, blood, or heart.

In some embodiments, the protein is expressed recursively in the cell.

In some embodiments, the half-life of the circular RNA in the cell is about 1 to about 7 days. For example, the half-life of the circular RNA may be about 1, about 2, about 3, about 4, about 5, about 6, about 7, or more days.

In some embodiments, the protein is produced in the cell for at least about 10%, at least about 20%, or at least about 30% longer than if the protein-coding nucleic acid sequence is provided to the cell using a viral vector encoding a linear RNA or as a linear RNA. In some embodiments, the protein is produced in the cell at a level that is at least about 10%, at least about 20%, or at least about 30% higher than if the protein-coding nucleic acid sequence is provided to the cell using a viral vector or as a linear RNA.

Use of the IRES sequences described herein to express a protein from a circular RNA may, in some embodiments, allow for continued expression of a protein from the circular RNA in a cell even under stress conditions. In response to one or more stress conditions, production of proteins from linear RNA is often suppressed. Accordingly, in some embodiments, circRNA can be used as an alternative for production of proteins from linear RNAs during stress conditions. In some embodiments, a protein expressed from a circular RNA in a cell is expressed under one or more stress conditions. In some embodiments, expression of a protein from a circular RNA in a cell is not substantially disrupted when the cell is exposed to one or more stress conditions. For example, exposure of the cell to one or more stress conditions may change expression of a protein from a circular RNA by less than 15%, less than 10%, less than 5%, less than 3%, less than 1%, or less than 0.5%. In some embodiments, a protein expressed from a circular RNA is expressed at a level under one or more stress conditions that is substantially the same as the level expressed in the same cell in the absence of the one or more stress conditions. In some embodiments, the level of expression of a protein from a circular RNA in a cell is at least about 70%, at least about 75%, at least about 80%, at least about 85%, at least about 90%, at least about 95%, or at least about 99%, relative to the level of expression in the absence of the one or more stress conditions. A non-limiting list of conditions which may cause cellular stress include changes in temperature (including exposure to extreme temperatures and/or heat shock), exposure to toxins (including viral or bacterial toxins, heavy metals, etc.), exposure to electromagnetic radiation, mechanical damage, viral infection, etc.

In some embodiments, the circRNAs described herein (including components thereof, such as the IRES sequences) facilitate cap-independent translation activity from the circRNA. Canonical translation via a cap-independent mechanism may be reduced in some human diseases. Accordingly, the use of circRNAs to express proteins may be particularly helpful for treating such diseases. In some embodiments, use of the circRNAs described herein facilitates cap-independent translation activity from the circRNA under conditions wherein cap-dependent translation is reduced or turned-off in a cell.

As discussed above, translation of the protein-coding nucleic acid sequence may occur in an infinite loop (i.e., recursively) when the IRES is in-frame with the protein-coding nucleic acid sequence and the protein-coding sequence lacks a stop codon. Thus, in some embodiments, the method of producing a protein in a cell produces a concatenated protein. Any prokaryotic or eukaryotic host cell described herein may be contacted with the recombinant circRNA molecule or a vector comprising the circRNA molecule. The host cell may be a mammalian cell, such as a human cell. In some embodiments, the cell is in vivo. In some embodiments, the cell is in vitro. In some embodiments, the cell is ex vivo. In some embodiments, the cell is in a mammal, such as a human.

In some embodiments, regardless of cell type chosen, 5′ cap-dependent translation is impaired in the cell (e.g., decreased, reduced, inhibited, or completely obliterated). In some embodiments, there is no substantial 5′ cap-dependent translation in the cell.

The circRNAs described herein may also be produced in vitro, such as by in vitro transcription or other cell-free transcription system. Typical in vitro transcription protocols comprise providing (i) a purified DNA template, wherein the DNA template encodes a circular RNA, (ii) ribonucleotide triphosphates, (iii) a buffer system that includes DTT and magnesium ions, and (iv) an appropriate phage RNA polymerase. The DNA template may comprise, for example, a plasmid construct engineered by cloning, a cDNA template generated by first- and second-strand synthesis from an RNA precursor (e.g., aRNA amplification), or a linear template generated by PCR or by annealing chemically synthesized oligonucleotides. These components are then combined, and incubated under conditions which allows the RNA polymerase to transcribe the DNA to RNA, typically a linear RNA. Commercial kits are available for performing in vitro transcription, such as the Invitrogen MAXIscript® or MEGAscript® kits. In some embodiments, a polyA tail may be added to an RNA produced using in vitro transcription. Linear RNAs produced in vitro may be circularized using one or more of the following exemplary methods. For example, linear RNAs produced in vitro may be circularized according to chemical methods, using a condensing agent such as cyanogen bromide. In some embodiments, linear RNAs produced in vitro may be circularized using an enzymatic method. For example, the linear RNAs may be circularized using RNA or DNA ligases (e.g., T4 RNA ligase I or II). Alternatively, the linear RNAs may be circularized using ribozymatic methods, such as methods which employ self-splicing introns.

In some embodiments, a protein is produced from a circular RNA in a cell free system. The cell-free system may comprise, for example, all factors required for transcribing circular RNA from DNA, circularizing the RNA, and translating the protein from therefrom. In some embodiments, the circular RNA is more stable than a linear RNA in a cell-free system, which allows for increased expression of a protein from the circular RNA.

In some embodiments, a method for producing a protein comprises contacting a circular RNA with a cell-free extract comprising protein translation initiation factors (e.g., eIF1, eIF2, eIF3, eIF5, eIF6), under conditions wherein the protein is expressed. In some embodiments, a method for producing a protein comprises: (i) providing a linear RNA encoding a protein of interest, (ii) circularizing the RNA, (iii) contacting the circular RNA with a cell-free extract comprising protein translation initiation factors, under conditions wherein the protein is expressed.

In some embodiments, a method for producing a protein comprises contacting a linear RNA with a cell-free extract comprising protein translation initiation factors, under conditions wherein the RNA is circularized and the protein is expressed. In some embodiments, the linear RNA may comprise self-splicing introns.

In some embodiments, a method for producing a protein comprises contacting a DNA with a cell-free extract comprising protein translation initiation factors, under conditions wherein a linear RNA is expressed, the linear RNA is circularized, and the protein is expressed. In some embodiments, the DNA may encode may comprise self-splicing introns.

The recombinant circular RNA molecule, a DNA molecule encoding same, or vectors comprising same, may be introduced into a cell by any method, including, for example, by transfection, transformation, or transduction. The terms “transfection,” “transformation,” and “transduction” are used interchangeably herein and refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); strontium phosphate DNA co-precipitation (Brash et al., Mol. Cell. Biol., 7: 2031-2034 (1987); and magnetic nanoparticle-based gene delivery (Dobson, J., Gene Ther, 13 (4): 283-7 (2006)).

Naked RNA, DNA molecules encoding circular RNA molecules, or vectors comprising the circular RNAs or DNAs encoding circular RNAs may be administered to cells in the form of a composition. In some embodiments, the composition comprises a pharmaceutically acceptable carrier. The choice of carrier will be determined in part by the particular circular RNA molecule, DNA sequence, or vector and type of cell (or cells) into which the circular RNA molecule, DNA sequence, or vector is introduced. Accordingly, a variety of formulations of the composition are possible. For example, the composition may contain preservatives, such as, for example, methylparaben, propylparaben, sodium benzoate, and benzalkonium chloride. A mixture of two or more preservatives optionally may be used. In addition, buffering agents may be used in the composition. Suitable buffering agents include, for example, citric acid, sodium citrate, phosphoric acid, potassium phosphate, and various other acids and salts. A mixture of two or more buffering agents optionally may be used. Methods for preparing compositions for pharmaceutical use are known to those skilled in the art and are described in more detail in, for example, Remington: The Science and Practice of Pharmacy, Lippincott Williams & Wilkins; 21st ed. (May 1, 2005).

In some embodiments, the composition containing the recombinant circular RNA molecule, DNA sequence, or vector, can be formulated as an inclusion complex, such as cyclodextrin inclusion complex, or as a liposome. Liposomes can be used to target host cells or to increase the half-life of the circular RNA molecule. Methods for preparing liposome delivery systems are described in, for example, Szoka et al., Ann. Rev. Biophys. Bioeng., 9: 467 (1980), and U.S. Pat. Nos. 4,235,871; 4,501,728; 4,837,028; and 5,019,369. The recombinant circRNA molecule may also be formulated as a nanoparticle.

A host cell can be contacted in vivo or in vitro with a recombinant circRNA molecule, a DNA sequence, or a vector, or compositions containing any of the foregoing. The term “in vivo” refers to a method that is conducted within living organisms in their normal, intact state, while an “in vitro” method is conducted using components of an organism that have been isolated from its usual biological context. When the method is conducted in vivo, in some embodiments the production of the protein is tissue-specific. By “tissue-specific” is meant that the protein is produced in only a subset of tissue types within an organism, or is produced at higher levels in a subset of tissue types relative to the baseline expression across all tissue types. The protein may be produced in any tissue type, such as, for example, tissues of muscle, liver, kidney, brain, lung, skin, pancreas, blood, or heart.

In some embodiments, circRNAs expressing a protein of interest are delivered to cells (e.g., in vitro or in vivo) using a charge-altering releasable transporter (CART) (See e.g., McKinlay et al., herein incorporated by reference in its entirety). CARTs address delivery challenges posed by the mRNA cargo. These dynamic materials function initially as polycations that noncovalently complex, protect, and deliver polyanionic mRNA and then subsequently lose their cationic charge through a controlled self-immolative degradation to a neutral small molecule. It is contemplated that this charge alteration reduces or eliminates the chelative electrostatic anion-binding ability of the originally cationic material, thereby facilitating endosomal escape and enabling free mRNA release into the cytosol for translation. The present disclosure is not limited to a particular CART. In some exemplary embodiments, the CART is 1:1 mixture of oleyl (0) and nonenyl-substituted (N) carbonate monomers followed by a block of α-amino ester monomers (A) (O₆-stat-N₆:A₉). In some embodiments, the CART comprises a block length of 6 nonenyl and 6 oleyl carbonate units and 9 cationic amino-ester units.

In some embodiments, circRNAs expressing a protein of interest are delivered to cells (e.g., in vitro or in vivo) using any of a selection of cell-penetrating complexes such as those described in International Publication WO2022/020810, the contents of which are incorporated herein by reference in their entirety. Such cell-penetrating complexes may contain any of the cationic amphipathic polymers as described in WO2022/020810 and may also contain any of the lipophilic polymers or polymer domains as described in WO2022/020810. Variations of such cell-penetrating complexes useful in the present disclosure are taught in, for example, the detailed disclosure, examples and claims of WO2022/020810 and may alternatively be referred to as charge-altering releasable transporters (CARTs).

In some embodiments, circRNAs expressing a protein of interest are delivered to cells (e.g., in vitro or in vivo) using any of a selection of immolative cell-penetrating complexes such as those described in International Publication WO2018/022930, the contents of which are incorporated herein by reference in their entirety. Such immolative cell-penetrating complexes include those defined starting materials or complexes or variations thereof taught in WO2018/022930 and specifically any of the enumerated Embodiments 1-93 or the cell-penetrating complexes claimed therein. Such cell-penetrating complexes may comprise a nucleic acid non-covalently bound to a cationic amphipathic polymer, said cationic amphipathic polymer comprising a pH sensitive immolation domain as described in WO2018/022930.

In some embodiments, circRNAs expressing a protein of interest are delivered to cells (e.g., in vitro or in vivo) using any of a selection of hybrid immolative cell-penetrating complexes such as those described in International Publication WO2020/097614, the contents of which are incorporated herein by reference in their entirety. Such hybrid immolative cell-penetrating complexes include those described in any of the enumerated “P Embodiments” 1-114 and/or any of the enumerated Embodiments 1-93 or the hybrid immolative cell-penetrating complexes claimed therein.

In some embodiments, circRNAs expressing a protein of interest are delivered to cells (e.g., in vitro or in vivo, specifically to lung cells) using any of a selection of immolative cell-penetrating complexes such as those described in International Publication WO2020/160511, the contents of which are incorporated herein by reference in their entirety. Such immolative cell-penetrating complexes include those described in any of the enumerated Embodiments or claimed therein.

In some embodiments, circRNAs expressing a protein of interest are delivered to cells (e.g., in vitro or in vivo) using any of a selection of co-oligomeric vehicles (co-oligomers) such as those described in International Publication WO2013/036532, the contents of which are incorporated herein by reference in their entirety. Such co-oligomers include those defined as formulas I, 11, III, IV or variations thereof taught in WO2013/036532 and specifically any of those listed in Table 1 therein.

In some embodiments, circRNAs expressing a protein of interest are delivered to cells (e.g., in vitro or in vivo) using any of a selection of cell-penetrating guanidinium-rich oliophosphotriester transporter compounds such as those described in International Publication WO2017/083637, the contents of which are incorporated herein by reference in their entirety. Such transporter compounds include those defined as formulas I, II, III, IV, V, VI, VII or variations thereof taught in WO2017/083637 and specifically any of those claimed therein.

In some embodiments, circRNAs are utilized to deliver a nucleic acid encoding a therapeutic peptide to a subject. In some embodiments, such circRNAs find use in treating a disease or condition. The present disclosure is not limited to particular therapeutic proteins, diseases, or conditions. The technology described herein finds use in the treatment or prevention of any number of diseases or conditions (e.g., metabolic disorders, cancer, and the like). In some embodiments, circRNAs are administered multiple times to the same subject (e.g., to treat a disease or condition). The present disclosure is not limited to particular dosing schedules. In some embodiments, a cirRNA is administered in a single dose. In some embodiments, administration is repeated one (e.g., 2, 3, 4, 5, 6, 7, 8, 9, 10 or more) times at regular or irregular intervals (e.g., daily, weekly, bimonthly, monthly, etc).

### Methods of Inducing Immunity

In some embodiments, provided herein is a method of inducing immunity in a subject, comprising administering a composition, comprising: a complex of a charge-altering releasable transporter (CART) and a circular RNA molecule to the subject such that the composition induces immunity in the subject. In some embodiments, the circular RNA molecule comprises a protein-coding sequence (e.g., an antigen or a therapeutic protein). In some embodiments, the composition further comprises an antigen. In some embodiments, the immunity is innate immunity or an antigen-specific T cell response.

Also provided is a method of immunizing a subject, comprising administering a composition, comprising: a) a complex of a charge-altering releasable transporter (CART) and a circular RNA molecule; and b) an antigen to the subject such that the subject is immunized against the antigen.

In some embodiments, the administering treats or prevents a disease or disorder in the subject (e.g., cancer). In some embodiments, the delivering is intraperitoneally, intranasally, or intravenously. In some embodiments, the subject is a human or a non-human animal.

In some embodiments, the disclosed compositions find use in treating cancer (e.g., as a cancer vaccine). For example, in some embodiments, provided herein is a method of treating cancer in a subject, comprising administering a composition, comprising: a) a complex of a charge-altering releasable transporter (CART) and a circular RNA molecule; and b) an antigen. In some embodiments, the antigen is a tumor antigen. In some embodiments, the administering induces antigen-specific T-cell-based cellular immunity in the subject.

Non-limiting examples of tumor antigens include but are not limited to alphafetoprotein (AFP), carcinoembryonic antigen (CEA), CA-125, MUC-1, tpithelial tumor antigen (ETA), tyrosinase, melanoma-associated antigen (MAGE), and abnormal products of ras and p53.

In some embodiments, the CART is optimized to deliver the circular RNA to a particular cell type or tissue.

### Mega Tables

The patent application contains a lengthy table section. Copies of the tables are available in electronic form from the USPTO web site. An electronic copy of the tables will also be available from the USPTO upon request and payment of the fee set forth in 37 CFR 1.19(b)(3).

### EXAMPLES

The following examples further illustrate the invention but, of course, should not be construed as in any way limiting its scope.

### Example 1: Viral IRES Screen

Development of a Modular circRNA Assembly Platform

Synthesis of circRNAs via intron-assisted splicing and RNaseR digestion has been previously described (Chen 2017) but rapid creation of different circRNA species was difficult. To address this need, a modular cloning (MoClo) platform including of a set of backbones and parts in a clearly-defined but adaptable format that is compatible with both Golden Gate cloning (Engler 2008) and Gibson cloning (Gibson 2009) was developed (FIGS. 1 and 7A). Through various iterations of backbones, versions enabling circRNA transcription after transient transfection in cellulo from a CAGGS promoter, in vitro transcription via T7 promoter, and easy digestion of precursor linear RNA with RNaseR were developed. The backbone incorporates homology sequences that assist with RNA circularization as well as low-structure regions that assist in RNaseR processivity and complete digestion. The Golden Gate overhangs were carefully chosen to produce robust assembly of all seven parts without error, which was assessed over the course of testing by sequencing of hundreds of clones selected by sequencing. The circRNA MoClo system includes a bacterially-expressed GFP dropout in its backbones to negative select for incorrect assemblies, creates in-frame glycine-serine linkers for fusion proteins between parts 3-5, and leaves residual consistent restriction enzyme cut sites at key junctions for subcloning purposes. The platform is amenable to synthesis of large libraries through combinatorial assembly of parts, which themselves can constitute pools of sequences.

Finally, the downstream circRNA synthesis and purification protocols were optimized for speed, ease, and robustness. A NanoLuciferase (NanoLuc)-based assay was adapted for assessment of circRNA translation strength because of its advantages of having a broad quantitative range (FIG. 7B), amenability to a multiwell plate format, and amenability for both secreted and intracellularly retained forms of NanoLuc.

### RNA Modifications Improve Translation Strength and Stability

RNA nucleoside modifications are important to the efficacy of RNA therapeutics due to their relevance in controlling mRNA (Karikó 2005, Durbin 2016, Svitkin 2017) and circRNA immunity (Chen 2019). As a baseline, an unmodified circRNA encoding NanoLuc driven by the coxsackievirus B3 (CVB3) IRES (iCVB3) from the picornavirus family, with the translation cassette flanked by 50 bp random sequence spacers was used. In separate syntheses, eight RNA modifications were incorporated-5-methylcytidine (5 mC), 5-methyluridine (5 mU), 5-methoxycytidine (5moC), 5-methoxyuridine (5moU), 5-hydroxymethylcytidine (5hmC), 5-hydroxymethyluridine (5hmU), pseudouridine (Ψ), and N1-methylpseudouridine (N1Ψ)—that have demonstrated relevancy in improving mRNA translation (Karikó 2005); N6-methyladenosine (m6A) because of its relevance in modulating circRNA immunity (Chen 2019); and five RNA modifications—N1-ethylpseudouridine (N1ethΨ), 2′-fluoro-2′-deoxycytidine (2′FdC), 2′-fluoro-2′-deoxyuridine (2′FdU), 2-thiouridine (2ThioU), and 2′-O-Methylcytidine (2′OMeC)—whose effects on RNA translation have not been described (FIG. 8A). On first-pass, all RNA modifications were tested at a 10% incorporation level to ensure a large effect size, and upon synthesis it was found that none of these modifications greatly reduced circRNA yield. When assayed for translation of NanoLuc, most modifications at 10% incorporation blunted translation compared to unmodified circRNA. However, 2ThioU and 2′OMeC inhibited translation to a lesser extent, indicating that further titration of their incorporation levels might improve translation strength.

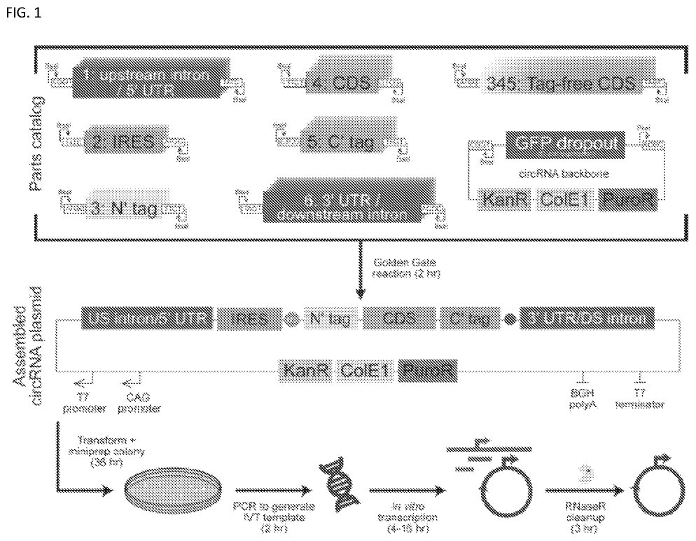

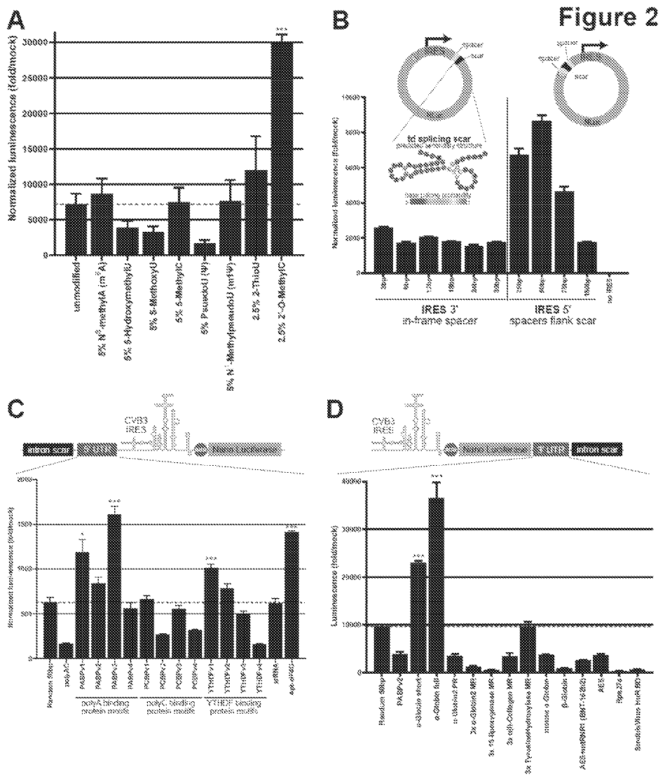

Following further titration of RNA modifications at 2.5% and 5% incorporation, optimized incorporation levels for eight RNA modifications in circRNAs were determined (FIG. 2A). Of these, 2′OMeC significantly improved translation while m6A and 2ThioU resulted in non-significant increases. Changes in translation were not due to differences in the amount of transfected RNA, which was equivalent among circRNA samples (FIG. 8B). Noticeably, nucleoside modifications known to improve mRNA translation such as NIT (Karikó 2005, Durbin 2016, Svitkin 2017) did not appear to have the same effect in circRNAs.

In separate preparations, the same circRNAs were synthesized with 5% m6A incorporation. Compared to unmodified circRNAs, circRNAs containing 5% m6A showed equivalent translation after transfection or electroporation in vitro (FIG. 8 A and B).

A fetal bovine serum (FBS) degradation assay which makes use of the endogenous RNases in FBS was developed (FIG. 8C). CleanCap and 100% NIP-modified mRNA, the industry standard for mRNA-based therapies, was fully degraded by 1% FBS alongside unmodified circRNA. Conversely, circRNA containing 5% m6A was more resistant to nucleases and was not fully degraded until 2% FBS. These results indicate that nucleoside modification of circRNAs can confer stability against nucleases (FIG. 8C), which may help extend translation duration. However, when circRNAs are delivered into cells, certain RNA modifications improve translation strength despite having equivalent intracellular RNA stability (FIG. 2A).

Although circRNA translation in vitro was greatest with 2.5% 2′OMeC, attempts to combine this modification with m6A to block immune recognition abrogated translation efficiency. Subsequent optimization efforts were focused on m6A-modified circRNAs, which are shielded from innate immunity in vivo (Chen 2019). To compare the expression kinetics of 5% m6A-modified circRNA with CleanCap and 100% NIT-modified mRNA, a time course using secreted NanoLuc as the reporter was performed (FIG. 8D). mRNA and circRNA were electrocuted into cells and media harvested at time points out to 24 days, at which the NanoLuc signal was indistinguishable from background. While mRNA yielded a stronger maximum translation signal, translation rapidly dropped after 48 hours. On the other hand, circRNA translation peaked at 48 hours but continued yielding detectable expression out to almost 20 days. Moving forward, 5% m6A was incorporated in every circRNA preparation unless otherwise explicitly stated.

Vector Topology and Spacer Requirements for circRNA Translation

Principles behind circRNA vector topology that are necessary for strong translation were investigated. First, circRNAs with the IRES downstream, or 3′, of the reporter NanoLuc gene, maintaining the reading frame through the residual scar formed by the self-splicing reaction of the T4 td intron were synthesized. In this orientation, translation through the splicing scar is unavoidable. Hypothesizing that the highly structured scar sequence may obfuscate the translation start site, circRNA variants with in-frame spacers of varying lengths between the translation start and the splicing scar were synthesized. The peptides encoded by these spacers reflected consensus viral leader peptide sequences from the rhinovirus family. Testing the expression of these circRNAs indicated that increasing the spacer length was non-beneficial for translation, and that the ribosome was unaffected by the td splicing scar's secondary structure (FIG. 2B).

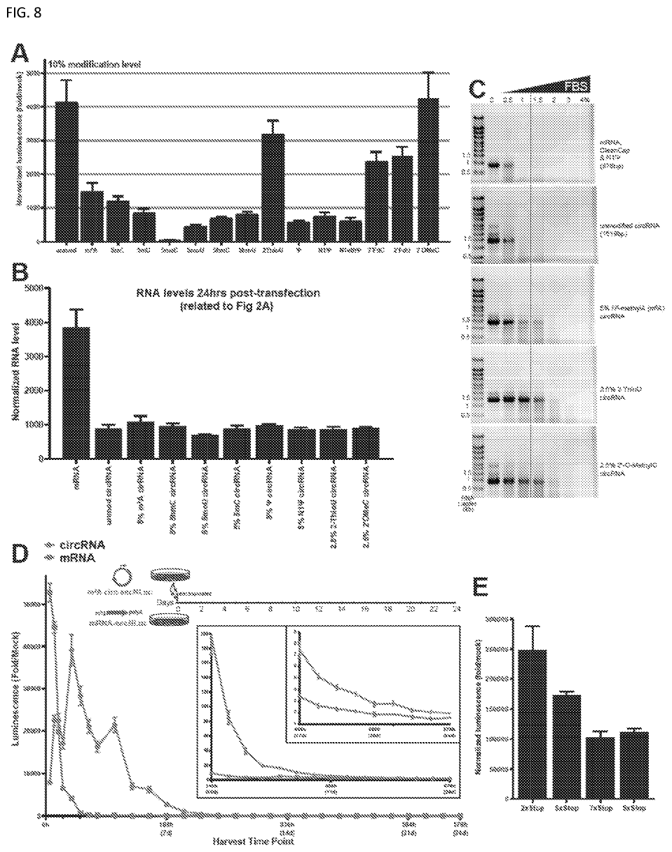

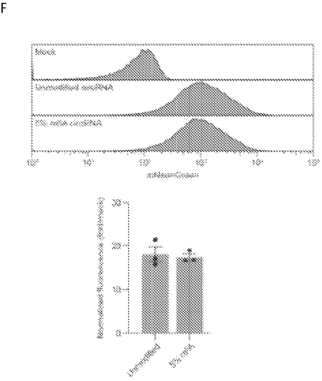

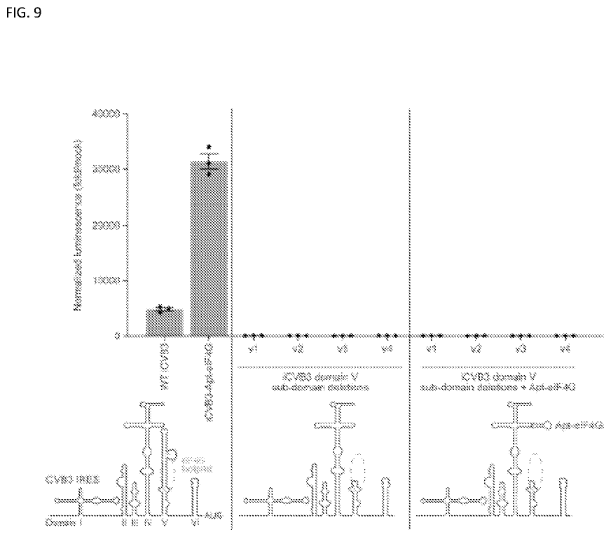

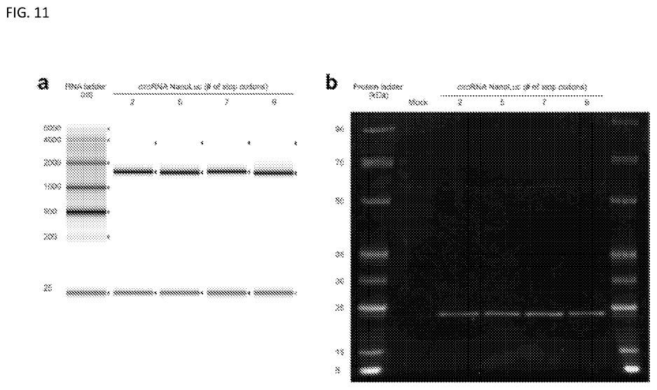

The topology of the circRNA vector was reversed, placing the IRES immediately upstream of the NanoLuc gene. Flanking this translation cassette, adding spacers derived from random 50% GC content sequences of varying lengths in the 5′ and 3′ untranslated regions (UTRs) of the circRNA were tested. When assayed for NanoLuc expression, it was found that circRNAs with spacers 50 bp in length yielded the strongest translation (FIGS. 2B and 9A,B). It was also tested whether the number of stop codons following the coding sequence affected circRNA expression and found that adding more than two stop codons (the number used in the MoClo platform) reduced translation strength (FIG. 8E) without affecting the size of the encoded protein (FIG. 8F and FIG. 11). The results indicate that IRES-mediated translation of circRNAs can occur readily through an intron splicing scar, though with reduced efficiency compared to the IRES being directly upstream of a gene. Furthermore, translation of circRNAs can be improved by the addition of 50 bp spacers separating the IRES and gene of interest from the splicing scar.

5′ and 3′ UTRs can Improve circRNA Translation

5′ and 3′ UTRs in mRNAs can recruit RNA-binding proteins (RBPs) that enable strong translation as well as aspects of post-transcriptional regulation (Jackson 2010). One such family of RBPs are poly(A)-binding proteins (PABPs), which interact with polyadenosine tracts of 12 bp or longer in the 3′ UTR and subsequently trigger binding of eIFs (Mangus 2003). Other well-characterized RBPs include poly(C)-binding proteins (PCBPs), which recruit ribosomal proteins and trans-activating factors to picornavirus RNAs (Blyn 1997, Gamarnik 1997, Graff 1998, Walter 1999, Luo 2014), as well as YTIDF family members, which bind m6A and have been shown to regulate mRNA translation and stability (Wang 2014, Shi 2017). Previously, strong circRNA translation was reported using 5′ and 3′ UTR sequences consisting of −50 bp sequences that are mostly adenosine (e.g., with 84% adenine) in 1-10 bp stretches and cytidine interspersed—termed polyAC spacers (Wesselhoeft 2018). It was investigated if specific sequences could be installed to improve translation. 50 bp spacers encoding RNA-binding motifs for the three aforementioned RBP families were synthesized as 5′ UTRs, designing several versions per motif to account for any sequence-specific variability. Additionally, two highly structured sequences with well defined effects: xrRNA ((AGCGUAACCUCCAUCCGAGUUGCAAGAGAGGGAAACGCAGUCUC)), an RNA hairpin found in dianthoviruses that blocks degradation by the 5′-3′ exonuclease Xm120, and Apt-eIF4G, an eIF4G-recruiting aptamer that has been shown to increase mRNA translation when added to the 5′ UTRs of transcripts, were tested. Apt-eIF4G (ACUCACUAUUUGUUUUCGCGCCCAGUUGCAAAAAGUGUCG), is an eIF4G-recruiting aptamer that has been shown to increase mRNA translation when added to the 5′ UTRs of transcripts (Tusup 2018). Upon incorporating these sequences into the 5′ UTR of circRNAs and assaying for NanoLuc expression, it was found that PABP motifs and the eIF4G aptamer improved translation the most (FIG. 2C).

The 3′ spacer downstream of the stop codons was optimized, drawing upon a wide array of 3′ UTRs. These included the human α-globin 1 (HBA1) 3′ UTR in its shortened (Truong 2019) and full-length forms (Richner 2017); the region of human α-globin 2 (HBA2) protected from RNase digestion by the α-complex, an RNA-protein complex implicated in mRNA stabilization (Holcik 1997); minimal regions for α-complex binding to HBA2, rabbit 15-lipoxygenase, human a(I)-collagen, and rat tyrosine hydroxylase tiled in triplicate (Holcik 1997); the human β-globin 3′ UTR truncated after the AAUAAA polyadenylation signal (Jiang 2006; herein incorporate by reference in its entirety); the amino-terminal enhancer of split (AES) 3′ UTR alone and in combination with mitochondrially encoded 12S rRNA (mtRNR1) (Orlandini von Niessen 2019); the 3′ UTR of mouse Rps27a which was highly expressed in Hep3B and 293T cells (Zeng 2020); and the HuR-binding region from Sindbis virus that protects its transcript from RNase digestion (Sokoloski 2010). When incorporated into circRNAs and assayed by NanoLuc expression, the majority of these 3′ UTRs that drive strong translation in an mRNA context failed to do so for circRNAs. However, replacing the 3′ spacer with either the short or full-length form of the HBAT 3′ UTR significantly improved translation strength (FIG. 2D).

### A Full-Length Viral IRES is Critical for Strong Translation

Viral IRESs are diverse and highly-structured RNA regions found primarily in viral 5′ UTRs that promote cap-independent translation (Kieft 2008, Filbin 2009, Martinez-Salas 2018). Because iCVB3, the baseline IRES used in this study, is nearly 750 bp, it was determined if it was possible to truncate an IRES while retaining circRNA translation. A previous structure map of iCVB3 divided the sequence into seven domains (Bailey 2007), beginning with domain I containing a cloverleaf structure thought to be critical for viral replication (Murray 2004). Domains II-V have also been reported to interact with multiple TRES trans-activating factors (ITAFs) (de Breyne 2009, Souii 2013, Sweeney 2013), while domain VI hosts an AUG upstream of the true translation initiation site that recruits the 43 S ribosomal preinitiation complex (Nicholson 1991, Yang 2003, Sweeny 2013).

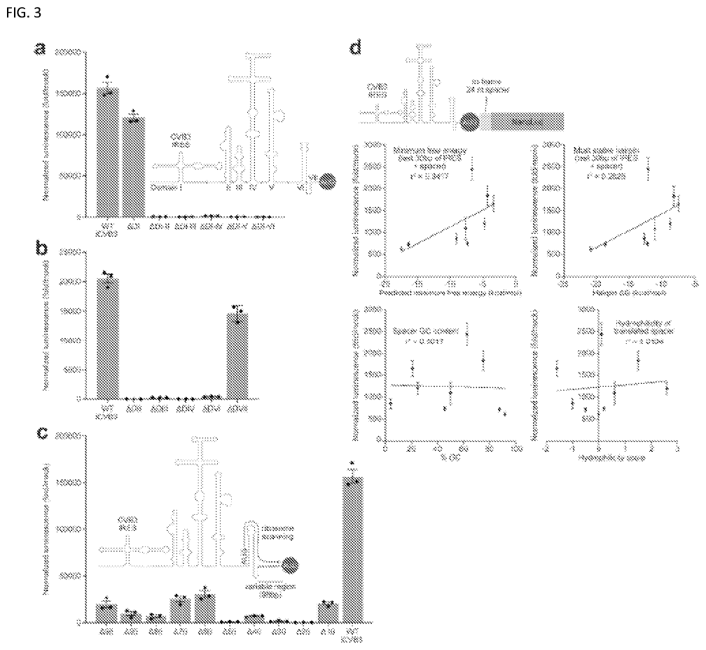

IRES domain truncations were performed starting from the 5′ end of iCVB3, choosing truncations at boundaries where there was little known secondary structure base pairing. Compared to the full-length IRES, deletion of domain I significantly cut circRNA translation by 25%, and further deletions completely eliminated translational activity (FIG. 3A). Deletions of other individual iCVB3 domains similarly reduced circRNA translation; removal of domain VII decreased luminescence by 29%, and loss of domain II, III, IV, or VI completely ablated protein production (FIG. 3B). Successive truncations of iCVB3 from the 3′ end were then performed. This region between domain VII and the start codon is highly variable in both sequence and length among different picornavirus IRESs, so it was contemplated that it would be amenable to shortening. 3′ deletion of as few as ten terminal nucleotides from this region nearly ablated circRNA translation (FIG. 3B). Together, these data show that a full-length IRES is necessary for strong circRNA translation.

### IRES-Coding Sequence Junction Secondary Structure Dictates Translation Strength

Coding sequence-specific factors that influence translation initiation in circRNAs were investigated. To assess this, circRNAs with nine different 24 bp N-terminal leader sequences in frame between the AUG start codon and the NanoLuc reporter were synthesized (FIG. 3D). Various features of these leader sequences—secondary structure, GC content, and translated hydrophilicity—were compared against the resulting NanoLuc reporter strength. Indicators of secondary structure stability, such as predicted minimum free energy and free energy change for the most stable hairpin, were most correlated with NanoLuc translation (Gruber 2008), with 34.2% and 28.3% of variation in translation strength explained by those factors, respectively. On the other hand, the GC content of the N-terminal leader and hydrophilicity of its encoded peptide were not predictive of translation efficiency. These findings indicate that in silico optimization of base-pairing interactions between an IRES and coding sequence can yield additional benefits for circRNA translation.

Disruption of eIF4G Binding to iCVB3 Abrogates Translation

eIF4G and eIF4A binding to domain V of iCVB3 is thought to be a key step in initiating translation from this IRES (de Breyne 2009). While it is unknown how these same eIFs contribute in the context of circRNAs, it was contemplated that interfering with their binding to iCVB3 might adversely affect translation. To disrupt eIF binding sites, locked nucleic acids (LNAs), modified nucleic acids with especially high antisense binding affinity (Wahlestedt 2000, Huston 2021) against a non-base-paired linker region between iCVB3 domains I and II (LNA #1), the footprint of eIF4A (LNA #2), the footprint of eIF4G (LNA #3), and a random sequence (NC LNA) were used.

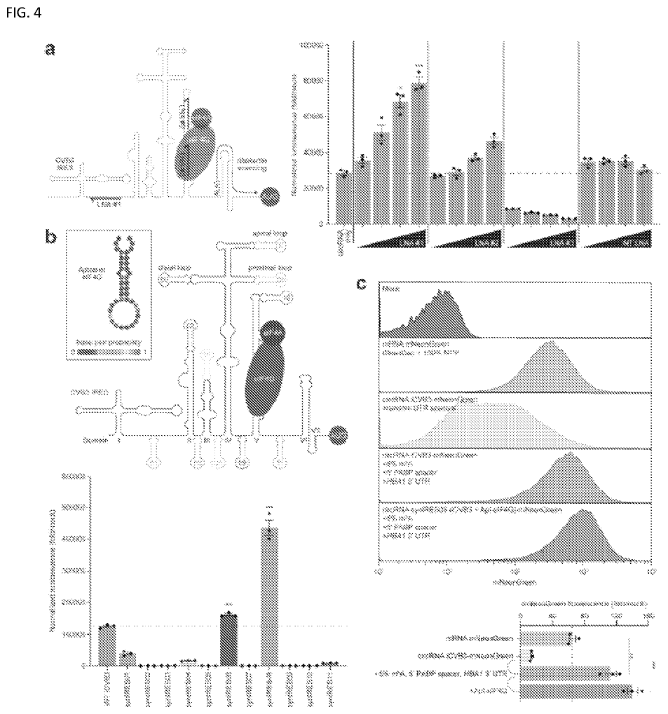

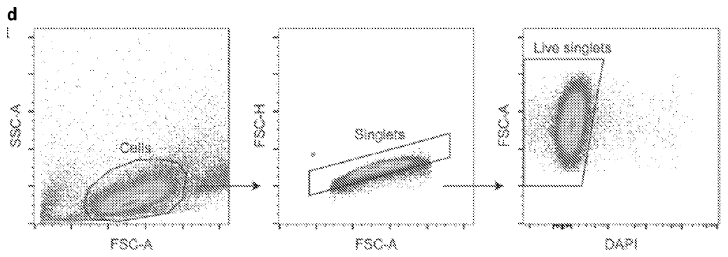

The effect of LNAs was tested across a range of concentrations, using NanoLuc as a readout for circRNA translation (FIG. 4A). NC LNA had minimal impact on the strength of iCVB3. In contrast, LNA #3 dose-dependently disrupted NanoLuc activity, implicating eIF4G sites in iCVB3 domain V as necessary for translating circRNAs. It was also found that locking the secondary structure of the domain I-II junction with LNA #1 improved translation in a dose-dependent manner. Because RNA flexibility is a hallmark of picornavirus IRESs (Martinez-Salas 2018), it is contemplated hat this increase in translation strength may be due to fewer unfavorable base-pairing interactions between this region and the circRNA backbone. The improvement was dose-dependent rather than reduction in translation with LNA #2, supporting that direct binding of eIF4A to iCVB3 domain V is not needed for circRNA translation. However, it is still possible that eIF4A in this context may directly interact with eIF4G.

Four variants of iCVB3 with sub-domain deletions of where eIF4G interacts with the upper stem of domain V were synthesized (FIG. 9). These variants differed in the position where the stem loop was truncated, but at a minimum all ablated the eIF4G footprint. As expected, deletion of this key portion of iCVB3 domain V completely abrogated translational activity.

Synthetic IRES Engineering with an eIF4G-Binding Aptamer

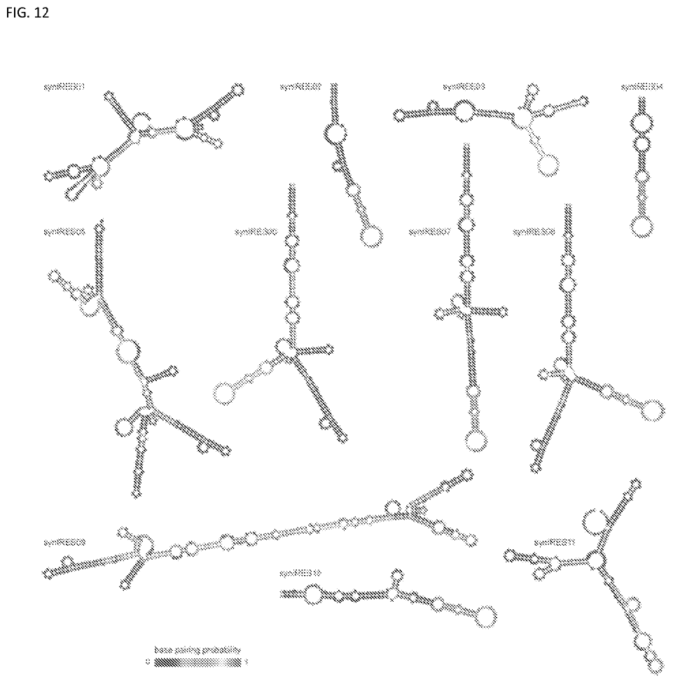

From the LNA experiments, it was concluded that eIF4G plays a pivotal role in initiating translation from IRESs in circRNAs. It was thus hypothesized that engineering iCVB3 to have greater affinity for eIF4G might result in stronger circRNA translation. Apt-eIF4G, an eIF4G-recruiting aptamer, can improve cap-dependent translation when inserted in the 5′ UTR of mRNAs (Tusup 2018). Synthetic variants of the iCVB3 where Apt-eIF4G was inserted at hypothetically permissible regions within the IRES (FIG. 4B) were generated. These positions were either within the flexible non-base-paired interdomain regions (synIRES01, 03, 05, 09, and 11), which were chosen to avoid aberrant Apt-eIF4G-linker interactions, or at the end of loop domains (synIRES02, 04, 06, 07, 08, and 10), with removal of several wild-type nucleotides to smoothly transition from the stem-loop structure into Apt-eIF4G's RNA stem. In all cases, rational engineering choices were informed by in silico RNA structure prediction (FIG. 12). Using the NanoLuc assay, it was found that domain IV's cruciform structure was the most permissive to Apt-eIF4G insertion. Both synIRES06 and synIRES08, where Apt-eIF4G was inserted in the distal and proximal loops of domain IV, respectively, showed significantly improved translation over wild-type iCVB3. Conversely, insertion at the apical loop of domain IV completely abrogated translation, consistent with reports of an essential internal C-rich loop and GNRA tetraloops at this site (Garmarnik 2000, Bhattacharyya 2006).

Using flow cytometry, the result was validated with a different reporter, mNeonGreen, a bright monomeric green fluorescent protein (Shaner 2013). Compared to CleanCap and 100% N1Ψ-modified mRNA or unmodified circRNA with random 5′ and 3′ UTRs, 5% m6A-modified circRNA with the 5′ PABP spacer and HBA1 3′ UTR exhibited greater mNeonGreen expression (FIG. 4C). This was further improved by aptamer engineering of iCVB3 to include Apt-eIF4G. For gating strategy, see FIG. 4D.

The rescue of iCVB3 domain V eIF4G footprint deletions through addition of Apt-eIF4G to the proximal loop of domain IV were assayed (FIG. 9). However, no recovery of translation was achieved by this strategy for any of the four variants. Prior toe-printing analysis deduced conformational changes in domain VI and the 3′ end of iCVB3 following the recruitment of eIF4G and eIF4A (de Breyne 2009). The results indicate that these RNA conformational changes are indeed crucial for proper ribosome assembly and that simply recruiting eIF4G locally is insufficient for translation initiation.

### Identification of Robust Higher-Strength IRESs

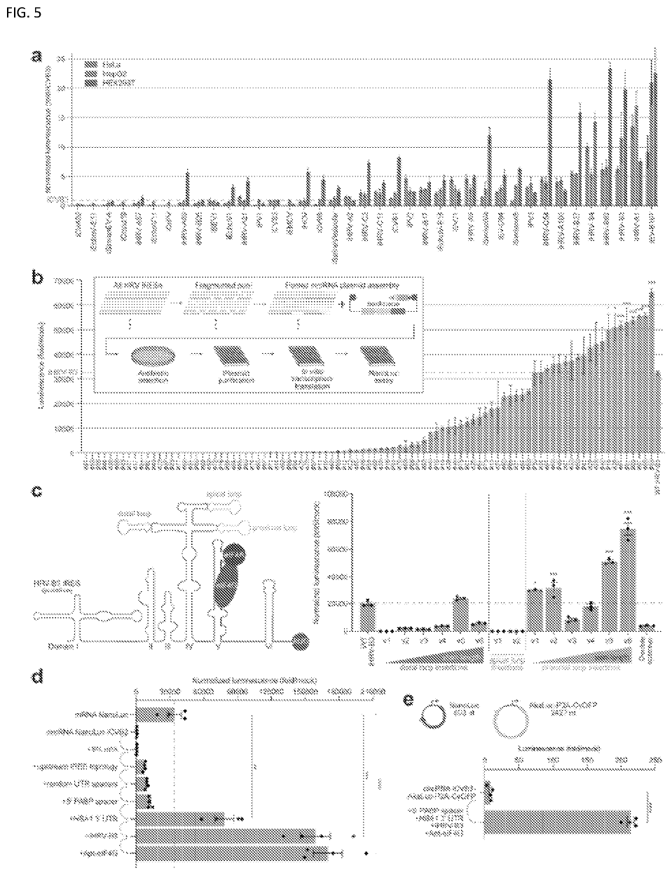

IRESs have evolved a variety of mechanisms to utilize host factors for initiating translation. Based on these mechanisms, IRESs have been categorized into several types—type 1 IRESs can be found in enteroviruses, type 2 in cardioviruses and aphthoviruses, type 3 in some picornaviruses, and type 4 in teschoviruses (Daijogo 2011). To further optimize circRNA expression, IRESs with stronger translation than those previously described in the literature (Mokrejs 2006, Wesselhoeft 2018) were identified. Over several rounds of synthesis and testing, a number of IRESs spanning different types and species in circRNAs were characterized. IRESs representing canonical IRES types (type in parenthesis), such as from CVB3 (1), poliovirus 1 (PV1) (1), human rhinovirus A1 (HRV-A1) (1), encephalomyocarditis virus (EMCV) (2), hepatitis C virus (HCV) (3), and cricket paralysis virus (CrPV) (4) were assayed. Type 1 IRESs appeared to drive strong translation in the context of circRNAs (FIG. 5A), (Filbin 2009). The screen was expanded to include a large set of putative type 1 IRESs from the enterovirus genus, which were incorporated into circRNAs and assayed for NanoLuc translation.

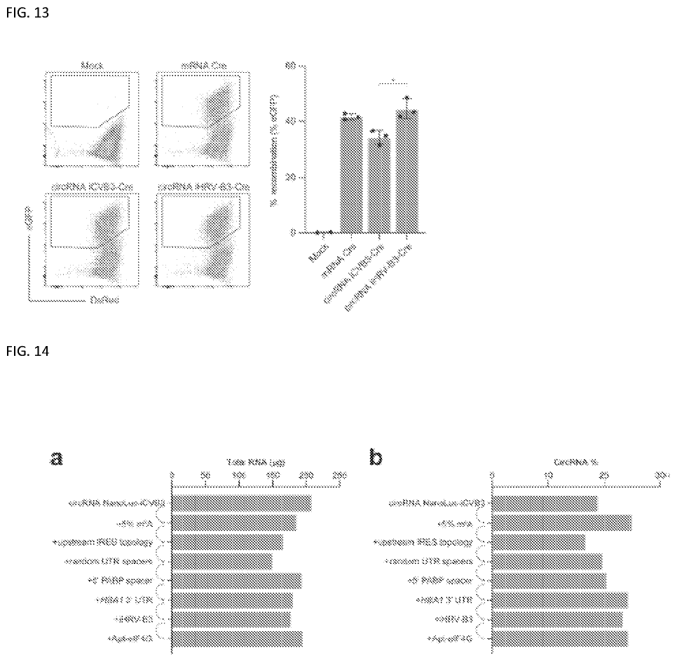

In the screen, many IRESs with stronger translation than iCVB3 across multiple cell lines were identified (FIG. 5A). In particular, IRESs from the human rhinovirus B (HRV-B) and enterovirus B (EV-B) species drove strong circRNA translation. To validate this result with a different transgene, a fluorescent reporter assay was used to assess Cre-mediated recombination after transfection of circRNAs encoding Cre recombinase (FIG. 13). At 24 hours post-transfection, greater recombination was observed with iHRV-B3 compared to iCVB3, supporting iHRV-B3 as a stronger IRES for circRNA translation.

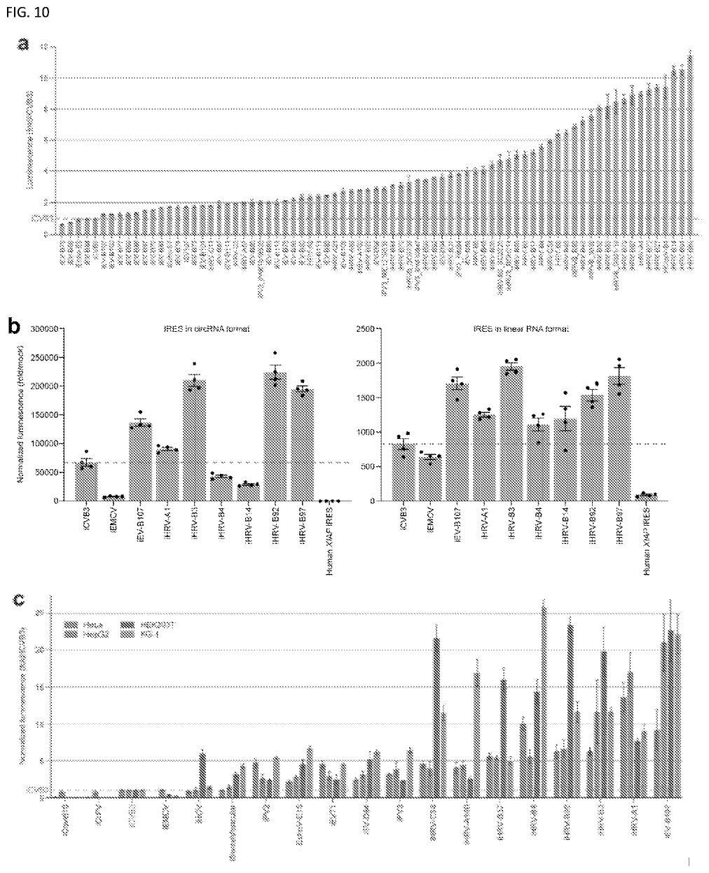

With this knowledge, IRESs from every HRV-B and EV-B subspecies with a publicly available sequence on NCBI Virus were synthesized and incorporated into circRNA expression plasmids. Given the scale of this screen, an in vitro coupled transcription-translation (IVTT) approach, using circRNA expression plasmids rather than purified circRNAs as the input material was used (FIG. 10A). In the IVTT-based NanoLuc assay, a large number of HRV-B and EV-B IRESs with greater translational activity than iCVB3 were found. These IRESs were validated in cellulo using purified circRNAs (FIG. 10B). While many hits turned out to be false positives, the discovery of iHRV-B92 and iHRV-B97 as higher-strength IRESs were recapitulated. When these same IRESs were also tested in a linear RNA format, relative differences in translation strength held, but with a 100-fold reduction in absolute expression compared to circRNAs (FIG. 10B). For the strongest IRESs, NanoLuc translation was tested in four different cell lines and found that the many drove efficient translation independent of cell type (FIG. 10C). At the same time, some IRESs demonstrated stronger translation in a specific cell type, such as HEK293T cells for iHCV and iHRV-C54 and KG-1 cells for iHRV-A100 and iHRV-B4.

The earlier circRNA optimizations (FIGS. 2 and 4) were compared to the improvement conferred by changing the IRES (FIG. 5B). The CVB3 IRES downstream of NanoLuc was used as a starting point and successively incorporated m6A, reversed the vector topology, added random 5′ and 3′ UTR spacers, modified the 5′ spacer to include a PABP motif, replaced the 3′ UTR spacer with the HBA1 3′ UTR, and switched the IRES to iHRV-B3. It was found that these changes progressively increased circRNA expression, with the final design exhibiting significantly more translation than CleanCap and 100% NIT-modified mRNA. To validate this with a separate transgene, a two-color fluorescent reporter assay was used to assess Cre-mediated recombination after transfection of circRNA encoding Cre recombinase (FIG. 13). Specifically, a transgenic HEK293T reporter cell line in which DsRed, a red fluorescent protein, is expressed before Cre recombination, and enhanced green fluorescent protein (eGFP) is expressed after was used. Twenty-four hours after transfection of circRNA encoding iCVB3-driven Cre recombinase, recombination was observed in 34.3% of cells (FIG. 5C). This was significantly improved to 44.6% with the stronger HRV-B3 IRES.

### Synthetic IRES Engineering Through Unbiased DNA Shuffling

DNA shuffling is an unbiased approach commonly used to generate large diverse libraries for selecting novel engineered proteins (Michnick 1999). Shuffling particularly makes sense over other library generating strategies, such as point mutagenesis, when a homologous family of related proteins is available to act as seed templates for the shuffling reaction. Because the strongest translation overall was observed with IRESs from HRV, DNA shuffling by fragmenting 41 HRV IRESs and cloning the resulting pool into circRNA plasmids (FIG. 5D). 93 circRNA expression plasmids with unique shuffled IRESs were used to measure their translation strength using an IVTT assay, with iHRV-B3 as an internal benchmarking control. From these 93 shuffled IRESs, nine were identified with significantly stronger translational activity than wild-type iHRV-B3, illustrating the ability of IRES shuffling to engineer improved IRESs for circRNA applications.

Validation of Apt-eIF4G IRES Engineering with iHRV-B3

It was hypothesized that the aptamer engineering approach with Apt-eIF4G might also improve translation for IRESs of indeterminate structure. To test this, the domain architecture of a strong IRES, iHRV-B3 was predicted in silico (Gruber 2008), which identified six domains including a cruciform structure in domain IV (FIG. 5E). Loops within this cruciform structure were used to perform Apt-eIF4G insertions at the distal, apical, and proximal loop locations, varying the length of the resulting stem by rationally inserting base-paired RNA nucleotides and validating the structure in silico. It was contemplated that by assessing a range of stem lengths, one might uncover a particular position for Apt-eIF4G most favorable to cooperative binding effects. It was found that Apt-eIF4G insertions at the proximal loop of domain IV significantly improved circRNA translation compared to wild-type iHRV-B3, demonstrating the broader utility of the aptamer engineering strategy to synthesize stronger IRESs. As with iCVB3, apical loop insertions of Apt-eIF4G also destroyed iHRV-B3 activity, consistent with a predicted GNRA tetraloop in this region. While a double aptamer insertion of Apt-eIF4G at both the distal and proximal loops was performed, this greatly reduced circRNA translation.

Quantification of Combined circRNA Optimizations

Each of the earlier circRNA optimizations was compared in a single experiment (FIG. 5d). Initially, iCVB3 downstream was of NanoLuc and successive incorporation of m6A, reversal of the vector topology, random 5′ and 3′ UTR spacers, modified 5′ spacer to include a PABP motif, replacement of the 3′ UTR spacer with the HBA1 3′ UTR, Switching of the IRES to iHRV-B3, and insertion of a proximal loop aptamer into iHRV-B3 were performed. It was found that these changes progressively increased circRNA expression without compromising RNA yield or circularization efficiency (FIG. 14A,B) with the final design exhibiting a 224-fold improvement relative to unoptimized circRNA and significantly more translation than CleanCap and 100% N1Ψ-modified mRNA.

To validate these findings with a larger transgene, circRNAs expressing AkaLuc-P2A-CyOFP, a coding sequence more than four times longer than NanoLuc, were prepared (FIG. 5e). When assayed for Aka luciferase (AkaLuc) activity, the combined additions of a 5′ PABP spacer, HBA1 3′ UTR, HRV-B3 IRES, and proximal loop Apt-eIF4G insertion again improved circRNA translation, supporting the generalizability of these optimizations.

In Vivo Expression of Optimized circRNAs

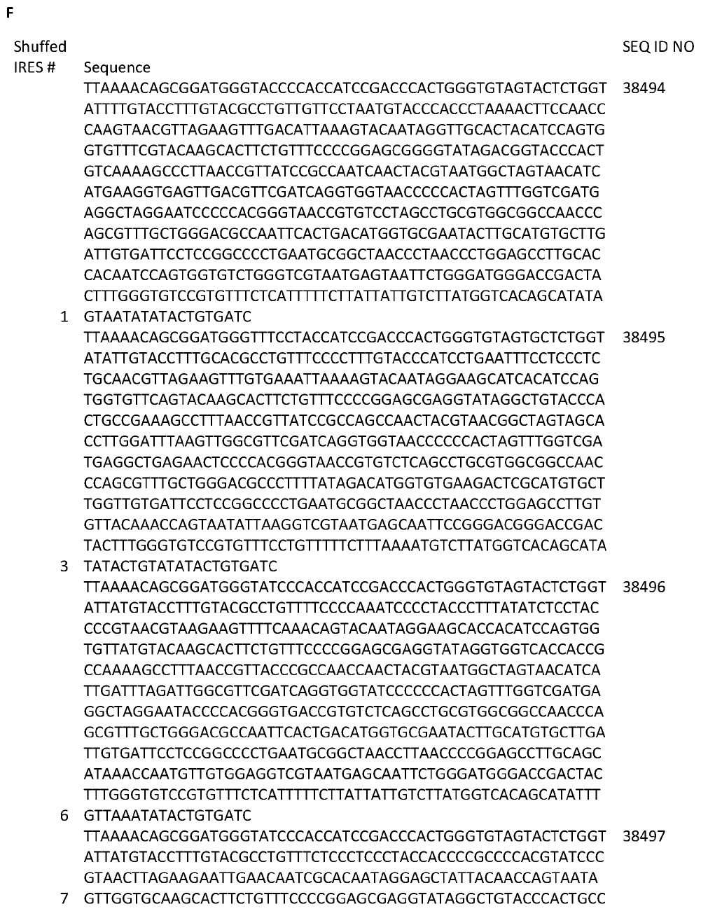

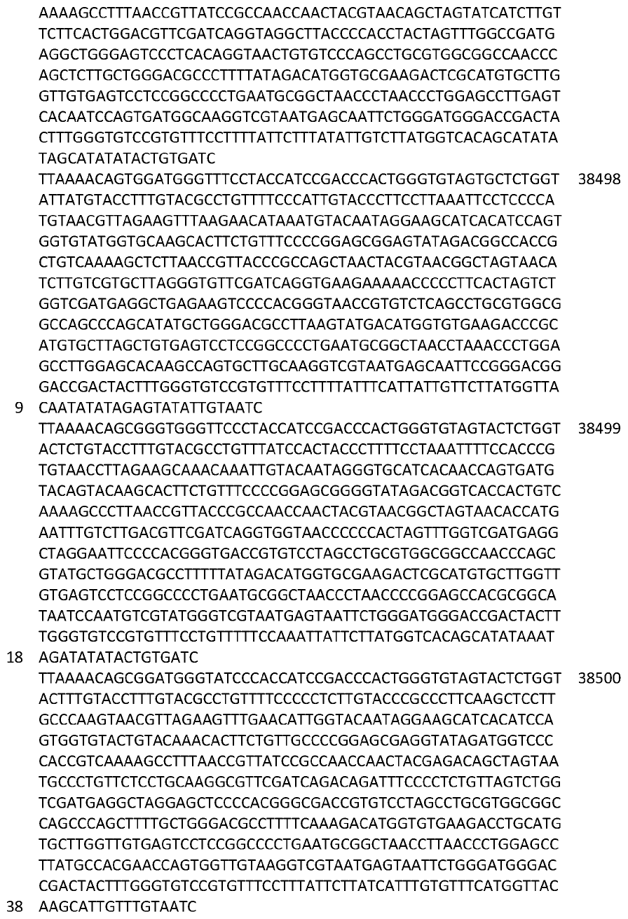

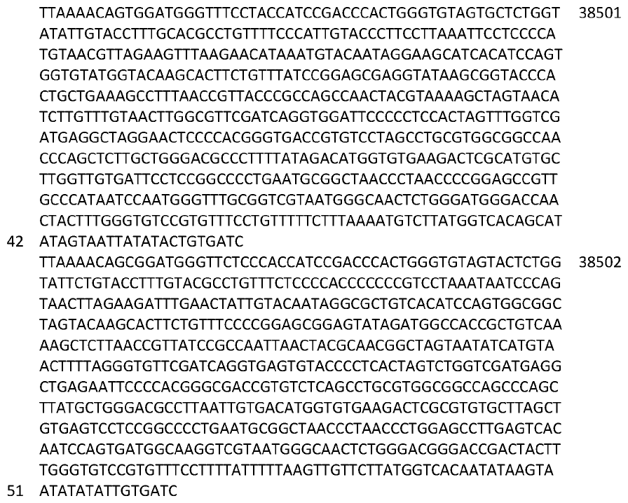

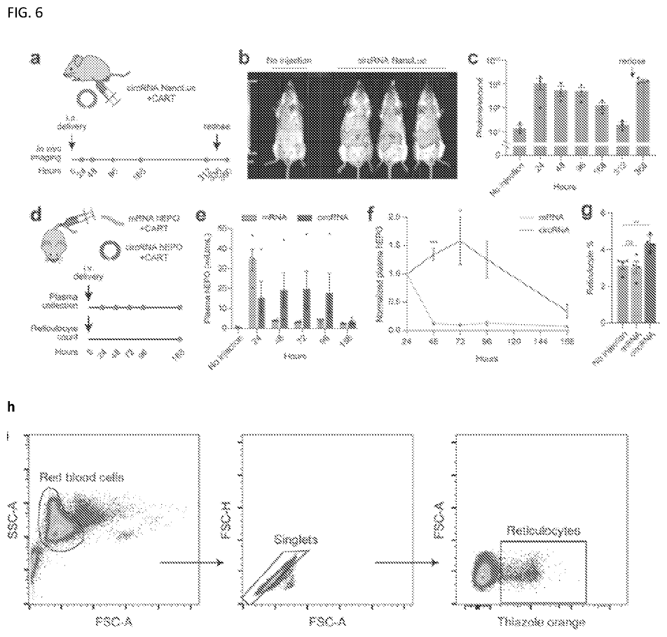

The above circRNA optimizations—5% m6A incorporation, upstream IRES topology, 5′ PABP spacer, HBA1 3′ UTR, and HRV-B3 IRES with proximal loop Apt-eIF4G insertion were combined to test the expression of optimized circRNA in vivo. To deliver RNAs, they were formulated them with charge-altering releasable transporters (CARTs), temporarily cationic molecules capable of mediating mRNA expression in mice (McKinlay 2017). A circRNA encoding an intracellularly expressed NanoLuc was administered it in mice via intraperitoneal injections (FIGS. 6A and 6B). Compared to untreated animals, those receiving circRNA showed greater luminescent activity for at least one week (FIG. 6C), indicating that engineered circRNAs can be expressed in vivo. When redosed two weeks after the first injection, NanoLuc expression was also indistinguishable from initial levels (FIG. 6C), supporting that repeat administration of circRNAs may be feasible.

A head-to-head comparison of optimized circRNA versus CleanCap and 100% N1Ψ-modified mRNA in vivo using RNAs encoding human erythropoietin (hEPO), a secreted protein used to treat anemia was performed. Following intravenous administration in mice, plasma hEPO levels from circRNA were initially less than those from mRNA (FIGS. 6D and 6E). However, while mRNA expression of hEPO declined rapidly within 48 hours, circRNA expression remained consistent until at least 96 hours post-injection (FIGS. 6E and 6F). Functionally, hEPO can elevate reticulocyte production in mice, although much higher concentrations are required than for mouse EPO52. Reticulocyte counts were significantly increased in mice that received a single dose of hEPO-encoding circRNA after one week, while reticulocyte levels after an equimolar dose of mRNA were no different than those from untreated animals (FIG. 6G). Together, the data show that engineered circRNAs can express in vivo at strengths comparable to modified mRNAs, and with greater duration. For gating strategy, see FIG. 6H.

### Methods

### Molecular Cloning

Part plasmids (see FIG. 7) were synthesized by cloning PCR products or premade DNA fragments (Integrated DNA Technologies) into a custom entry vector (pRC0569) via a Golden Gate reaction. Overhangs were included as indicated by FIG. 7. Parts and pRC0569 were combined stoichiometrically 2:1 to a total volume of 2 μL, after which 0.5 μL NEB Golden Gate Enzyme Mix (BsmBI-v2), 0.5 μL of T4 ligase buffer, and ddH2O to 5 μL were added. The reaction was incubated at 42° C. for 2 minutes followed by 16° C. for 2 minutes for 30 cycles, followed by 60° C. for 5 minutes before cooling to 4° C. Turbo Competent (NEB) cells were transformed using 2 μL of the reaction and plated onto carbenicillin plates. Non-green colonies were picked, miniprepped, and sequenced.

CircRNA plasmids were assembled by cloning parts 1-6 into a custom backbone (pRC0940) via a second Golden Gate reaction. Part plasmids and pRC0940 were combined stoichiometrically 2:1 to a total volume of 4 4 L, after which 0.5 μL NEB Golden Gate Enzyme Mix (BsaJ) and 0.5 μL of T4 ligase buffer were added. The reaction was incubated at 37° C. for 2 minutes followed by 16° C. for 2 minutes for 30 cycles, followed by 60° C. for 5 minutes before cooling to 4° C. Turbo Competent (NEB) cells were transformed using 2 μL of the reaction and plated onto carbenicillin plates. Non-green colonies were picked, miniprepped, and sequenced.

circRNA Synthesis

CircRNAs were synthesized using in vitro transcription (IVT) kits (HiScribe T7 High Yield RNA Synthesis Kit). IVT templates were PCR amplified (Q5 Hot Start High-Fidelity 2× Master Mix) for 30 cycles and column purified prior to RNA synthesis (DNA Clean & Concentrator-100). The following forward and reverse oligos were used

circBB-T7promoter F:
AAAAAAAAAAAAAAAAAAAAAAAAAAAggccagtgaat
tgtaatacgactcactataggg
circBB-intron-poly(A) R:
TTTTTTTTTTTTTTTTTTTTTTTTTTTTTTTTTTTTTT
TTTTTTTtagaaggcacagttaacgcggccgc

One microgram of circRNA template was used per 20 μL IVT reaction. Reactions were incubated overnight at 37° C. with shaking at 1,000 rpm with a heated lid. IVT templates were subsequently degraded with 2 μL of DnaseI per IVT reaction for 20 minutes at 37° C. with shaking at 1,000 rpm. The remaining RNA was column purified prior to further enzymatic reactions.

To isolate circRNAs, column purified RNA was digested with one unit of RnaseR per microgram of RNA for 60 minutes at 37° C. with shaking at 1,000 rpm. Samples were then column purified, quantified using a Nanodrop One spectrophotometer, and verified for complete digestion using an Agilent TapeStation. In some instances due to reagent shortages, verification was performed with agarose gel under formamide-based denaturing conditions (NEB B0363S). In cases of incomplete digestion of linear RNAs, RnaseR digestion was repeated.

mRNA Synthesis

IVT templates for mRNA synthesis were PCR amplified (Q5 Hot Start High-Fidelity 2× Master Mix) for 30 cycles and column purified prior to RNA synthesis (DNA Clean & Concentrator-100). The reverse primer in this reaction incorporated a 100 bp poly(A) tail after the 3′ UTR. mRNA was then synthesized using IVT kits (HiScribe T7 High Yield RNA Synthesis Kit) with the following modifications: CleanCap AG (TriLink N-7113) was added to a 4 mM final concentration, and N1Ψ (TriLink N-1019) was fully substituted for UTP. One microgram of mRNA template was used per 20 μL IVT reaction, Reactions were incubated for 2 hours at 37° C. with shaking at 1,000 rpm with a heated lid. IVT templates were subsequently degraded with 2 μL of DnaseI per IVT reaction for 20 minutes at 37° C. with shaking at 1,000 rpm. The remaining mRNA was column purified prior to use.

### RNA Gel Electrophoresis

1% agarose gels were prepared by melting RNase-free agarose in Tris-acetate-EDTA running buffer with addition of ethidium bromide. RNA was denatured in RNA loading buffer (Thermo Fisher) by diluting 1:1 volumetrically, heating to 72° C. for 3 minutes, and cooling on ice for 1 minute. RNA was loaded into each well and run at 100 V at room temperature until the bromophenol blue dye reached the edge of the gel. Images were taken using a Bio-Rad Gel Doc XR and Image Lab 5.2 software using the “SYBR-Safe” settings.

### Cell Culture and Transfection

HeLa (CCL-2), HEK293T (CRL-11268), HepG2 (HB-8065), and KG-1 (CCL-246) cells from ATCC were maintained with DMEM (Thermo Fisher) supplemented with 10% FBS (Gibco) and 1% penicillin-streptomycin (Gibco). For routine subculture, 0.25% TrypLE (Thermo Fisher) was used for cell dissociation. For the selection of transduced cells, puromycin (Thermo Fisher) was used at a final concentration of 1 μg/mL.

RNA delivery was achieved with TransIT-mRNA transfection, Lipofectamine transfection, or NEON electroporation. Within each experiment, the molar amount of mRNA or circRNA delivered and transfection method used was the same for all samples. For TransIT-mRNA transfections, 3 μL of TransIT-mRNA reagent (Mirus Bio) was used per microgram of circRNA. Besides this change, transfections were performed following manufacturer's instructions.

### In Vitro NanoLuciferase Assay

Cells were electroporated with the pGL4.54[luc2/TK] vector (Promega) expressing firefly luciferase and transfected with mRNA or circRNA 48 hours later. Cells were harvested at 24 hours post-transfection in 100 μL of passive lysis buffer (Promega) and lysed by rocking and pipetting for roughly 15 minutes at room temperature. Lysate was centrifuged at 4,000 ref for 10 minutes to clear debris, and 5 μL of clarified lysate was transferred into a 384-well white-bottom assay plate (Perkin Elmer). To each well, 10 μL of ONE-Glo EX from the Promega Nano-Glo Dual-Luciferase Reporter Assay System was added, after which the plate was vortexed for 1 minute, incubated at room temperature for an additional 2 minutes, and read on a TECAN Infinite Pro microplate reader.

Samples were first measured for firefly luminescence, which was used as a constitutive control. To each well, 10 μL of freshly-made NanoDLR Stop & Glo Reagent was then added, after which the plate vortexed for 1 minute and incubated at room temperature for an additional 9 minutes before NanoLuc luminescence was read. Normalized luminescence per well was calculated by dividing NanoLuc signal by firefly luminescence. Within each experiment, normalized luminescence was displayed in terms of fold change relative to mock (no RNA) transfections.

mNeonGreen Flow Cytometry Assay

CircRNAs and mRNAs expressing mNeonGreen driven by different iterations of RNA backbones were electroporated into HeLa cells via NEON electroporation. At 24 hours post-electroporation, cells were lifted using warmed TrypLE (Thermo Fisher), which was quenched with DMEM (Thermo Fisher), and incubated in PBS containing propidium iodide live-dead stain (Thermo Fisher) at room temperature for 15 minutes. Cells were analyzed via flow cytometry on an Attune NxT with the same voltages applied to all conditions. At least 50,000 live singlet cells were recorded per sample.

### Cre Reporter Assay

Cre reporter loxP-DsRED-STOP-loxP-eGFP (Addgene, #62732) was used to create a lentiviral-derived stable Cre reporter HEK293T cell line. For virus production, five million HEK293T cells were plated, and after 24 hours, plasmid encoding lentivirus Cre reporter was co-transfected with pMD2. G and psPAX2 using Lipofectamine 3000 (Thermo Fisher) following manufacturer's instructions. The supernatant containing viral particles was collected 48 hours after transfection, concentrated using Lenti-X concentrator (Clontech), and stored at −80° C. Viral particles were added to 25% confluent cells at a multiplicity of infection of 1. Cre reporter expressing HEK293T cells (CreR-293T) were selected 48 hours after transfection, maintained in selection media for seven days, and allowed to recover for one day before downstream experiments.

CircRNA and mRNA encoding Cre recombinase were synthesized as described above using the Cre sequence from LV-Cre pLKo.1 plasmid (Addgene #25997). Stable CreR-293T cells were seeded in a 12-well plate at 300,000 cells per well. CreR-293T cells were transfected with 4 μg of circRNA or mRNA encoding Cre using Lipofectamine 2000 (Thermo Fisher) following manufacturer instructions. Cre-mediated recombination was analyzed 24 hours after transfection via flow cytometry on an Attune NxT by measuring the fraction of eGFP-positive cells.

### In Vitro Transcription-Translation

Coupled IVTT was performed using the 1-Step Human Coupled IVT kit (Thermo Scientific) following manufacturer's instructions. Briefly, circRNA plasmids were incubated with HeLa lysate, accessory proteins, and the reaction mix for at least 90 minutes. An aliquot from each reaction was then used to measure NanoLuc activity as described above.

### AkaLuc Assay

CircRNAs expressing AkaLuc-P2A-CyOFP54 with different optimizations were electroporated into HeLa cells via NEON electroporation and plated in a 96-well plate. At 24 hours post-electroporation, cells were washed with PBS and incubated with 100 μL of TokeOni AkaLumine-HCl substrate (Sigma-Aldrich) diluted to 250 μM in Opti-MEM (Gibco) for 5 minutes at room temperature. Luminescence was read on a SpectraMax M5 Microplate Reader (Molecular Devices) using SoftMax Pro 7.1 software with an integration time of 1,000 ms.

### CART Synthesis

O₆-stat-N₆:A₉ CARTs, consisting of a 1:1 mixture of oleyl (O) and nonenyl-substituted (N) carbonate monomers followed by a block of α-amino ester monomers (A), were prepared as previously described (Haabeth, 2021). End group analysis of the polymer confirmed block lengths of 6 nonenyl and 6 oleyl carbonate units and 9 cationic amino-ester units.

In Vivo Delivery of circRNA and mRNA

All animal experiments were performed in 3-6 month old female BALB/c mice obtained from The Jackson Laboratory. To formulate RNAs, 10.7 ng/nucleotide of linear or circular RNA (equivalent to 10 μg of hEPO mRNA) were diluted in pH 5.5 phosphate-buffered saline, mixed with O₆-stat-N₆:A₉ CARTs at a 10:1 cation:anion ratio, and immediately injected either intraperitoneally or intravenously via the tail vein. Particle sizes for CART/circRNA complexes were −170 nm. A total volume of 150 μL was used per injection. A total volume of 150 μL was used per injection. All experimental procedures were approved by the Institutional Animal Care and Use Committee at Stanford University.

### NanoLuciferase In Vivo Imaging

In vivo circNanoLuc activity was measured using an Ami HT optical imaging system (Spectral Instruments Imaging). At each time point, mice were anesthetized with isoflurane and intraperitoneally injected with 200 μL of the fluorofurimazine substrate (Promega) reconstituted in 2.1 mL of PBS per vial. Mice were imaged after 10 minutes using default settings and an exposure time of 10 seconds. Luminescent activity was quantified using Aura 4.0 imaging software.

hEPO ELISA Assay

hEPO levels in mice were measured using the SimpleStep Human Erythropoietin ELISA kit (Abcam). At each time point, approximately 100 μL of blood was collected in heparinized capillary tubes from the tail vein of each mouse and transferred into an EDTA-coated tube. Blood was centrifuged at 2,000×g for 10 minutes with the resulting plasma used as input for the ELISA. Final concentrations for hEPO were adjusted based on the volume of plasma measured.

### Reticulocyte Counts

Reticulocytes in peripheral mouse blood were measured using the Reticulocyte Reagent System (BD Biosciences), which uses thiazole orange to label reticulocytes. Briefly, 10 μL of blood was collected from the tail vein of each mouse and immediately mixed with 1 mL of the reagent. After incubating in the dark at room temperature for 30 minutes, samples were analyzed on a BD LSR II flow cytometer with 100,000 events recorded per sample. Reticulocytes were defined as singlet red blood cells positive for thiazole orange.

### Western Blotting

HeLa cells were lysed 24 hours after electroporation using RIPA Lysis and Extraction Buffer (Thermo Fisher) containing Halt Protease and Phosphatase Inhibitor Cocktail (Thermo Fisher). The resulting lysate was clarified by centrifugation and quantified for protein using bicinchoninic acid. Subsequently, 10 μg of total protein from each sample was separated on a Bis-Tris gel and transferred to a nitrocellulose membrane using the iBlot 2 Gel Transfer Device. After blocking with 5% bovine serum albumin in 0.1% Tween-20 diluted in PBS for one hour at room temperature, the membrane was stained with a 1:500 dilution of anti-NanoLuc antibody (R&D Systems, MAB10026) in blocking buffer overnight at 4° C. Following washes, the membrane was then incubated with a 1:10,000 dilution of IRDye 680RD goat anti-mouse secondary antibody (LI-COR Biosciences, 926-68070) and visualized on an Odyssey CLx Imaging System (LI-COR Biosciences).

### RNA Structure Predictions

RNA structures were predicted using the RNAfold web server (rna.tbi.univie.ac.at/cgi-bin/RNAWebSuite/RNAfold.cgi) with default settings except for deselecting “avoid isolated base pairs.” The optimal secondary structure based on minimal free energy prediction was subsequently used to represent the RNA sequence.

### Example 1 References

Bailey, J. M., and Tapprich, W. E. (2007). Structure of the 5′Nontranslated Region of the Coxsackievirus B3 Genome:Chemical Modification and Comparative Sequence Analysis. J. Virol. 81, 650-668.
Bhattacharyya, S., and Das, S. (2006). An Apical GAGA Loop within 5′ UTR of the Coxsackievirus B3 RNA Maintains Structural Organization of the IRES Element Required for Efficient Ribosome Entry. Http://Dx.Doi.Org/10.4161/Rna.3.2.2990 3, 60-68.
Blyn, L. B., Towner, J. S., Semler, B. L., and Ehrenfeld, E. (1997). Requirement of poly(rC) binding protein 2 for translation of poliovirus RNA. J. Virol. 71, 6243-6246.
Chen, Y G., Chen, R., Ahmad, S., Verma, R., Kasturi, S. P., Amaya, L., Broughton, J. P., Kim, J., Cadena, C., Pulendran, B., et al. (2019). N6-Methyladenosine Modification Controls Circular RNA Immunity. Mol. Cell 76, 96-109.e9.
Chen, Y. G., Kim, M. V., Chen, X., Batista, P. J., Aoyama, S., Wilusz, J. E., Iwasaki, A., and Chang, H. Y. (2017). Sensing Self and Foreign Circular RNAs by Intron Identity. Mol. Cell 67, 228-238.e5.
Daijogo, S., and Semler, B. L. (2011). Mechanistic Intersections Between Picornavirus Translation and RNA Replication. Adv. Virus Res. 80, 1-24.
De Breyne, S., Yu, Y., Unbehaun, A., Pestova, T. V., and Hellen, C. U. T. (2009). Direct functional interaction of initiation factor eIF4G with type 1 internal ribosomal entry sites. Proc. Natl. Acad. Sci. 106, 9197-9202.
Durbin, A. F., Wang, C., Marcotrigiano, J., and Gehrke, L. (2016). RNAs Containing Modified Nucleotides Fail To Trigger RIG-I Conformational Changes for Innate Immune Signaling. MBio 7, e00833-16.
Engler, C., Kandzia, R., and Marillonnet, S. (2008). A one pot, one step, precision cloning method with high throughput capability. PLoS One 3.
Filbin, M. E., and Kieft, J. S. (2009). Toward a structural understanding of IRES RNA function. Curr. Opin. Struct. Biol. 19, 267-276.
Gamarnik, A. V., and Andino, R. (1997). Two functional complexes formed by KH domain containing proteins with the 5′ noncoding region of poliovirus RNA. Rna 3, 882-892.
Gamarnik, A. V., Boddeker, N., and Andino, R. (2000). Translation and Replication of Human Rhinovirus Type 14 and Mengovirus in Xenopus Oocytes. J. Virol. 74, 11983-11987.
Gibson, D. G., Young, L., Chuang, R. Y., Venter, J. C., Hutchison, C. A., and Smith, H. O. (2009). Enzymatic assembly of DNA molecules up to several hundred kilobases. Nat. Methods 6, 343-345.
Graff, J., Cha, J., Blyn, L. B., and Ehrenfeld, E. (1998). Interaction of Poly(rC) Binding Protein 2 with the 5′ Noncoding Region of Hepatitis A Virus RNA and Its Effects on Translation. J. Virol. 72, 9668-9675.
Gruber, A. R., Lorenz, R., Bernhart, S. H., Neubock, R., and Hofacker, I. L. (2008). The Vienna RNA Websuite. Nucleic Acids Res. 36, W70-W74.
Holcik, M., and Liebhaber, S. A. (1997). Four highly stable eukaryotic mRNAs assemble 3′ untranslated region RNA-protein complexes sharing cis and transcomponents. Proc. Natl. Acad. Sci. 94, 2410-2414.
Huston, N.C., Wan, H., Strine, M. S., de Cesaris Araujo Tavares, R., Wilen, C. B., and Pyle, A. M. (2021). Comprehensive in vivo secondary structure of the SARS-CoV-2 genome reveals novel regulatory motifs and mechanisms. Mol. Cell 81, 584-598.e5.
Jackson, R. J., Hellen, C. U. T., and Pestova, T. V. (2010). The mechanism of eukaryotic translation initiation and principles of its regulation. Nat. Rev. Mol. Cell Biol. 11, 113-127.
Jiang, Y., Xu, X. S., and Russell, J. E. (2006). A Nucleolin-Binding 3′ Untranslated Region Element Stabilizes β-Globin mRNA In Vivo. Mol. Cell. Biol. 26, 2419-2429.
Karikó, K., Buckstein, M., Ni, H., and Weissman, D. (2005). Suppression of RNA recognition by Toll-like receptors: The impact of nucleoside modification and the evolutionary origin of RNA. Immunity 23, 165-175.
Kieft, J. S. (2008). Viral IRES RNA structures and ribosome interactions. Trends Biochem. Sci. 33, 274-283.
Kuersten, S., and Goodwin, E. B. (2003). The power of the 3′ UTR: translational control and development. Nat. Rev. Genet. 2003 48 4, 626-637.
Luo, Z., Dong, X., Li, Y., Zhang, Q., Kim, C., Song, Y., Kang, L., Liu, Y., Wu, K., and Wu, J. (2014). PolyC-binding protein 1 interacts with 5′-untranslated region of enterovirus 71 RNA in membrane-associated complex to facilitate viral replication. PLoS One 9, 1-13.
Martinez-Salas, E., Francisco-Velilla, R., Femandez-Chamorro, J., and Embarek, A. M, (2018). Insights into structural and mechanistic features of viral IRES elements. Front. Microbiol. 8, 2629.
Mayr, C. (2019). What Are 3′ UTRs Doing?Cold Spring Harb. Perspect. Biol. 11, a034728.
McKinlay, C. J., Vargas, J. R., Blake, T. R., Hardy, J. W., Kanada, M., Contag, C. H., Wender, P. A., and Waymouth, R. M. (2017). Charge-altering releasable transporters (CARTs) for the delivery and release of mRNA in living animals. Proc. Natl. Acad. Sci. U.S.A 114, E448-E456.
Michnick, S. W., and Arnold, F. H. (1999). “Itching” for new strategies in protein engineering. Nat. Biotechnol. 1999 1712 17, 1159-1160.
Mokrejs, M., Vopilenskj, V., Kolenaty, O., Masek, T., Feketovi, Z., Sekyrova, P., Skaloudovi, B., Kriz, V., and Pospisek, M. (2006). IRESite: the database of experimentally verified IRES structures (www.iresite.org). Nucleic Acids Res. 34, D125-D130.
Murray, K. E., Steil, B. P., Roberts, A. W., and Barton, D. J. (2004). Replication of Poliovirus RNA with Complete Internal Ribosome Entry Site Deletions. J. Virol. 78, 1393-1402.
Nicholson, R., Pelletier, J., Le, S. Y., and Sonenberg, N. (1991). Structural and functional analysis of the ribosome landing pad of poliovirus type 2: in vivo translation studies. J. Virol. 65, 5886-5894.
Obi, P., and Chen, Y. G. (2021). The design and synthesis of circular RNAs. Methods 196, 85-103.
Orlandini von Niessen, A. G., Poleganov, M. A., Rechner, C., Plaschke, A., Kranz, L. M., Fesser, S., Diken, M., Löwer, M., Vallazza, B., Beissert, T., et al. (2019). Improving mRNA-Based Therapeutic Gene Delivery by Expression-Augmenting 3′ UTRs Identified by Cellular Library Screening. Mol. Ther. 27, 824-836.
Ramanathan, M., Majzoub, K., Rao, D. S., Neela, P. H., Zarnegar, B. J., Mondal, S., Roth, J. G., Gai, H., Kovalski, J. R., Siprashvili, Z., et al. (2018). RNA-protein interaction detection in living cells. Nat. Methods 2018 153 15, 207-212.
Richner, J. M., Himansu, S., Dowd, K. A., Butler, S. L., Salazar, V., Fox, J. M., Julander, J. G., Tang, W. W., Shresta, S., Pierson, T. C., et al. (2017). Modified mRNA Vaccines Protect against Zika Virus Infection. Cell 168, 1114-1125.e10.
Shaner, N.C., Lambert, G. G., Chammas, A., Ni, Y., Cranfill, P. J., Baird, M. A., Sell, B. R., Allen, JR., Day, R. N., Israelsson, M., et al. (2013). A bright monomeric green fluorescent protein derived from Branchiostoma lanceolatum. Nat. Methods 2013 105 10, 407-409.
Shi, H., Wang, X., Lu, Z., Zhao, B. S., Ma, H., Hsu, P. J., Liu, C., and He, C. (2017). YTHDF3 facilitates translation and decay of N 6-methyladenosine-modified RNA. Cell Res. 27, 315-328.
Sokoloski, K. J., Dickson, A. M., Chaskey, E. L., Garneau, N. L., Wilusz, C. J., and Wilusz, J. (2010). Sindbis Virus Usurps the Cellular HuR Protein to Stabilize Its Transcripts and Promote Productive Infections in Mammalian and Mosquito Cells. Cell Host Microbe 8, 196-207.
Souii, A., Ben M'Hadheb-Gharbi, M., and Gharbi, J. (2013). Role of RNA Structure Motifs in IRES-Dependent Translation Initiation of the Coxsackievirus B3: New Insights for Developing Live-Attenuated Strains for Vaccines and Gene Therapy. Mol. Biotechnol. 2013 552 55, 179-202.
Svitkin, Y. V., Cheng, Y. M., Chakraborty, T., Presnyak, V., John, M., and Sonenberg, N. (2017). N1-methyl-pseudouridine in mRNA enhances translation through eIF2α-dependent and independent mechanisms by increasing ribosome density. Nucleic Acids Res. 45, 6023-6036.
Sweeney, T. R., Abaeva, I. S., Pestova, T. V., and Hellen, C. U. T. (2014). The mechanism of translation initiation on Type 1 picornavirus IRESs. EMBO J. 33, 76-92.
Truong, B., Allegri, G., Liu, X. B., Burke, K. E., Zhu, X., Cederbaum, S. D., Haberle, J., Martini, P. G. V., and Lipshutz, G. S. (2019). Lipid nanoparticle-targeted mRNA therapy as a treatment for the inherited metabolic liver disorder arginase deficiency. Proc. Natl. Acad. Sci. U.S.A 116, 21150-21159.
Tusup, M., Kundig, T., and Pascolo, S. (2018). An eIF4G-recruiting aptamer increases the functionality of in vitro transcribed mRNA. EPH—Int. J. Med. Heal. Sci. (ISSN 2456-6063) 4, 29-37.
Wahlestedt, C., Salmi, P., Good, L., Kela, J., Johnsson, T., Hökfelt, T., Broberger, C., Porreca, F., Lai, J., Ren, K., et al. (2000). Potent and nontoxic antisense oligonucleotides containing locked nucleic acids. Proc. Natl. Acad. Sci. 97, 5633-5638.
Walter, B. L., Nguyen, J. H. C., Ehrenfeld, E., and Semler, B. L. (1999). Differential utilization of poly(rC) binding protein 2 in translation directed by picornavirus TRES elements. RNA 5, S1355838299991483.
Wang, X., Lu, Z., Gomez, A., Hon, G. C., Yue, Y., Han, D., Fu, Y., Parisien, M, Dai, Q., Jia, G., et al. (2014). N 6-methyladenosine-dependent regulation of messenger RNA stability. Nature 505, 117-120.
Wesselhoeft, R. A., Kowalski, P. S., and Anderson, D. G. (2018). Engineering circular RNA for potent and stable translation in eukaryotic cells. Nat. Commun. 9.
Yang, D., Cheung, P., Sun, Y., Yuan, J., Zhang, H., Carthy, C. M., Anderson, D. R., Bohunek, L., Wilson, J. E., and McManus, B. M. (2003). A Shine-Dalgarno-like Sequence Mediates in Vitro Ribosomal Internal Entry and Subsequent Scanning for Translation Initiation of Coxsackievirus B3 RNA. Virology 305, 31-43.
Zeng, C., Hou, X., Yan, J., Zhang, C., Li, W., Zhao, W., Du, S., Dong, Y., Zeng, C., Hou, X., et al. (2020). Leveraging mRNA Sequences and Nanoparticles to Deliver SARS-CoV-2 Antigens In Vivo. Adv. Mater. 32, 2004452.

### Example 2

### Methods

### CircRNA Design and In Vitro Transcription

CircRNA templates were synthesized by cloning DNA fragments into a custom entry vector which contains self-splicing introns, 5′ PABP spacer, HBA1 3′ UTR, and HRV-B3 IRES. CircRNA were synthesized using HiScribe T7 High Yield RNA Synthesis Kit (NEB E2040S). IVT templates were PCR amplified (Q5 Hot Start High-Fidelity 2×Master Mix) 440 and column purified (Zymo DNA Clean & Concentrator-100) prior to RNA synthesis as previously described (Chen and Wang, 2022). Briefly, 1 μg of circRNA PCR-template was used per 20 μL IVT reaction. Reactions were incubated overnight at 37° C. IVT templates were subsequently degraded with 2 μL of DnaseI (NWB M0303S) for 20 minutes at 37° C. The remaining RNA was column purified and digested with 1 U of RnaseR per microgram of RNA for 60 minutes at 37° C. Samples were then column purified, quantified using a Nanodrop One spectrophotometer, and verified for complete digestion using an Agilent TapeStation. When compared to linear RNA, the same sequence was used as IVT template with the addition of 100 bp poly(A) tail incorporated after the 3′ UTR. Linear RNA was then synthesized using the same IVT kit with 4 nM CleanCap AG (TriLink N-7113). CircRNA or mRNA were fluorescently labeled by incorporating 5% of Fluorescein-12-UTP (Sigma-Aldrich 11427857910) in the corresponding IVT reaction, or by post-transcriptional modification using Label ITR Nucleic Acid Labeling Kit (Mirus Bio Cy3, Cy5, Fluorescein, or AF488). In all experiments we used a mixture of unlabeled circRNA and fluorescently labeled-circRNA at 20:1 ratio. Three different circRNA were produced, circOVA which encodes Ova protein, circNanoLuc which encodes nanoluciferase protein, and circFOR that has a shift-frame mutation that interferes with protein translation. Both circOVA and circNanoLuc were enhanced for translation by adding 5% m6A modifications and 5% of 2′OMeC for in vivo delivery. Circular RNA elements and modifications are listed in Table 8.

### CircRNA Uptake by Human PBMCs

Human peripheral blood mononuclear cells (PBMCs) were isolated from buffy coat obtained from healthy donors at Stanford Blood Center on a Ficoll-Paque gradient. 10 ng/ul of circRNA either unlabeled or cy3 labeled was added to 1×105 cells in RPMI-1640 medium and incubated at 37° C. for 2 hours. Subsequently, the cells were washed with FACS buffer and stained with anti-CD14 (AF647, BioLegend 325611), anti-CD3 (AF647, BioLegend 300422), anti-CD19 (AF647, BioLegend 302222), anti-CD56 (AF647, BioLegend 318313), anti-CD15 (AF647, BioLegend 323012), or anti-CD41a (AF647, BioLegend 303725). After 30 minutes incubation on ice, the cells were then washed with FACS buffer and stained with DAPI. Uptake of circRNA in each cell subtype was analyzed by flow cytometry. Unlabeled and cy3-linear RNA were used as negative controls.

### CircRNA Uptake by Human Monocytes

Peripheral blood mononuclear cells were prepared from whole blood from healthy volunteers using Lymphoprep (StemCell Technologies 07801) following the manufacturer's protocol. Briefly, whole blood was diluted 1:1 in complete media (RPMI+2% FBS-HI (Heat Inactivated at 65 C for 30 min) and carefully layered onto an equal volume of Lymphoprep (15 ml), centrifuged at 800 g for 30 min at room temperature with brakes off, and buffy coat was carefully transferred to a fresh vial. After washing once with complete media, red blood cells were lysed at 4 C for 10 min, the cells were washed in complete media twice, and cells were frozen using a 1:1 dilution in freezing media (90% FBS-HI, 10% DMSO). Frozen PBMC were thawed, washed in media, and viability assessed using a Countess automated cell counter (ThermoFisher). 1×106 cells in 50 ul were aliquoted into each well of a 96-well plate, an equal volume of cy3-labeled circRNA dilutions. Cells were placed at 37° C. 5% CO₂ for the 484 indicated time points. At each time point, the cells were transferred to a fresh 96 well V-bottom 485 plate and washed with FACS buffer. Cells were incubated with the following at room temperature protected from light: ZombieRed live/dead stain (Biolegend 423110) for 10 min, Human TruStain FcX (Biolegend 422302) for 5 min, and the following antibody mixture for 30 min: anti-CD3e (FITC, Biolegend 300406), anti-CDllc (BV785, Biolegend 101245), anti-HLADR (BV650, Biolegend 307650), anti-CD16 (BV510, Biolegend 360733), anti-CD123 (BV421, Biolegend 306017), anti-CD19 (AF700, Biolegend 302225), anti-CD14 (AF647, Biolegend 325611). Cells were washed twice in FACS buffer and fixed in 4% PFA for 30 min. The fixed cells were washed with FACS buffer and stored at 4° C. until analyzed by flow cytometry.

### CircRNA Uptake by Human Macrophages

Human primary macrophages were differentiated as described previously28. Briefly, human PBMCs were enriched for monocytes using EasySep™ Human Monocyte Enrichment Kit without CD16 Depletion (STEMCELL Technologies 19059). The resulting monocytes were resuspended in IMDM Glutamax in the presence of 10% Human Serum and 1× Penicillin-Streptomycin at a density of 1×10⁶ cells/mL and cultured in a tissue culture dish at 37° C. for 6-7 days to obtain differentiated macrophages. Aliquot 2×10⁵ differentiated macrophage cells in 100 μl medium in a 24-well plate and add circRNA to the final concentrations as indicated. After incubation for 2 hr, the cells were harvested and cytospinned on the glass slides. The cells were stained by DAPI and the localization of the circRNA was analyzed under fluorescence microscopy.

### Cell Lines

The RAW264.7 (TIB-71) and J774A.1 (TIB-67) cell lines were purchased from ATCC and cultured in DMEM medium supplemented with 10% FBS and 1% penicillin/streptomycin (Thermo Fisher), Cells were passaged after reaching 90% confluence, detached with cell scraper, and subcultured at 1:8 ratio every two days. U937 (CRL-159.2) and THP-1 (TIB-202) cells were acquired from ATCC. Cells were maintained in suspension culture with RPMI 1640 medium supplemented with 2 mM glutamine, 10% FBS, and 1% penicillin-streptomycin. Cells were subcultured when cell concentration reached 8×105 cells/mL. MutuDC cells were purchased from Applied Biological Materials Inc. (abm T0528). Cells were maintained in EVIDM-Glutamax (Gibco 31980) medium supplemented with 10% FBS, 1% penicillin-515 streptomycin, 10 mM Hepes (Gibco 15630), and 50 μM β-mercaptoethanol (GIBCO 31350). KG-1 (CCL-246) cells were acquired from ATCC. Cells were maintained in DMEM medium supplemented with 20% FBS and 1% penicillin-streptomycin. Calu-3 (HTB-55), IMR-90 (CCL-186), and Hep G2 (HB-8065) cells were acquired from ATCC and maintained in EMEM medium supplemented with 10% FBS, 1% penicillin-streptomycin. For routine subculture, 0.25% Trypsin-EDTA (Thermo Fisher) were used for cell dissociation. All cell lines were kept in culture at 37° C. in a humidified incubator with 5% CO₂, and regularly tested for mycoplasma contamination (Lonza LT07-318).

circRNA Uptake and Transfection

Cell lines were seeded at 1×10⁵ cells per well in a 96-well plate in complete media. After 24 hours cells were washed twice with serum-free media and circRNA was added at 1 μg/pl or at the indicated concentrations. Cells were placed at 37° C. and 5% CO₂ for 18 h or the indicated time points. Media containing circRNA was then removed, cells were transferred to a v-bottom plate and washed twice with PBS, stained with Live/dead NIR fixable dye and analyzed by flow cytometry. To reduce background surface binding, we perform a mild trypsin treatment before every flow cytometry analysis, to ensure the removal of membrane bound dyes. CircRNA or mRNA transfection was performed using TransIT-mRNA transfection kit (Mirus MIR 2250), with 3 μL of TransIT-mRNA reagent (Mirus Bic) per microgram of RNA.

Inhibitors and Promoters of circRNA Uptake

RAW264 cells were seeded as previously described and incubated with 100 nM cy3-circRNA in serum free media in combination with either: 1.5 μM of unlabeled circRNA, linRNA, plasmid DNA, Poly(I:C) (Sigma-Aldrich P9582), tRNA (Roche TRNABAK-RO) or heparin (Sigma-Aldrich H3149). After 24-hour incubation, cells were analyzed by flow cytometry as previously described. RAW264 cells were seeded as previously described and treated with increasing concentrations of sodium azide (Sigma-Aldrich S2002). After 30 min pre-incubation, with sodium azide, cells were washed twice with PBS and lug of cy5-circRNA was added into serum-free media or transfected into cells. 24 hours after treatment, cells were analyzed by flow cytometry as previously described. RAW264 cells were seeded as previously described and treated for 30 min with 20 mM of 2-Deoxy-D-glucose (Tocris 4515) or 2 mg/ml of oligomycin (Sigma-Aldrich 04876). Cells were then washed twice with PBS and lug of cy5-circRNA in serum-free media was added onto cells. RAW264 cells were seeded as previously described and 1 μg/pl of cy5-circRNA in serum-free media was added onto cells in combination with 20 ng/ml of LPS (Sigma-Aldrich L4516), 0.75 μg/ml of CD40 L (AcroBiosystems M5248), or 5 ng/ml of PMA (Sigma-Aldrich P1585). 24 hours after treatment, cells were analyzed by flow cytometry as previously described.

### In Vitro NanoLuciferase Assay

Cells were harvested at 24 hours post-uptake or transfection in 100 μL of passive lysis buffer (Promega) and lysed by rocking and pipetting for roughly 15 minutes at room temperature. Lysate was centrifuged at 4,000 rcf for 10 minutes to clear debris, and 5 μL of clarified lysate was transferred into a 384-well white-bottom assay plate (Perkin Elmer). To each well, 10 μL of ONE Glo EX from the Promega Nano-Glo Dual-Luciferase Reporter Assay System was added, after which the plate was vortexed for 1 minute, incubated at room temperature for an additional 2 minutes, and read on a TECAN Infinite Pro microplate reader.

### T Cell Proliferation Assay

OT-I CD8 T cells were purified from TCR-transgenic mice OT-I by negative selection using immunomagnetic beads (Miltenyi Biotech). For direct MHC-I antigen presentation assays, MutuDC lines were seeded at 10,000 cells per well in round-bottom 96-well plates. For MHC-I restricted antigen presentation assays, MutuDC were incubated for 2 h with 1 nM SIINFEKL (OVA257-264, Sigma-Aldrich S7951), 1 mg/ml of Ovalbumin protein (InvivoGen vac-pova), 1 μg of circFOR, or 1 μg of circOVA, in the presence or absence of 1 μM CpG (ODN 1585, InvivoGen). Cells were washed three times in medium and incubated with 50,000 purified OT-I CD8 T cells (CFSE-labeled). T cell proliferation was measured after 60 h of culture by flow cytometry analysis excluding doublets and dead cells. OT-I CD8 T cells were gated as CD8+Va2+ cells. Live dividing T cells were detected as low for cell proliferation dyes (CFSE low). MutuDC were similarly transfected with circOVA with or without CART reagent at the indicated concentrations.

qRT-PCR Measurement of Immune Receptors

MutuDC cells were seeded as previously described and treated with 1 M CpG, lug circRNA in media or delivered with TransIT. 24 hours after treatment total RNA was isolated from cells using TRIzol (Invitrogen, 15596018) and Direct-zol RNA Miniprep (Zymo Research, R2052) with on column DNase I digestion, following the manufacturer's instructions. RT-qPCR analysis was performed in triplicate using Brilliant II SYBR Green qRT-PCR Master Mix (Agilent, 600825) and a LightCycler 480 (Roche). The relative RNA level was calculated by the ddCt method compared to β-Actin control. Primer sequences are listed in Table 9.

### Flow Cytometry Analysis of Cytokines and Surface Receptors

MutuDC cells were seeded as previously described and treated with luM CpG, lug circRNA in media or delivered with TransIT. 24 hours after treatment cell supernatant was collected and the cytokines levels were quantified using the cytometric bead array kit for mouse inflammatory cytokines (CBA; BD Biosciences). Similarly, cell suspensions transferred to a v-bottom plate and washed twice with PBS, stained with Live/dead NIR fixable dye and stained with anti-MHC-II (redFluor 710 Tonbo 80-5321-U025), anti-MCH-I (PE, eBioscience 12-5958-82), anti-CD86 (APCFire/750, BioLegene 105045), anti-CD40 (PerCP-eFluor 710, eBioscience 46-0401-80), and anti-CD80 (Pe-cy5, sBioscience 15-0801-82). After 30 minutes incubation on ice, the cells were then washed with FACS buffer and analyzed by flow cytometry.

### Mice and Immunizations

C57BL/6, mice were purchased from Jackson Laboratories. Mice were matched for sex and aged between 8 and 14 weeks. For immunization, mice were injected intranasally with 30 μl of circRNA (25 μg per mouse), intravenously with 100 μl of circRNA (25 or 85 μg per mouse), subcutaneously at the base of the tail with 100 μl of circRNA (25 or 50 μg per mouse), and intraperitoneally with 100 μl of CART-circOVA (9 μg per mouse). When indicated, 50 μg of Ovalbumin protein (InvivoGen vac-pova) were also delivered in combination with 30 μg of Poly(I. C) (HMW VacciGrade, InvivoGen vac-pic) or 50 ug Addavax (InvivoGen). All mice in this study were maintained under specific pathogen-free conditions, a 12-h light/12-h dark cycle and temperatures of ˜18-23° C. with 40-60% humidity.

### Flow Cytometry Analysis of Innate Immune Subsets

Draining inguinal lymph nodes from immunized mice were collected and digested with 1 mg/ml collagenase type IV (Worthington) for 20 min at 37° C., followed by smashing with a 100 μm strainer to make a single-cell suspension. Single-cell samples were then stained with Zombie UV (BUV496, BioLegend 423107), anti-Ly6C (BV780, BioLegend 128041), anti-Ly6G (APC Cy7, BioLegend 127624), anti-CD19 (BUV395, BD 563557), anti-CD3 (BB700, BD742175), anti-MHCII (AF700, eBioscience 56-5321-82), anti-CD11b (BV650, BioLegend 101239), anti-CD11c (BV421, BioLegend 117330), anti-CD86 (A647, BioLegend 105020), anti-Siglec-F (PE615 CF594, BD 562757), anti-CD45 (BV610, BioLegend 103140), anti-CD169 (PE-Cy7, BioLegend 142412), anti-PDCA-1 (BUV563, BD 749275), anti-CD8a (BUV805, BD 612898), anti-CD103 (PE, eBioscience 12-1031-82), anti-NK1.1 (BV510, BioLegend 108738) and anti-F4/80 (BUV737, BD 749283).

### CD8 T Cell Flow Cytometry Analysis

Whole lung or spleen from immunized mice were collected and digested with 1 mg/ml collagenase type IV (Worthington) for 20 min at 37° C., followed by smashing with a 100-μm strainer to make a single-cell suspension. Red blood cells were lysed before staining. Single-cell samples were then stained with Zombie Yellow (BUV570, BioLegend 423103), anti-CD3 (clone 145-2C11, BioLegend), anti-CD8ct (clone 53-6.7, BioLegend), anti-CD4 (clone RM4-5, BioLegend), anti CD44 (clone IM7, BioLegend), anti-CD45 (clone 30-F11, BioLegend), anti-CD69 (clone H1.2F3, BioLegend), anti-CD103 (clone 2E7, BioLegend) and Ova-specific Tetramer (residues 257-264).

### Antibody ELISA

Ovalbumin (Ova) protein was purchased from InvivoGen. High-binding 96-well plates were coated with 100 ng of Ova protein diluted at a concentration of 10 μg/ml in PBS. The plates were washed once and blocked with 3% non-fat milk for 1 h at 37° C. Sera samples serially diluted in 1% non-fat milk containing PBS were added to the plates and incubated at 37° C. for 1 h. The plates were washed 3× with PBS-T, and horseradish peroxidase-conjugated goat anti-mouse IgG, IgA (SouthemBiotech, 1:5,000 dilution) in PBS-T containing 1% non-fat milk was added and incubated for 1 h at 37° C. Wells were washed three times with PBS-T before addition of 3,3′,5,5′-tetramethylbenzidine substrate solution (Thermo Pierce). The reaction was stopped after 5 min by addition of 0.16 M sulfuric acid. The optical density at 450 nm was measured with a Bio-Rad microplate reader.

### Luminex Assay

This assay was performed by the Human Immune Monitoring Center at Stanford University. Mouse 48 plex Procarta kits (EPX480-20834-901) were purchased from Thermo-Fisher/Life Technologies, Santa Clara, California, USA, and used according to the manufacturer's recommendations with modifications as described. Briefly: Beads were added to a 96 well plate and washed in a BioTek ELx405 washer. Samples were added to the plate containing the mixed antibody-linked beads and incubated overnight at 4° C. with shaking. Cold (4° C.) and Room temperature incubation steps were performed on an orbital shaker at 500-600 rpm. Following the overnight incubation plates were washed in a BioTek ELx405 washer and a biotinylated detection antibody added for 60 minutes at room temperature with shaking. Plate was washed as described and streptavidin-PE was added for 30 minutes at room temperature. Plate was washed as above and reading buffer was added to the wells. Each sample was measured in duplicate or single wells. Plates were read using a Luminex 200 or a FM3D FlexMap instrument with a lower bound of 50 beads per sample per cytokine. Custom Assay Chex control beads were purchased from Radix BioSolutions, Georgetown, Texas; and added to all wells.

CART Synthesis and circRNA Complex

O6-stat-N6:A9 CARTs (in this example referred simply as CART), comprising a block of on average 12 subunits made up of a statistical 1:1 mixture of oleyl (0) and nonenyl-substituted (N) carbonate subunits followed by a block of on average 9 α-amino ester subunits (A), were prepared as previously described26. circRNA was diluted in PBS pH 5.5 and mixed with CART at 1:10 charge ratio immediately before in vitro transfection or intraperitoneal delivery into mice.

### Transcriptome Data Analysis

MutuDC cells were incubated with naked circRNA as previously described or transfected with CART-circRNA complex. After 24 hours of incubation, total RNA was extracted from the cells using the RNeasy Mini Kit (Qiagen) according to the manufacturer's protocol. The integrity of the total RNA was analyzed using an Agilent 2100 Bio-analyzer and RIN numbers were above 9. RNA libraries were prepared with the Illumina Ribo-Zero Plus rRNA Depletion Kit. The adaptor ligated libraries were sequenced using an Illumina NextSeq 500. Transcript abundances were calculated by pseudocounts using Salmon (version 1.4.0). Normalization and differential gene expression analyses were performed by the DESeq2 package (v1.32.0) in R (version 4.1.1). Enrichment analysis for GO terms were performed by the ClusterProfiler package (v4.0.5) and graphs were produced by the ggplot2 package (v3.3.5).

### Mouse Model of Subcutaneous Melanoma

B16-F10-OVA cells were harvested for injection in PBS at 1×10⁶ cells/ml. 100 ul of cell suspension (1×10⁵ cells/mouse) were subcutaneously injected into C57BL/6 mice. Mice were monitored daily for tumor incidence and growth. When palpable, tumors were measured every other day using digital calipers and measured in two dimensions. Tumor volume (V) was determined by using the formula V=L×W×D×3.14/6. Mice were sacrificed before the tumors became necrotic in the center.

### Statistical Analysis

All other statistical analysis was performed with Prism (GraphPad Software v9.2.0). For comparing two groups, P values were determined using Student's t-tests (two-tailed). For comparing more than two groups, one-way ANOVAs followed by Tukey's test were applied. Differences between groups were considered significant for P values<0.05. No statistical methods were used to predetermine sample sizes. Mice were assigned to the various experimental groups randomly. Data collection and analysis were not performed blind to the conditions of the experiments.

### Results

Naked circRNA are Taken Up by Specific Human Immune Cells

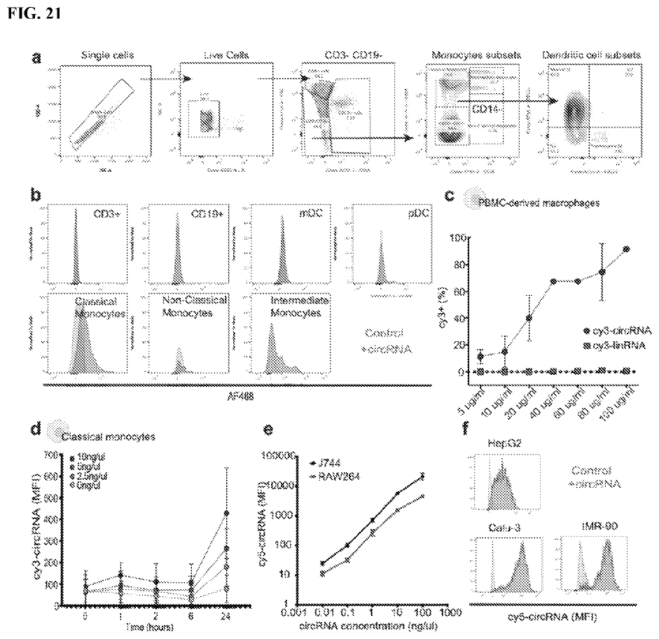

To understand the interactions between circRNAs and the hematopoietic system, circRNA uptake by human blood cells was assayed using in vitro-transcribed circRNA as previously described 11, with the incorporation of fluorescently labeled UTPs (FIG. 15a). The possibility of dye-specific uptake was minimized by swapping different fluorescent dyes such as 78 cy5, cy3 and fluorescein throughout the experiments, and similar results were obtained. Primary human peripheral blood mononuclear cells (PBMCs) were isolated from healthy donors and incubated for 2 hours with fluorescently labeled circRNA in serum-free medium, which prevents circRNA binding to albumin21, and cellular uptake of circRNA was 81 quantified by flow cytometry. Uptake of cy3-labeled circRNA (cy3-circRNA) was observed in monocytes, neutrophils, B cells, and platelets based on the expression of the cell surface markers CD14, CD15, CD19 and CD41a, respectively, on cy3+ cells (FIG. 15b). Notably, the uptake of circRNA by monocytes was highly significant compared to the rest of the subsets. In contrast, no cellular uptake was detected in any of the PBMC subsets when a Cy3-labeled linear RNA (cy3-linRNA) with the same sequence as its circRNA counterpart was used, possibly due to the unstable nature of linear RNAs. Examination of specific human myeloid subsets showed that classical monocytes (defined here as CD14+CD16−) showed higher uptake of circRNA over a 24 hour time course (FIG. 15c; FIG. 21a,b).

Differentiated Macrophages Efficiently Take Up circRNA in a Dose-Dependent Manner

CircRNA uptake was imaged via confocal microscopy, which can distinguish RNA internalization compared to cell surface binding and provides an orthogonal validation to flow cytometry. Considering that classical monocytes can differentiate into macrophages, which are specialized phagocytic cells, uptake and cellular localization of circRNA by human primary macrophages was examined, Different concentrations of cy3-circRNA were incubated with macrophages for 2 hours. As shown in FIG. 15d, the uptake of cy3-circRNA by the macrophages was dose dependent and was saturated at 40 μg/ml, which was consistent with the results obtained by flow-based uptake assay (FIG. 21c). Cy3-circRNA was detected in the cytoplasm of human macrophages, and no uptake was detected with cy3-linRNA, which was used as a negative control (FIG. 15d).

To establish a baseline model to study the molecular dynamics of circRNA uptake, circRNA uptake was measured in a panel of human and murine myeloid cell lines. CircRNA uptake levels were cell line-specific, and the strongest circRNA uptake was observed in differentiated macrophage lines mouse RAW264 and J774, human KG-1, and mouse dendritic cells line MutuDC (DCs) (FIG. 15e). Low levels of circRNA uptake were observed in monocyte like human cell lines, THP1 and U937. CircRNA uptake was concentration dependent as observed in both human classical monocytes (FIG. 21d) and macrophages cell lines J774 and RAW264 (FIG. 21e). Because many RNA therapeutics localize to liver hepatocytes when delivered in vivo22, circRNA uptake was evaluated in tissue-specific cell lines and observed strong uptake of cy5-labeled circRNA (cy5-circRNA) in the human hepatoma cell line HepG2, lung epithelial cell line Calu-3, and lung fibroblasts IMR-90 (FIG. 210.

### CircRNA Uptake by Macrophages is Fast, Saturable, and ATP-Dependent

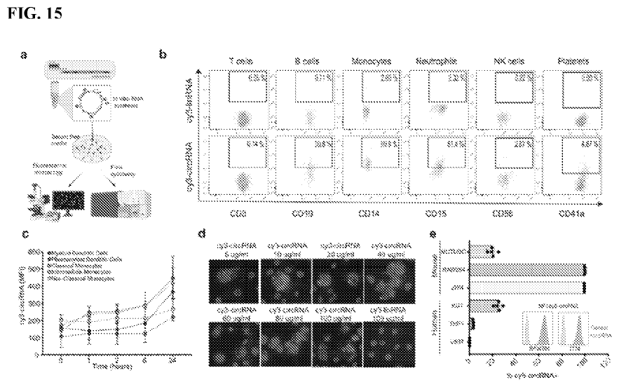

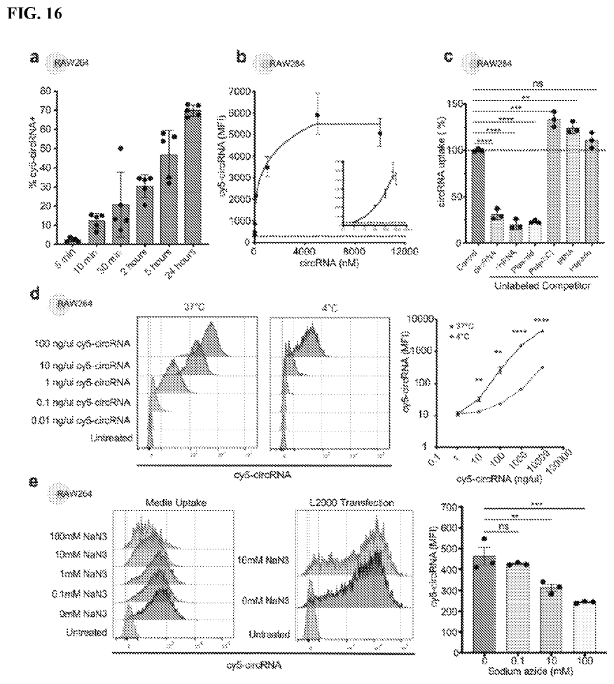

The kinetics and potential mechanisms of circRNA uptake was quantitated using the macrophage cell line RAW264. Uptake of cy5-circRNA was observed immediately after 5 min, and continued to accumulate for 24 hours, indicating a fast and continuous process (FIG. 16a). The percentage of fluorescently positive cells increased linearly over time (R2=0.7805, p value=0.0196, y=0.03877×+18.17), which may indicate the involvement of a fluid-phase endocytosis mechanism. Additional hallmarks of receptor-mediated endocytosis are (i) saturation at high ligand concentrations and (ii) specific competition for uptake by an excess of the same or related ligands. When uptake of circRNA was analyzed at increasing concentrations of cy5-circRNA, one observed a saturable process that plateaus at 1 mM circRNA concentration (FIG. 16b). To determine the specificity of circRNA uptake, a competition assay was performed. As illustrated in FIG. 16c, unlabeled circRNA, linRNA and plasmid DNA efficiently competed for cy5-circRNA uptake. In contrast, Poly(I. C), which is structurally similar to double-stranded RNA, and tRNA did not compete with circRNA uptake and instead had a positive effect in circRNA uptake. In addition, heparin, which is comparable in mass and charge to some oligonucleotides, failed to interfere with circRNA uptake. These results indicate that macrophages take up circRNAs via a saturable and ligand-selective process, likely involving one or more receptors.

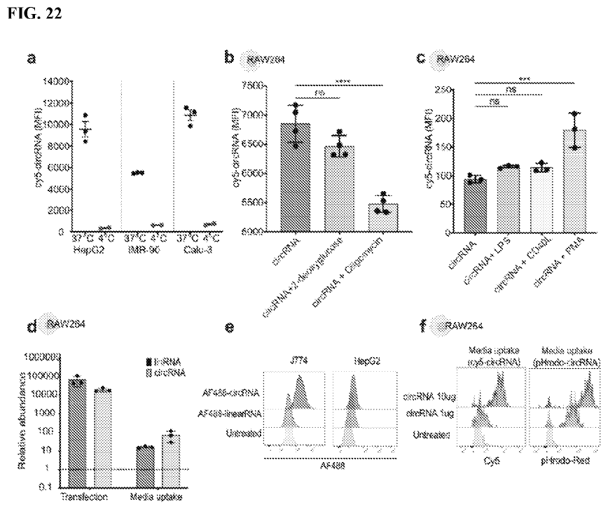

A cellular uptake process is termed active if it requires energy. This is typically determined by inhibiting energy production with cold temperature or metabolic blockade. CircRNA uptake was severely inhibited at 4° C. compared to 37° C. incubation across multiple ligand concentrations (FIG. 16d), indicating that circRNA uptake in RAW264 cells is energy dependent. CircRNA uptake was also substantially blunted at 4° C. in HepG2, IMR-90, and Calu3 cells (FIG. 22a). When RAW264 cells were treated with ATP inhibitor, sodium azide, circRNA uptake was reduced by 50% (FIG. 16e). In contrast, no effect was observed in circRNA delivery when cy5-circRNA was delivered by lipid-mediated transfection in RAW264 cells (FIG. 16e). Similar results were confirmed with other metabolic inhibitors such as oligomycin that inhibits ATP synthase, and 2-deoxyglucose that interferes with d-glucose metabolism (FIG. 22b). Conversely, it was tested if circRNA uptake is promoted by activating macrophages and thus increasing their phagocytosis mechanisms. PMA, but not LPS or CD40 L treatment, resulted in significant increase of cy5-circRNA uptake (FIG. 22c).

To quantify circRNA uptake in the absence of fluorescent dyes, qRT-PCR was performed in RAW264 cells after 24-hour incubation with unlabeled circRNA using primers that specifically target the back splice regions of circRNA. CircRNA levels after uptake were comparable to lipofectamine transfection (FIG. 22d). Uptake of linear RNA was also detectable by qRT-PCR, a more sensitive assay than fluorescence. (FIG. 22d). CircRNA uptake was greater than linRNA uptake in RAW264 and J774 cells but comparable in HepG2 cells (FIG. 22e). To track internalization of circRNA in live cells, circRNA that was covalently bound to the intracellular pH indicator pHrodo-Red, a fluorogenic probe that is weakly fluorescent at neutral pH and increasingly fluorescent in acidic conditions was used. A close correlation of pHrodo-Red signal and cy5 fluorescence (FIG. 22f). These results corroborate intracellular localization of circRNA and might indicate that some fraction of circRNA is localized in acidic compartments such as endosomes or lysosomes.

### CircRNA Uptake by Dendritic Cells Results in Translation and Presentation to T Cells

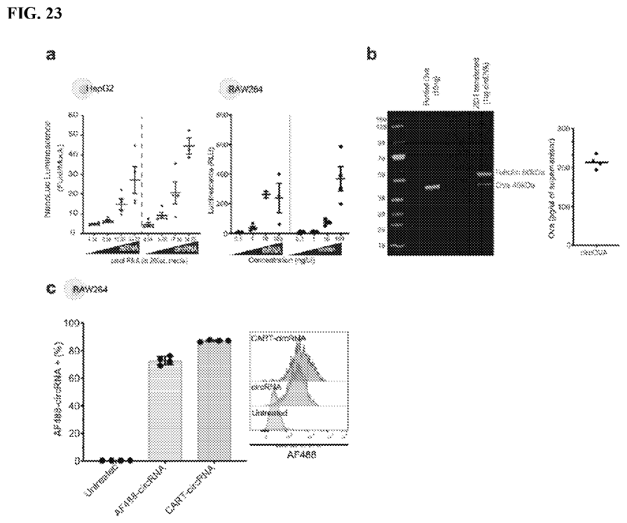

To determine if circRNA can be translated after being taken up by macrophages and dendritic cells, two circRNA molecules were designed, one encoding the reporter protein Nano luciferase (hereafter named circNanoLuc), and the other encoding a model antigen, chick Ovalbumin (hereafter named circOVA). To maximize circRNA translation, previously optimized elements were used for circRNA design and transcription11. These elements include optimized RNA chemical modification, 5′ and 3′ untranslated regions, internal ribosome entry sites (IRESs), and synthetic aptamers shown to increase circRNA translation over mRNA after a single transfection. Compared to lipid-mediated transfection, circNanoLuc uptake resulted in similar luminescent readouts 24 hours after transfection/uptake, which indicated circRNA is stable and readily available for translation after uptake in RAW264 cells (FIG. 17a). circRNA translation was also comparable to the mRNA form of the same protein, as observed in RAW264 and HepG2 cells after incubation with increasing concentrations of the corresponding mRNA or circRNA encoding NanoLuciferase (FIG. 23a).

An essential process for the initiation of cytotoxic immune responses is antigen cross presentation. Only dendritic cells, but not macrophages, contain a specialized cross-presenting transport system required for MHC class I antigen-processing. To determine if circRNA can be translated and processed for antigen presentation by dendritic cells, the antigen presentation capacity of MutuDCs was tested after incubation with circOVA. Protein translation capacity of circOVA was verified in 293T cells. 24 hours after transfection with circOVA, Ovalbumin protein was detected in cell lysate and supernatant (FIG. 23b). Using specific MHC tetramers, ovalbumin-derived peptide SIINFEKL bound to H-2 Kb of MHC class I on MutuDC cells after 24 hours incubation with circOVA, SIINFEKL peptide, or Ova protein was tested. CircOVA incubation resulted in 5-fold increase of antigen presentation capacity compared to control (FIG. 17b). Furthermore, the capability of antigen-primed dendritic cells to induce T-cell specific proliferation in vitro was measured. CircOVA uptake in MutuDC cells resulted in the strongest induction of antigen-specific T-cell proliferation after 3-day co-culture of OT-1-transgenic CD8 T cells, even above OVA protein or SINFEKL peptide incubation (FIG. 17c). This effect was enhanced after addition of CpG oligodeoxynucleotides, short synthetic single-stranded DNA, known to induce dendritic cell maturation 23. In combination, these results indicate that circRNA can be taken up, translated in the cytosol, and protein encoded by circRNA can be processed and presented to the immune system.

CircRNA Uptake Vs. CART-Mediated Delivery

To further optimize circRNA translation, circRNA delivery was tested using charge-altering releasable transporters (CARTs), a class of synthetic biodegradable materials shown to complex, protect, and efficiently deliver mRNA and circRNA intracellularly, leading to highly efficient protein translation (11,22). Efficient delivery of circRNA with CART was validated by measuring fluorescence of RAW264 cells 24 hours after delivery of AF488-labeled circRNA (FIG. 23c). The T-cell proliferation assay was performed with different amounts of circOVA delivered with or without CART into MutuDC cells. A ˜100-fold reduction in the input material required to achieve antigen-specific T-cell proliferation when circRNA is complexed with CART was observed (FIG. 17d). This observation indicated that most of the naked circRNA that is naturally internalized by dendritic cells is not efficiently translated.

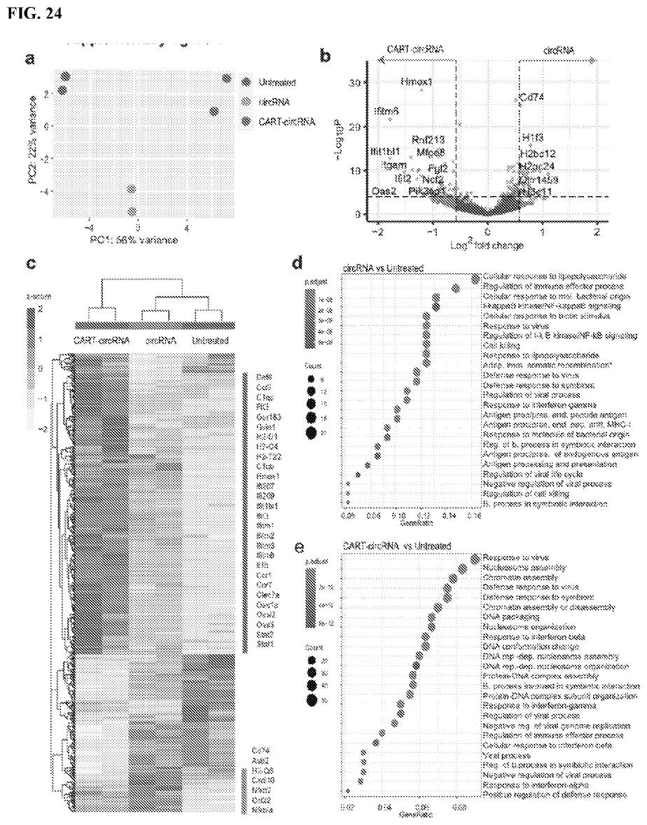

To further investigate the differences in translation efficiency after circRNA uptake compared to CART delivery, the transcriptome profile of MutuDC cells was examined after delivery of naked circRNA (hereafter referred to as circRNA) or in complex with CART (hereafter referred to as CART-circRNA). Principal component analysis (PCA) revealed each condition is easily distinguished from each other which indicates distinct transcriptome profiles characterize the path of circRNA delivery (FIG. 24a). CircRNA uptake was characterized by upregulation of CD74, a critical chaperone in antigen processing, which directs transport to the endosomal/lysosomal system (FIG. 24b). On the other hand, CART-circOVA resulted in significant upregulation of Hmox1, a membrane-bound enzyme with cytoprotective effects, in addition to upregulation of several interferon induced transmembrane proteins (FIG. 24b). Differential expression analysis between treatments revealed specific upregulation of cell membrane components involved in substrate recognition and antigen processing and presentation (CD74, Asb2, MHC-I splice variants) in the circRNA group, whereas the CART-circOVA condition resulted in significant upregulation of cytoplasmic receptors (Dhx58, Oas1 g, Oas2) (FIG. 24c). Functional annotation of differentially expressed genes after circRNA uptake compared to control resulted in significant association with “Cellular response to LPS” (FIG. 24d), indicative of Toll-like receptor signaling from endosomes. Conversely, the functional profile of CART-circOVA treated cells was more related to “Response to virus” and dsRNA signaling from cytosol (FIG. 24e). Chromatin assembly and organization, which may correlate with the CART reagent forming nanoparticle aggregates with DNA, was also among the top biological processes (FIG. 24e). In combination, the GO enrichment analysis supports that naked circRNA is localized/detected in the cell membrane or endosomal compartments, whereas circRNA delivered with CART is localized in the cytosol and readily available for translation. These results indicate that depending on the internalization method, circRNA is localized to distinct cellular compartments, and thus is recognized and processed differently. However, both treatments lead to activation of viral responses and immune effector processes. Next, the immune phenotype of antigen presenting cells after delivery of circRNA by distinct routes was determined. CircRNA uptake in MutuDC cells induced a significant increase in the mRNA levels of the cytosolic RNA sensor RIG-I and MDA5, and transcripts encoding IL1-B, TNFa and IL-6, cytokines required for dendritic cell differentiation and maturation24. Neither circRNA transfection nor CpG treatment induced these (FIG. 17e). Moreover, measurement of secretion of inflammatory cytokines and the level of activation markers on the surface of MutuDCs after circRNA uptake indicated a significant increase in TNFa and IL-6 proteins only after circRNA uptake (FIG. 17f), in addition to increased levels of MCP1, monocyte chemoattractant protein-1, which has the ability to drive the chemotaxis of myeloid and lymphoid cells25. CircRNA uptake significantly increased surface expression of DC activation markers MHC-II, MHC-1, CD80, CD40, CD86 compared to untreated samples or circRNA transfection, but comparable to CpG control (FIG. 17g). These results indicate that circRNA uptake leads to innate immune receptor activation14, leading to dendritic cell maturation. The differences observed in innate immune responses when circRNA is delivered by naked uptake or lipid encapsulation highlights the differences of the internalization mechanisms involved and the activation of distinct receptors and signaling pathways.

circRNA Induces Activation of Innate Immune Cells when Injected into Mice.

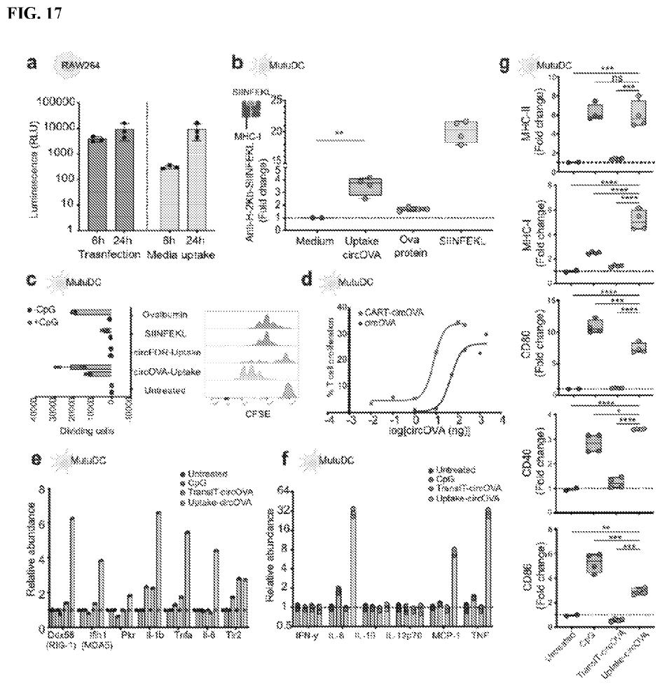

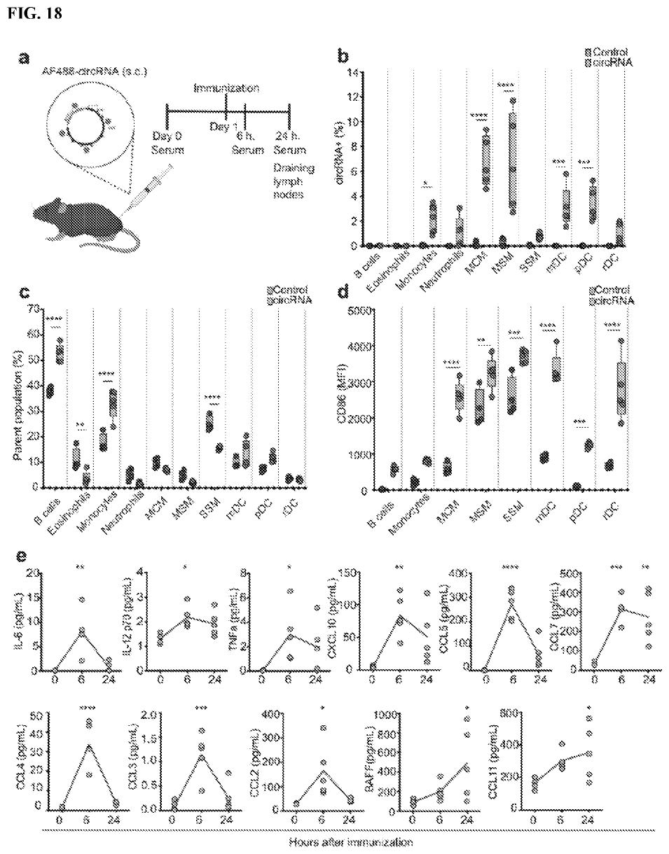

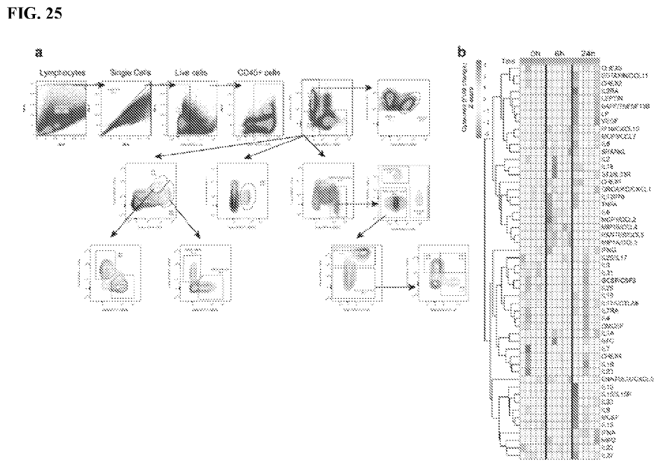

To examine the biodistribution of circRNAs when delivered in vivo, its uptake and the subsequent activation of the innate immune response following immunization were examined. g circRNA was conjugated to the fluorophore AF488 (AF488-circRNA) and subcutaneously (s.c.) injected it into C57BL/6 mice. Serum was analyzed by a Luminex panel of innate cytokines at 6- and 24-hours following immunization (FIG. 18a), while innate immune cell subsets in the draining inguinal lymph nodes (iLNs) were analyzed via flow cytometry at 24 hours following immunization (FIG. 18a, FIG. 25a). Monocytes were defined as CD11b+Ly6C+ cells, dendritic cells as CD11chigh MHCII high cells, with DC subsets further subdivided into migratory CD103+ or CD11b+DCs (mDC) and resident CD8a+ or CD11b+DCs (rDC). Lymph node (LN) macrophages were identified as CD11b+Ly6CloF4/80+/−CD169+/− and plasmacytoid dendritic cells (pDCs) as CD11b-PDCA-1+ cells. Lastly, neutrophils were defined as CD11b+Ly6G+, and eosinophils as CD11b+Signlec-F+(FIG. 25a). Innate cell activation was measured by the upregulation of the activation marker CD86 on each cell subset. At 24 hours following s.c. injection, circRNA was detected in monocytes, dendritic cells, and several macrophage subsets in the draining lymph nodes. The macrophage subsets taking up circRNAs include marginal cord macrophage (MCMs), marginal sinus macrophage (MSMs), and subcapsular sinus macrophages, (SSM), with MCMs and MSMs having the most significant uptake of circRNA (FIG. 18b). These results are similar to those observed with human primary cells in vitro (FIG. 15b), where macrophages and monocytes showed uptake of circRNA. While circRNA uptake was observed in human B cells in vitro (FIG. 15b), mouse iLN B cells did not show uptake of circRNA at 24 hours following immunization. Even though no circRNA uptake was observed by B cells in mice, B cell frequencies in the iLNs significantly increased at 24 hours post immunization (FIG. 18c), and B cell activation, as measured by CD86 upregulation, increased as well (FIG. 18d). A significant increase was observed in the frequency (FIG. 18c) and activation (FIG. 18d) of monocytes in the iLNs, as well as activation of all macrophage and dendritic cell subsets (FIG. 18d).

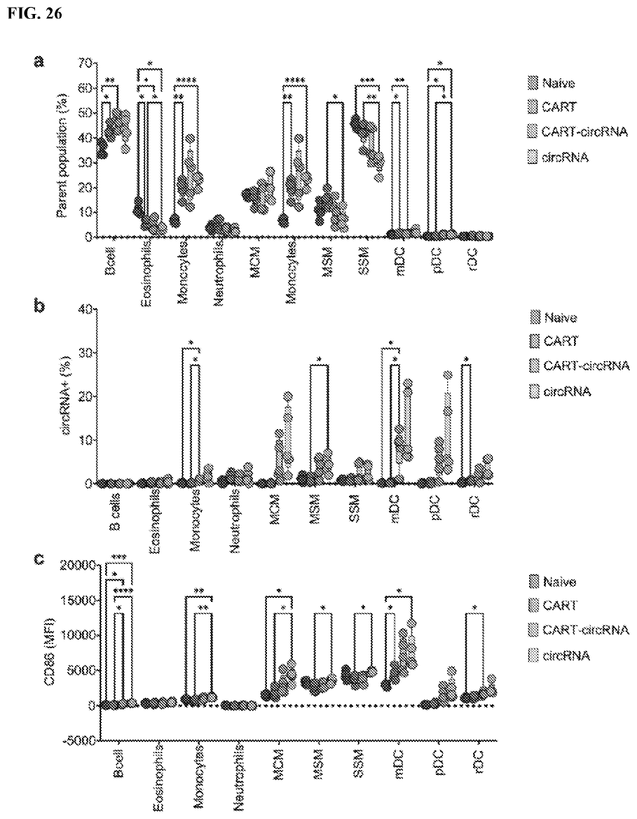

To investigate whether the innate activation and circRNA uptake is different between naked and CART-mediated circRNA delivery, mice were s.c. immunized with CART alone, naked circRNA, or CART-circRNA. Innate cell frequencies, activation and circOVA uptake was measured in the inguinal LNs at 24 h following immunization. While immunization with CART alone induced some innate immunity, such as increased monocyte frequencies in the iLNs (FIG. 26a), as well as increased CD86 expression on mDCs (FIG. 26c), its addition to circRNA did not significantly alter circRNA uptake by immune cells (FIG. 26b) or the innate cell infiltration and activation in the iLNs (FIG. 26a,c). Thus, CART does not impact the innate immune activation and uptake of circRNA.

To examine the serum cytokine response to immunization with circRNA, sera from immunized mice were analyzed at 6 and 24 hours post immunization with a Luminex panel for innate cytokines (FIG. 25b). Significant production of chemokines: CCL5, CCL4, CCL3, CCL7, CXCL10, CCL2; and cytokines: IL-6, TNFa, IL-12; were observed, with a peak at 6 hours after immunization, followed by a decrease at 24 hours (FIG. 18e). BAFF and CCL11 showed a continued increase with a peak at 24 hours (FIG. 18e). Taken together, the data supports that naked circRNA is taken up by innate immune cells when injected into mice and induces subsequent activation of several innate immune cell types and release of multiple chemokines. This observation that circRNA activates innate immunity prompted the investigation of the capacity of circRNA to induce adaptive immune responses—both when used as an adjuvant and when encoding an antigenic sequence.

circRNA Acts as a Potent Vaccine Adjuvant when Combined with Soluble Protein, Inducing Strong T Cell and Antibody Responses with Multiple Routes of Immunization

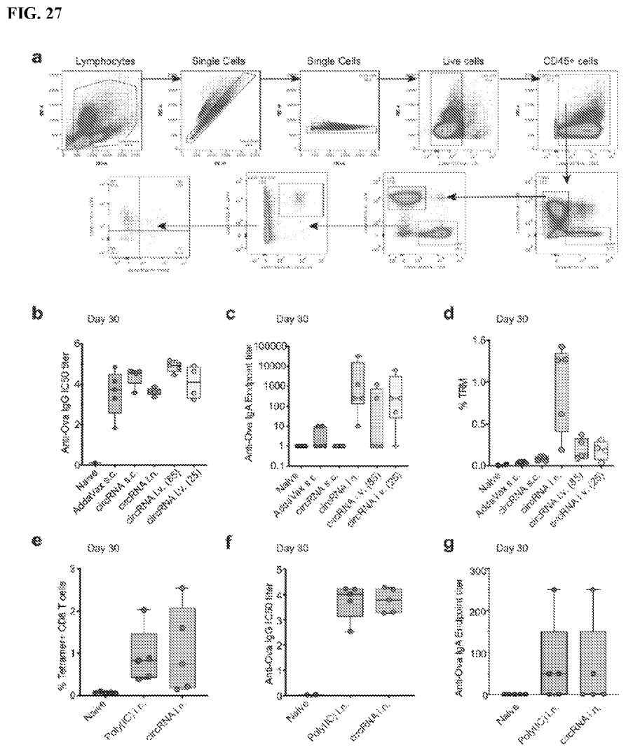

circRNA can act as an adjuvant when combined with soluble Ova protein14. To further evaluate the adjuvant capacity of circRNA, mice were immunized with immunogenic circRNA (lacking m6A modification (14)) and Ova protein (hereafter referred as OVAp) and the T cell and antibody responses were measured in the spleen, draining lymph nodes and lungs at Day 7 and Day 30 post-boost (FIG. 19). The magnitude of T cell and antibody responses were compared with three delivery strategies: subcutaneously (s.c), intranasally (i.n.), and intravenously (i.v.). In addition, circRNA was compared to the common vaccine adjuvants AddaVax, as well as Poly(I:C). It was observed that at Day 7 post-boost, subcutaneous injection of circRNA+OVAp induced comparable T cell responses to AddaVax+OVAp in the lungs, spleens and LNs (as measured by the frequency of MHC class I tetramer+CD8 T cells) (FIG. 27a). Intranasal inoculation of circRNA+OVAp induced the highest responses in the lungs at Day 7 post-boost, with the frequencies of tetramer positive cells as high as 40% in some mice (FIG. 19b).

It was observed that subcutaneous delivery of circRNA+OVAp induced a 2-fold higher frequency of antigen-specific CD8 T cells (˜10%) compared to AddaVax+OVAp (˜5%) at Day 30 post-boost (FIG. 19c), indicating a potentially enhanced memory T cell induction by circRNA compared to AddaVax. In addition, strong lung memory CD8 T cell responses were observed with the intranasal and intravenous delivery methods (FIG. 19c), with the induction of lung resident memory CD8 T cells (TRM) of both CD69+ and CD69+CD103+ subsets (FIG. 27d). To measure the antibody responses following immunization, mice were bled 30 days post-boost. Similar levels of anti-Ova IgG antibodies were observed in serum among all routes of delivery, however, only intranasal and i.v. immunization induced in anti-OVA IgA antibodies in serum (FIG. 27b,c).

Lastly, to compare circRNA and Poly(I:C) as intranasal adjuvants, mice were immunized with either adjuvant in combination with soluble Ova. circRNA and Poly(J: C) induced comparable frequencies of antigen specific CD8 T cells in the lungs at 30 days post boost (FIG. 17e), including CD69+ and CD69+CD103+ antigen-specific resident memory T cells (FIG. 19d and FIG. 27d). In addition, similar levels of anti-321 Ova IgG and IgA antibodies were induced in serum (FIG. 27f,g). Taken together, the results indicate that circRNA can be used as a potent vaccine adjuvant in many routes of immunization and induce comparable responses to Poly(I:C) and AddaVax. Moreover, mucosal immunization with circRNA as an adjuvant induces potent resident memory CD8 T cell (TRM) responses.

### CircRNA Encoding Antigen Induces Strong T Cell Responses In Vivo.

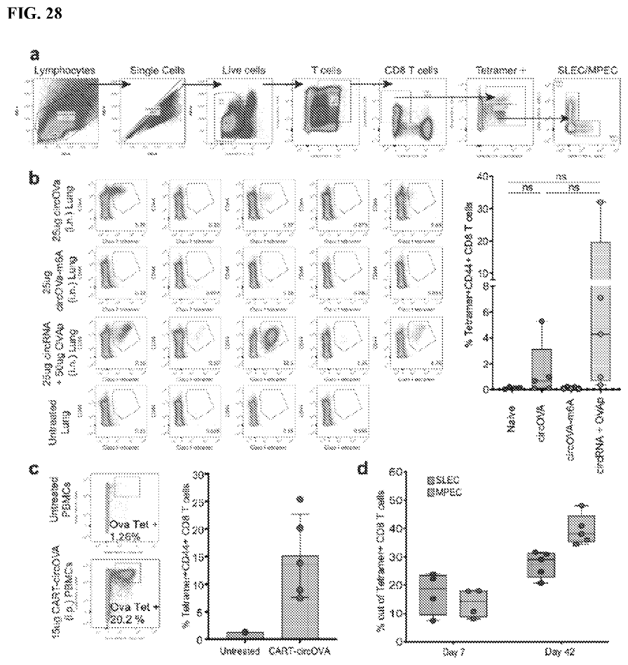

To optimize how circRNA encoding an antigen sequence can induce adaptive immune responses in vivo, the immunogenicity of circOVA was compared to soluble Ova protein combined with circRNA (hereafter referred to as circRNA+OVAp). Mice were intranasally immunized with either m6A-modified or unmodified circOVA, and 30 days post-boost lungs were analyzed for antigen-specific T cell responses. We have previously shown that the presence of m6A abrogates circRNA immunity (16), and as expected, the naked delivery of m6A-modified circOVA did not induce any OVA-specific T cell responses (FIG. 28b). For this reason, unmodified circOVA was used for the next experiments. A subset of animals in the unmodified circOVA group did generate potent OVA-specific CD8 T cell responses after naked circRNA delivery, but many other animals did not (FIG. 28b). This phenomenon indicated inefficient expression of naked circOVA when delivered in vivo and demonstrated that a delivery vehicle may be required for optimal immune responses induced by circOVA.

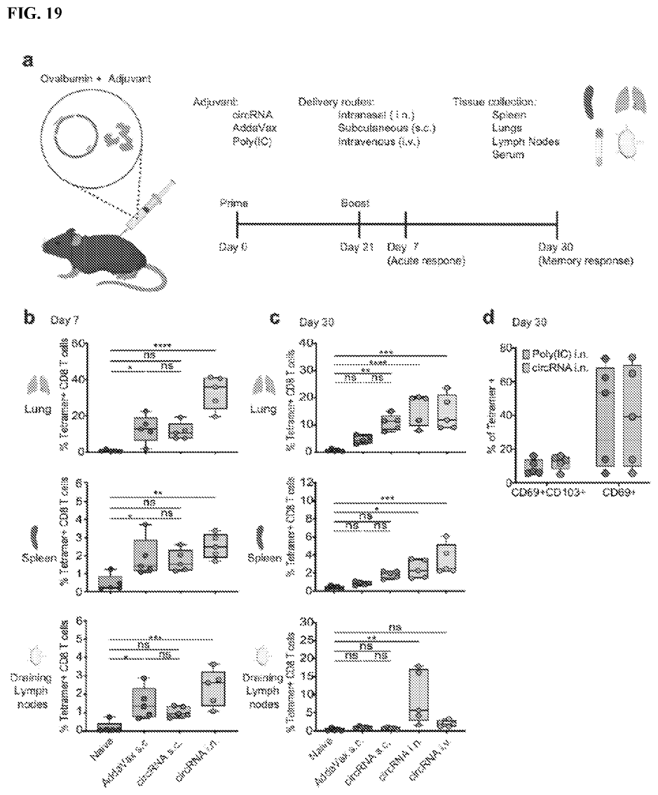

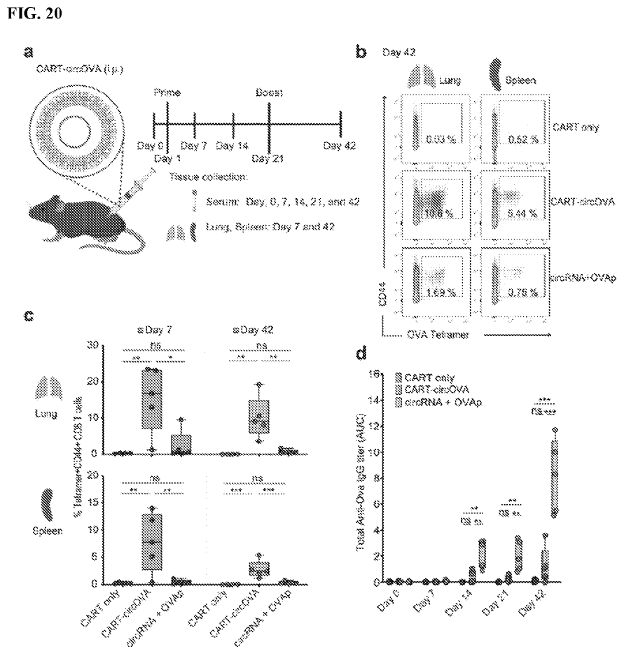

To deliver circRNAs for in vivo translation, circOVA was complexed with CART. CARTs have been shown to work effectively in mice, have a high encapsulation efficiency, are well tolerated and non-immunogenic (22,26). Three groups of mice were intraperitoneally immunized with either CART alone (vehicle only control), CART-circOVA, and circRNA+OVAp at Days 0 and 21. Antigen-specific CD8 T cell responses were assessed by MHC class I tetramer staining of lung, spleen and blood T cells at Day 7 (7 days post-prime) and Day 42 (21 days post-boost). It was observed that CART-circOVA induced potent CD8 T cell responses in the lungs, spleen (FIG. 20a, c) and blood (FIG. 20c) at 7 days following one immunization. Three weeks after the booster immunization (Day 42), significant levels of CD8 T cell responses could be seen in the CART-circOVA group in spleens and lungs (FIGS. 20b and 20c), whereas circRNA+OVAp (i.p.) did not result in strong CD8 T cell responses at this time point. In addition, KLRG1-CD127+ memory cells represented around 40% of the antigen-specific T cell population in the CART-circOVA group (FIG. 28a,d). Thus, circOVA complexed with CART induces potent T cell responses in mice. Even though circRNA uptake and innate cell activation with and without CART was not significantly different (FIG. 25c), the T cell responses were significantly enhanced with CART, indicating that the events following the circRNA uptake could be differentially influenced by CART delivery.

circRNA Uptake could be Differentially Influenced by CART Delivery.

To measure the antibody responses following immunization, mice were bled at Days 0, 7, 21 (pre-boost) and 42. It was observed that while circRNA+OVAp induces consistent and significantly higher anti-Ova IgG compared to CART-circOVA, anti-Ova antibodies were still detectable but in limited amounts in the CART-circOVA group (FIG. 20d). The lack of consistent antibody responses might result from a reduced protein secretion after CART-circOVA immunization. It was not possible to detect Ova protein in blood 24 h after immunization. This indicates the immunization strategy leads to a T-cell bias response and further optimization may provide strong antibody responses to circOVA. Taken together, the data indicates that synthetic circular RNAs can encode both the antigen and adjuvant activity required for immunization, and the route, dose, and manner of circRNA delivery impacts the potency, consistency, and memory of the programmed immune response.

### CircRNA Vaccine Induces Antitumor Efficacy.

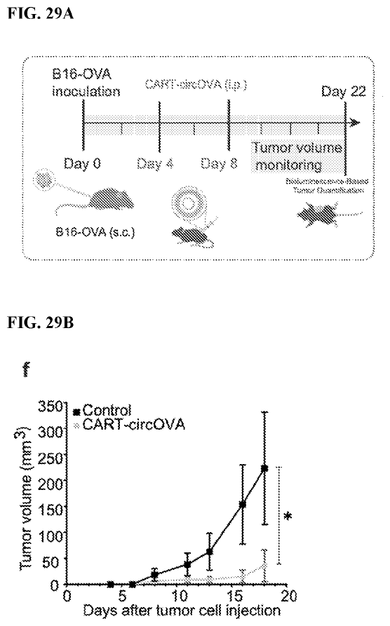

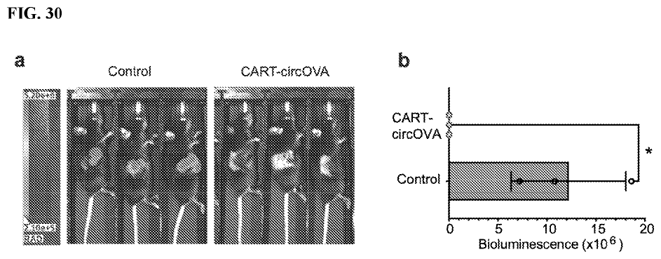

Cancer vaccination aims to induce antigen-specific T-cell-based cellular immunity capable of targeting and clearing tumor cells (44). The strong cytotoxic T-cell responses observed systemically in tissue and blood after immunization with circOVA complexed with CART prompted an investigation of circRNA as a cancer vaccine. It was contemplated that that vaccine-induced OVA-specific T cells would eradicate OVA-expressing tumor. Moreover, the anti-tumor response may be systemic and elicited by vaccinating at a site distant from the tumor (i.e., abscopal effect). The antitumor efficacy of the CART-circRNA vaccine was tested in a therapeutic regime. C57BL/6 mice were randomly assigned into two groups: a control group (untreated) and a CART-circOVA (vaccine) group. Syngeneic B16-F10-OVA melanoma cells were inoculated subcutaneously on the backs of all mice. CART-circOVA formulations were injected intraperitoneally 4 and 8 days after tumor cell inoculation (FIG. 29a). The circRNA vaccine group showed a significant tumor growth inhibition compared to the untreated group (FIG. 29b). Bioluminescence imaging confirmed eradication of luciferase labeled cancer cells (FIG. 30 a, b). These results indicate that circRNA immunization serves as an effective cancer immunotherapy to inhibit tumor growth in vivo.

TABLE 8

| | | | RNA | | | | |
|---|---|---|---|---|---|---|---|
| FIG. | circRNA | CDS | modification | IRES | 5′UTR | 3′UTR | Labeling |
| FIG. | circForeign | GFP (out of | none | iCVB3 | none | none | cy3 |
| 15b-d | | frame) | | | | | |
| FIG. | circForeign | GFP (out of | none | iCVB3 | none | none | cy5 |
| 15e | | frame) | | | | | |
| FIG. | circForeign | GFP (out of | none | iCVB3 | none | none | cy5 |
| 16a, b | | frame) | | | | | |
| FIG. | circForeign | GFP (out of | none | iCVB3 | none | none | none |
| 16c | | frame) | | | | | |
| FIG. | circForeign | GFP (out of | none | iCVB3 | none | none | cy5 |
| 16d, e | | frame) | | | | | |
| FIG. | circNanoLuc | Nanoluciferase | 5% m6A | syn1RE S- | 5′ | HBA1 | none |
| 17a | | | | RC25 | PABP | | |
| | | | | (iHRVB3- | spacer | | |
| | | | | based) | | | |
| FIG. | circOVA | Ovalbumin | 5% m6A | synIRE S- | 5′ | HBA1 | none |
| 17b | | | | RC25 | PABP | | |
| | | | | (iHRVB3- | spacer | | |
| | | | | based) | | | |
| FIG. | circOVA | ovalbumin | 5% m6A | synIRE S- | 5′ | HBA1 | none |
| 17c | | | | RC25 | PABP | | |
| | | | | (iHRVB 3- | spacer | | |
| | | | | based) | | | |
| FIG. | circForeign | GFP (Out of | none | iCVB3 | none | none | none |
| 17c | | frame) | | | | | |
| FIG. | circOVA | Ovalbumin | 5% m6A | synIRE S- | 5′ | HBA1 | none |
| 17d-g | | | | RC25 | PABP | | |
| | | | | (iHRVB3- | spacer | | |
| | | | | based) | | | |
| FIG. | circForeign | GFP (out of | none | iCVB3 | none | none | AF488 |
| 18a-e | | frame) | | | | | |
| FIG. | circOVA | Ovalbumin | 5% m6A | syn1RE S- | 5′ | HBA1 | none |
| 19a-d | | | | RC25 | PABP | | |
| | | | | (iHRVB3- | spacer | | |
| | | | | bascd) | | | |
| FIG. | circForeign | GFP (out of | none | iCVB4 | none | none | none |
| 20a-d | | frame) | | | | | |
| FIG. | circOVA | Ovalbumin | 5% 2′-O- | synIRE S- | 5′ | HBA1 | none |
| 20a-d | | | MethylIC | RC25 | PABP | | |
| | | | | (iHRVB3- | spacer | | |
| | | | | based) | | | |
| FIG. | circForeign | GFP (out of | none | iCVB3 | none | none | cy3 |
| 21 a-d | | frame) | | | | | |
| FIG. | circForeign | GFP (out of | none | iCVB3 | none | none | cy5 |
| 21e-f | | frame) | | | | | |
| FIG. | circForeign | GFP (out of | none | iCVB3 | none | none | cy5 |
| 22a-c | | frame) | | | | | |
| FIG. | circForeign | GFP (out of | none | iCVB3 | none | none | none |
| 22d | | frame) | | | | | |
| FIG. | circForeign | GFP (out of | none | iCVB3 | none | none | AF488 |
| 22e | | frame) | | | | | |
| FIG. | circForeign | GFP (out of | none | iCVB3 | none | none | cy5 |
| 22f | | frame) | | | | | |
| FIG. | circNanoLuc | Nanoluciferase | 5% m6A | syn1RES- | 5′ | HBA1 | none |
| 23a | | | | RC25 | PABP | | |
| | | | | (iHRVB 3- | spacer | | |
| | | | | based) | | | |
| FIG. | circOVA | Ovalbumin | 5% m6A | syn1RES- | 5′ | HBA1 | none |
| 23b | | | | RC25 | PABP | | |
| | | | | (iHRVB 3- | spacer | | |
| | | | | based) | | | |
| FIG. | circForeign | GFP (out of | none | iCVB3 | none | none | AF488 |
| 23c | | frame) | | | | | |
| FIG. | circForeign | GFP (out of | none | iCVB3 | none | none | none |
| 24a-c | | frame) | | | | | |
| FIG. | circForeign | GFP (out of | none | iCV83 | none | none | AF488 |
| 25a-c | | frame) | | | | | |
| FIG. | circOVA | Ovalbumin | none | syn1RES- | 5′ | HBA1 | AF488 |
| 26a-c | | | | RC25 | PABP | | |
| | | | | (iHRVB 3- | spacer | | |
| | | | | based) | | | |
| FIG. | circForeign | GFP (out of | none | iCVB3 | none | none | none |
| 27a-f | | frame) | | | | | |
| FIG. | circOVA | Ovalbumin | 5% 2′-O- | syn1RES- | 5′ | HBA1 | none |
| 28a-d | | | MethylIC | RC25 | PABP | | |
| | | | | (iHRVB3- | spacer | | |
| | | | | based) | | | |
| FIG. | circForeign | GFP (out of | none | iCV83 | none | none | none |
| 28a-d | | frame) | | | | | |

TABLE 9

| Primer name | Sequence | SEQ ID NO: |
|---|---|---|
| qRTPCR-mus-RIGI-R | CAG ATC CGA GAC ACT AAA GGG A | 33182 |
| qRTPCR-mus-RIG1-F | TCC TCA TCA GCC TTG CTT TCA | 33183 |
| qRTPCR-mus-MDA5-F | ATG GAC GCA GAT GTT CGT GG | 33184 |
| qRTPCR-mus-MDA5-R | TCC CTT CTC GAA GCA AGT GTC | 33185 |
| qRTPCR-mus-PKR-F | ATG CAC GOA GTA GCC ATT ACG | 33186 |
| qRTPCR-mus-PKR-R | TGA CAA TCC ACC TTG IT TCG T | 33187 |
| qRTPCR-mus-IL1B-F | AAG GGC TGC TTC CAA ACC TTT GAC | 33188 |
| qRTPCR-mus-IL1B-R | ATA CTG CCT GCC TGA AGC TCT TGT | 33189 |
| qRTPCR-mus-TNF-A-F | TCT CAT GCA CCA CCA TCA AGG ACT | 33190 |
| qRTPCR-mus-TNF-A-R | ACC ACT CTC CCT TTG CAG AAC TCA | 33191 |
| qRTPCR-mus-IL6-F | TCCAGTTGCCTTCTTGGGAC | 33192 |
| qRTPCR-mus-1L6-R | GTACTCCAGAAGACCAGAGG | 33193 |
| qRTPCR-mus-TLR2-F | ACAGCAAGGTCTTCCTGGTTCC | 33194 |
| qRTPCR-mus-TLR2-R | GCTCCCTTACAGGCTGAGTTCT | 33195 |
| qRTPCR-mus-B-ACT-F | GAC TAC CTC ATG AAG ATC CTG ACC | 33196 |
| qRTPCR-mus-B-AGT-R | CTC AGT AAC AGT CCG CCT AGA AG | 33197 |

### Example 2 References

1. Kristensen, L. S. et al. The biogenesis, biology and characterization of circular RNAs. Nat Rev Genet 1-17 (2019). doi:10.1038/s41576-019-0158-7
2. Memczak, S. et al. Circular RNAs are a large class of animal RNAs with regulatory potency. Nature 495, 333-338 (2013).
3. Hansen, T. B. et al. Natural RNA circles function as efficient microRNA sponges. Nature 495, 384-388 (2013).
4. Ashwal-Fluss, R. et al. circRNA biogenesis competes with pre-mRNA splicing. Molecular Cell 56, 55-66 (2014).
5. Huang, A., Zheng, H., Wu, Z., Chen, M. & Huang, Y. Circular RNA-protein interactions: functions, mechanisms, and identification. Theranostics 10, 3503-3517 (2020).
6. Yang, Q. et al. A circular RNA promotes tumorigenesis by inducing c-myc nuclear translocation. Cell Death Differ 24, 1609-1620 (2017).
7. Pamudurti, N. R. et al. Translation of CircRNAs. Molecular Cell 66, 9-21.e7 (2017).
8. Chen, C. K. et al. Structured elements drive extensive circular RNA translation. Molecular Cell 81, 4300-4318.e13 (2021).
9. Yang, Y. et al. Extensive translation of circular RNAs driven by N6-methyladenosine. Cell Res. 27, 626-641 (2017).
10. Wesselhoeft, R. A., Kowalski, P. S. & Anderson, D. G. Engineering circular RNA for potent and stable translation in eukaryotic cells. Nature Communications 9, 2629 (2018).
11. Chen, R. & Wang, S. K. Engineering circular RNA for enhanced protein production. Nature Biotechnology 1-40 (2022).
12. Liu, C. X. et al. Structure and Degradation of Circular RNAs Regulate PKR Activation in Innate Immunity. CELL 177, 865-880.e21 (2019).
13. Feng, Z. et al. Functions and Potential Applications of Circular RNAs in Cancer Stem Cells. Front Oncol 9, 500 (2019).
14. Chen, Y. G. et al. N6-Methyladenosine Modification Controls Circular RNA Immunity. Molecular Cell 76, 96-109.e9 (2019).
15. Liu, C. X. & Chen, L. L. Expanded regulation of circular RNA translation. Molecular Cell 81, 4111-4113 (2021).
16. Zhang, Z., Yang, T. & Xiao, J. Circular RNAs: Promising Biomarkers for Human Diseases. EBioMedicine 34, 267-274 (2018).
17. Lasda, E. & Parker, R. Circular RNAs Co-Precipitate with Extracellular Vesicles: A Possible Mechanism for circRNA Clearance. PLoS ONE 11, e0148407-11 (2016).
18. Vausort, M. et al. Myocardial Infarction-Associated Circular RNA Predicting Left Ventricular Dysfunction. J Am Coll Cardiol 68, 1247-1248 (2016).
19. Hansen, E. B. et al. The transcriptional landscape and biomarker potential of circular RNAs in prostate cancer. Genome Med 14, 8-16 (2022).
20. Qu, L. et al. Circular RNA vaccines against SARS-CoV-2 and emerging variants. CELL 185, 1728-1744.e16 (2022).
21. Alinovskaya, L. I., Sedykh, S. E 913., Ivanisenko, N. V., Soboleva, S. E. & Nevinsky, G. A. How human serum albumin recognizes DNA and RNA. Biol Chem 399, 347-360 (2018).
22. McKinlay, C. J. et al. Charge-altering releasable transporters (CARTs) for the delivery and release of mRNA in living animals. Proc. Natl. Acad. Sci. U.S.A. 114, E448-E456 (2017).
23. Askew, D., Chu, R. S., Krieg, A. M. & Harding, C. V. CpG DNA induces maturation of dendritic cells with distinct effects on nascent and recycling MHC-II antigen-processing mechanisms. J. I. 165, 6889-6895 (2000).
24. Wu, X., Xu, F., Liu, J. & Wang, G. Comparative study of dendritic cells matured by using IL-1β, IL-6, TNF-α and prostaglandins E2 for different time span. Exp Ther Med 14, 1389-1394 (2017).
25. Cortez, A. & Muxfeldt, E. Monocyte chemoattractant protein-1 and hypertension: An overview. Hipertens Riesgo Vasc 39, 14-23 (2022).
26. Haabeth, O. A. W. et al. An mRNA SARS-CoV-2 Vaccine Employing Charge-Altering Releasable Transporters with a TLR-9 Agonist Induces Neutralizing Antibodies and T Cell Memory. ACS Cent Sci 7, 1191-1204 (2021).
27. Chen, Y. G. et al. Sensing Self and Foreign Circular RNAs by Intron Identity. Molecular Cell67, 228-238.e5 (2017).
28. Liu, J. et al. Targeting macrophage checkpoint inhibitor SIRPα for anticancer therapy. JCI Insight 5, (2020).

Various embodiments of this invention are described herein, including the best mode known to the inventors for carrying out the invention. Variations of these embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

SEQUENCE APPENDIX

| Description | Sequence | SEQ ID NO: |
|---|---|---|
| iPV1 IRES | ttaaaacagctctggggttgtacccaccccagaggcccacgtggc | 33045 |
| | ggctagtactccggtattgcggtacccttgtacgcctgttttata | |
| | ctcccttcccgtaacttagacgcacaaaaccaagttcaatagaag | |
| | ggggtacaaaccagtaccaccacgaacaagcacttctgtttcccc | |
| | ggtgatgtcgtatagactgcttgcgtggttgaaagcgacggatcc | |
| | gttatccgcttatgtacttcgagaagcccagtaccacctcggaat | |
| | cttcgatgcgttgcgctcagcactcaaccccagagtgtagcttag | |
| | gctgatgagtctggacatccctcaccggtgacggtggtccaggct | |
| | gcgttggcggcctacctatggctaacgccatgggacgctagttgt | |
| | gaacaaggtgtgaagagcctattgagctacataagaatcctccgg | |
| | cccctgaatgcggctaatcccaacctcggagcaggtggtcacaaa | |
| | ccagtgattggcctgtcgtaacgcgcaagtccgtggcggaaccga | |
| | ctactttgggtgtccgtgtttccttttattttattgtggctgctt | |
| | atggtgacaatcacagattgttatcataaagcgaattggattggc | |
| | catccggtgaaagtgagactcattatctatctgtttgctggatcc | |
| | gctccattgagtgtgtttactctaagtacaatttcaacagttatt | |
| | tcaatcagacaattgtatcata | |
| iPV2 IRES | ttaaaacagctctggggttgttcccaccccagaggcccacgtggc | 33046 |
| | ggccagtacactggtatcgcggtacctttgtacgcctgttttata | |
| | ctcccttcccccgtaacttagaagcacaacgtccaagttcaatag | |
| | aagggggtacaaaccagtaccaccacgaacaagcacttctgttcc | |
| | cccggtgaggctgtataggctgtttccacggctaaaagcggctga | |
| | tccgttatccgctcatgtacttcgagaagcctagtatcaccttgg | |
| | aatcttcgatgcgttgcgctcaacactcaaccccagagtgtagct | |
| | taggtcgatgagtctggacgttcctcaccggcgacggtggtccag | |
| | gctgcgttggcggcctacctgtggcccaaagccacaggacgctag | |
| | ttgtgaacaaggtgtgaagagcctattgagctacctgagagtcct | |
| | ccggcccctgaatgcggctaatcctaaccacggagcaggcagtgg | |
| | caatccagcgaccagcctgtcgtaacgcgcaagttcgtggcggaa | |
| | ccgactactttgggtgtccgtgtttccttttatttttacaatggc | |
| | tgcttatggtgacaattattgatagttatcataaagcaaattgga | |
| | ttggccatccggtgagaatttgattattaaattactctcttgttg | |
| | ggattgctcctttgaaatcctgtgcactcacacctattggaatta | |
| | cctcattgttgagatattattaccact | |
| iPV3 IRES | ttaaaacagctctggggttgctcccaccccagaggcccacgtggc | 33047 |
| | ggccagtactctggtattgcggtacctttgtacgcctgttttata | |
| | ctccctccccccgtgcaactttagaagaaatccacaaagttcaat | |
| | agagggggtgcaaaccagcaccaccacgaacaagcacttctgttt | |
| | ccccggtgatgtcgtataagctgtacccacggctgaaggcgacgg | |
| | atccgttatccgcttgagtacttcgagaagcctagtattaccttg | |
| | gaatcttcgacgcgttgcgctcagcactctaccccgagtgtagct | |
| | taggtcgatgagtctgggcacgccccaccggcgacggtggcccag | |
| | gctgcgttggcggcctacccatggctatcaccatgggacgctagt | |
| | tgtgaacaaggtgtgaagagcctattgagctacccaagagtcctc | |
| | cggcccctgaatgcggctaatcctaaccacggagcaagtgtcctc | |
| | aacccaggggatggcttgtcgtaacgcgaaagtctgtggcggaac | |
| | cgactactttgggtgtccgtgtttccttttatttttatgtatggc | |
| | tgcttatggtgacaatcaaaggttgttaccataaagcaatttgga | |
| | ttggccatccggtgagaatcaaacatattatctacctgtttgttg | |
| | ggttttcttcttttacttgaacaatacctctaataataactgcta | |
| | tattgtcaataagacattatcatcaca | |
| iHRV1 IRES | ttaaaactgggtgtgggttgttcccacccacaccacccaatgggt | 33048 |
| | gttgtactctgttattccggtaactttgtacgccagtttttccct | |
| | cccctccccatccttttacgtaacttagaagttttaaatacaaga | |
| | ccaatagtaggcaactctccaggttgtctaaggtcaagcacttct | |
| | gtttccccggttgatgttgatatgctccaacagggcaaaaacaac | |
| | agataccgttatccgcaaagtgcctacacagagcttagtaggatt | |
| | ctgaaagatctttggttggtcgttcagctgcatacccagcagtag | |
| | accttgcagatgaggctggacattccccactggtaacagtggtcc | |
| | agcctgcgtggctgcctgcgcacctctcatgaggtgtgaagccaa | |
| | agatcggacagggtgtgaagagccgcgtgtgctcactttgagtcc | |
| | tccggcccctgaatgcggctaaccttaaacctgcagccatggctc | |
| | ataagccaatgagtttatggtcgtaacgagtaattgcgggatggg | |
| | accgactactttgggtgtccgtgtttcactttttcctttattaat | |
| | tgcttatggtgacaatatatatattgatatatattggcatc | |
| iHRV2 IRES | ttaaaactggatccaggttgttcccacctggatttcccacaggga | 33049 |
| | gtggtactctgttattacggtaactttgtacgccagttttatctc | |
| | ccttcccccatgtaacttagaagtttttcacaaagaccaatagcc | |
| | ggtaatcagccagattactgaaggtcaagcacttctgtttccccg | |
| | gtcaatgttgatatgctccaacagggcaaaaacaactgcgatcgt | |
| | taaccgcaaagcgcctacgcaaagcttagtagcatctttgaaatc | |
| | gtttggctggtcgatccgccatttcccctggtagacctggcagat | |
| | gaggctagaaataccccactggcgacagtgttctagcctgcgtgg | |
| | ctgcctgcacaccctatgggtgtgaagccaaacaatggacaaggt | |
| | gtgaagagccccgtgtgctcgctttgagtcctccggcccctgaat | |
| | gtggctaaccttaaccctgcagctagagcacgtaacccaatgtgt | |
| | atctagtcgtaatgagcaattgcgggatgggaccaactactttgg | |
| | gtgtccgtgtttcactttttcctttatatttgcttatggtgacaa | |
| | tatatacaatatatatattggcacc | |
| iHRV-B3 IRES | ttaaaacagcggatgggtaccccaccatccgacccactgggtgta | 33050 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggtggatttcccctc | |
| | cactagtttggtcgatgaggctaggaattccccacgggtgaccgt | |
| | gtcctagcctgcgtggcggccaacccagcttatgctgggacgcct | |
| | ttttatagacatggtgtgaagactcgcatgtgcttggttgtgatt | |
| | cctccggcccctgaatgcggctaaccttaaccctggagccttgtg | |
| | tcacaaaccagtgatgataaggtcgtaatgagcaattccgggacg | |
| | ggaccgactactttgggtgtccgtgtttcttatttttcttattat | |
| | tgtcttatggtcacagcatatatataacatatactgtgatc | |
| iCVA1 IRES | ttaaaacagctctggggttgttcccaccccagaggcccacgtggc | 33051 |
| | ggccagtactctggtattgcggtacctttgtacgcctgttttata | |
| | tcccctccccaagtaactttagaagcaattcacaaagttcaatag | |
| | agggggtacaaaccagtaccaccatgaacaagcacttctgtttcc | |
| | ccggtgaagctgtatagactgttcccacggtcgaaaatggcagat | |
| | ccgttatccgctcacgtacttcgagaagcctagtatcatcttgga | |
| | atcttcgacgcgttgcgctcagcactctaccccgagtgtagctta | |
| | ggccgatgagtctgggcataccccaccggcgacggtggcccaggc | |
| | tgcgttggcggcctacccatggctaatgtcatgggacgctagttg | |
| | tgaacaaggtgtgaagagcctattgagctacttgagagtcctccg | |
| | gcccctgaatgcggctaatcctaaccatggagcaagtgcccacaa | |
| | gccagtgggtggcttgtcgtaatgcgcaagtctatggcggaaccg | |
| | actactttgggtgtccgtgtttccttttattcttattatggctgc | |
| | ttatggtgacaacctaagattgttgtcatatagcttttggattgg | |
| | ccatccggtgatctttgagattaatataccttccttattacttca | |
| | tagtactcatttattcattacactactgtagttaca | |
| iCVA16 IRES | ttaaaacagcctgtgggttgttcccacccacagggcccactgggc | 33052 |
| | gctagcacactgattctacgggatccttgtgcgcctgttttataa | |
| | tccttcccccaagtagcaacttagaagctttgtacaatcacgacc | |
| | agtagtgggcgtggcgcgccagtcacgtcttggtcaagcacttct | |
| | gtatccccggactgagtatcaatagactgctcacgcggttgaagg | |
| | agaaaacgttcgttacccggctaactacttcgagaaacccagtag | |
| | caccgtgaaagttgcggagtgtttcgctcagcacttcccccgtgt | |
| | agatcaggtcgatgagtcactgtaaaccccacgggcgaccgtgac | |
| | agtggctgcgttggcggcctgcccatggggtaacccatgggacgc | |
| | tctaatacagacacggtgtgaagagtctattgagctagttagtag | |
| | tcctccggcccctgaatgcggctaatcctaactgcggagcgcgca | |
| | ccctcaacccagggggcggcgtgtcgtaatgggtaactctgcagc | |
| | ggaaccgactactttgggtgtccgtgtttccttttattccttatt | |
| | ggctgcttatggtgacaattgaaaagttgttaccatatagctatt | |
| | ggattggccatccggtgtctaatagagctattgtttacttattta | |
| | ttggatacgtccctcttaatctcaaagtcattcaaactcttgatt | |
| | atatattgctccttaactgtaagaa | |
| iCVB2 IRES | ttaaaacagcctgtgggttgatcccacccacagggcccactgggc | 33053 |
| | gctagcacactggtattacggtacctttgtgcgcctgttttatct | |
| | acccttgcccaatgcaacatagaagcttagtccaaaaggtcaata | |
| | ggcagcccagtgcaccaactgggtccagaccaagcacttctgtta | |
| | ccccggaccaagtatcagtaggctgctcacgcggtgaaggagaaa | |
| | acgttcgttacccggccaactacttcgagaaacctagtagcacca | |
| | tgaaggttgcgcagtgtttcgctccgcacagccccagtgtagatc | |
| | aggtcgatgagccaccgcattccccacggggtgaccgtggcgggt | |
| | ggctgcgttggcggcctgcccatggggtaacccatgggacgcttc | |
| | aatactgacatggtgtgaagagtctattgagctagttggtagtcc | |
| | tccggcccctgaatgcggctaatcctaactgcggagcgtgcgctt | |
| | gcaacccagtgagtagcacgtcgtaatgggtaactctgcagcgga | |
| | accgactactttgggtgtccgtgtttcctttattccttgtctggc | |
| | tgcttatggtgacaattgagagattgttgccatatagctattgga | |
| | ttggccatccggtatctaacagggcgatcatttacctgtttgttg | |
| | gtttcatacccttgaatcttgaggtttataataccctcactctta | |
| | taataaaactcaatacatcaag | |
| iCVB3 IRES | ttaaaacagcctgtgggttgatcccacccacagggcccattgggc | 33054 |
| | gctagcactctggtatcacggtacctttgtgcgcctgttttatac | |
| | cccctcccccaactgtaacttagaagtaacacacaccgatcaaca | |
| | gtcagcgtggcacaccagccacgttttgatcaagcacttctgtta | |
| | ccccggactgagtatcaatagactgctcacgcggttgaaggagaa | |
| | agcgttcgttatccggccaactacttcgaaaaacctagtaacacc | |
| | gtggaagttgcagagtgtttcgctcagcactaccccagtgtagat | |
| | caggtcgatgagtcaccgcattccccacgggcgaccgtggcggtg | |
| | gctgcgttggcggcctgcccatggggaaacccatgggacgctcta | |
| | atacagacatggtgcgaagagtctattgagctagttggtagtcct | |
| | ccggcccctgaatgcggctaatcctaactgcggagcacacaccct | |
| | caagccagagggcagtgtgtcgtaacgggcaactctgcagcggaa | |
| | ccgactactttgggtgtccgtgtttcattttattcctatactggc | |
| | tgcttatggtgacaattgagagattgttaccatatagctattgga | |
| | ttggccatccggtgaccaatagagctattatatatctctttgttg | |
| | ggtttataccacttagcttgaaagaggttaaaacattacaattca | |
| | ttgttaagttgaatacagcaaa | |
| iEV-A71 IRES | ttaaaacagcctgtgggttgcacccactcacagggcccacgtggc | 33055 |
| | gctagcactctggttctacggaacctttgtgcgcctgttttacgc | |
| | cccttcccccaatttgcaacttagaagcaatacacaacactggtc | |
| | aacagtaggcatggcgcaccagccatgtcttgatcaagcacttct | |
| | gtttccccggaccgagtatcaatagactgctcacgcggttgaagg | |
| | agaaagcgtccgttatccggctaactacttcgagaaacttagtaa | |
| | caccattgaagctgcagagtgtttcgctcggcacttcccccgtgt | |
| | agatcaggtcgatgagtcactgcaatccccacgggtgaccgtggc | |
| | agtggctgcgctggcggcctgcctatggggtaacccataggacgc | |
| | tctaatgcggacatggtgcgaagagtctattgagctagttagtgg | |
| | tcctccggcccctgaatgcggctaatcctaactgcggagcacatg | |
| | ccttcaatccagagggtagtgtgtcgtaatgggcaactctgcagc | |
| | ggaaccgactactttgggtgtccgtgtttccttttattcttatat | |
| | tggctgcttatggtgacaattacagaattgttaccatatagctat | |
| | tggattggccatccggtgtgcaatagagctattatatacctattt | |
| | gttggctttgtgccactaaccttaaaatctataaccaccctcgac | |
| | tttatactaaccctcaatacagtcaaac | |
| iEV-A120 IRES | ttaaaacagcctgtgggttgtacccacccacagggcccactgggc | 33056 |
| | gctagcacactgatttcacggaatccttgtgcgcctgttttacac | |
| | cccctccccccaactcagtaacttagaagttgtaaacacacgtgg | |
| | ccaacagcgggcgtagcgcaccagctatgtcttggtcaagcactt | |
| | ctgtttccccggacagagtatcaataagctgcttgcgcggctgaa | |
| | ggagaaaatgttcgttacccggtcaactacttcgagaaacctagt | |
| | agcatcatgaatgttgcgaggcgtttcgctcagcacttcccccgt | |
| | gtagatcaggtcgatgagtcactgcactccccacgggcgaccgtg | |
| | gcagtggctgcgttggcggcctgcccatggggtgacccatgggac | |
| | gcttcaatacggacatggtgtgaagagtctattgagctagttgat | |
| | aatcctccggcccctgaatgcggctaatcctaactgcggagcaca | |
| | taccctcaacccagggggcggtgtgtcgtaacgggcaactctgca | |
| | gcggaaccgactactttgggtgtccgtgtttccttttatacttat | |
| | actggctgcttatggtgacaattgaagaattgttaccatatagct | |
| | attggattggccatccggtgactaacagagctattatatatttat | |
| | ttgttggatttgttccattaacatacacatctttcagcactttac | |
| | acctcattttacaatacaatacaagaaa | |
| iEV71 IRES | ttaaaacagcctgtgggttgcacccactcacagggcccactgggc | 33057 |
| | gcaagcactctggtacctcggtacctttgtgcgcctgttttacac | |
| | cccccccccagtgaaacttagaagcagcaaaccacgatcaatagc | |
| | gggcataacgctccagttatgtcttgatcaagcacttctgtttcc | |
| | ccggactgagtatcaatagactgctcgcgcggttgaaggagaaaa | |
| | cgttcgttatccggctagctacttcgggaaacctagtaacaccat | |
| | gaaagttgcggagagcttcgttcagcactcccccagtgtagatca | |
| | ggtcgatgagtcaccgcattccccacgggcgaccgtggcggtggc | |
| | tgcgttggcggcctgcccatggggtaacccatggggcgctctaat | |
| | acggacatggtgtgaagagtctactgagctagttagtagtcctcc | |
| | ggcccctgaatgcggctaatcccaactgcggagcacacgcccaca | |
| | agccagcgggtagcgtgtcgtaacgggtaactctgcagcggaacc | |
| | gactactttgggtgtccgtgtttctttttatctttatactggctg | |
| | cttatggtgacaattaaagaattgttaccatatagctattggatt | |
| | ggccatccggtgtgcaacagagcaattgtttacctattcattggt | |
| | ttcgtaccattaaccttgaagtctgtgaccacccttaactacatc | |
| | ttgacccttaacacagctaaac | |
| iEV-B107 IRES | ttaaaacagcctgtgggttgttcccacccgcagggcccactgggc | 33058 |
| | gctagcacactggtatcccggtacccttgtgcgcctgttttatat | |
| | accctcccccttatgtaacttagaagtatgattcaaacggtcgac | |
| | aggcggctcagtgcaccaactgagtcatgaccaagcacttctgtt | |
| | accccggactgagtatcaataagctgttcacacggctgaaggaga | |
| | aaacgttcgttacccggccaattacttcgagaaacctagtaccac | |
| | catgaaggttgcgcagtgtttcgctccacacaaccccagtgtaga | |
| | tcaggtcgatgagtcaccgcattccccacgggcgaccgtggcggt | |
| | ggctgcgttggcggcctgcccatggggcaacccatgggacgcttc | |
| | aatactgacatggtgtgaagagtctattgagctaattggtagtcc | |
| | tccggcccctgaatgcggctaatcccaactgtggagcagatactc | |
| | acaaaccagtgagcggtctgtcgtaacgggcaactccgcagcgga | |
| | accgactactttgggtgtccgtgtttctttttattcttacattgg | |
| | ctgcttatggtgacaattgacaaattgttaccatatagctattgg | |
| | attggccatccggtgacaaacagagctattgtttacttgtttgtt | |
| | ggtttcataccattaaattacaaggtcttagaaactctcaacttt | |
| | attttgacactcaatacagcaaa | |
| iHRV-A9 IRES | ttaaaactggatctgggttgttcccactcagatctcccacgtgga | 33059 |
| | gtagtacactattattacggtaatcttgtacgccagttttataat | |
| | ccccttacccaagtaacttagaagataaacacaaagaccaatagg | |
| | agatgatcatccagatcatcataggtcaagcacttctgtttcccc | |
| | ggtcaaggttgatatgctctaacagggcaaaaacagctgagatcg | |
| | ttatccgcaaagcgcctacgcaaagcctagtaactatctggaagt | |
| | tgcttggttggtcgctccgccatatcccatggtagacctggcaga | |
| | tgaggctagaaattccccactggtgacagtgttctagcctgcgtg | |
| | gctgcctgcacaccctctgggtgtgaaaccaagtaatggacaggg | |
| | tgtgaagagccccgtgtgctcgctttgagtcctccggcccctgaa | |
| | tgtggctaaccttaaccctgcagctagggcacacaatccagtgtg | |
| | tatctagtcgtaatgagcaattgcgggatgg tgggaccaactac | |
| | tttgggtgtccgtgtttcttgtttttcttttatgtttgcttatgg | |
| | tgacaatatatagtaatatattttggcatc | |
| iHRV-A21 IRES | ttaaaactgggtccaggttgttcccacctggatctcctattggga | 33060 |
| | gttgtactctattattccggtaattttgtacgccagttttatctc | |
| | cccttccccaattgcaacttagaagtttatcaatatgaccaatag | |
| | gcggtagttagccaaactaccagaggtcaagcacttctgtttccc | |
| | cggtcaaagttgatatgctccaacagggcaaaaacaactgagatc | |
| | gttatccgcaaagtgcctacgcaaagcctagtaacacctttgaag | |
| | atttatggttggtcgttccgctatttcccatagtagacctggcag | |
| | atgaggctagaaattccccactggcgacagtgttctagcctgcgt | |
| | ggctgcctgcgcaccccttgggtgcgaagccatacattggacaag | |
| | gtgtgaagagccccgtgtgctcactttgagtcctccggcccctga | |
| | atgtggctaaccttaaccctgcagctagtgcatgtaatccaacat | |
| | gttgctagtcgtaatgagcaattgcgggacgggaccaactacttt | |
| | gggtgtccgtgtttcactttttccttttaatattgcttatggtga | |
| | caatatatataaacatatatattgacacc | |
| iHRV-A57 IRES | ttgtttgatgccagttttatctccccccccaattgcaacttagaa | 33061 |
| | gatgtacacaaagaccaataggcagtggtcacccagaccactgaa | |
| | ggtcaagcacttctgtctccccggtcaaagttgatatgctccaac | |
| | agggcaaaaacaacttagatcgttatccgcaaagtgcctacgcaa | |
| | agcttagtaccatctttgagagcctatggttggtcgctccactga | |
| | accccacagtagacctggcagatgaggctagaagacccccactgg | |
| | cgacagtgttctagcctgcgtggctgcctgcacacccttacgggt | |
| | gtgaagccatatgtttgacaaggtgcgaagagccccgtgtgctca | |
| | ctttgagtcctccggcccctgaatgtggctaaccttaaccctgca | |
| | gctagtgcacacaagccagtgtgttgctagtcgtaatgagcaatt | |
| | gcgggatgggaccaactactttgggtgtccgtgtttcacttttta | |
| | cctttattttgcttatggtgacaatatatatagtatatatattgg | |
| | cacc | |
| iHRV-A89 IRES | ttaaaactgggagtgggttgttcccactcactccacccatgcggt | 33062 |
| | gttgtactctgttattacggtaactttgtacgccagtttttccca | |
| | cccttccccataatgtaacttagaagtttgtacaatatgaccaat | |
| | aggtgacaatcatccagactgtcaaaggtcaagcacttctgtttc | |
| | cccggtcaatgaggatatgctttacccaaggcaaaaaccttagag | |
| | atcgttatccccacactgcctacacagagcccagtaccatttttg | |
| | atataattgggttggtcgctccctgcaaacccagcagtagacctg | |
| | gcagatgaggctggacattccccactggcgacagtggtccagcct | |
| | gcgtggctgcctgctcacccttcttgggtgagaagcctaattatt | |
| | gacaaggtgtgaagagccgcgtgtgctcagtgtgcttcctccggc | |
| | ccctgaatgtggctaaccttaaccctgcagccgttgcccataatc | |
| | caatgggtttgcggtcgtaatgcgtaagtgcgggatgggaccaac | |
| | tactttgggtgtccgtgtttcctgtttttcttttgattgcatttt | |
| | atggtgacaatttatagtgtatagattgtcatc | |
| iHRV-A100 | ttaaaactgaatccagattgttcccatctggatttcctacatgga | 33063 |
| IRES | gttgtactctattattccggtaattttgtacgccagttttatcac | |
| | cccttcccccgtaacttagaagtttgaaacaaaagaccaatagga | |
| | ggtaactatccaagttactataggtcaagcacttctgtttccccg | |
| | gtcaaagttgatatgctccaccagggcaaaaacaattgagatcgt | |
| | tatccgcaaagtgcctacgcaaagcctagtagtatcttgaaaagc | |
| | atgtggttggttgctccgctgtaccccacagtaaacctggcagat | |
| | gaggctagaagttccccactggtgacagtgttctagcctgcgtgg | |
| | ctgcctgcgcactctttgagtgcgaagccatatgtttgacaaggt | |
| | gtgaagagccccgtgtgctcactttgagtcctccggcccctgaat | |
| | gtggctaaccttaaccctgcagctagtgcatgcaatccagcatgt | |
| | ggctagtcgtaatgagcaattgcgggatgggaccaactactttgg | |
| | gtgtccgtgtttcacttttttccttttataattgcttatggtgac | |
| | aatatatagtgatatatattgacacc | |
| iHRV-B4 IRES | ttaaaacagcggatgggtttcccaccatccgacccactgggtgta | 33064 |
| | gtgctctggtattttgtacctttgcacgcctgtttcccatttgta | |
| | cccttccttaatctccttcccccgtaacgttagaagttttggaat | |
| | tttaaagtacaataggaagcgccacatccagtggtgttgcgtaca | |
| | aatacttctgtttacccggagcgaggtataggttgtacccacggc | |
| | caaaagcctttaaccgttatccgccaatcaactacgtaacggcta | |
| | gtatcatcttgcttttgatttggtgttcgatcaggtggtatcccc | |
| | cactagtctggtcgatgaggctaggaattccccacgggcgaccgt | |
| | gtcctagcctgcgtggcggccagcccagcttttgctgggacgcct | |
| | tttcaaagacatggtgtgaagacctgcatgtgcttggttgtgagt | |
| | cctccggcccctgaatgcggctaaccttaaccctggagcccagca | |
| | gcataatccaatgttgtttgggtcgtaatgagcaattccgggacg | |
| | ggaccgactactttgggtgtccgtgtttccttttattcttacatt | |
| | gtcttatggtcacagcatatatattatatatactgtgatc | |
| iHRV-B17 IRES | tttaaacagcggatgggttccccaccatccgacccactgggtgta | 33065 |
| | gtactctggtattttgtacctttgtacgcctgtttctcccctacc | |
| | tcccaacctaaacaatcctggtaacttagaagacttaaatcatcg | |
| | tacaataggtgctgtcacatccagtgacggctagtacaagcactt | |
| | ctgtttccccggagcggagtataaatggccaccgctgtcaaaagc | |
| | tcttaaccgttatccgccaattaactacgcaacggctagtaacat | |
| | cttgttatttttagggcgttcgatcaggtgagtaaacccctcact | |
| | agtctggtcgatgaggctgagaattccccacgggcgaccgtgtct | |
| | cagcctgcgtggcggccagcccagctaatgctgggacgccttaat | |
| | tgtgacatggtgtgaagacccacgtgtgcttaattgtgagtcctc | |
| | cggcccctgaatgcggctaacctaaaccctggagccttgagacac | |
| | aatccagtgttggcaaggtcgtaatgagtaattccgggacgggac | |
| | cgactactttgggtgtccgtgtttccttttattttcaaattgttc | |
| | ttatggtcacaatataagtaatatattgtgatc | |
| iHRV-B26 IRES | ttaaaacagcgggtgggtatcccaccacccgacccactgggtgtt | 33066 |
| | gtacactggtattttgtacctttgtatgcctgtttgcacctcccc | |
| | accccttccaattacccttacccgaattttattatgcggtaacat | |
| | tagaagaagtgaacacagtgcaataggacgtatcacatccagtga | |
| | tataaagcacaagcaattcttgttccccggagctggatatagact | |
| | gctaacgtggttgaaggtccttaaccgttatccgccaaccaacta | |
| | cgagacggctagtaatatcatgtttgtctttgagcgttcgatcag | |
| | gtgaattccccattcactagtttggtcgatgaggctgagaactcc | |
| | ccacaggtgactgtgtctcagcctgcgtggcggccaacccagcca | |
| | cggctgggacgcccactgatagacatggtgtgaagacccaattgt | |
| | gcttggttgtgactcctccggcccctgaatgcggctaacctcaac | |
| | cccggagccttgtagtgtaagccaacacatacaaggtcgtaatgg | |
| | gcaactctgggacgggaccgactactttgggtgtccgtgtttcct | |
| | ttatttttatcttttgtgtcttatggttacaagtattgattgtaa | |
| | cc | |
| iHRV-B37 IRES | tttaaacagcggatgggtatcccaccatccgacccactgggtgta | 33067 |
| | gtactctggtattttgtacctttgtacgcctgttgttcctaatgt | |
| | acccaccctaaaacttcctacccaagtaacgttagaagtttcatc | |
| | aacaagtacaataggaagcatcacatccagtggtgttttgtacaa | |
| | gcacttctgtttccccggagcgaggtataggctgtacccactgcc | |
| | gaaagcctttaaccgttatccgccaaccaactacgtaaaagctag | |
| | tatcatcatgttttaaaataggcgttcgatcaggtggatcccccc | |
| | tccactagtttggtcgatgaggctaggaactccccacgggtgacc | |
| | gtgtcctagcctgcgtggcggccaacccagcttctgctgggacgc | |
| | ctttttatggacatggtgtgaagactcgcatgtgcttggttgtga | |
| | ctcctccggcccctgaatgcggctaaccttaaccccggagccctg | |
| | tgttgcaatccagtaacattagggtcgtaatgagcaatttcggga | |
| | cgggaccgactactttgggtgtccgtgtttctcatttttcttatt | |
| | attgtcttatggtcacagcatatatataacgtatatactgtgatc | |
| iHRV-B92 IRES | ttaaaacagcggatgggtatcccaccatccggcccactgggtgta | 33068 |
| | gtactctggtacattgtacctttgtacgcctgttttccccctctt | |
| | gtacccgcccttcaagctccttgcccaagtaacgttagaagtttg | |
| | aacattggtacaataggaagcatcacatccagtggtgtactgtac | |
| | aaacacttctgttgccccggagcgaggtatagatggtccccaccg | |
| | tcaaaagcctttaaccgttatccgccaatcaactacgtaatggct | |
| | agtagcaccttggatttaagttggcgttcgatcaggtggtaaccc | |
| | ccactagtttggtcgatgaggctaggaattccccacgggtgaccg | |
| | tgtcctagcctgcgtggcggccaacccagcatccgctgggacgcc | |
| | aatttaatgacatggtgtgaagacctgcatgtgcttgattgtgag | |
| | tcctccggcccctgaatgcggctaaccctaaccccggagccttgc | |
| | agcacaatccagtgttgttaaggtcgtaatgagcaattctgggat | |
| | gggaccgactactttgggtgtccgtgtttcttatttttcttgaat | |
| | ttttcttatggtcacagcatatatacattatatactgtgatc | |
| iHRV-C3 IRES | ttaaagctggatcatggttgttcccaccatgattacccacgcggt | 33069 |
| | gcagtggtcttgtattacggtacatttccataccagttttataca | |
| | ccccaccccgaaactcatagaagtttgtacacaatgaccaatagg | |
| | tggtggccatccaggtcgctaatggtcaagcacttctgtttcccc | |
| | ggcacccttgtatacgcttcacccgaggcgaaaaatgaggttgtc | |
| | gttatccgcaaagtgcctacgaaaagcctagtaacactttgaaaa | |
| | cccatggttggtcgctcagctgtttacccaacagtagacctggca | |
| | gatgaggctagacattccccaccagcgatggtggtctagcctgcg | |
| | tggctgcctgcacaccctgccgggtgtgaagccagaaagtggaca | |
| | aggtgtgaagagcctattgtgctcactttgagtcctccggcccct | |
| | gaatgtggctaaccctaaccccgtagctgttgcatgtaacccaac | |
| | atgtatgcagtcgtaatgggcaactatgggatgggaccaactact | |
| | ttgggtgtccgtgtttcctgttttactttttcattgcttatggtg | |
| | acaattgtatctgatacacttgttacc | |
| iHRV-C11 IRES | ttaaaactggatacaggttgttcccacctgtatcacccaagtggt | 33070 |
| | gtggtgctcttgtatttcggtacgtttgcacgccagtttgctacc | |
| | ccttcccttttacgtaacttagaagtttacacaaagaccaatagg | |
| | cggtggtaaatccataccactaacggtcaaatacttctgtttccc | |
| | cggcatgcgaggaataggctccaaaagggctgaagccactagtgt | |
| | cgttatccgcattggtactacgcaaagcctagtattaccttgaaa | |
| | atttcttggctggtcgctccaccagataccccactggtagacctg | |
| | gcagatgaggcaggacttaccccactggcgacagtggtcctgcct | |
| | gcgtggctgcctgcacaccccttacggggtgtgaagcccagaaac | |
| | agacaaggtgtgaagagccccgtgtgctactagtgagtcctccgg | |
| | cccctgaatgcggctaatcttaccccacagctgttgcacgcaaac | |
| | cagcgtgtatgcagtcgtaatgagcaattgtgggatggaaccgac | |
| | tactttgggtgtccgtgtttctttttattcctattatttgcttat | |
| | ggtgacaatattgatattatcagtgttgtcatc | |
| iHRV-C54 IRES | ttaaaacagctgtgtggttgttcccaccaccaggcacactgtgcg | 33071 |
| | ttgtacactgggattccggtcactttgtacgcctgtttgctatcc | |
| | cccccaacttatgtaatttagaagatgtacacaacgcccattagg | |
| | atgcggccaaaccaggtccgcttagggcaagcacttctgtttccc | |
| | cgggtgtgtgaatagactctaacagggttgaagctgtagcactcg | |
| | ttatccgcgcaactactacgcgaatgttagtagcatcctgtgttg | |
| | catttgggatttcgctccgcagaaaaccccatctgtagattaggg | |
| | caatgaggctacacataccccactggcgacagtggtgtagcctgc | |
| | gtggtgccctacccaggccttcttggcctgggattccacttacaa | |
| | gacagggtgtgaaggcactagtgtgctagttgtgagtcctccggc | |
| | ccctgaatgcggctaatcttaaccccgtagcccccgcaagtaaac | |
| | caacttgtaggtggtcgtaatgagtaattacgggatggaaccgac | |
| | tactttgggtgtccgtgtttccttttattctttatatttgcatcc | |
| | tatggttacaacataagtaatc | |
| iCVB1 IRES | ttaaaacagcctgtgggttgttcccacccacaggcccattggggc | 33072 |
| | tagcactctggtatcacggtacctttgtgcgcctgttttacatcc | |
| | cctccccaaattgtaatttagaagtttcacacaccgatcattagc | |
| | aagcgtggcacaccagccatgttttgatcaagcacttctgttacc | |
| | ccggactgagtatcaatagaccgctaacgcggttgaaggagaaaa | |
| | cgttcgttacccggccaactacttcgaaaaacctagtaacaccat | |
| | ggaagttgcggagtgtttcgctcagcactaccccagtgtagatca | |
| | ggtcgatgagtcaccgcgttccccacgggcgaccgtggcggtggc | |
| | tgcgttggcggcctgcctacggggaaacccgtaggacgctctaat | |
| | acagacatggtgcgaagagtctattgagctagttggtaatcctcc | |
| | ggcccctgaatgcggctaatcctaactgcggagcacataccctca | |
| | aaccagggggcagtgtgtcgtaacgggcaactctgcagcggaacc | |
| | gactactttgggtgtccgtgtttcattttattcctatactggctg | |
| | cttatggtgacaattgacaggttgttaccatatagttattggatt | |
| | ggccatccggtgactaacagagcaattatatatctctttgttggg | |
| | tttataccacttagcttgaaagaggttaaaacactacatctcatc | |
| | attaaactaaatacaacaaa | |
| iCVB5 IRES | ttaaaacagcctgtgggttgttcccacccgcagggcccactgggc | 33073 |
| | gccagcactctggtatcacggtacctttgtgcgcctgttttaaaa | |
| | ccctctccccaatttgaaacttagaagcaatacacctcgatcaat | |
| | agtaggcatgacacgccagccatgtcttgatcaagcacttctgtt | |
| | tccccggactgagtatcaataaactgcttgcgcggtcgaaggaga | |
| | aaacgtccgttacccgactaactacttcgagaaacccagtaacac | |
| | catggaaattgcggagtgtttcactcagcacattcccagtgtaga | |
| | tcaggtcgatgagtcaccgcattccccacgggtgaccgtggcggt | |
| | ggctgcgctggcggcctgcccatggggcaacccatgggacgcttc | |
| | aatatggacatggtgtgaagagtctattgagctagttagtagtcc | |
| | tccggcccctgaatgcggctaatcctaactgcggagcacgtgcct | |
| | ccattccagggggtggcgtgtcgtaacgggcaactctgcagcgga | |
| | accgactactttgggtgtccgtgtttcttttaattttatactggc | |
| | tgcttatggtgacaattgaaagattgttgccatatagctattgga | |
| | ttggccatccggtatccaacagagcaattgtgtacctttttgttg | |
| | gatttgtaccacttaccagaacaagttttcatacactgtgttaca | |
| | ttattagactaaacacagaaaa | |
| iEchoV1 IRES | ttaaaacagcctgtgggttgttcccacccacagggcccactgggc | 33074 |
| | gttagcacactggtatcacggtacctttgtgcgcctgttttatac | |
| | tcccccccctaaggaaactttagaagcaaagcaattgtgatcaat | |
| | agtgggtatggcacaccagtcatatcttgatcaagcacttctgtt | |
| | cccccggacttagtaccaatagactgctcaagcggttgaagggga | |
| | aaacgttcgttatccggccaactacttcgagaaacctagtagcac | |
| | catgaaagttgcggagtgtttcgctcagcacttcccccgtgtaga | |
| | tcaggctgatgagtcaccgtattccccacgggcgaccgtgacggt | |
| | ggctgcgttggcggcctgcccatggggtaacccatgggacgctct | |
| | aaaacagacacggtgcgaagagtctattgagctagttggtagtcc | |
| | tccggcccctgaatgcggccaatcctaactgcggagcacatactc | |
| | ccaatccagggagcagtgtgtcgtaatgggtaactctgcagcgga | |
| | accgactactttgggtgtccgtgtttccttttattctcacattga | |
| | ctgcttatggtgacaattgaaagattgttaccatatagctattgg | |
| | attggtcatccggtgagcaatagagctattgtttatcaatttgtt | |
| | ggatttgtaccactcaacttttctgttttgagaacactcaactac | |
| | atcttactgctaaacacatcaaa | |
| iEchoV-E11 | ttaaaacagcctgtgggttgtacccacccacagggcccactgggc | 33075 |
| IRES | gctagcacactggtatcacggtacctttgtgcgcctgttttatac | |
| | ccccttcccgcaaccgcaaatttagaagcaaagctaacccgatcg | |
| | atagcggatgcgcatgccagccgcattttgatcaagtacttctgt | |
| | ttccccggaccgagtatcaatagactgctcacgcggttgaaggag | |
| | aaaacgtccgttacccgaccaactacttcgagaaacctagtaaca | |
| | tcatgaatgttgcagggcgtttcgatcagcacgaccctggtgtag | |
| | atcaggctgatgagtcaccgcattccccacgggtgaccgtggcgg | |
| | tggctgcgttggcggcctgcctatggggtgacccataggacgctc | |
| | taatacggacatggtgcgaagagtctattgagctagttggtagtc | |
| | ctccggcccctgaatgcggttaatcctaactgcggacgacatacc | |
| | cctaatccaaggggcagtgtgtcgtaacgggcaactctgcagcgg | |
| | aaccgactactttgggtgtccgtgtttccttttatttttatactg | |
| | gctgcttatggtgacaatctcagagttgttaccatatagctattg | |
| | gattggccatccggtgagcaacagagctgtcatttatcagtttgt | |
| | tggctttatacctctaaatcacacggttttttttttttggaacgc | |
| | ttgtattcatcttaaccctcaataaggcaaa | |
| iEchoV-E15 | ttaaaacagcctgtgggttgtacccacccacagggcccactgggc | 33076 |
| IRES | gctagcactctggtattacggtacccttgtgcgcctgttttatat | |
| | accccgccccaagcaaacgctagatgtaacgcacttatgatcaat | |
| | agcaggcgtggcactccagccacgttatgatcaagcacttctgtc | |
| | tccccggaccgagtatcaatagactgctcacgcggtcgaaggaga | |
| | aaacgttcgttacccgaccagctacttcgagaaacctagtaactc | |
| | catggaggttgcagagtgtttcgttcagcacttcccccgtgtaga | |
| | tcaggctgatgagtcaccgcgttcctcacgggcgaccgtggcggt | |
| | ggctgcgttggcggcctgcctatgggtatacccataggacgctct | |
| | aatactgacatggtgcgaagagtctattgagctagttggtagtcc | |
| | tccggcccctgaatgcggctaatcctaactgcggagcacataccc | |
| | ccaacccagggggcagtgtgtcgtaacgggtaactctgcagcgga | |
| | accgactactttgggtgtccgtgtttccttttattcttacattgg | |
| | ctgcttatggtgacaattgagagattgttaccatatagctattgg | |
| | attggccatccagtgactaacagagctattatttacctttttgtt | |
| | ggcttcgtatcacttggtttaaaagaggttagtactttatattgc | |
| | attatattactaaacacgagaaa | |
| iCVA20 IRES | ttaaaacagctctggggttgtacccaccccagaggcccacgtggc | 33077 |
| | ggctagtactccggtattacggtacccttgtacgcctgttttata | |
| | ctccctccctcgtaacttagaagcacaaaaccaagttcaatagaa | |
| | gggggtgcaaaccagtaccaccacgaacaagcatttctgtttccc | |
| | cggtgatgttgtatagactgcccgcgcggttgaaagcaacggatc | |
| | cgttacccgctcaagtacttcgagaagcctagtattaccttggaa | |
| | tcttcgatgcgttgcgttcagcactcgaccctggagtgtagctta | |
| | ggctgatgagtctggacgtccctcaccggtgacggtggtccaggc | |
| | tgcgttggcggcctacctatggctaacgccataggacgctagttg | |
| | tgaacaaggtgtgaagagcctattgagctacttgagagtcctccg | |
| | gcccctgaatgcggctaatcctaaccatggagcaggcggtcacag | |
| | accagtgactagcttgtcgtaatgcgcaagtctatggcggaaccg | |
| | actactttgggtgtccgtgtttccttttatttttattatggctgc | |
| | ttatggtgacaatcattgattgttatcataaagcgaattggattg | |
| | gccatccggtgaaagcgagacttactatttacttacttgttggac | |
| | ttaccacacttaatacatttattctaggtgtcacttgtatagcaa | |
| | ttagaatcaaacagttgcatcata | |
| iEV-D94 IRES | ttaaaacagcctgtgggttgttcccaccccagaggcccacgtggc | 33078 |
| | ggccagtactctggtatcacggtacctttgtacgcctgttttata | |
| | tccccttcccccgcaacttagaagaaaacaaatcaagttcactag | |
| | gagggggtacaaaccagtaccaccacgaacaagcacttctgtttc | |
| | cccggtgatgtcgtatagactgtaaccacggttgaaaacgattga | |
| | tccgttatccgctcttgtacttcgaaaagcccagtatcaccttgg | |
| | aatcttcgatgcgttgcgctcagcactcaaccccagagtgtagct | |
| | taggtcgatgagtctggacactcctcaccggcgacggtggtccag | |
| | gctgcgttggcggcctacctgtggtccaaagccacaggacgctag | |
| | ttgtgaacaaggtgtgaagagcctattgagctacaagagaatcct | |
| | ccggcccctgaatgcggctaatcctaaccacggagcaagggtaca | |
| | caaaccagtgtatatcttgtcgtaacgcgcaagtctgtggcggaa | |
| | ccgactactttgggtgtccgtgtttccttttgtttttatcatggc | |
| | tgcttatggtgacaatctaagattgttatcatatagctgttggat | |
| | tggccatccggtaatttattgagatttgagcatttgcttgtttct | |
| | tcaacaatttcacctattcattgcatttcagcagtcaaa | |
| iSimianEV-A | gagtgttcccacccaacaggcccactgggtgttgtactctggtat | 33079 |
| IRES | tacggtacctttgtacgcctattttatttcccccccctttttgaa | |
| | acttagaagttaataataaacacgctcactaggtgcactacatcc | |
| | agtagtgtaatgagcaagcacttctgtctyccccgggagggatat | |
| | atggtacgctgtgcaaacggcggaaattaatcctaccgttaaccg | |
| | cccacctactccgagaagcctagtacctaattggatttatcaatg | |
| | gagttgcgctcagcaggtgaccctgacctgccagctccggctgat | |
| | ggacctgggctttccccacaggcgactgtggcccaggtcgcgtgg | |
| | cggccggcccacccccctgggtgggacgccttgataatgacaagg | |
| | tgggaagagcctattgggctagctggtttcctccggcctcctgaa | |
| | tgcggctaaccttaaccccagagcatatggtagcaacccagctac | |
| | tagtatgtcataatgcgtaagtctgggatgggaccgactactttg | |
| | gagagtccgtgtttctattgtttctttaatcaatcttatggtgac | |
| | aatttatagtgccctgagtattgattggttgttgcttttgacaat | |
| | tattgagacatcacatagacata | |
| iSimianA5 IRES | ttaaaatagcctgtgggttgctcccacccacagggcccaagtggc | 33080 |
| | gtagtacttggtattccggtacctttgtacacctatttacaaacc | |
| | ctaccccttgtaaccttagaagcaattatttaaccgctcactagg | |
| | gggtgtgctatccaagcacatcaagagcaagcacttctgtctccc | |
| | cgggaggggctaatggtacgctgtgcccacggcggaaatgagccc | |
| | taccgttaaccggcagtctacttcgggaagcccagtaactacatt | |
| | gaaactttgaggcgttacactcagcacataaccccaatgtgtagt | |
| | tctggtcgatgagccttggcatcccccacaggcgactgtggccaa | |
| | ggctgcgttggcggccagcctgcggaccaaaagtccgtaggacgc | |
| | ctaattgtggacatggtgtgaagagcctactgagctagactgtag | |
| | tcctccggcccctgaatgcggctaatcctaaccctggagcatccg | |
| | cgtgcaacccagtacgtagggtgtcgtaatgcgtaagtctgggat | |
| | ggaaccgactactttgggtgtccgtgtttcttgtttttcatactg | |
| | ggtcgcttatggttacaactaattgttgtaatcattggcagtgcg | |
| | cgctgaccacgcgattattgatatttccatttgttggatactcca | |
| | atagtgtcaactcatatacacaacttttaccactgatcaagataa | |
| | aa | |
| iSimian V4 IRES | ttaaaatagctgctgggttgttcccatccagcaggcccactgggc | 33081 |
| | gtgatactctggtattgcggtacctttgtatgcctattttacctc | |
| | ccttccccctgaaacttagaagaaagaaacaaacacgctcactag | |
| | gtacactgcatccagcagtgtaatgagcaagcacttctgtttccc | |
| | cggaagggatatatggtacgctgtgcaaacggcggaaattaatcc | |
| | taccgttaaccgcccatctactccgagaagcctagtacctaattg | |
| | aacttatcaatggagttgcgctcagcaggtgaccctgacctgcca | |
| | gctccggctgatggacctgggcatcccccacaggcgactgtggcc | |
| | caggtcgcgtggcggccggcccactcttttgagtgggacgccttg | |
| | ataatgacaaggtgggaagagcctattgggctagctggtttcctc | |
| | cggcctcctgaatgcggctaaccctaaccccggagcatatggtgg | |
| | caacccagccactggtatgtcgtaacgcgtaagtctgggatggga | |
| | ccgactactttggagagtccgtgtttccattattttctttattga | |
| | atcttatggtgacaaattgggagttactcgggtgacgattgatta | |
| | ttacttctgaaaacgtagagatatagaatcaacaca | |
| iSwine Vesciular | ttaaaacagcctgtgggttgttcccacccacagggcccactgggc | 33082 |
| IRES | gctagcacactggtatcacggtacctttgtgcgcctgtttgactt | |
| | accctccccaaacgcaacttagaagcacaacttaaatggtcaata | |
| | ggcggctcagtatgccaactgagtctcgatcaagcacttctgtta | |
| | ccccggactgagtaccaataggctgctcacgcggctgaaggggaa | |
| | accgttcgttacccgactaactacttcgagaaacctagtaccacc | |
| | atgaaagttgcgcagcgtttcgctccgcacaaccccagtgtagat | |
| | caggtcgatgagtcaccgcaaaccccacgggcgaccgtggcggtg | |
| | gctgcgctggcggcctgcccatggggcaactcatgggacgcttca | |
| | atactgacatggtgcgaagagtctattgagctagttggtagtcct | |
| | ccggcccctgaatgcggctaatcctaactgcggagcagataccca | |
| | cgcaccagtgggcagtctgtcgtaatgggcaactctgcagcggaa | |
| | ccgactactttgggtgtccgtgtttccttttgttcttatactggc | |
| | tacttatggtgacaattgagagattgtaaccatattgctattgga | |
| | ttggccacccggcgacgaatagaacagttgcttacctgtttgtta | |
| | gtctcgtatcactgaactacaaatccttaaacaccctttaatttc | |
| | gtcataacgctcaatacgttaaa | |
| iHRV-B3 SC2220 | ttaaaacagcggatgggtatcccaccatccgacccactgggtgta | 33083 |
| IRES | gtactctggtactttgtacctttgtacgcctgttcttcccattgt | |
| | acccttcctaaacttccaacccaagtaacgttagaagctcaactt | |
| | tcagtacaataggaagcaccacatccagtggtgtttagtacaagc | |
| | acttctgtttccccggagcgaggtataggctgtacccactgccaa | |
| | aagcctttaaccgttatccgccaaccaactacgtaaaagctagta | |
| | acatcatgtttgtaaatgggtgttcgatcaggtggatttcccctc | |
| | cactagtttggtcgatgaggctaggaattccccacgggtgaccgt | |
| | gtcctagcctgcgtggggccaacccagcgtatgctgggacgcctt | |
| | tttatagacatggtgcgaagactcgcatgtgcttggttgtgagtc | |
| | ctccggcccctgaatgcggctaaccctaaccctggagccttgagt | |
| | cacaatccagtgatggcaaggtcgtaatgagcaattccgggatgg | |
| | gaccgactactttgggtgtccgtgtttctcatttttcttattatt | |
| | gtcttatggtcacagcatatatagtatatatactgtgatc | |
| iHRV-B5 IRES | ttaaaacagcgggtgggtatcccaccacccgacccactgggtgtg | 33084 |
| | gtacactggtactttgtacctttgtatgcctgtttgcttccactc | |
| | ccctttacctaatattcttccccaagtatattttgcggtaacgtt | |
| | agaagaagcaaagttagtgcaataggatgtatcacaaccagtggt | |
| | ataaagcacaagcaattcttgttccccggagctggatatagactg | |
| | ctaacgtggtcaaaagtccttaaccgttatccgccagctaactgc | |
| | gagaagactagtaacaccatgtttgtttttagacgttcgatcagg | |
| | tgaataccccattcactagtttggtcgatgaggctgagaataccc | |
| | cacaggtaactgtgtctcagcctgcgtggcggccaacccgactca | |
| | tgtcgggacgcctactaatagacatggtgtgaagatcctattgcg | |
| | cttagttgtgagtcctccggcccctgaatgcggctaaccctaacc | |
| | ccggagccttgtggtgtaaaccaacatttgcaaggtcgtaatgag | |
| | taattctgggatgggaccgactactttgggtgtccgtgtttcctt | |
| | tatttttattgtgtgtgtcttatggttacaaatacaatttgtaat | |
| | c | |
| iHRV-B6 IRES | ttaatacagcggatgggtatcccaccatccgacccactgggtgta | 33085 |
| | gtactctggtattatgtacctttgtacgcctgttcttcccattgt | |
| | acccttcccaaatttccaacccaagtaacgttagaagtttgacat | |
| | taaagtacaataggaggcaccacatccagtggtgttttgtacaag | |
| | cacttctgtttccccggagcgaggtataggctgtacccactgcca | |
| | aaaacctttaaccgttacccgccagccaactacgtaaaagctagt | |
| | aacatcttgtttgtaacttggcgttcgatcaggtggattccccct | |
| | ccactagtttggtcgatgaggctaggaactccccacgggtgaccg | |
| | tgtcctagcctgcgtggcggccaacccagctcttgctgggacgcc | |
| | cttttatagacatggtgtgaagactcgcatgtgcttggttgtgat | |
| | tcctccggcccctgaatgcggctaaccctaaccctggagccttgt | |
| | gttacaaaccagtaatattaaggtcgtaatgagcaattccgggac | |
| | gggaccgactactttgggtgtccgtgtttctcatttttcttatta | |
| | ttgtcttatggtcacagcatatttgttaaatatactgtgatc | |
| iHRV-B14 IRES | tactctggtattatgtacctttgtacgcctgtttcttccctacaa | 33086 |
| | ccccttcctaaaactcccacccatgaaacgttagaagcttgacat | |
| | ttaagtacaataggtggcaccacatccagtggtgtctacgtacaa | |
| | gcacttctgtttccccggagcgaggtataggctgtacccactgcc | |
| | aaaagcctttaaccgttatccgccaaccaactacgtaacagctag | |
| | tatcatcttgttcttcactggacgttcgatcaggtggattccccc | |
| | tccactagtttggtcgatgaggctaggagctccccacgggtgacc | |
| | gtgtcctagcctgcgtggcggccaacccagctcatgctgggacgc | |
| | ccttttaaggacatggtgtgaagactcgcatgtgcttggttgtga | |
| | gtcctccggcccctgaatgcggctaaccttaaccctggagcctta | |
| | tgccacgaaccagtggttgtaaggtcgtaacgagcaattccggga | |
| | tgggaccgactactttgggtgtccgtgtttcctatttttctttat | |
| | attgtcttatggtcacagcatatatataagtatatactgtgatc | |
| iHRV-B27 IRES | ttaaaacagcggatgggtatcccaccatccgacccacagggtgta | 33087 |
| | gtgctctggtattttgtacctttgcacgcctgtttccccattgta | |
| | cccctccttaaatttcctccccaagtaacgttagaagtttaagga | |
| | aacaaatgtacaataggaagcatcacatccagtggtgttatgtac | |
| | aagcacttctgtttccccggagcgaggtataagtggtacccaccg | |
| | ccgaaagcctttaaccgttatccgccaatcaactacgtaatggct | |
| | agtattaccatgtttgtgacttggtgttcgatcaggtggttcccc | |
| | ccactagtttggtcgatgaggctaggaactccccacgggtgaccg | |
| | tgtcctagcctgcgtggcggccaacccagcttttgctgggacgcc | |
| | tttttacagacatggtgtgaagacctgcatgtgcttgattgtgag | |
| | tcctccggcccctgaatgcggctaaccttaaccccggagccttgc | |
| | aacataatccaatgttgttgaggtcgtaatgagtaattctgggat | |
| | gggaccgactactttgggtgtccgtgtttccttttattctttata | |
| | ttgtcttatggtcacagcatatatagcatatatactgtgatc | |
| iHRV-B35 IRES | ttaaaacagcggatgggttccctaccatccgacccactgggtgta | 33088 |
| | gtactctggtactctgtacctttgtacgcctgtttatccactacc | |
| | cttttcctaaattttccacccgtgtaaccttagaagcaaacaaat | |
| | tgtacaatagggtgcatcacaaccagtgatgtacagtacaagcac | |
| | ttctgtttccccggagcggggtatagacggtcaccactgtcaaaa | |
| | gcccttaaccgttacccgccaaccaactacgtaacggctagtaac | |
| | accatgaatttgtcttgacgttcgatcaggtggtaaccccccact | |
| | agtttggtcgatgaggctgagaactccccacgggtaaccgtgtct | |
| | cagcctgcgtggcggccaacccagcttctgctgggacgccaattc | |
| | attgacatggtgtgaagacctgcatgtgcttggttgtgattcctc | |
| | cggcccctgaatgcggctaaccctaaccccggagccacgcggcat | |
| | aatccaatgtcgtatgggtcgtaatgagtaattctgggatgggac | |
| | cgactactttgggtgtccgtgtttcctgtttttccaaattattct | |
| | tatggtcacagcatataaatagatatatactgtgatc | |
| iHRV-B42 IRES | ttaaaacagcgggtgggtatcccaccacccgacccactgggtgtg | 33089 |
| | gtacactggtactttgtacctttgtatgcctgtttgcatcccctt | |
| | ccctttacctaacatccctccccaagttatattttgcggtaacgt | |
| | tagaagaagtgaacAtagtgcaacaggaagtatcaccaccagtga | |
| | tataacgcacaagcaattcttgttccccggagctggatatagact | |
| | gctaacgtggttgaaagtccttaaccgttatccgccaATCaactg | |
| | cgaaaagactagtaacatcatgcacatttctaggtgttcgatcag | |
| | gtgaaaaccccattcactagtttggtcgatgaggctgagagtacc | |
| | ccacaggcgactgtgtctcagcctgcgtggcggccaacccgactt | |
| | atgtcgggacgcctattaatagacatggtgtgaagatcctattgc | |
| | gcttagctgtgagtcctccggcccctgaatgcggctaaccttaac | |
| | cccggagtcttgtgatgtaagccaacatttgcaaggtcgtaatga | |
| | gtaattctgggatgggaccgactactttgggtgtccgtgtttcct | |
| | ttattcttatcatttgtgtttcatggttacaagcattgtttgtaa | |
| | tc | |
| iHRV-B48 IRES | ttaaaacagcggatgggtatcccaccatccgacccactgggtgta | 33090 |
| | gtactctggtactctgtacctttgtacgcctgtttctcctcaacc | |
| | ctcccctacccgttatatatctcagtaacttagaagaATtcatta | |
| | ccgcacaatagggaccgttgtaaccagcaacggtcagtgcaagca | |
| | cttctgtttccccggagcggagtatagacggccactgctgtcaaa | |
| | agctcttaaccgttacccgccagtATactacacaacagctagtaa | |
| | catcttgttgttcttagggcgttcgatcaggtgagtttaccccct | |
| | cactagtctggtcgatgaggctgagaaatccccacgggtgaccgt | |
| | gtctcagcctgcgtggcggccagcccagcttatgctgggacgcct | |
| | taagtatgacatggtgtgaagactcgcgtgtgcttagctgtgagt | |
| | cctccggcccctgaatgcggctaacctaaaccccggagccttgag | |
| | gcacaatccagtgcccgcgaggtcgtaacgagaaattctgggacg | |
| | ggaccgactactttgggtgtccgtgtttccttttatctttttgat | |
| | gttcttatggttacaatatataaagtatattgtaatc | |
| iHRV-B52 IRES | ttaaaacagcggatgggtatcccaccatccgacccactgggtgta | 33091 |
| | gtactctggtattatgtacctttgtacgcctgtttctccccccca | |
| | ttacccctccccacatatcccagtaacttagaagaattaaattat | |
| | cgcacaataggagctattacaaccagtaatagttagtgcaagtac | |
| | ttctgtttccccggagcggagtatagatggccaccgctgtcaaaa | |
| | gctcttaaccgttatccgccagtATactacgtaacggctagtaac | |
| | atcttgttgtgcttaaggtgttcgatcaggtgaagagacaccccc | |
| | ttcactagtctggtcgatgaggctgagaaatccccacgggcgacc | |
| | gtgtctcagcctgcgtggcggccagcccagcttatgctgggacgc | |
| | cttaagtatgacatggtgtgaagacccgcatgtgcttaactgtga | |
| | gtcctccggcccctgaatgcggctaaccttaaccctggagccttg | |
| | gggtacaatccagtgctaacaaggtcgtaacgagcaattccggga | |
| | cgggaccgactactttgggtgtccgtgtttccttttatttcatta | |
| | ttgttcttatggttacaatatatagagtatattgtaatc | |
| iHRV-B69 IRES | ttaaaacagcggatgggtatcccaccatccgacccactgggtgta | 33092 |
| | gtactctggtactttgtacctttgtacgcctgtttctccctccct | |
| | tcccaacctaaacaatccttgtaacttagaaggtttcgattatcg | |
| | cacaatagggactattgcaaccagcaatagtcagtgcaagcactt | |
| | ctgtttccccggagcggagtgtagatggccaccgctgtcaaaagc | |
| | tcttaaccgttatccgccagtATactacgcaacggctagtagtgc | |
| | cttgttgcttttatggcgttcgatcaggcgagatttaccccctcg | |
| | ctagtctggtcgatgaggctgagaattccccacgggcgaccgtgt | |
| | ctcagcctgcgtggcggccagcccagcctttgctgggacgccttg | |
| | attgcgacatggtgtgaagacccgcgtgtgcttaactgtgagtcc | |
| | tccggcccctgaatgcggctaacctaaaccctggagccttgaaac | |
| | acaacccagtgttcgcaaggtcgtaacgagcaattccgggacggg | |
| | accgactactttgggtgtccgtgtttcctttattctttcaattgt | |
| | tcttatggtcacaatataagtgttatattgtgatc | |
| iHRV-B70 IRES | ttaaaacagctgatgggttccccaccatccgacccaatgggtgta | 33093 |
| | gtactctggtattctgtacctttgtacgcctgtttctccccaccc | |
| | ccccgtcctaaataatcccagtaacttagaagatttgaattatcg | |
| | tacaataggtgctgtcacatccagtgatggctagtacaagcactt | |
| | ctgtttccccggagcggagtatagatggccaccgctgtcaaaagc | |
| | tcttaaccgttatccgccaattaactacgcaacggttagtaacat | |
| | cttgtcatttttgaggcgttcgatcaggtgagtacacccctcact | |
| | agtctggtcgatgaggctgagaattccccacgggcgaccgtgtct | |
| | cagcctgcgtggcggccagcccagcttatgctgggacgccttaat | |
| | tgtgacatggtgcgaagacccacgtgtgcttaattgtgagtcctc | |
| | cggcccccgaatgcggctaacctaaaccccggagccttgagacac | |
| | aatccagtgttagcaaggtcgtaacgagcaattctgggacgggac | |
| | cgactactttgggtgtccgtgtttcctgttattctcaaattgttc | |
| | ttatggtcacaatataagtaatatatatattgtgatc | |
| iHRV-B72 IRES | ttaaaacagcggatgggttctcccaccatccgacccactgggtgt | 33094 |
| | agtactctggtatgatgtacctttgtacgcctgtttattcccaaa | |
| | cccccacccgttattcgcccaacccctgtaacgttagaagtttgg | |
| | aacttaattgtacaatagggagcaccatatccaatggtgtaacgt | |
| | acaagcacttctgtttccccggagcgaggtataggtggtacccac | |
| | tgccgaaagcctttaaccgtcatccgccaatcaactacgtaacag | |
| | ctagtaacatcttgttgttgattggacgttcgatcaggtggttca | |
| | ccccccactagtttggtcgatgaggctgggaatcccccacgggtg | |
| | accgtgtcccagcctgcgtggcggccaacccagcttatgctggga | |
| | cgccctttcaatgacatggtgtgaagactcgcatgtgcttgattg | |
| | tgagtccgccggcccctgaatgcggctaaccctaaccctggagcc | |
| | ttgcaccacaatccagtggtgtctgggtcgtaatgagtaattctg | |
| | ggatgggaccgactactttgggtgtccgtgtttctcatttttctt | |
| | ttaattgtcttatggtcacagcatatagtattatatactgtgatc | |
| iHRV-B79 IRES | ttaaaacagcggacgggtatcccaccatccggctcactgagcgta | 33095 |
| | gtactctggtattgtgtacctttgtacgcctgttttccccaaatc | |
| | ccctaccctttatatctcctaccccgtaacgtaagaagttttcaa | |
| | acagtacaataggaagcaccacatccagtggtgttatgtacaagc | |
| | acttctgtttccccggagcgaggtataggtggtcaccaccgccaa | |
| | aagcctttaaccgttacccgccaaccaactacgtaatggctagta | |
| | acatcattgatttagattggcgttcgatcaggtggtatcccccca | |
| | ctagtttggtcgatgaggctaggaataccccacgggtgaccgtgt | |
| | cctagcctgcgtggcggccaacccagcatacgctgggacgccagt | |
| | ttactgacatggtgcgaagacttgcatgtgcttggttgagagtcc | |
| | tccggcccctgaatgcggctaaccttaaccccggagcctcgcagc | |
| | ataatccaatgttgtagaggtcgtaacgagtaattctgggatggg | |
| | accgactactttgggtgtccgtgtttcctgtttttctttaaaatg | |
| | tcttatggtcacagcatatatactgtatatactgtgatc | |
| iHRV-B83 IRES | gtacaataggttgcactacatccagtggtgtttcgtacaagcact | 33096 |
| | tctgtttccccggagcggggtatagacggtacccactgtcaaaag | |
| | cccttaaccgttatccgccaatcaactacgtaatggctagtaaca | |
| | tcatgaaggtgagttgacgttcgatcaggtggtaacccccactag | |
| | tttggtcgatgaggctaggaatcccccacgggtaaccgtgtccta | |
| | gcctgcgtggcggccaacccagcgtttgctgggacgccaattcac | |
| | tgacatggtgcgaatacttgcatgtgcttgattgtgattcctccg | |
| | gcccctgaatgcggctaaccttaaccccggagccttgcagcataa | |
| | accaatgttgtggaggtcgtaatgagcaattctgggatgggaccg | |
| | actactttgggtgtccgtgtttcctgtttttctcaaattgtctta | |
| | tggtcacagcatatatattgtatatactgtgatc | |
| iHRV-B84 IRES | ttaaaacagcggatgggtatcccaccatccggcccactgggcgta | 33097 |
| | gtacactggtattctgtacctttgtacgcctgtttgccgcccctc | |
| | ccccttttattaccacatttgtggtcgctgcaacgttagaagatt | |
| | ttaaagtagtgcaataggaagcatcacaaccagtggtgttatgca | |
| | caaacacttctgtttccccggagcgaggtatagacggtccccact | |
| | gtcaaaagcctttaaccgttatccgccaaccaactacgagacagc | |
| | tagtaatgccctgttctcctgcaaggcgttcgatcagacagattt | |
| | cccctctgttagtctggtcgatgaggctagaagtaccccacaggt | |
| | aactgtgttctagcctgcgtggcggccagcccagcttctgctggg | |
| | acgcctttcaagagacatggtgtgaagactcaattgtgcttggtt | |
| | gtgagtcctccggcccctgaatgcggctaaccttaaacccggTAc | |
| | catgctatgcaaaccagcatagttatggtcgtaatgggcaactct | |
| | gggatgggaccgactactttgggtgtccgtgtttccatttttact | |
| | ttttgtgtcatttatggttacaattaagttgtaacc | |
| iHRV-B86 IRES | ttaaaacagcggatgggtaccctaccatccgacccactgggtgta | 33098 |
| | gtacactggtacattgtacctttgtacgcctgtttctccctattg | |
| | taccttccctcacaatcctaacccctgtaacgttagaagatgtgc | |
| | aactcgtgcaataggagatactgcaaccagtggtatcaagcacaa | |
| | gaacttctgtttcctcggagcggagtatagatggtccccaccgtc | |
| | aaaagctcttaaccgttatccgccaatcaactacgtgacgactgg | |
| | tagca Tcattttgacactttgacgttcgatcaggtaggcttacc | |
| | ccacctactagtttggccgatgaggctgggagtccctcacaggta | |
| | actgtgtcccagcctgcgtggcggccaacccagcaattgctggga | |
| | cgccaatttgttgacatggtgtgaagatcttaatgtgcttggttg | |
| | tgagtcctccggcccctgaatgcggctaaccttaaccccggagcc | |
| | ttgtgtcacaagccagtgacattaaggtcgtaatgagtaatttcg | |
| | ggatgggaccgactactttgggtgtccgtgtttcctgtttttctt | |
| | tatgattgtcttatggtcacagcatatagtaattatatactgtga | |
| | tc | |
| iHRV-B91 IRES | ttaaaacagcggatgggtttcccaccatccgacccactgggtgta | 33099 |
| | gtactctggtacattgtacctttgtacgcctgtttctcccctccc | |
| | tcccaacctatacaatcccggcaacttagaagatttgaactattg | |
| | tacaataggcgctgtcacatccagtggcggctagtacaagcactt | |
| | ctgtttccccggagcggagtatagatggccaccgctgtcaaaagc | |
| | tcttaaccgttatccgccaattaactacgcaacggctagtaatat | |
| | catgtaacttttagggtgttcgatcaggtgagtgtacccctcact | |
| | agtctggtcgatgaggctgagaattccccacgggcgaccgtgtct | |
| | cagcctgcgtggcggccagcccagcttatgctgggacgccttaat | |
| | tgtgacatggtgtgaagacccacgtgtgcttaattgtgagtcctc | |
| | cggcccctgaatgcggctaacctaaaccctggagccttgaaacac | |
| | aatccagtgttagcaaggtcgtaatgagtaattccgggacgggac | |
| | cgactactttgggtgtccgtgtttccttttatttttaagttgttc | |
| | ttatggtcacaatataagtaatatatattgtgatc | |
| iHRV-B93 IRES | ttaaaacagtggatgggtttcctaccatccgacccactgggtgta | 33100 |
| | gtgctctggtatattgtacctttgcacgcctgttttcccattgta | |
| | cccttccttaaattcctccccatgtaacgttagaagtttaagaac | |
| | ataaatgtacaataggaagcatcacatccagtggtgtatggtaca | |
| | agcacttctgtttatccggagcgaggtataagcggtacccactgc | |
| | tgaaagcctttaaccgtATaccgccaatcaactacgtaacggcta | |
| | gtattaccttgtttgcaatctggcgttcgatcaggtggttccccc | |
| | cactagtttggtcgatgaggctaggagttccccacgggcgaccgt | |
| | gtcctagcctgcgtggcggccaacccagcatttgctgggacgcct | |
| | ttttgtagacatggtgtgaagacctgcatgtgcttgattgtgagt | |
| | cctccggcccctgaatgcggctaaccttaaccccggagccttgtg | |
| | acataatccaatgtcattaaggtcgtaatgagcaattctgggacg | |
| | ggaccgactactttgggtgtccgtgtttctctttattctttacat | |
| | tgtcttatggtcacagcatatataatacatatatactgtgatc | |
| iHRV-B97 IRES | ttaaaacagcggatgggtttcccaccatccgacccactgggtgta | 33101 |
| | gtgctctggtattttgtacctttgcacgcctgtttcccctttgta | |
| | cccatcctgaatttcctccctctgcaacgttagaagtttgtgaaa | |
| | ttaaaagtacaataggaagcatcacatccagtggtgttcagtaca | |
| | agcactcctgtttccccggagcgaggtataggttgtacccacgac | |
| | cgaaagcctttaaccgttatccgccaatcaactacataacggcta | |
| | gtatcatcatgtttttgatctggcgttcgatcaggtggtttcccc | |
| | cactagtctggtcgatgaggctaggatttccccacgggcgaccgt | |
| | gtcctagcctgcgtggcggccagcccagcttatgctgggacgcct | |
| | ttttaaagacatggtgtgaagacctgcatgtgcttgattgtgagt | |
| | cctccggcccctgaatgcggctaaccttaaccctggagcccgaca | |
| | gcataatccaatgttgtttgggtcgtaatgagcaattccgggatg | |
| | ggaccgactactttgggtgtccgtgtttctttttattcttatatt | |
| | gtcttatggtcacagcatatatagtatatatactgtgatc | |
| iHRV-B99 IRES | ttaaaacagcgggtgggtatcccaccacccgacccactgggtgta | 33102 |
| | gtacactggtattttgtacctttgtatgcctgtttgccatccctc | |
| | cctctcctcttacccttacccttattttgcggtaactttagaagt | |
| | ttgtaaatcacgcgcaatagggtataccacatccagtggtatgaa | |
| | gcacaagcaattcttgttccccggagctggatataaactgctaac | |
| | gtggttgaaagtccttaaccgttatccgccagtTAactgcgagaa | |
| | gactagtaactccatgtttgtttttaagcgttcgatcaggtggat | |
| | accccattcactagtttggtcgatgaggctgggaatcccccacag | |
| | gtaactgtgtctcagcctgcgtggcggccaacccagcttttgctg | |
| | ggacgcttactgacagacatggtgtgaagatcctattgcgcttaa | |
| | ctgtgagtcctccggcccctgaatgcggctaacctcaaccccgga | |
| | gccttgtggtgtaaaccaacactcgcaaggtcgtaacgagtaatt | |
| | ctgggacgggaccgactactttgggtgtccgtgtttcctttattt | |
| | cttattgattgtctcctatggttacaaatttactgtaatc | |
| iHRVB_SC0739 | ttaaaacagcgggtgggtatcccaccacccgacccactgggtgtg | 33103 |
| IRES | gtacactggtactttgtacctttgtatgcctgtttgcatcccttt | |
| | cccctcgcctaacatccttccccaaagtatattttgcggtaacgt | |
| | tagaagaagtgaactcagtgcaacaggaagtatcaccaccagtga | |
| | tataatgcacaagcaattcttgttccccggagctggatatagact | |
| | gctaacgtggttgaaagtccttaaccgttatccgccagctaactg | |
| | cgagaagactagtaacatcatgtacatttctagacgttcgatcag | |
| | gtgaataccccattcactagtttggtcgatgaggctgagagttcc | |
| | ccacaggtgactgtgtctcagcctgcgtggcggccaacccgactt | |
| | atgtcgggacgcctattaatggacatggtgcgaagatcctattgc | |
| | gcttagctgtgagtcctccggcccctgaatgcggctaaccttaac | |
| | cccggagccttgtggtgcaaaccagcatttgcaaggtcgtaatga | |
| | gcaattctgggatgggaccgactactttgggtgtccgtgtttcct | |
| | ttattcttattatttgtgtttcatggttacaagcatagtttgtaa | |
| | tc | |
| iHRVB_G001-19 | TTAAAACAGCGGatgggtttcccaccatccgacccactgggtgta | 33104 |
| IRES | gtgctctggtattttgtacccttgcacgcctgtttcccatttgta | |
| | cccttccttaatttcctacccctgtaacattagaagttttggaat | |
| | tttacggtacaataggaagcgccacatccagtggtgttgtgtaca | |
| | agcacttctgtttacccggagcgaggtataggttgtacccacggc | |
| | caaaaaccttcaaccgttatccgccaaccaactacgtaacggcta | |
| | gtatcatcttgtatttgacttggcgttcgatcaggtggtatcccc | |
| | cactagtctggtcgatgaggctaggaattccccacgggcgaccgt | |
| | gtcctagcctgcgtggcggccagcccagctttagctgggacgcct | |
| | tttcaaagacatggtgtgaagacctgcatgtgcttggttgtgagt | |
| | cctccggcccctgaatgcggctaaccttaaccccggagcccagtg | |
| | acataatccaatgttatttgggtcgtaatgagtaattccgggatg | |
| | ggaccgactactttgggtgtccgtgtttccttttattctttatat | |
| | tgtcttatggtcacagcatatatagtatatatactgtgatc | |
| iHRVB_R93 | ttaaaacagcggatgggttctcccaccatccgacccactgggtgt | 33105 |
| IRES | agtactctggtattatgtacctttgtacgcctgtttattcccaaa | |
| | ccccctcccgttacatttccaacccctgtaacgttagaagtttgg | |
| | aacttaacagtacaataggaggcaccatatccaatggtgtaatgt | |
| | acgagcacttctgtttccccggagcgaggtataggtggtacccac | |
| | cgctgaaagcctttaaccgttatccgccaatcaactacgtaacag | |
| | ctagtagcattttgttgttgactggacgttcgatcaggtggttta | |
| | cccccccactagtttggtcgatgaggctggaaattccccacgggt | |
| | gaccgtgttccagcctgcgtggcggccaacccagctcatgctggg | |
| | acgccctttcaatgacatggtgtgaagactcgcatgtgcttgatt | |
| | gtgaatcctccggcccctgaatgcggctaaccctaaccccggagc | |
| | cttgcatcacaatccagtgatgttagggtcgtaatgagtaattct | |
| | gggatgggaccgactactttgggtgtccgtgtttctcatttttct | |
| | ttgaattgtcttatggtcacagcatatagtaatatatactgtgat | |
| | c | |
| iHRVB_BCH214 | ttaaaacagcgggtgggtatcccaccacccgacccactgggtgtg | 33106 |
| IRES | gtacactggtactttgtacctttgtatgcctgtttgcatcccttt | |
| | tcccttcacctaacatcctcccccaaagtatattttgcggtaacg | |
| | ttagaagaagtgaactcagtgcaacaggaagtatcaccaccagtg | |
| | atataacgcacaagcaattcttgttccccggagctggatatagac | |
| | tgctaacgtggtcaaaagtccttaaccgttatccgccagctaact | |
| | gcgagaagactagtaacatcatgcacatttctagacgttcgatca | |
| | ggtgaataccccattcactagtttggtcgatgaggctgagagttc | |
| | cccacaggtgactgtgtctcagcctgcgtggcggccaacccgact | |
| | tatgtcgggacgcctattaatggacatggtgtgaagatcctattg | |
| | cgcttagctgtgagtcctccggcccctgaatgcggctaaccttaa | |
| | ccccggagccttgtggtgcaaaccagcatttgcaaggtcgtaatg | |
| | agcaattctgggatgggaccgactactttgggtgtccgtgtttcc | |
| | tttattcttattatttgtgtttcatggttacaagcatagtttgta | |
| | atc | |
| iHRVB_3039 | ttaaaacagcggatgggtatcccaccatccgacccactgggtgta | 33107 |
| IRES | gtactctggtattatgtacctttgtacgcctgtttctccctccct | |
| | accaccccgccccacgtatcccgtaacttagaagaattgaacaat | |
| | cgcacaataggagctattacaaccagtaatagttggtgcaagcac | |
| | ttctgtttccccggagcggagtatagacggccaccgctgtcaaaa | |
| | gctcttaaccgttacccgccagCtaactacgtaacggctagtaac | |
| | atcttgtcgtgcttagggtgttcgatcaggtgaagaaaaaccccc | |
| | ttcactagtctggtcgatgaggctgagaagtccccacgggtaacc | |
| | gtgtctcagcctgcgtggcggccagcccagcatatgctgggacgc | |
| | cttaagtatgacatggtgtgaagacccgcatgtgcttagctgtga | |
| | gtcctccggcccctgaatgcggctaacctaaaccctggagccttg | |
| | gagcacaagccagtgcttgcaaggtcgtaatgagcaattccggga | |
| | cgggaccgactactttgggtgtccgtgtttccttttatctcatta | |
| | ttgttcttatggttacaatatatagagtatattgtaatc | |
| iEV-B69 IRES | ttaaaacagcctgtgggttgtacccacccacagggcccactgggc | 33108 |
| | gctagcacactggtatcacggtacctttgtgcgcctgttttatac | |
| | cccctaccccaactttaaacttagaagcaaagcaaacccgatcaa | |
| | tagcagatgtagcaagccagttgcatcttgatcaagtacttctgt | |
| | ctccccggactgagtatcaatagactgctcacgcggttgaaggag | |
| | aaaacgttcgttacccgaccaactacttcgagaaacctagtaaca | |
| | ccatgaaagtagcagggtgtttcgatcagcacaaccccagtgtag | |
| | atcaggctgatgagtcaccgcatcccccacgggcgaccgtggcgg | |
| | tggctgcgctggcggcctgcccatggggcaacccatgggacgctc | |
| | taatatggacatggtgtgaagagtctattgagctagttggtagtc | |
| | ctccggcccctgaatgcggctaatcctaactgcggagcacatacc | |
| | ccaaagccaaggggcagtgtgtcgtaacgggcaactctgcagcgg | |
| | aaccgactactttgggtgtccgtgtttccttttattctcaaactg | |
| | gctgcttatggtgacaatctcagagttgttaccatatagctattg | |
| | gattggccatccggtgagcaacagagcaattgtctaccaatttgt | |
| | tggatttattccactcaatctagcggttttcagaacattgaatta | |
| | tattctaaccctcaacaagacgaa | |
| iEV-B73 IRES | ttaaaacagcctgtgggttgtacccacccacagggcccactgggc | 33109 |
| | gctagcacactggtatcacggtacctttgtgcgcctgttttatac | |
| | cccctaccccaactttaaacttagaagcaaagcaaacccgatcaa | |
| | tagcagatgtagcaagccagttgcatcttgatcaagtacttctgt | |
| | ctccccggactgagtatcaatagactgctcacgcggttgaaggag | |
| | aaaacgttcgttacccgaccaactacttcgagaaacctagtaaca | |
| | ccatgaaagtagcagggtgtttcgatcagcacaaccccagtgtag | |
| | atcaggctgatgagtcaccgcatcccccacgggcgaccgtggcgg | |
| | tggctgcgctggcggcctgcccatggggcaacccatgggacgctc | |
| | taatatggacatggtgtgaagagtctattgagctagttggtagtc | |
| | ctccggcccctgaatgcggctaatcctaactgcggagcacatacc | |
| | ccaaagccaaggggcagtgtgtcgtaacgggcaactctgcagcgg | |
| | aaccgactactttgggtgtccgtgtttccttttattctcaaactg | |
| | gctgcttatggtgacaatctcagagttgttaccatatagctattg | |
| | gattggccatccggtgagcaacagagcaattgtctaccaatttgt | |
| | tggatttattccactcaatctagcggttttcagaacattgaatta | |
| | tattctaaccctcaacaagacgaa | |
| iEV-B74 IRES | ttaaaacagcctgtgggttgttcccactcacagggcccactgggc | 33110 |
| | gctagcacactggtatcacggtacccttgtgcgcctgttttattc | |
| | accccccccaaatgtaacttagaagcataacccaaatggtcaata | |
| | gacagcccagtacaccagctgggtttcggccaagtacttctgtta | |
| | ccccggaccgagtatcaataggctgctcacgcggctgaaggagaa | |
| | aacgttcgttatccggccaattacttcgagaaacctagtaacacc | |
| | atgaaagttgcgcagtgtttcgctccgcacaaccccagtgtagat | |
| | caggtcgatgagtcaccgcgttccccacgggcgaccgtggcggtg | |
| | gctgcgttggcggcctgcccatggggcaacccatgggacgcttca | |
| | atactgacatggtgcgaagagtctattgagctaattggtagtcct | |
| | ccggcccctgaatgcggctaatcctaactgcggagcagataccca | |
| | catgccagtgggcggtctgtcgtaacgggcaactccgcagcggaa | |
| | ccgactactttgggtgtccgtgtttccttttattcttaatctggc | |
| | tgcttatggtgacaattgaaagattgttgccatatagctattgga | |
| | ttggccatccggtgacaaacagggcgattatttatttgtttgttg | |
| | gttttgttccattaaactatagagttttgaatactctagaattta | |
| | ttataacactcaacacaacaaa | |
| iEV-B75 IRES | tttaaaacagtcctgtgggttgttacccacccacagggcccactg | 33111 |
| | ggcgccagcacactggtatcacggtacctttgtgcgcctgtttta | |
| | tatacccttcccctactaagtaacttagaagcaattaaacaaaga | |
| | ccaacagtaagcacagcgcaccagctgtgtttcggtcaagcactt | |
| | ctgttaccccggactgagtatcaataggctactcacgtggctgaa | |
| | ggagaaaacgttcgttacccgaccaattacttcgagaagcctagt | |
| | aacaccatggaggttgcgcggcgtttcgctccacacaaccccagt | |
| | gcagatcaggtcgatgagccaccgcattccccacgggcgaccgtg | |
| | gcggtggctgcgttggcggcctgcccatggggcaacccatgggac | |
| | gcttcaatactgacatggtgcgaagagtctattgagctaattggt | |
| | agtcctccggcccctgaatgcggctaatcccaactgcggagcaga | |
| | tacccacgcgccagtgggcagtctgtcgtaatgggcaactctgca | |
| | gcggaaccgactactttgggtgtccgtgtttcctttattcttata | |
| | ctggctgcttatggtgacaattgagagattgttaccatatagcta | |
| | ttggattggccatccggtgacgagcagagctattatttacctatt | |
| | tgttggttttataccattgaactataaagttttaaaaaccttaaa | |
| | cttcatcttagtactcaatacagcaaa | |
| iEV-B77 IRES | ttaaaacagcctgtgggttgtacccacccacagggcccattgggc | 33112 |
| | gctagcacactggtattccggtacccttgtgcgcctgttttatct | |
| | acccttccctactgcaacttagaagttaaacagtcacggtcgata | |
| | ggcggttcagtaaaccaactggatcttgaccaagcacttctgtta | |
| | ccccggactgagtatcaataagctgcttacgtggctgaaggagaa | |
| | aacgttcgttacccggccaattacttcgagaaacctagtaccacc | |
| | atgaaggttgcgcagcgtttcgctccgcacaaccccagtgtagat | |
| | caggtcgatgagtcaccgcactcctcacgggcgaccgtggcggtg | |
| | gctgcgctggcggcctgcccatggggcaacccatgggacgcttca | |
| | atactgacatggtgcgaagagtctattgagctaattggtaatcct | |
| | ccggcccctgaatgcggctaatcccaactgcggagcaggtaccca | |
| | cacaccagtgggcagcctgtcgtaacgggcaactctgcagcggaa | |
| | ccgactactttgggtgtccgtgtttccttttattcctatactggc | |
| | tgcttatggtgacaattgaaagattgttaccatatagctattgga | |
| | ttggccatccagtgacaaacagagcaattgtctatctatttgtcg | |
| | gttttgtaccaataaatttcaaagtcctaaaaaccctcaatttca | |
| | tattgttacttaacacaacaag | |
| iEV-B79 IRES | ttaaaacagcctgtgggttgtacccacccacagggcccattgggc | 33113 |
| | gctagcacactggtatcacggtacccttgtgcgcctgttttatcc | |
| | gcccctccccattgtaacttagaagataactaaacacggtcgtta | |
| | ggtggctcagtacgccaactgagtcttgaccaagcacttctgtta | |
| | ccccggactgagtatcaataggctgctcacgcggctgaaggagaa | |
| | aacgttcgttatccggccaattacttcgagaaacctagtaccacc | |
| | atggaggttgcgcagtgtttcgctccgcacaaccccagtgtagat | |
| | caggccgatgagtcaccgcattccccacgggcgaccgtggcggtg | |
| | gctgcgctggcggcccgcccatgggtaacccatgggacgcttcaa | |
| | tactgacatggtgtgaagagtctattgagctaattggtagtcctc | |
| | cggcccctgaatgcggctaatcccaactgcggagcagatacccac | |
| | acaccagtgggcagtctgtcgtaacgggcaactctgcagcggaac | |
| | cgactactttgggtgtccgtgtttccttttattccaatactggct | |
| | gcttatggtgacaattgaaagattgttaccatatagctattggat | |
| | tggccatccggtgacaaacagagcgattgtttacctatttgtagg | |
| | tttcataccattgaattttaaagttttaagaacccttaactttat | |
| | attagcacttaacacaacaaa | |
| iEV-B80 IRES | ttaaaacagcctgtgggttgatcccacccacagggcccactgggc | 33114 |
| | gtcagcactctggtatcacggtacctttgtgcgcctgttttatac | |
| | tccctccccccaaccgtaacttagaagcagcactaactgatcaat | |
| | agccagcgcggcatgccagccacgcttcgatcaagcacttctgtt | |
| | accccggactgagtatcaatagactgctcacgcggttgaaggaga | |
| | aaacgttcgtcatccggccaactacttcgagaaacctagtaacac | |
| | catggaagttgcggagtgtttcgctcagcactaccccagtgtaga | |
| | tcaggtcgatgagtcaccgcattccccacaggcgactgtggcggt | |
| | ggctgcgttggcggcctgcccatggggaaacccatgggacgctct | |
| | tatatagacatggtgcgaagagtctattgagctagttggtagtcc | |
| | tccggcccctgaatgcggctaatcctaactgcggagcatacaccc | |
| | tcaaaccagagggcagtgtgtcgtaatgggcaactctgcagcgga | |
| | accgactactttgggtgtccgtgtttcactttatttctaaattgg | |
| | ctgcttatggtgacaattgagagattgttaccatatagctattgg | |
| | attggccatccggtgaccaatagagctattatatatctctttgtt | |
| | gggttcataccacttagcttgaaagaggttaaaactctgcattac | |
| | attatactattgaattccgcaaa | |
| iEV-B81 IRES | ttaaaacagcctgtgggttgtacccacccacagggcccattgggc | 33115 |
| | gcttgcacactggtattccggtacctttgtgcgcctgttttatac | |
| | atcctacccccgtaacttagaagcaagatcacccgatcaccagga | |
| | aatgtggcaaaccagtcacattctgatcaagcacttctgtttccc | |
| | cggactgagtatcaataggctgttcacacggctgaaggagaaaac | |
| | gttcgttacccggccaactacttcgaaaaacctagtatcaccgtg | |
| | aacgttgcagagtgtttcgctcagcactttccccgtgtagatcag | |
| | gtcgatgagtcaccgcattccccacgggcgaccgtggcggtggct | |
| | gcgttggcggcctgcctgcggggcaacccgtaggacgctctaata | |
| | cagacacggtgtgaagagtctattgagctagttggtagtcctccg | |
| | gcccctgaatgcggctaatcctaactgcggagcgagcgcttgtaa | |
| | tccaacaggtagctcgtcgtaacgggtaactctgcagcggaaccg | |
| | actactttgggtgaccgtgtttccttttatttcattactggctgc | |
| | ttatggtgacaatcacagagttgttaccatatagctattggattg | |
| | gccatccggtgtcaaatagagcaattgtgtttctttttgttgggt | |
| | ttgtaccacttgacttgaccagctacaacactctgtgctacattg | |
| | ttagattgaacacagcaaa | |
| iEV-B82 IRES | ttaaaacagcctgtgggttgcacccacccgcagggcccactgggc | 33116 |
| | gctagtacactggtaccccggtacccttgtacgcctgttttattt | |
| | ctccctcccccaaccagtaacctagaagtaaagcaaacccgatca | |
| | atagcaggtgcgacacaccagtcgtatcttgatcaagcacttctg | |
| | taaccccgggccgagtatcaatagactgctcacgcggttgaggga | |
| | gaaaacgctcgttatccggctgactacttcgagaaacccagtaac | |
| | accatagaagttgcagagtgtttcgctcagcactttccccgtgta | |
| | gatcaggtcgatgagtcaccgcattccccacgggtgaccgtggcg | |
| | gtggctgcgttggggcctgcctatggggcaacccatgggacgctc | |
| | taatacagacatggtgtgaagagtcaattgagctagtcagtattc | |
| | ctccggcccctgaatgcggctaatcctaactgcggagcacgtgcc | |
| | tccaatccagggggtggcgtgtcgtaacgggtaactctgcagcgg | |
| | aaccgactactttgggtgtccgtgtttccttttattcttatcttg | |
| | gctgcttatggtgacaattgaaggattgttaccatatagctattg | |
| | gattggccatccggtgactaacagagcaatcatataccaatttgt | |
| | tggttttataccattgaattacaaagttctaatcacccttaaatt | |
| | cattttaacacttaatacgtcaaa | |
| iEV-B83 IRES | tttaaaacagcctgtgggttgttcccacccacagggcccactggg | 33117 |
| | cgctagcactctggtatcacggtacccttgtgcgcctgttttata | |
| | tccccaccccagtggaacattagaagcagcacacctatgatcaat | |
| | agtgggcgtggcatccagtcacgttatgatcaagcacttctgtga | |
| | ccccggactgagtatcaatagactgctcacgcggttgaaggagaa | |
| | aacgttcgttacccggccagttacttcgagaaacccagtaacacc | |
| | atgaatgttgcagagtgtttcgctcagcacttcccccgtgtagat | |
| | caggtcgatgagccactgcattccccacgggcgaccgtggcagtg | |
| | gctgcgttggcggcctgcctatggggtcacccataggacgctcta | |
| | atactgacatggtgcgaagagtctattgagctaactggtagtcct | |
| | ccggcccctgaatgggctaatcctaactgtggagcacattccccc | |
| | aatccagggggcagtgtgtcgtaacgggcaactctgcagcggaac | |
| | cgactactttgggtgtccgtgtttctttttattctcatattggct | |
| | gcttatggtgacaattacagaattgttaccatatagctattggat | |
| | tggccatccggtgactaacagagcaattatttatcaattcgttgg | |
| | atttgtcccactcgatttcaaagcagtcaatacactctcatatat | |
| | cgttacactcaacacaacaaa | |
| iEV-B84 IRES | ttaaaacagcctgtgggttgttcccacccacagggcccaccgggc | 33118 |
| | gccagcacactggtatcacggtacccttgtgcgcctgttttatcc | |
| | accccttcccacagtaacttagaagcacaccatgtatacggtcaa | |
| | taggcggctcagtacaccaactgggccacgaccaagcacttctgt | |
| | taccccggactgagtatcaataagctgctcacgcggctgaaggag | |
| | aaaacgttcgttacccggccaattacttcgagaaacccagtacca | |
| | ccatgaaggttgcgcagcgtttcgttccgcacaaccccagtgtag | |
| | atcaggtcgatgagtcaccgcgttcctcacgggtgaccgtggcgg | |
| | tggctgcgttggcggcctgcccatggggcaacccatgggacgctt | |
| | caatactgacatggtgcgaagagtctattgagctaattggtagtc | |
| | ctccggcccctgaatgcggctaatcctaactgcggagcagatacc | |
| | cacaaaccagtgggcagtctgtcgtaacgggcaactctgcagcgg | |
| | aaccgactactttgggtgtccgtgtttccttttatccttacactg | |
| | gctgcttatggtgacaattgagaaattgttaccatatagctattg | |
| | gattggccatccggtgacaaacagagcagttgtttacctatttgt | |
| | tggtttcgtaccgctgaaccttaaagttctaaagaccctcaattt | |
| | tatcttagcacttaacacagcaaa | |
| iEV-B85 IRES | ttaaaacagcctgtgggttgttcccacccacagggcccaccgggc | 33119 |
| | gctagcacactggtattgcggtacctttgtgcgcctgttttatac | |
| | acccctccttaatgtaactcagaagtacaacattaccggtcaaca | |
| | gatgactcagtaaaccaactgagcctcgaccaagcacttctgttt | |
| | ccccggaccgagtatcaataggctgctcacgcggctgaaggagaa | |
| | agtgttcgttacccggccaattacttcgagaagcccagtaccacc | |
| | atggaggttgcgcggcgtttcgctccgcacatccccagtgtagat | |
| | caggctgatgagtcaccgcgttccccacgggtgaccgtggcggtg | |
| | gctgcgctggcggcctgcccatggggtaacccatgggacgcttca | |
| | atactgacatggtgtgaagagcctattgagctaattggtagtcct | |
| | ccggcccctgaatgcggctaatcctaactgcggagcagataccca | |
| | cacaccagcgggcagtctgtcgtaacgggcaactctgcagcggaa | |
| | ccgactactttgggtgtccgtgtttccttttatttttatgctggc | |
| | tgcttatggtgacaattgaggaattgttaccatatagctattgga | |
| | ttggccatccagtgtccaacagagcgatcatttacctgttcgttg | |
| | ggttcgtaccactaaaccacaagtcactcagcacacttaattaca | |
| | ttataactctcaacacaacaag | |
| iEV-B86 IRES | ttaaaacagcctgtgggttgtacccacccacagggcccactgggc | 33120 |
| | gctagcacactggtattacggtacccttgtgcgcctgttttattt | |
| | acccctcccccacgcaacctagaagcatgacttaagcggtcaata | |
| | gatggctcagtatgccaactgagttatgaccaagcacttctgtta | |
| | ccccggaccgagtatcaataggctgctcacgcggctgaaggagaa | |
| | agtgttcgttacccggccagttacttcgagaaacctagtatcatc | |
| | atgaaggttgcgaggcgtttcgctccacacgaccccggtgtagat | |
| | caggctgatgagtcaccgcattccccacgggcgaccgtggcggtg | |
| | gctgcgctggcggcctgcccatggggcaacccatgggacgcttca | |
| | atattgacatggtgcgaagagtctattgagctaactggtagtcct | |
| | ccggcccctgaatgcggctaatcctaactgcggagcagacaccca | |
| | catgccagtgggcagtctgtcgtaacgggcaactctgcagcggaa | |
| | ccgactactttgggtgtccgtgtttccttttattcttatattggc | |
| | tgcttatggtgacaattgaaagattgttgccatatagctattgga | |
| | ttggccatccagtgactaacagagcaattgtctacctgttcgttg | |
| | gttacgtaccactaaattataaagttttgaacacacttaatttta | |
| | ttatagcacttaacaccttaaa | |
| iEV-B87 IRES | ttaaaacagcctgtgggttgttcccacccacagggcccatagggc | 33121 |
| | gccagcactctggtatcccggtacctttgtgcgcctgttttatcc | |
| | acccttccccaagcgtaacttagaagcccatcatacacggtcaat | |
| | aggtggttcagtatgccaactgagtcttgaccaagcacttctgtt | |
| | accccggactgagtatcaataagctgctcacgcggctgaaggaga | |
| | aaacgttcgttatccggccaattacttcgagaaacctagtaacac | |
| | catgaaggttgcgcagcgtttcgctccgcacaaccccagtgtaga | |
| | tcaggtcgatgagtcaccgcactccccacgggcgaccgtggcggt | |
| | ggctgcgttggcggcctgcccatggggcaacccatgggacgcttc | |
| | aatactgacatggtgcgaagagtctattgagataattggtagtcc | |
| | tccggcccctgaatgcggctaatcctaactgcggagcagataccc | |
| | acatgccagtgggcagtctgtcgtaacgggcaactctgcagcgga | |
| | accgactactttgggtgtccgtgtttccttttatttctatactgg | |
| | ctgcttatggtgacaattgacagattgttaccatatagctattgg | |
| | attggccatccggtgacgaacagagcaattatttacttatttgtt | |
| | ggttttataccattaaatttcaaagttttaaaaaccctcaactta | |
| | gttttgtcactcaactcagcaaa | |
| iEV-B88 IRES | ttaaaacagcctgtgggttgatcccacccacagggcccactgggc | 33122 |
| | gctagcacactggtattccggtacttttgtgcgcctgttttatac | |
| | acccttccccttcatgtaacttagaagtaaaacacaaacggtcaa | |
| | taggtagctcagcaaaccagctgagcctcggccaagcacttctgt | |
| | taccccggactgagtatcaataagctgttaacacggctgaaggag | |
| | aaaacgttcgttatccggccaattacttcgagaaacctagtaaca | |
| | ccatgaaggttgcgtagcgtttcgctccacacaaccccagtgtag | |
| | atcaggtcgatgagtcaccgctctccccacgggtgaccgtggcgg | |
| | tggctgcgttggcggcctgcccatggagcactccatgggacgctt | |
| | caatactgacatggtgtgaagagtctattgagctaattggtagtc | |
| | ctccggcccctgaatgcggctaatcctaactgcggagcagacacc | |
| | cacacgccagtgggcagtctgtcgtaatgggcaactctgcagcgg | |
| | aaccgactactttgggtgtccgtgtttccttttattcttacattg | |
| | gctgcttatggtgacaatcaaggaattgttaccatatagctattg | |
| | gattggccatccggtatctaacagagcaattgtttacctcttcac | |
| | cggttttgtaccattgaattgcaatagtctcaacaccctttgcta | |
| | tatcattaaattgaactcagcaaa | |
| iEV-B93 IRES | aaagcaggctttaaaacagctctggggttgttcccacccacaggg | 33123 |
| | cccactgggcgctagcactctggtatcacggtacctttgtgcgcc | |
| | tgtttttccacccctccccatacgcaacttagaagagagactcaa | |
| | ccggtcaatagacagcctagtacaccaactaggttctgaccaaga | |
| | acttctgtttccccggaccgagtatcaataggctgcttacgcggc | |
| | tgaaggagaaaacgttcgttacccggccaattacttcgagaaacc | |
| | tagtatcaccatggaagttgcgcagtgtttcgctccgcacaaccc | |
| | cagtgtagatcaggtcgatgagtcaccgcattccccacaggcgac | |
| | tgtggcggtggctgcgttggcggcctgcccgtggggcaacccacg | |
| | ggacgcttcaataccgacatggtgcgaagagtctattgagctaat | |
| | tggtagtcctccggcccctgaatgcggctaatcctaactgtggag | |
| | cagatacccacgcgccagtgggcagtctgtcgtaatgggcaactc | |
| | cgcagcggaaccgactactttgggtgtccgtgtttccttttatcc | |
| | tcataatggctgcttatggtgacaattgaaagattgttaccatat | |
| | agctattggattggccatccggtgactaacagagcaattatttat | |
| | ttgtttgttgggtttgtaccattgaactacaaggtccttaatact | |
| | ctaaactacattataagtcttaatacagtgca | |
| iEV-B97 IRES | ttaaaacagcctgtgggttgttcccacccacagggcccactgggc | 33124 |
| | gccagcacactggtatcacggtacctttgtgcgcctgttttatcc | |
| | acccttccccaatgtaacttagaagtaaaactagaaagatcaata | |
| | gcaagcacagtatgccaactgtgttatgatcaagcacttctgtta | |
| | ccccggactgagtatcaataagctgctaaagcggctgaaggagaa | |
| | aacgttcgttacccggccaactacttcgagaaacccagtaccacc | |
| | atgaaggttgcgcagtgtttcgctccacacaatcccagtgtagat | |
| | caggtcgatgagtcaccgcgttccccacgggcgaccgtggcggtg | |
| | gctgcgttggcggcctgcccatgggatgacccatgggacgcttca | |
| | atactgacatggtgcgaagagtctattgagctagttggtagtcct | |
| | ccggcccctgaatgcggctaatcctaactgcggagcagataccca | |
| | cacaccagtgggcagtctgtcgtaacgggtaactctgcagcggaa | |
| | ccgactactttgggtgtccgtgtttctttttatcctcaaattggc | |
| | tgcttatggtgacaattgataagttgttaccatatagctattgga | |
| | ttggccatccggtgtctaacagagctattgtttatttatttgttg | |
| | gtttcgtgccactaaattacaaagtttttgaaactctcaacttca | |
| | ttttgacactcaataaagcaaa | |
| iEV-B98 IRES | ttaaaacagcctgtgggttgttcccacccacagggcccaatgggc | 33125 |
| | gccagcactctggtattacggtacctttgtgcgcctgttttatat | |
| | cccctcccccaattgtaacttagaagcaacacgcaccgaccaaca | |
| | gtgggcgtggcacaccagccacgtcttggtcaagcacatctgttt | |
| | ccccggactgagtatcaatagactgctcacgcggtcgaaggagaa | |
| | aacgttcgttatccggccaactacttcgagaagcccagtaacacc | |
| | atgaatattgcgaagtgtttcgctcagcacactcccagtgtagat | |
| | caggtcgatgagtcaccgcattccccacgggcgaccgtggcggtg | |
| | gctgcgttggcggcctgcctatggggcaacccgtaggacgcttca | |
| | atatggacatggtgtgaagagtctattgagctagttggtagtcct | |
| | ccggcccctgaatgcggctaatcctaaccgcggagcaggcaccca | |
| | caaaccagtgggcagcttgtcgtaacgggtaactctgcggcggaa | |
| | ccgactactttgggtgtccgtgtttccttttattcttatactggc | |
| | tgcttatggtgacaattgagagattgttgccatatagctattgga | |
| | ttggccatccggtgacaaacagagcaattgtttatctatttgttg | |
| | gttttataccgttaagttttaaagtcctaaaaacccttaacttgg | |
| | tcatactactcaactcagcaaa | |
| iEV-B100 IRES | ttaaaacagtctgtgggttgttcccacccacagggcccacgtggc | 33126 |
| | gccagcacactggtatctcggtacctttgtgcgcctgttttatcc | |
| | accctaaaccccgttgtaacttagaagatgagcaacactgatcaa | |
| | tagtgggtgcaacatgccagttgcatcacgatcaagcacttctgt | |
| | atccccggactgagtatcaataggctgctcacgcggctgaaggag | |
| | aaaacgttcgttatccggccagttacttcgagaaacctagtaaca | |
| | tcatggaggttgcgcagtgtttcgctccgcacacccccagtgtag | |
| | atcaggtcgatgagtcaccgcgttccccacgggcgaccgtggcgg | |
| | tggctgcgttggcggcctgcccatgggataacccatgggacgctt | |
| | caatactgacatggtgtgaagagtctattgagctaactggtagtc | |
| | ctccggcccctgaatgcggctaatcctaactgtggagcagacacc | |
| | cacatgccagtgggcagtctgtcgtaacgggcaactccgcagcgg | |
| | aaccgactactttgggtgtccgtgtttccttttattcctatattg | |
| | gctgcttatggtgacaattgaaagattgttaccatatagctattg | |
| | gattggccatccggtgtcaaacagagcaatcatttatttgtttgt | |
| | tggttttatacctctgaactttaaagtgctaaagactctcaattt | |
| | tatattatatctcaacaaatcagg | |
| iEV-B101 IRES | ttaaaacagcctgtgggttgttcccacccacagggcccactgggc | 33127 |
| | gccagcactctggtattacggtacctttgtgcgcctgttttatat | |
| | ccccctccccacttgaaacttagaagtgaaacacactgatcaata | |
| | gttgacatggcaagccagccatgtcttgatcaagcacctctgttt | |
| | ccccggacctagtatcaatagactgctcgcgcggttgaaagagaa | |
| | agcgttcgttacccggctaactacttcgagaaacttagtaacacc | |
| | atggaagttgcggagtgtttcgctcagcacatccccagtgtagat | |
| | caggccgatgagtcaccgcattccccacgggcgaccgtggcggtg | |
| | gctgcgttggcggcctgcctgtggggcaacccacaggacgctcta | |
| | atacaaacatggtgcgaagagtctattgagctagttagtagtcct | |
| | ccggcccctgaatgcggctaatcctaactgcggagcacgtgcttc | |
| | caatccaggaggtggcgtgtcgtaacgggtaactctgcagcggaa | |
| | ccgactactttgggtgtccgtgtttccttttattcttgcattggc | |
| | tgcttatggtgacaattgagagattgttaccatatagctattgga | |
| | ttggccatccggtgacaaacagagcaattatatatctctttgttg | |
| | gatacattccattgaactacaaggagttaaaaaccctcaattata | |
| | ttctgttcttgaattccgcaag | |
| iEV-B106 IRES | ttaaaacagcctgtgggttgttcccacccacagggcccatggggc | 33128 |
| | gccagcacactggtattgcggtacctttgtgcgcctgttttattc | |
| | accctttcccaatgaaacttagaagcttaactcaaccggtcgata | |
| | gacagcccagtacaccaactgggttttgaccaagtactcctgttt | |
| | ccccggactgagtatcaataagctgctcgcgcggctgaaggagaa | |
| | aacgttcgttacccggccaattacttcgagaaacctagtaccacc | |
| | atgaacgttgcgcagtgtttcgctccgcacaaccccagtgtagat | |
| | caggtcgatgagtcaccgcaatccccacgggcgaccgtggcggtg | |
| | gctgcgctggcggcctgcccatggggcatcccatgggacgcttca | |
| | atactgacatggtgcgaagagtctattgagctaattggtaatcct | |
| | ccggcccctgaatgcggctaatcctaactgcggggcagacaccca | |
| | cacgccagtgggcagtctgtcgtaacgggtaactccgcagcggaa | |
| | ccgactactttgggtgtccgtgtttccttttatctttatattggc | |
| | tgcttatggtgacaattgaaagattgttgccatatagctattgga | |
| | ttggccatccggtgtctaatagagcaattatatatctctttgttg | |
| | gcttcatcccgttaaataaagtgagctaccagaccctgtgctata | |
| | ttatcaaattgaacacaataaa | |
| iEV-B107_ | ttaaaacagcctgtgggttgttcccacccgcagggcccactgggc | 33129 |
| TN94-0349 | gctagcacactggtatcccggtacccttgtgcgcctgttttatat | |
| IRES | accctcccccttatgtaacttagaagtatgattcaaacggtcgac | |
| | aggcggctcagtgcaccaactgagtcatgaccaagcacttctgtt | |
| | accccggactgagtatcaataagctgttcacacggctgaaggaga | |
| | aaacgttcgttacccggccaattacttcgagaaacctagtaccac | |
| | catgaaggttgcgcagtgtttcgctccacacaaccccagtgtaga | |
| | tcaggtcgatgagtcaccgcattccccacgggcgaccgtggcggt | |
| | ggctgcgttggcggcctgcccatggggcaacccatgggacgcttc | |
| | aatactgacatggtgtgaagagtctattgagctaattggtagtcc | |
| | tccggcccctgaatgcggctaatcccaactgtggagcagatactc | |
| | acaaaccagtgagcggtctgtcgtaacgggcaactccgcagcgga | |
| | accgactactttgggtgtccgtgtttctttttattcttacattgg | |
| | ctgcttatggtgacaattgacaaattgttaccatatagctattgg | |
| | attggccatccggtgacaaacagagctattgtttacttgtttgtt | |
| | ggtttcataccattaaattacaaggtcttagaaactctcaacttt | |
| | attttgacactcaatacagcaaa | |
| iEV-B111 IRES | ttaaaacagcctgtgggttgttcccacccacagggcccactgggc | 33130 |
| | gccagcacactggtatcacggtacccttgtgcgcctgttttataa | |
| | acccttccccaatgcaacttagaagctcaactttaccggtcaata | |
| | gacagtccagtacgccaactgggcttcgatcaagcacttctgtta | |
| | ccccggactaagtatcaataggctgctcacgcggctgaaggagaa | |
| | aacgttcgttacccggccaattacttcgagaaacccagtaacact | |
| | atggaggttgcgcagtgtttcgttccacacaaccccagtgtagat | |
| | caggtcgatgagtcaccgctttccccacgggcgaccgtggcggtg | |
| | gctgcgttggcggcctgcccatggggcaacccatgggacgcttca | |
| | atactgacatggtgtgaagagtctattgagctaattggtattcct | |
| | ccggcccctgaatgcggctaatcctaactgcggagcagatactca | |
| | cgagccagtgggcagtctgtcgtaacgggtaactctgcagcggaa | |
| | ccgactactttgggtgtccgtgtttccttttattcatatattggc | |
| | tgcttatggtgacaattgagagattgttaccatatagctattgga | |
| | ttggccatccggtgactaatagagcaattgtttacctatttgttg | |
| | gctttgtgccgttgaactacaaggttttaaaaactattaactaca | |
| | tcctagtacttaacacgttgaa | |
| iEV-B113 IRES | actctggtattgcggtacctttgtacgcctgttttatatcccttc | 33131 |
| | cccatgtaactttagaagcaattcacaaggttcaatagagggggt | |
| | acaaaccagtaccaccacgaacaagcacttctgtttccccggtga | |
| | agctgtatagactgtccccacggtcaaaaacggcagatccgttat | |
| | ccgcttatgtacttcgagaagcctagtatcatcttggaatcttcg | |
| | atggttgcgttcagcactctatcccgagtgtagcttaggtcgatg | |
| | agtctgggcattccccaccggcgacggtggcccaggctgcgttgg | |
| | cggcctacccatggctaacgccatgggacgctagttgtgaacaag | |
| | gtgtgaagagcctattgagctacatgagagtcctccggcccctga | |
| | atgcggctaatcccaaccacggagcaagtactcacaacccagtgg | |
| | gcagcttgtcgtaacgcgtaagtctgtggcggaaccgactacttt | |
| | gggtgtccgtgtttcctttttatttttgatatggctgcttatggt | |
| | gacatctaagattgttatcatatagcttttggattggccatccgg | |
| | tgatttttgagactaaaacgcttgtttgccttatcacacaattta | |
| | gtattttgttatattcatacggtcata | |
| iEV-D68 IRES | ttaaaacagctctggggttgttcccactcaagggcccacgtggcg | 33132 |
| | gctagtactctggtatctcggtacctttgtacgcctgttttaatt | |
| | ccctccccaacgtaacttagaagcttttaaaccaaagctcaatag | |
| | gtggagcgcaaaccagcgctcttatgagcaagcacttctgtctcc | |
| | ccggtgtggttgtatagactgtccccacggttgaaaacaacttat | |
| | ccgttaCccgctatagtacttcgagaaacctagtattgccttcgg | |
| | agtgttgatgcgttgcgctcagcacactaacccgtgtgtagcttg | |
| | ggtcgatgagtctggacgtaccccactggcgacagtggtccaggc | |
| | tgcgttggcggcctactcatggtgaaaaccatgGgacgctagaca | |
| | tgaacaaggtgtgaagagtctattgagctgctatagagtcctccg | |
| | gcccctgaatgcggctaatcctaaccatggagcaagtgctcacaa | |
| | accagtgagttacttgtcgtaacgcgcaagtccgtggcggaaccg | |
| | actactttgggtgtccgtgtttcactttttacttttatgactgct | |
| | aatggtgacaatttaatattgttaccatttggcttgtcgaattga | |
| | tcacataagatctatagttttgttcactgatttgctttgaaataa | |
| | tctcacctcaaaacctccagtacataacatttaaagagtttaaac | |
| | ttatttataaca | |
| iEV-D70 IRES | ttaaaacagctctggggttgttcccaccccagaggcccacgtggg | 33133 |
| | gctagtactccggtaccccggtacccttgtacgcctgttttatac | |
| | tccctttcccaagtaactttagaagaaataaactaatgttcaaca | |
| | ggagggggtacaaaccagtaccaccacgaacacacacttctgttt | |
| | ccccggtgaagttgcatagactgtacccacggttgaaagcgatga | |
| | atccgttacccgcttaggtacttcgagaagcctagtatcatcttg | |
| | gaatcttcgatgcgttgcgatcagcactctaccccgagtgtagct | |
| | tgggtcgatgagtctggacaccccacaccggcgacgtggtccagg | |
| | ctgcgttggcggcctacccatggctagcaccatgggacgctagtt | |
| | gtgaacaaggtgcgaagagcctattgagctacctgagagtcctcc | |
| | ggcccctgaatgcggctaatcccaaccacggagcaaatgctcaca | |
| | atccagtgagtggtttgtcgtaatgcgcaagtctgtggcggaacc | |
| | gactactttgggtgtccgtgtttccttttatttttattatggctg | |
| | cttatggtgacaatctgagattgttatcatatagctattggatta | |
| | gccatccggtgaCatcttgaaattttgccataactttttcacaaa | |
| | tcctacaacattacactacactttctcttgaataattgagacaac | |
| | tcata | |
| iEV-D94 IRES | ttaaaacagcctgtgggttgttcccaccccagaggcccacgtggc | 33134 |
| | ggccagtactctggtatcacggtacctttgtacgcctgttttata | |
| | tccccttcccccgcaacttagaagaaaacaaatcaagttcactag | |
| | gagggggtacaaaccagtaccaccacgaacaagcacttctgtttc | |
| | cccggtgatgtcgtatagactgtaaccacggttgaaaacgattga | |
| | tccgttatccgctcttgtacttcgaaaagcccagtatcaccttgg | |
| | aatcttcgatgcgttgcgctcagcactcaaccccagagtgtagct | |
| | taggtcgatgagtctggacactcctcaccggcgacggtggtccag | |
| | gctgcgttggcggcctacctgtggtccaaagccacaggacgctag | |
| | ttgtgaacaaggtgtgaagagcctattgagctacaagagaatcct | |
| | ccggcccctgaatgcggctaatcctaaccacggagcaagggtaca | |
| | caaaccagtgtatatcttgtcgtaacgcgcaagtctgtggcggaa | |
| | ccgactactttgggtgtccgtgtttccttttgtttttatcatggc | |
| | tgcttatggtgacaatctaagattgttatcatatagctgttggat | |
| | tggccatccggtaatttattgagatttgagcatttgcttgtttct | |
| | tcaacaatttcacctattcattgcatttcagcagtcaaa | |
| iEV-D111 IRES | ttcccaccctagaggcccacgtggcggctagtactctggtattac | 33135 |
| | ggtacctttgtacgcctgttttatatccctttccccattgtaact | |
| | tagaagtttcaaagtacagttcaatagacagtagtacaaaccagt | |
| | actgctacgaacaagcacttctgttgccccggtgtagttgcatag | |
| | actgtacccacggttgaaagcaactgatccgttatccgctttagt | |
| | acttcgagaagcctagtagcaccttgggatcttcgatgcgttgcg | |
| | ctcagcactcaaccccagagtgtagcttgggtcgatgagtctgga | |
| | cattcctcaccggcgacggtggtccaggctgcgttggcggcctac | |
| | ctgtggcccaaagccacaggacgctagttgtgaacaaggtgtgaa | |
| | gagcctattgagctactaaagagtcctccggcccctgaatgcggc | |
| | taatcctaaccacggagcaagtgctcacagtccagtgagtagctt | |
| | gtcgtaatgagcaattctgtggcggaaccgactactttgggtgtc | |
| | cgtgtttccttttatttttatcattggctgcttatggtgacaatc | |
| | taaggttgccatcatatagcttttggattggccatcccggtgata | |
| | tttcgaattattcaatttgctgtttgcagctccttttaaacagta | |
| | ttcactcaccagcattcatattttactcttgagttattaacaaat | |
| | agtcaaaa | |
| iHCosV-B1 IRES | ttgccagttttttgaaacgtgtggtttgacgtctaaccattatgg | 33136 |
| | cagtgggtggagtactgcaaagatgtcaccgtgctttacacggtt | |
| | tttgaaccccacaccggctgtttggcgcttgcaggacagcaggtt | |
| | tattttcttttaactctctctttctagccacacacgatctatgtg | |
| | tgtgggcggagtgatactcccgttccttcttggacaggcggcctc | |
| | cacgccctttgtggatcttaaggctaccaagtcactggtgttgga | |
| | aagtgaagagaaaggagttccttgggaactacatgtggcattgac | |
| | agaggttgtagcgatgctgtgtgtgtgtgcggattacccccgtgg | |
| | cgacacggaccccacaggccaaaagccctgtccgaaaggacccac | |
| | acagtggagcaaccccagctcccctcttcaatgttttgtgttagc | |
| | aaccttggtattattttctctcaagcttccaatacaccgggcccc | |
| | aaagatgtcctgaaggtaccccgtgtatctgaggatgagcaccat | |
| | caactacccggacttgttctttcgagaacagacgcatgtggtaac | |
| | ccagccccgatcctaaggggtcggggcttttgctcactcagcaca | |
| | ggatctgatcaggagacctcccccccctgctttacagggggcggg | |
| | ggtttaaaaattgcccaaggcctggcaaataacctaggggactag | |
| | gttttcctttttattttaaagttgtcaat | |
| iEcho-E7 IRES | ttcccacccacagggcccactgggcgctagcacactggtatcacg | 33137 |
| | gtacctttgtgcgcctgttttatccaccctcccccaatgtaactt | |
| | agaagcacaccaaacgcggtcaataggcggctcagtacaccaact | |
| | gagtctcgaccaagcacttctgttaccccggactgagtatcaata | |
| | ggctgctcacgcggctgaaggagaaaacgttcgttacccggccaa | |
| | ttacttcgagaaacctagtaccaccatgaaggttgcgcggcgttt | |
| | cgctccacacaaccccagtgtagatcaggtcgatgagccaccgca | |
| | ttccccacgggcgaccgtggcggtggctgcgctggcggcctgccc | |
| | atggggcaacccatgggacgcttcaatactgacatggtgtgaaga | |
| | gtctattgagctaattggtagtcctccggcccctgaatgcggcta | |
| | atcctaactgcggagcagacacccacatgccagtgggcagtctgt | |
| | cgtaacgggtaactctgcagcggaaccgactactttgggtgtccg | |
| | tgtttccttttattctcatactggctgcttatggtgacaattgaa | |
| | agattgttgccatatagctattggattggccatccggtgacaaac | |
| | agagcggttgtttacctatttgtcggttttataccattgaattac | |
| | aaggttctgaaaaccctcaatttcattttagcacttaataccaca | |
| | aa | |
| iEcho-E9 IRES | ttaaaacagcctgtgggttgttcccacccacagggcccattgggc | 33138 |
| | gctagcacactggtatcacggtacccttgtgcgcctgttttatcc | |
| | acccctccccatcgtaacctagaagcatgactccaacggtcaata | |
| | gacagctcagtaaaccaattgagccttgaccaagcacttctgtta | |
| | ccccggactgagtatcaataggctgctcacgcggccgaaggagaa | |
| | aatgttcgttacccggccaattacttcgagaaacctagtaccacc | |
| | atgaaggttgcgcggtgtttcgctcagcacaaccccagtgtagat | |
| | caggccgatgagtcaccgcattccccacgggtgaccgtggcggtg | |
| | gctgcgctggggcctgcccatggggcaacccatgggacgcttcaa | |
| | tactgacatggtgtgaagagtctattgagctaattggtagtcctc | |
| | cggcccctgaatgcggctaatcctaactgcggagcagatacccac | |
| | gcaccagtgggcagtctgtcgtaacgggcaactctgcagcggaac | |
| | cgactactttgggtgtccgtgtttccttttattcttgtactggct | |
| | gcttatggtgacaattgagagattgttaccatatagctattggat | |
| | tggccatccagtgtcaaacagagcgattgtctacttgttcattgg | |
| | ttttataccattgagttacaaggttctagccactcttaacttcat | |
| | tttaacactcaatacatcaaa | |
| iPV3_PAK1019536 | tggggttaaaacagctctggggttgttcccaccccagaggcccac | 33139 |
| IRES | gtggcggctagtactctggtatcatggtacctttgtacgcctgtt | |
| | ttatacccctaccccacgtaactttagaagcaactcaacaagttc | |
| | aatagggggggtacaaaccagtaccaccacgaacaagcactcctg | |
| | ttaccccggtgatcttgcataagctgtgcccacggctgaaggcga | |
| | gagatccgttatccgcttgagtacttcgagaagcctagtatcacc | |
| | ttgggatcttcgacgcgttgcgctcagcactccacccgagtgtag | |
| | cttaggctgatgagcctgggcattccccaccggcgacggtggccc | |
| | aggctgcgttggcggcctacccatggctaacaccatgggacgcta | |
| | gatgtgaacaaggtgtgaagagcctattgagctactcaagagtcc | |
| | tccggcccctgaatgcggctaatcccaaccacggagcaggtgtct | |
| | tcaacccagaaggtagcctgtcgtaacgcgtaagtctgtggcgga | |
| | accgactactttgggtgtccgtgtttccttttatcttttatatgg | |
| | ctgcttatggtgacaatcacagattgttatcataaagcgatttgg | |
| | attggccatccggtgaagtacaaacacattatctacttgtttgtt | |
| | ggatttactccactcacacaccccacccccaatataacatctatt | |
| | gttttgttGacaagaaattactttcgaa | |
| iPV3_NIE1219535 | gttaaaacagctctggggttgttcccaccccagaggcccaagtgg | 33140 |
| IRES | cggctagtactccggtatcacggtacccttgtacgcctgttttat | |
| | actcccttcccccgtaacttagaagagttaaaccaagttcaatag | |
| | aagggggtacaaaccagtaccaccacgaacaagcacttctgtttc | |
| | cccggtgatattgtatagactgtacccacggtcgaaaatgattga | |
| | tccgttatccgctctcatacttcgagaagcctagtatcatcttgg | |
| | aatcttcgatgcgttgcgctcggcactcgatcccggagtgtagct | |
| | taggtcgatgagtctggacactcctcaccggtgacggtggtccag | |
| | actgcgttggcggcctacctgtggtccaaagccacaggacgctag | |
| | ttgtgaacaaggtgtgaagagcctattgagctatgagagagtcct | |
| | ccggcccctgaatgcggctaatcccaaccacggagcgagagtgcg | |
| | caagccagcgtatatctcgtcgtaacgcgcaagtctgtggcggaa | |
| | ccgactactttgggtgtccgtgtttccttttatttttgtattggc | |
| | tgcttatggtgacaatcacagattgttatcataaagcgaattgga | |
| | ttggccatccggtgaaagtgaaatacattatttgcttatttattg | |
| | gattcagtccacctaattgctatactcacgaacccaccacagccg | |
| | ttttgttGactgaaagccggtaccaata | |
| iPV3_SWI10947 | ttaaaacagctctggggttgttcccaccccagaggcccacgtggc | 33141 |
| IRES | ggctagtactccggtatcacggtacccttgtacgcctgttttata | |
| | ctcccttccccgtaacttagaagtatacaaaccaagttcaataga | |
| | agggggtacaaaccagtaccaccacgaacaagcacttctgtttcc | |
| | ccggtgaagttgcatagattgtccccacggtcgaaagcgactgat | |
| | ccgttatccgctcttgtacttcgggaagcctagtatcaccttcgg | |
| | atcttcgatgcgttgcgctcagcactcaaccccagagtgtagctt | |
| | aggtcgatgagtctggacgaacctcactggtgacagtggtccagg | |
| | ctgcgttggcggcctacctgtggcccaaagccacaggacgctagt | |
| | tgtgaacaaggtgtgaagagcctactgagctacatgagagtcctc | |
| | cggcccctgaatgcggctaatcccaaccacggagcaggtggtcac | |
| | aaaccagtggtttgcctgtcgtaacgcgaaagtctgtggcggaac | |
| | cgactactttgggtgtccgtgtttccttttattcttattatggct | |
| | gcttatggtgacaatcttagattgttatcataaagcgaattggat | |
| | tggccatccggggaaaattaagcatatcatacacttgtttgttgg | |
| | attcacccctctgattacacatactcttggcttgattaggattgt | |
| | tgcattgataagacactattgttgca | |
| iPV3_FIN84 | gggttgttcccaccccagaggcccacgtggcggctagtactccgg | 33142 |
| IRES | tatcacggtacccttgtacgcctgttttatactcccttccccgta | |
| | acttagaagtatacaagccaagttcaatagaagggggtacaaacc | |
| | agtaccaccacgaacaagcacttctgtttccccggtgaagttgca | |
| | tagattgttcccacggtcgaaagcgactgatccgttacccgctca | |
| | tgtacttcgggaagcctagtatcaccttcggatcttcgatgcgtt | |
| | gcgctcagcactcaaccccagagtgtagcttaggtcgatgagtct | |
| | ggacaaacctcactggcgacagtggtccaggctgcgttggcggcc | |
| | tacctgtggcccaaagccacaggacgctagttgtgaacaaggtgt | |
| | gaagagcctactgagctacatgagagtcctccggcccctgaatgc | |
| | ggctaatctcaaccacggagcaggtggtcacaaaccagtgatttg | |
| | cctgtcgtaacgcgaaagtctgtggcggaaccgactactttgggt | |
| | gtccgtgtttccttttactctcattatggctgcttatggtgacaa | |
| | tcctagattgttatcataaagcgagttggattggccatccggtga | |
| | aaatcaagcacatcatatatttgtttgttgggtttaaccctctga | |
| | ttgcacgcactcttggcttggttaggattattgtgtggaaaaggc | |
| | accatagttaca | |
| Apt-eIF4G | ACTCACTATTTGTTTTCGCGCCCAGTTGCAAAAAGTGTCG | 33143 |
| synIRES-RC01 | ttaaaacagcctgtgggttgatcccacccacaggcccattgggcg | 33144 |
| | ctagcactctggtatcacggtacctttgtgcgcctgttttatacc | |
| | ccctACTCACTATTTGTTTTCGCGCCCAGTTGCAAAAAGTGTCGc | |
| | ccccaactgtaacttagaagtaacacacaccgatcaacagtcagc | |
| | gtggcacaccagccacgttttgatcaagcacttctgttaccccgg | |
| | actgagtatcaatagactgctcacgcggttgaaggagaaagcgtt | |
| | cgttatccggccaactacttcgaaaaacctagtaacaccgtggaa | |
| | gttgcagagtgtttcgctcagcactaccccagtgtagatcaggtc | |
| | gatgagtcaccgcattccccacgggcgaccgtggcggtggctgcg | |
| | ttggcggcctgcccatggggaaacccatgggacgctctaatacag | |
| | acatggtgcgaagagtctattgagctagttggtagtcctccggcc | |
| | cctgaatgcggctaatcctaactgcggagcacacaccctcaagcc | |
| | agagggcagtgtgtcgtaacgggcaactctgcagcggaaccgact | |
| | actttgggtgtcgtgtttcattttattcctatactggctgcttat | |
| | ggtgacaattgagagatcgttaccatatagctattggattggcca | |
| | tccggtgactaatagagctattatatatccctttgttgggtttat | |
| | accacttagcttgaaagaggttaaaacattacaattcattgttaa | |
| | gttgaatacagcaaa | |
| synIRES-RC02 | ttaaaacagcctgtgggttgatcccacccacaggcccattgggcg | 33145 |
| | ctagcactctggtatcacggtacctttgtgcgcctgttttatacc | |
| | ccctcccccaactgtaacttagaagtaacacacaccgatcaacag | |
| | tcagcgtgACTCACTATTTGTTTTCGCGCCCAGTTGCAAAAAGTG | |
| | TCGcacgttttgatcaagcacttctgttaccccggactgagtatc | |
| | aatagactgctcacgcggttgaaggagaaagcgttcgttatccgg | |
| | ccaactacttcgaaaaacctagtaacaccgtggaagttgcagagt | |
| | gtttcgctcagcactaccccagtgtagatcaggtcgatgagtcac | |
| | cgcattccccacgggcgaccgtggcggtggctgcgttggcggcct | |
| | gcccatggggaaacccatgggacgctctaatacagacatggtgcg | |
| | aagagtctattgagctagttggtagtcctccggcccctgaatgcg | |
| | gctaatcctaactgcggagcacacaccctcaagccagagggcagt | |
| | gtgtcgtaacgggcaactctgcagcggaaccgactactttgggtg | |
| | tcgtgtttcattttattcctatactggctgcttatggtgacaatt | |
| | gagagatcgttaccatatagctattggattggccatccggtgact | |
| | aatagagctattatatatccctttgttgggtttataccacttagc | |
| | ttgaaagaggttaaaacattacaattcattgttaagttgaataca | |
| | gcaaa | |
| synIRES-RC03 | ttaaaacagcctgtgggttgatcccacccacaggcccattgggcg | 33146 |
| | ctagcactctggtatcacggtacctttgtgcgcctgttttatacc | |
| | ccctcccccaactgtaacttagaagtaacacacaccgatcaacag | |
| | tcagcgtggcacaccagccacgttttgatcaagcacttctgttac | |
| | cACTCACTATTTGTTTTCGCGCCCAGTTGCAAAAAGTGTCGccgg | |
| | actgagtatcaatagactgctcacgcggttgaaggagaaagcgtt | |
| | cgttatccggccaactacttcgaaaaacctagtaacaccgtggaa | |
| | gttgcagagtgtttcgctcagcactaccccagtgtagatcaggtc | |
| | gatgagtcaccgcattccccacgggcgaccgtggcggtggctgcg | |
| | ttggcggcctgcccatggggaacccatgggacgctctaatacaga | |
| | catggtgcgaagagtctattgagctagttggtagtcctccggccc | |
| | ctgaatgcggctaatcctaactgcggagcacacaccctcaagcca | |
| | gagggcagtgtgtcgtaacgggcaactctgcagcggaaccgacta | |
| | ctttgggtgtcgtgtttcattttattcctatactggctgcttatg | |
| | gtgacaattgagagatcgttaccatatagctattggattggccat | |
| | ccggtgactaatagagctattatatatccctttgttgggtttata | |
| | ccacttagcttgaaagaggttaaaacattacaattcattgttaag | |
| | ttgaatacagcaaa | |
| synIRES-RC04 | ttaaaacagcctgtgggttgatcccacccacaggcccattgggcg | 33147 |
| | ctagcactctggtatcacggtacctttgtgcgcctgttttatacc | |
| | ccctcccccaactgtaacttagaagtaacacacaccgatcaacag | |
| | tcagcgtggcacaccagccacgttttgatcaagcacttctgttac | |
| | cccggactgagtatcaatagactgcACTCACTATTTGTTTTCGCG | |
| | CCCAGTTGCAAAAAGTGTCGgcggttgaaggagaaagcgttcgtt | |
| | atccggccaactacttcgaaaaacctagtaacaccgtggaagttg | |
| | cagagtgtttcgctcagcactaccccagtgtagatcaggtcgatg | |
| | agtcaccgcattccccacgggcgaccgtggcggtggctgcgttgg | |
| | cggcctgcccatggggaaacccatgggacgctctaatacagacat | |
| | ggtgcgaagagtctattgagctagttggtagtcctccggcccctg | |
| | aatgcggctaatcctaactgcggagcacacaccctcaagccagag | |
| | ggcagtgtgtcgtaacgggcaactctgcagcggaaccgactactt | |
| | tgggtgtcgtgtttcattttattcctatactggctgcttatggtg | |
| | acaattgagagatcgttaccatatagctattggattggccatccg | |
| | gtgactaatagagctattatatatccctttgttgggtttatacca | |
| | cttagcttgaaagaggttaaaacattacaattcattgttaagttg | |
| | aatacagcaaa | |
| synIRES-RC05 | ttaaaacagcctgtgggttgatcccacccacaggcccattgggcg | 33148 |
| | ctagcactctggtatcacggtacctttgtgcgcctgttttatacc | |
| | ccctcccccaactgtaacttagaagtaacacacaccgatcaacag | |
| | tcagcgtggcacaccagccacgttttgatcaagcacttctgttac | |
| | cccggactgagtatcaatagactgctcacgcggttgaaggagaaa | |
| | gcgttcgttatACTCACTATTTGTTTTCGCGCCCAGTTGCAAAAA | |
| | GTGTCGccggccaactacttcgaaaaacctagtaacaccgtggaa | |
| | gttgcagagtgtttcgctcagcactaccccagtgtagatcaggtc | |
| | gatgagtcaccgcattccccacgggcgaccgtggcggtggctgcg | |
| | ttggcggcctgcccatggggaaacccatgggacgctctaatacag | |
| | acatggtgcgaagagtctattgagctagttggtagtcctccggcc | |
| | cctgaatgcggctaatcctaactgcggagcacacaccctcaagcc | |
| | agagggcagtgtgtcgtaacgggcaactctgcagcggaaccgact | |
| | actttgggtgtcgtgtttcattttattcctatactggctgcttat | |
| | ggtgacaattgagagatcgttaccatatagctattggattggcca | |
| | tccggtgactaatagagctattatatatccctttgttgggtttat | |
| | accacttagcttgaaagaggttaaaacattacaattcattgttaa | |
| | gttgaatacagcaaa | |
| synIRES-RC06 | ttaaaacagcctgtgggttgatcccacccacaggcccattgggcg | 33149 |
| | ctagcactctggtatcacggtacctttgtgcgcctgttttatacc | |
| | ccctcccccaactgtaacttagaagtaacacacaccgatcaacag | |
| | tcagcgtggcacaccagccacgttttgatcaagcacttctgttac | |
| | cccggactgagtatcaatagactgctcacgcggttgaaggagaaa | |
| | gcgttcgttatccggccaactacttcgaaaaacctagtaacaccg | |
| | tggaagttgcagagtgtttcgctcagcactACTCACTATTTGTTT | |
| | TCGCGCCCAGTTGCAAAAAGTGTCGagtgtagatcaggtcgatga | |
| | gtcaccgcattccccacgggcgaccgtggcggtggctgcgttggc | |
| | ggcctgcccatggggaaacccatgggacgctctaatacagacatg | |
| | gtgcgaagagtctattgagctagttggtagtcctccggcccctga | |
| | atgcggctaatcctaactgcggagcacacaccctcaagccagagg | |
| | gcagtgtgtcgtaacgggcaactctgcagcggaaccgactacttt | |
| | gggtgtcgtgtttcattttattcctatactggctgcttatggtga | |
| | caattgagagatcgttaccatatagctattggattggccatccgg | |
| | tgactaatagagctattatatatccctttgttgggtttataccac | |
| | ttagcttgaaagaggttaaaacattacaattcattgttaagttga | |
| | atacagcaaa | |
| synIRES-RC07 | ttaaaacagcctgtgggttgatcccacccacaggcccattgggcg | 33150 |
| | ctagcactctggtatcacggtacctttgtgcgcctgttttatacc | |
| | ccctcccccaactgtaacttagaagtaacacacaccgatcaacag | |
| | tcagcgtggcacaccagccacgttttgatcaagcacttctgttac | |
| | cccggactgagtatcaatagactgctcacgcggttgaaggagaaa | |
| | gcgttcgttatccggccaactacttcgaaaaacctagtaacaccg | |
| | tggaagttgcagagtgtttcgctcagcactaccccagtgtagatc | |
| | aggtcgatgagtcaccgcACTCACTATTTGTTTTCGCGCCCAGTT | |
| | GCAAAAAGTGTCGgcggtggctgcgttggcggcctgcccatgggg | |
| | aaacccatgggacgctctaatacagacatggtgcgaagagtctat | |
| | tgagctagttggtagtcctccggcccctgaatgcggctaatccta | |
| | actgcggagcacacaccctcaagccagagggcagtgtgtcgtaac | |
| | gggcaactctgcagcggaaccgactactttgggtgtcgtgtttca | |
| | ttttattcctatactggctgcttatggtgacaattgagagatcgt | |
| | taccatatagctattggattggccatccggtgactaatagagcta | |
| | ttatatatccctttgttgggtttataccacttagcttgaaagagg | |
| | ttaaaacattacaattcattgttaagttgaatacagcaaa | |
| synIRES-RC08 | ttaaaacagcctgtgggttgatcccacccacaggcccattgggcg | 33151 |
| | ctagcactctggtatcacggtacctttgtgcgcctgttttatacc | |
| | ccctcccccaactgtaacttagaagtaacacacaccgatcaacag | |
| | tcagcgtggcacaccagccacgttttgatcaagcacttctgttac | |
| | cccggactgagtatcaatagactgctcacgcggttgaaggagaaa | |
| | gcgttcgttatccggccaactacttcgaaaaacctagtaacaccg | |
| | tggaagttgcagagtgtttcgctcagcactaccccagtgtagatc | |
| | aggtcgatgagtcaccgcattccccacgggcgaccgtggcggtgg | |
| | ctgcgttggcggcctgcccatgggACTCACTATTTGTTTTCGCGC | |
| | CCAGTTGCAAAAAGTGTCGcccatgggacgctctaatacagacat | |
| | ggtgcgaagagtctattgagctagttggtagtcctccggcccctg | |
| | aatgcggctaatcctaactgcggagcacacaccctcaagccagag | |
| | ggcagtgtgtcgtaacgggcaactctgcagcggaaccgactactt | |
| | tgggtgtcgtgtttcattttattcctatactggctgcttatggtg | |
| | acaattgagagatcgttaccatatagctattggattggccatccg | |
| | gtgactaatagagctattatatatccctttgttgggtttatacca | |
| | cttagcttgaaagaggttaaaacattacaattcattgttaagttg | |
| | aatacagcaaa | |
| synIRES-RC09 | ttaaaacagcctgtgggttgatcccacccacaggcccattgggcg | 33152 |
| | ctagcactctggtatcacggtacctttgtgcgcctgttttatacc | |
| | ccctcccccaactgtaacttagaagtaacacacaccgatcaacag | |
| | tcagcgtggcacaccagccacgttttgatcaagcacttctgttac | |
| | cccggactgagtatcaatagactgctcacgcggttgaaggagaaa | |
| | gcgttcgttatccggccaactacttcgaaaaacctagtaacaccg | |
| | tggaagttgcagagtgtttcgctcagcactaccccagtgtagatc | |
| | aggtcgatgagtcaccgcattccccacgggcgaccgtggcggtgg | |
| | ctgcgttggcggcctgcccatggggaaacccatgggacgctctaa | |
| | tacagacatggtgcgaagagtctattgagctagttggtagtcACT | |
| | CACTATTTGTTTTCGCGCCCAGTTGCAAAAAGTGTCGCCCCctcc | |
| | ggcccctgaatgcggctaatcctaactgcggagcacacaccctca | |
| | agccagagggcagtgtgtcgtaacgggcaactctgcagcggaacc | |
| | gactactttgggtgtcgtgtttcattttattcctatactggctgc | |
| | ttatggtgacaattgagagatcgttaccatatagctattggattg | |
| | gccatccggtgactaatagagctattatatatccctttgttgggt | |
| | ttataccacttagcttgaaagaggttaaaacattacaattcattg | |
| | ttaagttgaatacagcaaa | |
| synIRES-RC10 | ttaaaacagcctgtgggttgatcccacccacaggcccattgggcg | 33153 |
| | ctagcactctggtatcacggtacctttgtgcgcctgttttatacc | |
| | ccctcccccaactgtaacttagaagtaacacacaccgatcaacag | |
| | tcagcgtggcacaccagccacgttttgatcaagcacttctgttac | |
| | cccggactgagtatcaatagactgctcacgcggttgaaggagaaa | |
| | gcgttcgttatccggccaactacttcgaaaaacctagtaacaccg | |
| | tggaagttgcagagtgtttcgctcagcactaccccagtgtagatc | |
| | aggtcgatgagtcaccgcattccccacgggcgaccgtggcggtgg | |
| | ctgcgttggcggcctgcccatggggaaacccatgggacgctctaa | |
| | tacagacatggtgcgaagagtctattgagctagttggtagtcctc | |
| | cggcccctgaatgcggctaatcctaactgcggagcacacaccctc | |
| | ACTCACTATTTGTTTTCGCGCCCAGTTGCAAAAAGTGTCGgaggg | |
| | cagtgtgtcgtaacgggcaactctgcagcggaaccgactactttg | |
| | ggtgtcgtgtttcattttattcctatactggctgcttatggtgac | |
| | aattgagagatcgttaccatatagctattggattggccatccggt | |
| | gactaatagagctattatatatccctttgttgggtttataccact | |
| | tagcttgaaagaggttaaaacattacaattcattgttaagttgaa | |
| | tacagcaaa | |
| synIRES-RC11 | ttaaaacagcctgtgggttgatcccacccacaggcccattgggcg | 33154 |
| | ctagcactctggtatcacggtacctttgtgcgcctgttttatacc | |
| | ccctcccccaactgtaacttagaagtaacacacaccgatcaacag | |
| | tcagcgtggcacaccagccacgttttgatcaagcacttctgttac | |
| | cccggactgagtatcaatagactgctcacgcggttgaaggagaaa | |
| | gcgttcgttatccggccaactacttcgaaaaacctagtaacaccg | |
| | tggaagttgcagagtgtttcgctcagcactaccccagtgtagatc | |
| | aggtcgatgagtcaccgcattccccacgggcgaccgtggcggtgg | |
| | ctgcgttggcggcctgcccatggggaaacccatgggacgctctaa | |
| | tacagacatggtgcgaagagtctattgagctagttggtagtcctc | |
| | cggcccctgaatgcggctaatcctaactgcggagcacacaccctc | |
| | aagccagagggcagtgtgtcgtaacgggcaactctgcagcggaac | |
| | cgactactttgggtgtcgtgtttcattttACTCACTATTTGTTTT | |
| | CGCGCCCAGTTGCAAAAAGTGTCGattcctatactggctgcttat | |
| | ggtgacaattgagagatcgttaccatatagctattggattggcca | |
| | tccggtgactaatagagctattatatatccctttgttgggtttat | |
| | accacttagcttgaaagaggttaaaacattacaattcattgttaa | |
| | gttgaatacagcaaa | |
| synIRES-RC12 | ttaaaacagcggatgggtaccccaccatccgacccactgggtgta | 33155 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggCACTCACTATTTG | |
| | TTTTCGCGCCCAGTTGCAAAAAGTGTCGGCCagtttggtcgatga | |
| | ggctaggaattccccacgggtgaccgtgtcctagcctgcgtggcg | |
| | gccaacccagcttatgctgggacgcctttttatagacatggtgtg | |
| | aagactcgcatgtgcttggttgtgattcctccggcccctgaatgc | |
| | ggctaaccttaaccctggagccttgtgtcacaaaccagtgatgat | |
| | aaggtcgtaatgagcaattccgggacgggaccgactactttgggt | |
| | gtccgtgtttcttatttttcttattattgtcttatggtcacagca | |
| | tatatataacatatactgtgatc | |
| synIRES-RC13 | ttaaaacagcggatgggtaccccaccatccgacccactgggtgta | 33156 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggCGACTCACTATTT | |
| | GTTTTCGCGCCCAGTTGCAAAAAGTGTCGCGCCagtttggtcgat | |
| | gaggctaggaattccccacgggtgaccgtgtcctagcctgcgtgg | |
| | cggccaacccagcttatgctgggacgcctttttatagacatggtg | |
| | tgaagactcgcatgtgcttggttgtgattcctccggcccctgaat | |
| | gcggctaaccttaaccctggagccttgtgtcacaaaccagtgatg | |
| | ataaggtcgtaatgagcaattccgggacgggaccgactactttgg | |
| | gtgtccgtgtttcttatttttcttattattgtcttatggtcacag | |
| | catatatataacatatactgtgatc | |
| synIRES-RC14 | ttaaaacagcggatgggtaccccaccatccgacccactgggtgta | 33157 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggCGGACTCACTATT | |
| | TGTTTTCGCGCCCAGTTGCAAAAAGTGTCGCCGCCagtttggtcg | |
| | atgaggctaggaattccccacgggtgaccgtgtcctagcctgcgt | |
| | ggcggccaacccagcttatgctgggacgcctttttatagacatgg | |
| | tgtgaagactcgcatgtgcttggttgtgattcctccggcccctga | |
| | atgcggctaaccttaaccctggagccttgtgtcacaaaccagtga | |
| | tgataaggtcgtaatgagcaattccgggacgggaccgactacttt | |
| | gggtgtccgtgtttcttatttttcttattattgtcttatggtcac | |
| | agcatatatataacatatactgtgatc | |
| synIRES-RC15 | ttaaaacagcggatgggtaccccaccatccgacccactgggtgta | 33158 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggtggaACTCACTAT | |
| | TTGTTTTCGCGCCCAGTTGCAAAAAGTGTCGtccactagtttggt | |
| | cgatgaggctaggaattccccacgggtgaccgtgtcctagcctgc | |
| | gtggggccaacccagcttatgctgggacgcctttttatagacatg | |
| | gtgtgaagactcgcatgtgcttggttgtgattcctccggcccctg | |
| | aatgcggctaaccttaaccctggagccttgtgtcacaaaccagtg | |
| | atgataaggtcgtaatgagcaattccgggacgggaccgactactt | |
| | tgggtgtccgtgtttcttatttttcttattattgtcttatggtca | |
| | cagcatatatataacatatactgtgatc | |
| synIRES-RC16 | ttaaaacagcggatgggtaccccaccatccgacccactgggtgta | 33159 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggtggaGACTCACTA | |
| | TTTGTTTTCGCGCCCAGTTGCAAAAAGTGTCGCtccactagtttg | |
| | gtcgatgaggctaggaattccccacgggtgaccgtgtcctagcct | |
| | gcgtggcggccaacccagcttatgctgggacgcctttttatagac | |
| | atggtgtgaagactcgcatgtgcttggttgtgattcctccggccc | |
| | ctgaatgcggctaaccttaaccctggagccttgtgtcacaaacca | |
| | gtgatgataaggtcgtaatgagcaattccgggacgggaccgacta | |
| | ctttgggtgtccgtgtttcttatttttcttattattgtcttatgg | |
| | tcacagcatatatataacatatactgtgatc | |
| synIRES-RC17 | ttaaaacagcggatgggtaccccaccatccgacccactgggtgta | 33160 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggtggaGGACTCACT | |
| | ATTTGTTTTCGCGCCCAGTTGCAAAAAGTGTCGCCtccactagtt | |
| | tggtcgatgaggctaggaattccccacgggtgaccgtgtcctagc | |
| | ctgcgtggcggccaacccagcttatgctgggacgcctttttatag | |
| | acatggtgtgaagactcgcatgtgcttggttgtgattcctccggc | |
| | ccctgaatgcggctaaccttaaccctggagccttgtgtcacaaac | |
| | cagtgatgataaggtcgtaatgagcaattccgggacgggaccgac | |
| | tactttgggtgtccgtgtttcttatttttcttattattgtcttat | |
| | ggtcacagcatatatataacatatactgtgatc | |
| synIRES-RC18 | ttaaaacagcggatgggtaccccaccatccgacccactgggtgta | 33161 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggtggatttcccctc | |
| | cactagtttggtcgatgaggctaggaACTCACTATTTGTTTTCGC | |
| | GCCCAGTTGCAAAAAGTGTCGtcctagcctgcgtggcggccaacc | |
| | cagcttatgctgggacgcctttttatagacatggtgtgaagactc | |
| | gcatgtgcttggttgtgattcctccggcccctgaatgcggctaac | |
| | cttaaccctggagccttgtgtcacaaaccagtgatgataaggtcg | |
| | taatgagcaattccgggacgggaccgactactttgggtgtccgtg | |
| | tttcttatttttcttattattgtcttatggtcacagcatatatat | |
| | aacatatactgtgatc | |
| synIRES-RC19 | ttaaaacagcggatgggtaccccaccatccgacccactgggtgta | 33162 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggtggatttcccctc | |
| | cactagtttggtcgatgaggctaggaattccccacggACTCACTA | |
| | TTTGTTTTCGCGCCCAGTTGCAAAAAGTGTCGccgtgtcctagcc | |
| | tgcgtggcggccaacccagcttatgctgggacgcctttttataga | |
| | catggtgtgaagactcgcatgtgcttggttgtgattcctccggcc | |
| | cctgaatgcggctaaccttaaccctggagccttgtgtcacaaacc | |
| | agtgatgataaggtcgtaatgagcaattccgggacgggaccgact | |
| | actttgggtgtccgtgtttcttatttttcttattattgtcttatg | |
| | gtcacagcatatatataacatatactgtgatc | |
| synIRES-RC20 | ttaaaacagcggatgggtaccccaccatccgacccactgggtgta | 33163 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggtggatttcccctc | |
| | cactagtttggtcgatgaggctaggaattccccacgggtgaccgt | |
| | gtcctagcctgcgtggcggccaacccACTCACTATTTGTTTTCGC | |
| | GCCCAGTTGCAAAAAGTGTCGgggacgcctttttatagacatggt | |
| | gtgaagactcgcatgtgcttggttgtgattcctccggcccctgaa | |
| | tgcggctaaccttaaccctggagccttgtgtcacaaaccagtgat | |
| | gataaggtcgtaatgagcaattccgggacgggaccgactactttg | |
| | ggtgtccgtgtttcttatttttcttattattgtcttatggtcaca | |
| | gcatatatataacatatactgtgatc | |
| synIRES-RC21 | ttaaaacagcggatgggtaccccaccatccgacccactgggtgta | 33164 |
| | gtactcctggtacttcgtacctttgtacgcctgttcttcccattg | |
| | tacccttcctgaacttccaacccaagtaacgttagaagctcaaca | |
| | tttagtacaacaggaagcaccacatccagtggtgtttagtacaag | |
| | cacttctgtttccccggagcgaggtataggctgtacccactgcca | |
| | aaaacctttaaccgttatccgccaaccaactacgtaaaagctagt | |
| | agtattatgtttttaactaggcgttcgatcaggtggatttcccct | |
| | ccactagtttggtcgatgaggctaggaattccccacgggtgaccg | |
| | tgtcctagcctgcgtggcggccaacccGACTCACTATTTGTTTTC | |
| | GCGCCCAGTTGCAAAAAGTGTCGCgggacgcctttttatagacat | |
| | ggtgtgaagactcgcatgtgcttggttgtgattcctccggcccct | |
| | gaatgcggctaaccttaaccctggagccttgtgtcacaaaccagt | |
| | gatgataaggtcgtaatgagcaattccgggacgggaccgactact | |
| | ttgggtgtccgtgtttcttatttttcttattattgtcttatggtc | |
| | acagcatatatataacatatactgtgatc | |
| synIRES-RC22 | ttaaaacagcggatgggtaccccaccatccgacccactgggtgta | 33165 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggtggatttcccctc | |
| | cactagtttggtcgatgaggctaggaattccccacgggtgaccgt | |
| | gtcctagcctgcgtggcggccaacccGGACTCACTATTTGTTTTC | |
| | GCGCCCAGTTGCAAAAAGTGTCGCCgggacgcctttttatagaca | |
| | tggtgtgaagactcgcatgtgcttggttgtgattcctccggcccc | |
| | tgaatgcggctaaccttaaccctggagccttgtgtcacaaaccag | |
| | tgatgataaggtcgtaatgagcaattccgggacgggaccgactac | |
| | tttgggtgtccgtgtttcttatttttcttattattgtcttatggt | |
| | cacagcatatatataacatatactgtgatc | |
| synIRES-RC23 | ttaaaacagcggatgggtaccccaccatccgacccactgggtgta | 33166 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggtggatttcccctc | |
| | cactagtttggtcgatgaggctaggaattccccacgggtgaccgt | |
| | gtcctagcctgcgtggcggccaacccagcACTCACTATTTGTTTT | |
| | CGCGCCCAGTTGCAAAAAGTGTCGgctgggacgcctttttataga | |
| | catggtgtgaagactcgcatgtgcttggttgtgattcctccggcc | |
| | cctgaatgcggctaaccttaaccctggagccttgtgtcacaaacc | |
| | agtgatgataaggtcgtaatgagcaattccgggacgggaccgact | |
| | actttgggtgtccgtgtttcttatttttcttattattgtcttatg | |
| | gtcacagcatatatataacatatactgtgatc | |
| synIRES-RC24 | ttaaaacagcggatgggtaccccaccatccgacccactgggtgta | 33167 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggtggatttcccctc | |
| | cactagtttggtcgatgaggctaggaattccccacgggtgaccgt | |
| | gtcctagcctgcgtggcggccaacccagcCACTCACTATTTGTTT | |
| | TCGCGCCCAGTTGCAAAAAGTGTCGGgctgggacgcctttttata | |
| | gacatggtgtgaagactcgcatgtgcttggttgtgattcctccgg | |
| | cccctgaatgcggctaaccttaaccctggagccttgtgtcacaaa | |
| | ccagtgatgataaggtcgtaatgagcaattccgggacgggaccga | |
| | ctactttgggtgtccgtgtttcttatttttcttattattgtctta | |
| | tggtcacagcatatatataacatatactgtgatc | |
| synIRES-RC25 | ttaaaacagcggatgggtaccccaccatccgacccactgggtgta | 33168 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggtggatttcccctc | |
| | cactagtttggtcgatgaggctaggaattccccacgggtgaccgt | |
| | gtcctagcctgcgtggcggccaacccagcCCACTCACTATTTGTT | |
| | TTCGCGCCCAGTTGCAAAAAGTGTCGGGgctgggacgccttttta | |
| | tagacatggtgtgaagactcgcatgtgcttggttgtgattcctcc | |
| | ggcccctgaatgcggctaaccttaaccctggagccttgtgtcaca | |
| | aaccagtgatgataaggtcgtaatgagcaattccgggacgggacc | |
| | gactactttgggtgtccgtgtttcttatttttcttattattgtct | |
| | tatggtcacagcatatatataacatatactgtgatc | |
| synIRES-RC26 | ttaaaacagcggatgggtaccccaccatccgacccactgggtgta | 33169 |
| | gtactctggtacttcgtacctttgtacgcctgttcttcccattgt | |
| | acccttcctgaacttccaacccaagtaacgttagaagctcaacat | |
| | ttagtacaacaggaagcaccacatccagtggtgtttagtacaagc | |
| | acttctgtttccccggagcgaggtataggctgtacccactgccaa | |
| | aaacctttaaccgttatccgccaaccaactacgtaaaagctagta | |
| | gtattatgtttttaactaggcgttcgatcaggtggaACTCACTAT | |
| | TTGTTTTCGCGCCCAGTTGCAAAAAGTGTCGtccactagtttggt | |
| | cgatgaggctaggaattccccacgggtgaccgtgtcctagcctgc | |
| | gtggcggccaacccagcACTCACTATTTGTTTTCGCGCCCAGTTG | |
| | CAAAAAGTGTCGgctgggacgcctttttatagacatggtgtgaag | |
| | actcgcatgtgcttggttgtgattcctccggcccctgaatgcggc | |
| | taaccttaaccctggagccttgtgtcacaaaccagtgatgataag | |
| | gtcgtaatgagcaattccgggacgggaccgactactttgggtgtc | |
| | cgtgtttcttatttttcttattattgtcttatggtcacagcatat | |
| | atataacatatactgtgatc | |

1. A composition, comprising: a complex of a charge-altering releasable transporter and a circular RNA molecule comprising a protein-coding sequence.

2. (canceled)

3. The composition of claim 1, wherein the charge-altering releasable transporter comprises a mixture of oleyl (O) and nonenyl-substituted (N) carbonate monomers followed by a block of α-amino ester monomers (A).

4. (canceled)

5. (canceled)

6. The composition of claim 1, wherein the circular RNA molecule comprises an internal ribosome entry site (IRES) sequence operably linked to the protein-coding sequence, a 5′ UTR, and a 3′ UTR;
wherein the IRES sequence is a viral sequence; and
wherein the protein-coding sequence encodes a non-viral protein.

7. (canceled)

8. (canceled)

9. The composition of claim 6, wherein the IRES comprises a locked nucleic acid (LNA) against a non-base-paired linker region between domains of said IRES.

10. (canceled)

11. The composition of claim 6, wherein the IRES is a Type 1 IRES or a synthetic IRES.

12.-18. (canceled)

19. The composition of claim 6, wherein the synthetic IRES sequence comprises one or more aptamers.

20.-22. (canceled)

23. The composition of claim 19, wherein the aptamer is positioned within the secondary structure of the IRES so that itis spatially proximal to a portion of the IRES responsible for translation initiation.

24. (canceled)

25. The composition of claim 19, wherein the aptamer is an eIF4G-binding aptamer encoded by the sequence of SEQ ID NO: 33143.

26. (canceled)

27. The composition of claim 1, wherein the circular RNA molecule comprises at least one 2-thiouridine (2ThioU) or at least one 2′-O-methylcitidine (2OMeC).

28. (canceled)

29. A nucleic acid that encodes the circular RNA molecule of claim 6.

30. A host cell comprising the nucleic acid of claim 29.

31. A method of producing a protein, the method comprising contacting a cell or cell-free extract with the composition of claim 1 under conditions whereby the protein-coding nucleic acid sequence of the circular RNA is translated and the protein is produced.

32. The method of claim 31, wherein the cell is an immune cell.

33. The method of claim 32, wherein the immune cell is a macrophage, a dendritic cell or a monocyte.

34. A method of delivering a protein to a subject, comprising: administering the composition of claim 1 to a subject.

35.-38. (canceled)

39. A method for expressing a protein in a subject comprising administering to the subject the composition of claim 1, wherein the circular RNA molecule comprises a protein coding sequence that is operably linked to an internal ribosome entry site (IRES) sequence.

40. (canceled)

41. A method of treating cancer in a subject, wherein the method comprises
administering a composition, comprising: a complex of a charge-altering releasable transporter and a circular RNA molecule comprising a sequence that encodes a tumor antigen and an IRES operably linked to the sequence encoding the tumor antigen.

42.-43. (canceled)

44. A method of inducing an immune response in a subject in need comprising administering to the subject a composition comprising a complex of a charge-altering releasable transporter and a circular RNA molecule, wherein the charge-altering releasable transporter and the circRNA molecule are complexed at a 1:10 charge ratio, and wherein the circRNA is directed to immune cells.

45. The method of claim 44, wherein the circular RNA molecule comprises a sequence encoding a therapeutic protein and an IRES linked to the sequence encoding the therapeutic protein.

46.-50. (canceled)

51. The method of claim 44, wherein the immune cells are macrophages, dendritic cells and/or monocytes.
